(19) United States
(12) Patent Application Publication
(10) Pub. No.: US 2025/0032635 A1
(43) Pub. Date: Jan. 30, 2025

(54) LIGAND-POLAR DRUG CONJUGATES

(71) Applicant: Tripartite Therapeutics Inc., Taipei City (TW)

(72) Inventors: Young-Sun Lin, Taipei City (TW); Er-Yi Huang, Taipei City (TW); Hsueh-Yi Lu, Taipei City (TW)

(21) Appl. No.: 18/205,841

(22) Filed: Jun. 5, 2023

(51) Int. Cl. A61K 47/68; A61K 47/64; A61K 47/65; A61P 35/00

(52) U.S. Cl. CPC A61K 47/6855; A61K 47/643; A61K 47/65; A61K 47/68031; A61K 47/68033; A61K 47/68035; A61K 47/6849; A61P 35/00

(57) ABSTRACT

Disclosed are compounds of formula I:

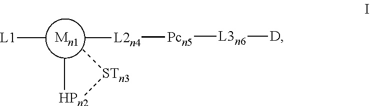

in which all the variables are defined. Also disclosed are ligand-PD conjugates formed of a compound of formula I, a method of treating cancer using such a conjugate, and a pharmaceutical composition containing the conjugate.

### BACKGROUND

Antibody-Drug Conjugates (ADCs) provide promising antibody-based therapeutics that harness both the antibody specificity and small molecule tumor cytotoxicity. They deliver highly potent therapeutic agents specifically to the target cells and thus minimize side effects commonly seen in cancer treatment.

Typical therapeutic agents are hydrophobic cytotoxic drugs such as maytansinoids, auristatins, calicheamicins, duocarmycins, and pyrrolobenzodiazepines (PBDs). In an ADC, a therapeutic agent is linked to an antibody through a chemically or enzymatically cleavable linker, or a non-cleavable linker (see Su et al., 2021). One of the major challenges in ADC development is the application of a suitable linker that connects a therapeutic agent (i.e., a payload) to a ligand, stabilizes the ADC in circulation, and also releases the payload to a specific site of a cancer cell.

An ADC can include an enzymatically cleavable linker (“cleavable linker”) or a non-cleavable linker.

The cleavable linker has one or more cutting sites, which is served to release the payload by a lysosomal enzyme such as Cathepsin B, Glycosidases, Phosphatases, and Sulfatases (see Jiang et al., 2015). Many lysosomal enzymes appear to, in addition to lysosome, be present in other locations, for example, in circulation (Zhloba and Dunaevskiǐ, 1996), in the extracellular space and on the cell surface (Wu et al., 2010), as well as in the endosomal compartment (Guha et al., 2008). Obviously, ADCs with a cleavable linker are subjected to release their payloads prematurely before being proceeded to lysosome. Indeed, premature release of payloads has been confirmed in preclinical and clinical studies regarding ADCs with a cleavable linker (Diamantis and Banerji, 2016; Bargh et al., 2019; Su et al., 2021; and Sheyi et al., 2022).

On the other hand, a non-cleavable linker contains no cutting site. An ADC containing a non-cleavable linker relies on the slow degradation of the antibody ligand in lysosome to release an ionic active metabolite, i.e., a covalently linked tripartite entity composed of a payload, the linker, and the antibody-derived amino acid residue (see Oroudjev et al., 2010). In addition, being ionic and thus less capable of crossing cell membrane, the active metabolites liberated from killed tumor cells are less toxic to normal cell populations in the tumor and elsewhere, compared to their hydrophobic counterparts.

ADCs with a non-cleavable linker are prepared by conjugating the payload via a linker to the antibody. As such, the available conjugation sites are limited to the side chain of exposed cysteine, lysine or glutamine residue(s) of antibody (Schneider et al., 2020). Further, due to the steric hindrance posed by the antibody ligand and/or the transglutaminase enzyme used for the conjugation, the linker selection suffers further restrictions (Leal et al., 2015). Other disadvantages of conventional ADCs with a non-cleavable linker are: (a) their active metabolites are confined to cysteine, lysine or glutamine adducts and could be released only after the complete degradation of antibody in lysosome (Mckertish and Kayser, 2021), a rather slow process in a very detrimental environment where the active metabolites will be more or less damaged; (b) the DAR (drug/antibody ratio) value is low (about 2-4) (Rathi et al., 2021); and (c) half-life is short (Lu et al., 2014).

Obviously, to better exploit the power of ADCs with a non-cleavable linker for cancer treatment, there is great room for improvement.

Tumor vasculature is immature and leaky which leads to EPR (enhanced permeability and retention) (McDonald and Baluk. 2002; Nagy et al., 2009) and TIFP (tumor interstitial fluid pressure) (Libutti et al., 2018), characteristics of tumor microenvironment. Many tumors resist the interstitial penetration of anticancer agents due to TIFP. Such resistance may help explain why drugs that eradicate tumor cells in a laboratory environment often fail to eliminate malignancies in the body.

Another problem in cancer chemotherapy is multi-drug resistance (MDR), the development of a simultaneous resistance to a variety of structurally irrelevant cytotoxic drugs (Catalano et al., 2022). MDR mainly arises from the increased expression of membrane ABC transporters that mediate unidirectional energy-dependent efflux of chemotherapeutic agents. As a result, ABC transporters interfere with the bioavailability, pharmacokinetics, and pharmacodynamics of a chemotherapeutic agent. Among ABC transporters, the overexpression of MDR1 protein (also known as P-glycoprotein or ABCB1) is mainly responsible for MDR observed in many cancer cells. MDR1 has been shown to transport a great variety of compounds sharing little structural similarities. In fact, many cancer drugs known to be MDR1 substrates are hydrophobic, such as mitomycin C, vinblastine, vincristine, vinorebline, vindesine, etoposide, paclitaxel, doxorubicin, daunorubicin, epirubicin, idarubicin, docetaxel, irinotecan, SN-38, topotecan, and methotrexate (Zhou et al., 2008; Waghray and Zhang, 2018; Gala et al., 2020), due to the cavity of MDR1 involved in the efflux of chemotherapeutic agents is hydrophobic and thus prefers to accommodate hydrophobic compounds.

There is a need to develop a conjugate that delivers a therapeutic agent efficiently to a desired site within a cancerous cell and releases it rapidly to kill the cell.

### SUMMARY OF THE INVENTION

The present invention provides ligand-polar drug conjugates (LPDCs) having great pharmacokinetics, bioavailability, and pharmacodynamics to treat cancers.

In one aspect, the invention relates to compounds of formula I:

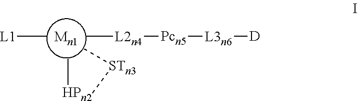

As shown in formula I, the compound comprises a ligand linker L1, an Mₐ₁ linker complex, a drug linker L2, a first polar drug (PD) moiety PD1 represented by Pcₙ₅-L3ₙ₆-D, optionally one to five hydrophilic polymer moieties HP, and optionally one to five secondary targeting moieties ST.

In formula I above, n1 is an integer from 1 to 15; n2 is an integer from 0 to 5; n3 is an integer from 0 to 5; n4 is 1 or 2; n5 is 0 or 1; n6 is 0 or 1; one of ---- is a covalent bond and the other ---- is deleted or both are deleted when n2 and n3 are 0; each of L1, L2, and HP, when present, is bonded to the Mai linker complex via a linker bond; ST, when present, is bonded to HP or the Mₐ₁ linker complex via a linker bond; L2 is bonded to PD via an amide bond or a glycoside bond; L3, when present, is bonded to D via a stable bond; Pc, when present, is bonded to L3 or D via a stable bond; each of the monomer M within the Mₙ₁ linker complex is bonded to its adjacent monomer M via a stable bond; D is a drug moiety; M in each occurrence is independently a multifunctional moiety; L1 is a bifunctional crosslinker containing a coupling moiety capable of reacting with a ligand via sulfhydryl, amino, glutamine, or formyl contained in the ligand; L2 is a bifunctional crosslinker; L3 when present is a bifunctional crosslinker; HP in each occurrence when present, independently, is a hydrophilic polymer moiety; Pc when present contains a polar group; ST in each occurrence when present, independently, is a secondary targeting moiety; a linker bond is an amide bond, a glycoside bond, an ester bond, a disulfide bond, a C—S bond, a C—O bond, a C—N bond, a carbonate moiety (—O—C(O)—O—), a carbamate moiety (—O—C(O)—NH—), a urea moiety (—NH—C(O)—NH—), or a triazole moiety

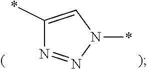 [SMILES: *c1cn(*)nn1]

and a stable bond is a stable amide bond, a stable glycoside bond, a stable ester bond, a stable disulfide bond, a C—S bond, a C—O bond, a C—N bond, a urea moiety (—NH—C(O)—NH—), or a triazole moiety

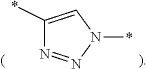 [SMILES: *c1cn(*)nn1]

Preferably, L1 contains amino (NH₂), iodo (I), bromo (Br), a maleimide moiety, an N-hydroxysuccinimide (NHS) moiety, or an aminooxy moiety; M in each occurrence is a moiety derived from a natural amino acid, a non-natural amino acid, a monosaccharide, a bifunctional crosslinker, a polyamine having at least one secondary or tertiary amine group, 3,6-bis-(4-aminobutyl)piperazine-2,5-dione, a cyclic peptide, or a diamino dicarboxylic acid; Pc is a moiety derived from a monosaccharide, a polyamine, a hydroxycarboxylic acid, a linear or cyclic dipeptide, a diamino dicarboxylic acid, a natural amino acid, or a non-natural amino acid; and ST is a moiety derived from glucose, folic acid, iRGD peptide, a cancer-specific peptide ligand (AGM-330), a human fibronectin extra-domain B (EDB)-specific aptide (“APTEDB”), a centyrin, an F3 peptide, a DVN peptide, pasireotide, afamelanotide, etelcalcetide, somatostatin, a PD-1/PD-L1 interaction inhibitor (“BMS-1166”), a cyclic peptide, a single-chain antibody, or an aptamer.

Any compound described above optionally contains a second polar drug moiety PD2 represented by Pc′_{n5′}-(L₃′)ₙ₅-D′, which is bonded to the Mₙ₁ linker complex via an amide bond or a glycoside bond; n5′ is 0 or 1; n6′ is 0 or 1; D′ is a drug moiety; L3′ when present is a bifunctional crosslinker, and Pc′ when present contains a polar group.

The Mₙ₁ linker complex contains one or more (i.e., n1) M, each of which is a monomeric unit. M in each occurrence, independently, is a moiety derived from a diamino dicarboxylic acid, a monosaccharide, 3,6-bis-(4-aminobutyl)piperazine-2,5-dione, a cyclic peptide, an amino acid, a dicarboxylic acid, a mercapto carboxylic acid, or an aminothiol.

Preferably, the Mₙ₁ linker complex contains a monomeric unit derived from a diamino dicarboxylic acid, 3,6-bis-(4-aminobutyl)piperazine-2,5-dione, or a cyclic peptide. In these preferred embodiments, the compound optionally contains, between L2 and PD, a releasable moiety having a self-immolative spacer, an enzymatically cleavable amino acid, an enzymatically cleavable peptide of 2-8 amino acids, a combination of the self-immolative spacer and the enzymatically cleavable amino acid, or a combination of the self-immolative spacer and the enzymatically cleavable peptide.

Typically, the Mₙ₁ linker complex contains a moiety selected from the group consisting of:

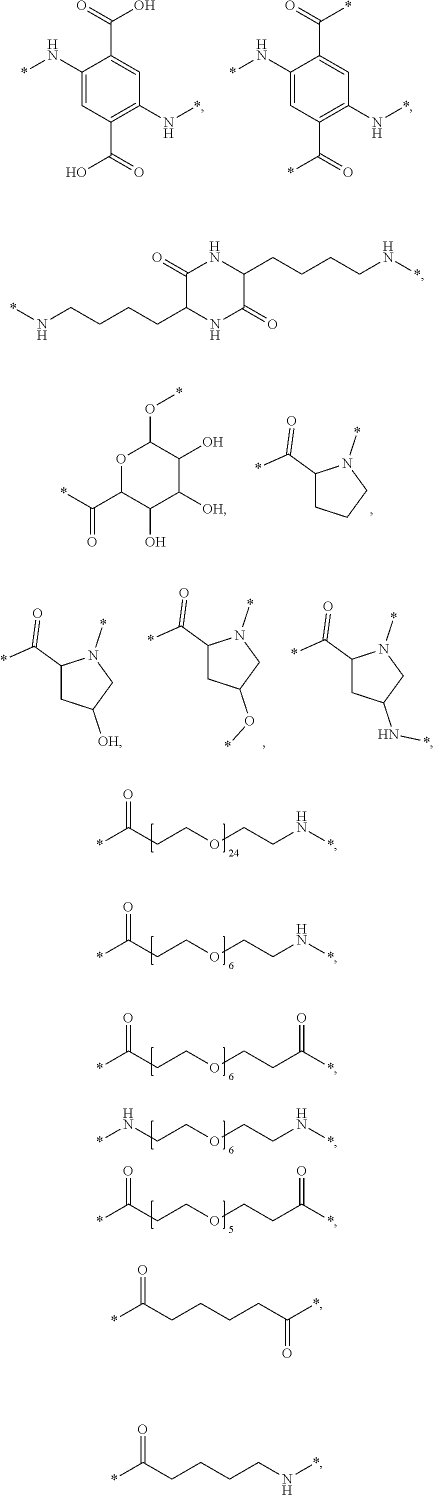

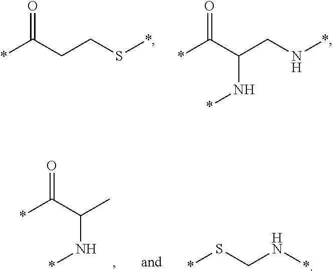

Preferably, the Mₙ₁ linker complex contains a moiety selected from the group consisting of:

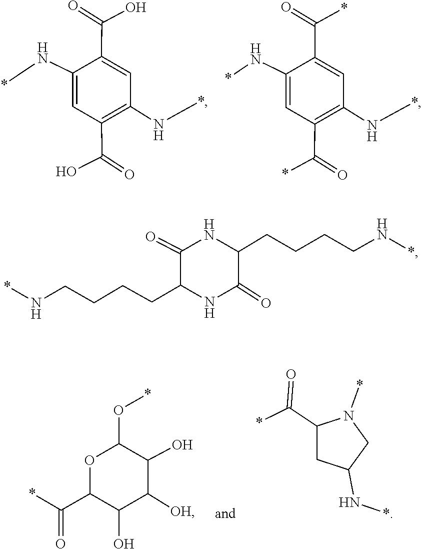

More preferably, the Mₙ₁ linker complex contains:

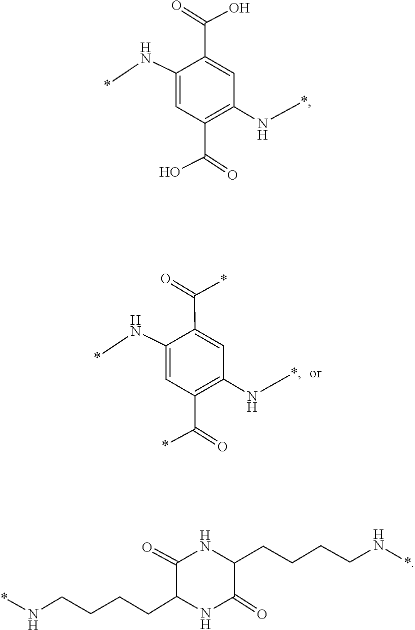

Subsets of the compounds of formula I can have one or any combinations of the following features:
(i) L1 is

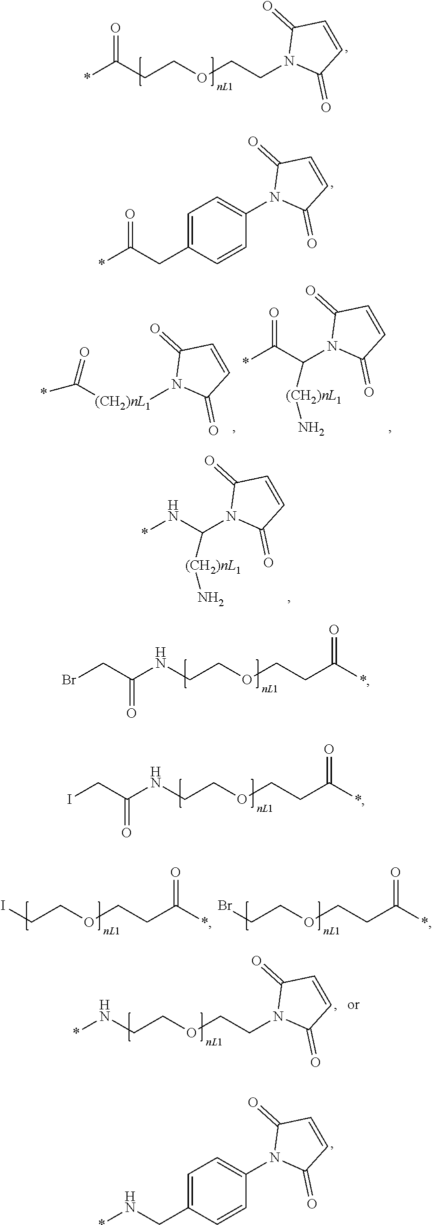

in which nL1 is an integer from 0 to 10;
(ii) L2 is

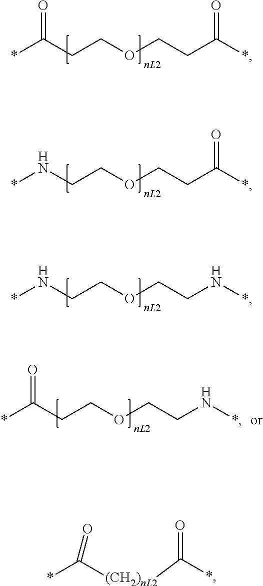

in which nL2 is an integer from 0 to 10;
(iii) Pc is a moiety derived from an amino acid, a linear or cyclic dipeptide, a monosaccharide, a bifunctional polyethylene glycol, or an aminophenol, such as:

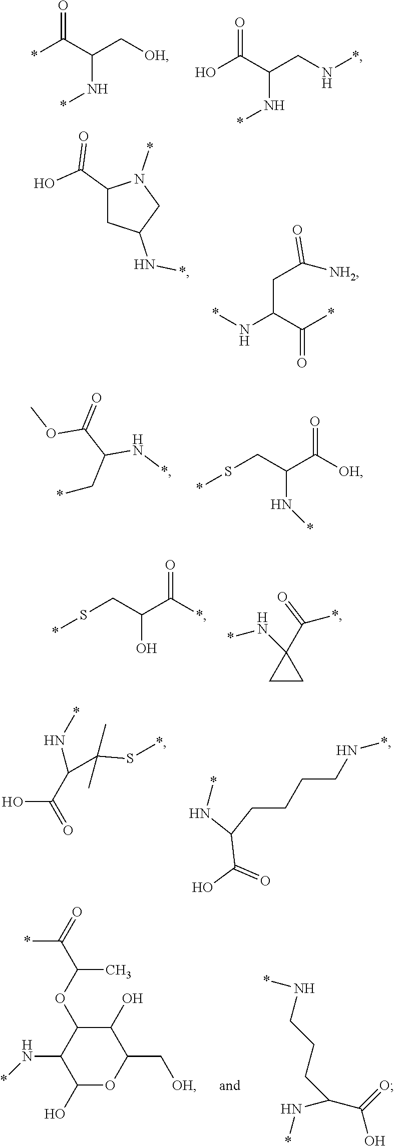

(iv) L3 is

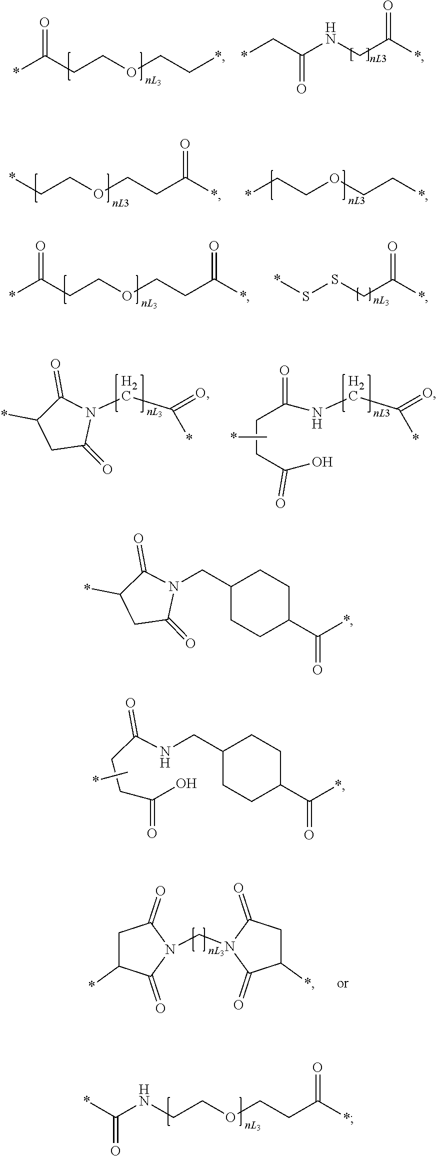

and nL3 is 0 to 10;
(v) HP is

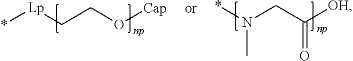

in which np is an integer from 5 to 50; Cap is (i) a reactive group capable of reacting with a ST molecule to bond the ST moiety to the HP moiety or (ii) a terminal group selected from the group consisting of C₁-C₁₀ alkyl, C₂-C₁₀ alkyl-CO₂H, C₂-C₁₀alkyl-OH, C₂-C₁₀alkyl-NH₂, C₂-C₁₀alkyl-NH(C₁-C₃alkyl), and C₂-C₁₀ alkyl-N(C₁-C₃ alkyl)₂; and Lp is a linker moiety. Exemplary HP moieties include

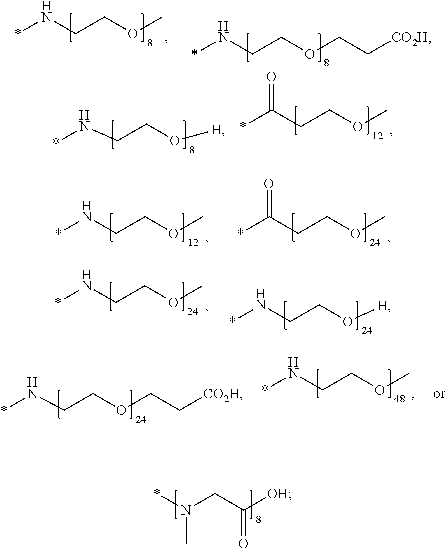

(vi) D is a drug moiety derived from mertansine (DM1), ravtansine (DM4), N-methyl-L-Ala-maytansinol, monomethyl auristatin E, 7-ethyl-10-hydroxycamptothecin (SN38), or TLR7/8 agonist Ag; and
(vii) the secondary targeting moiety is derived from folic acid, glucose, or acetyl-RHGAMVYLK.

Shown below are the structures of the 36 exemplary compounds of the present invention, i.e., Compounds 1-36.

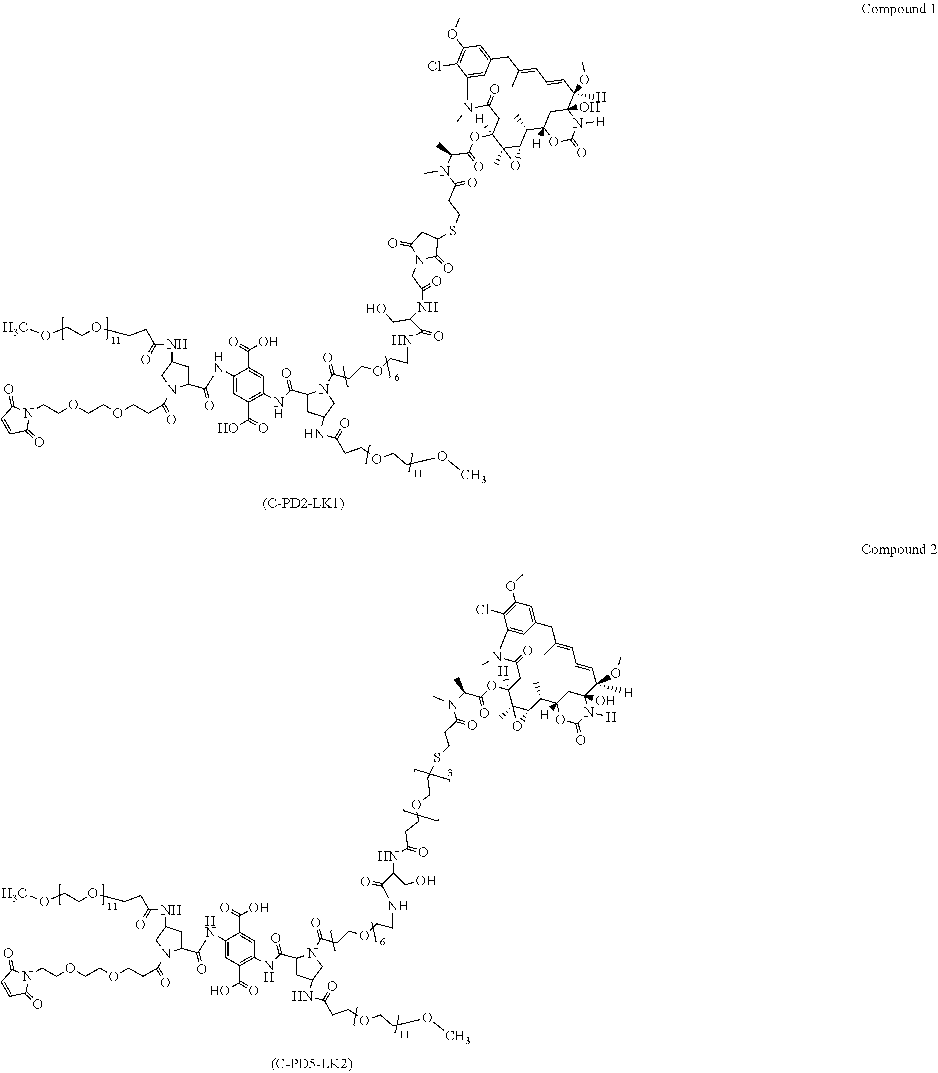

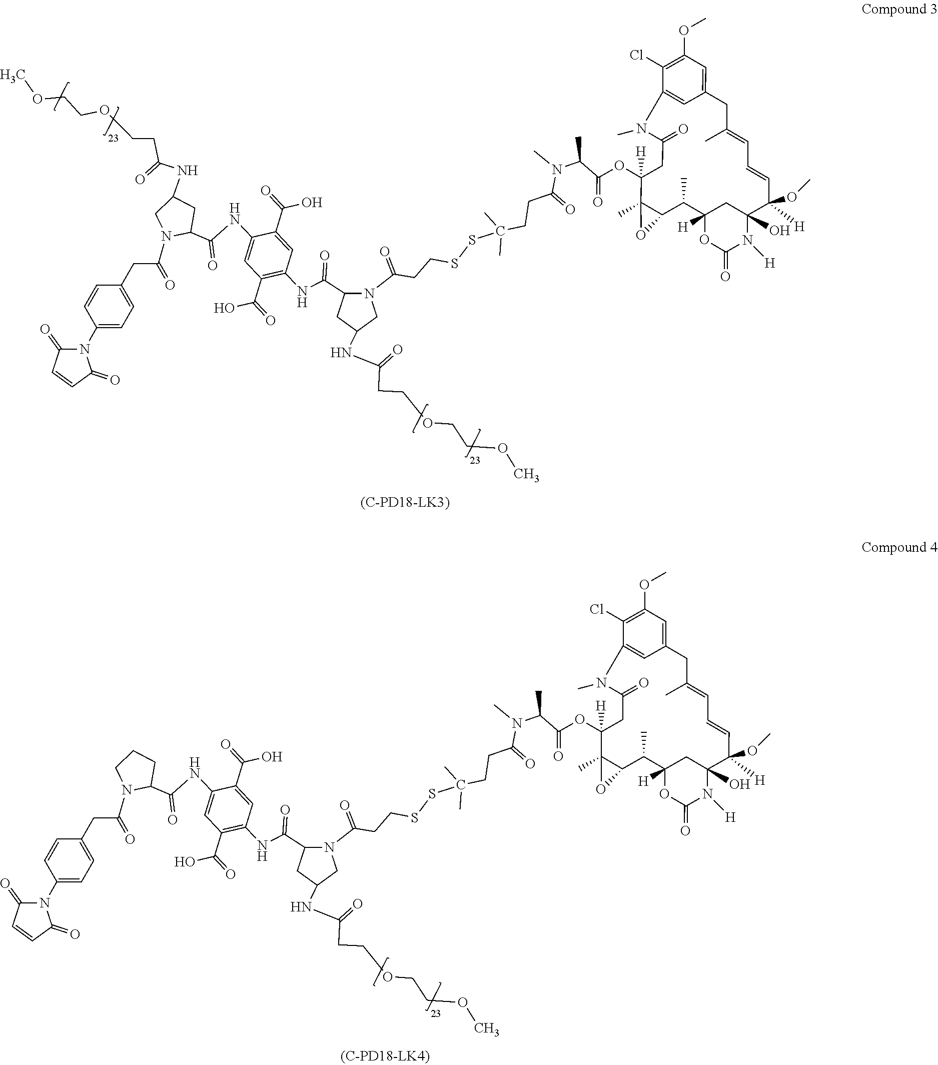

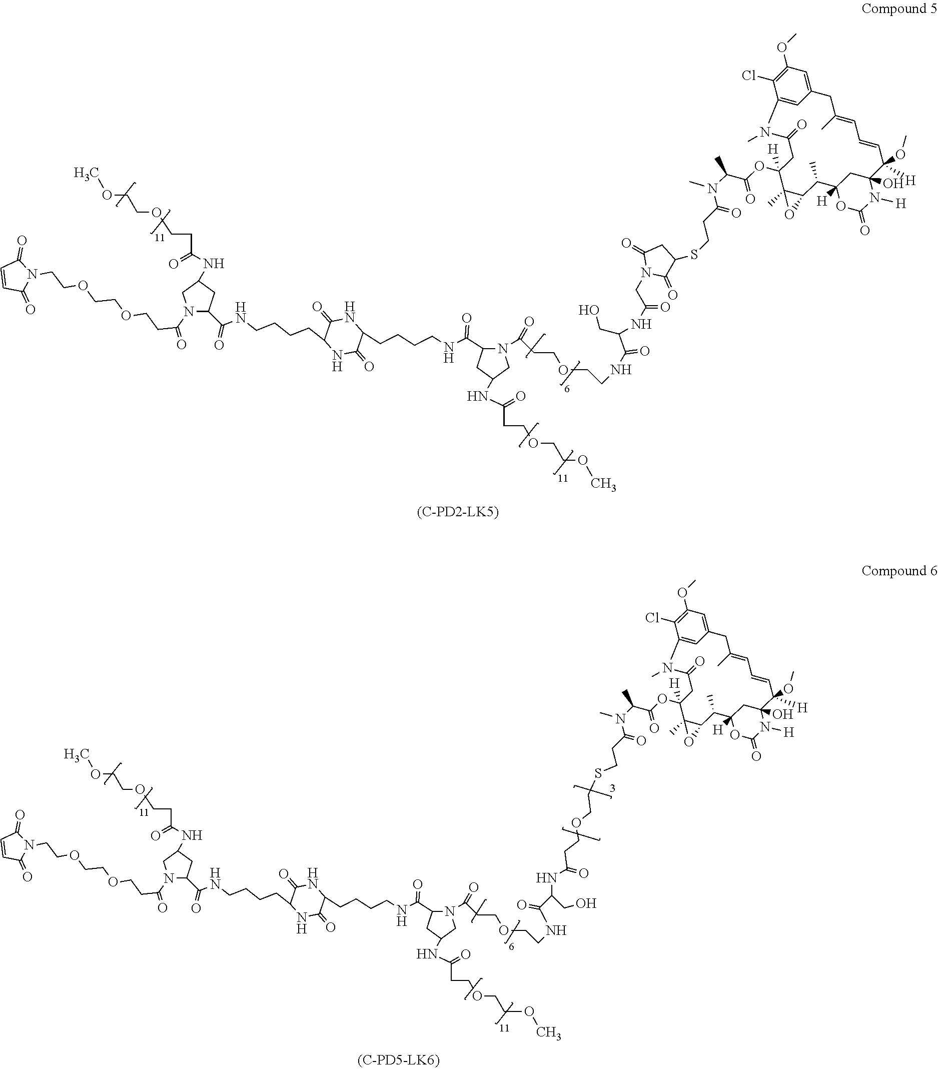

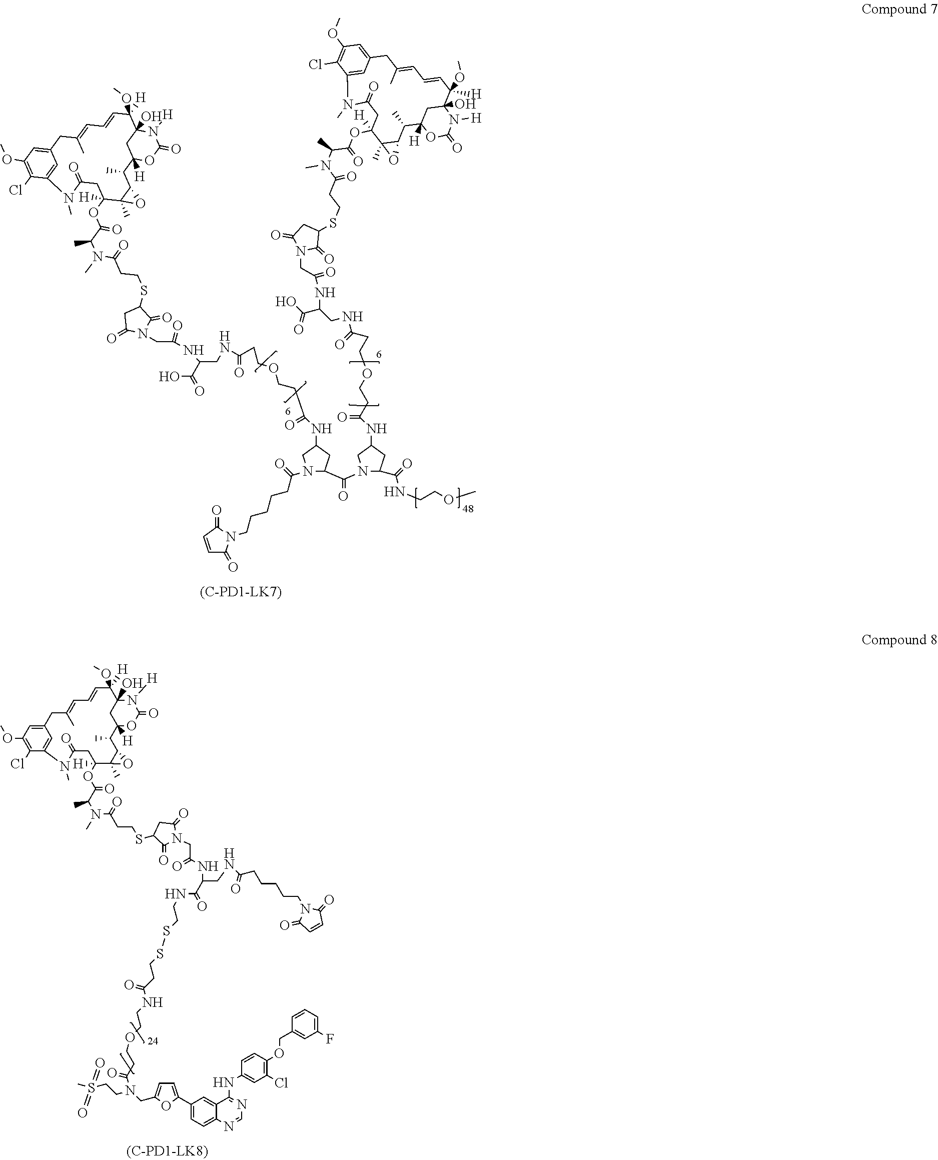

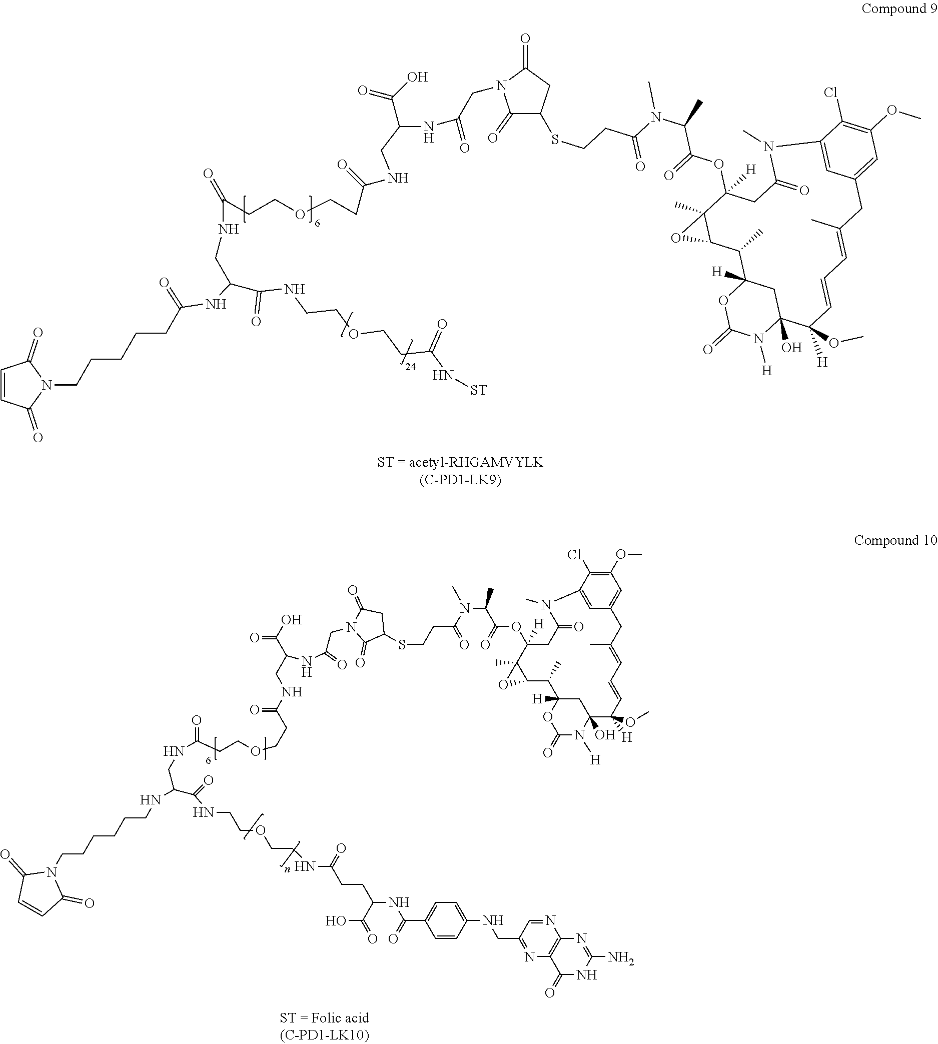

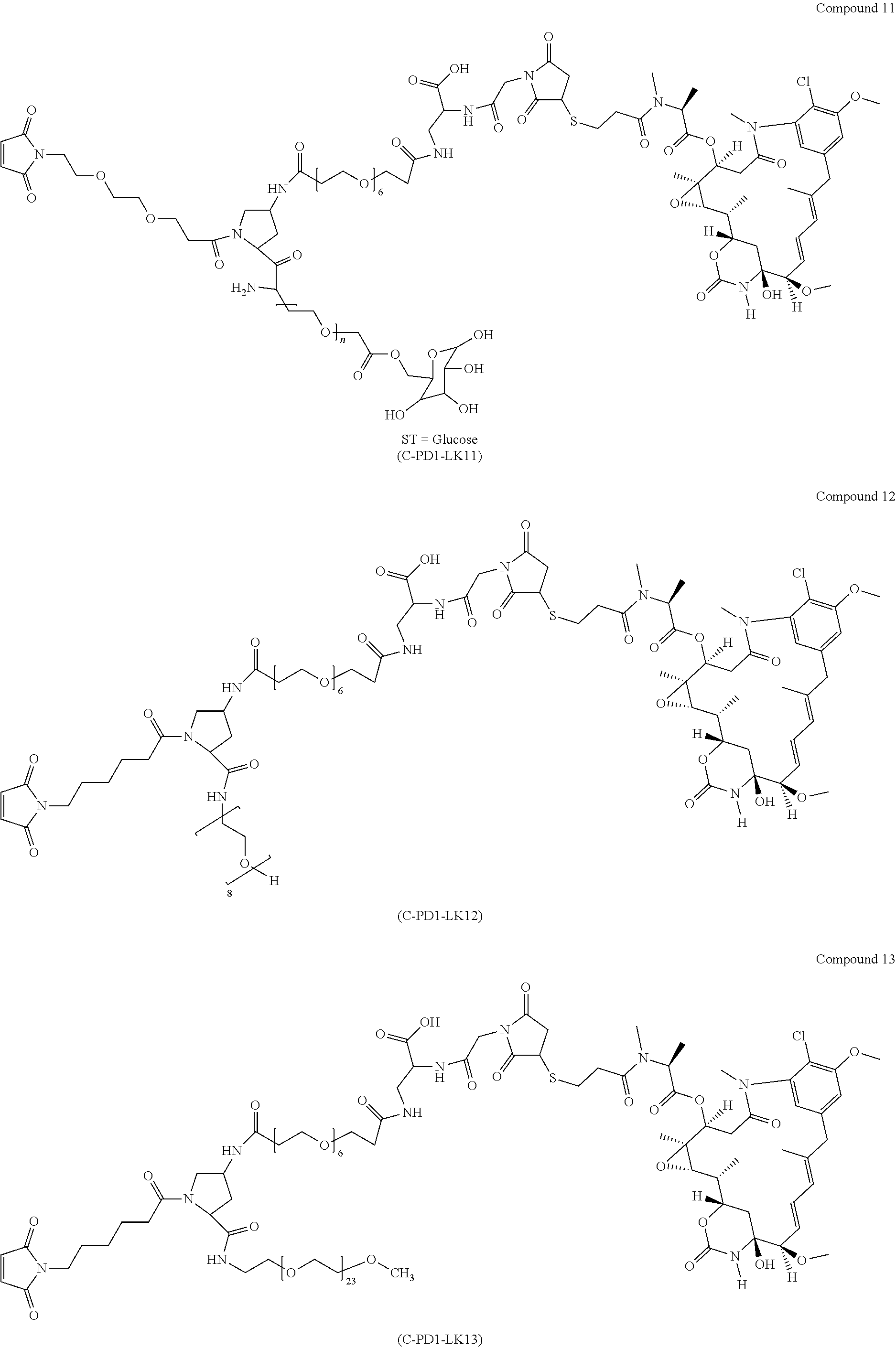

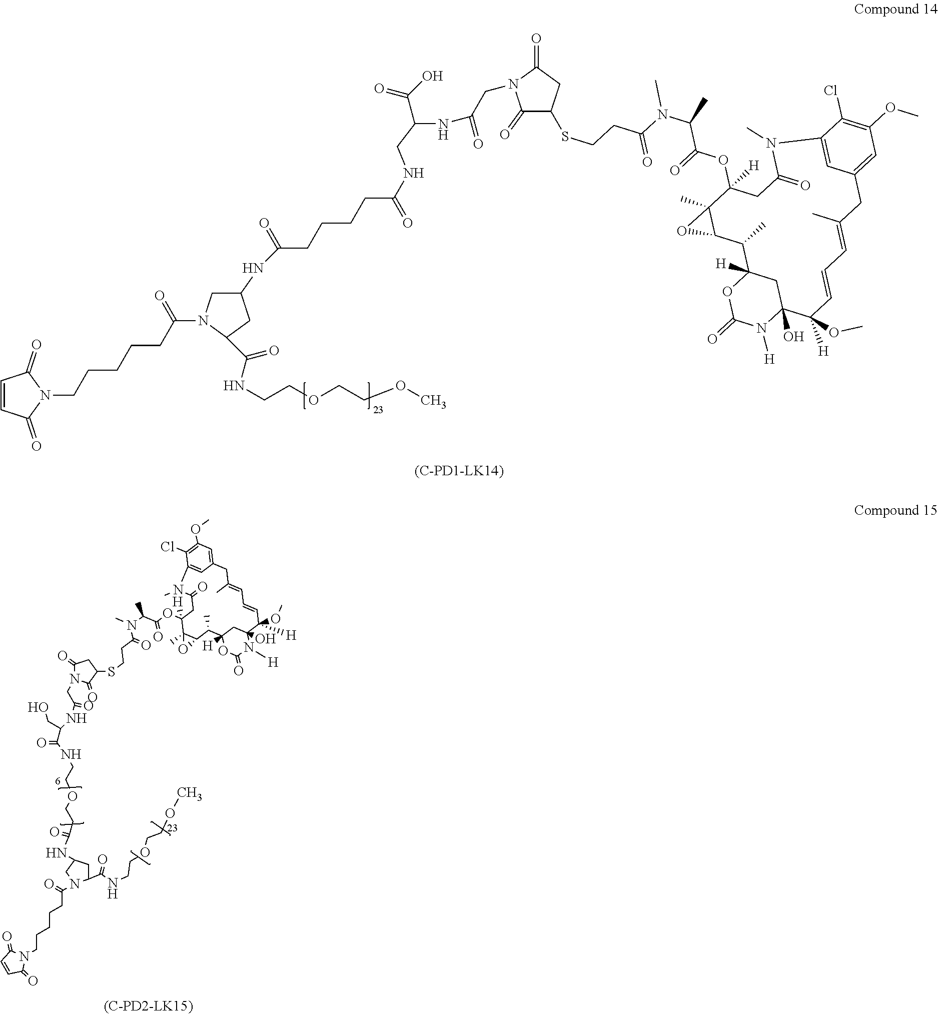

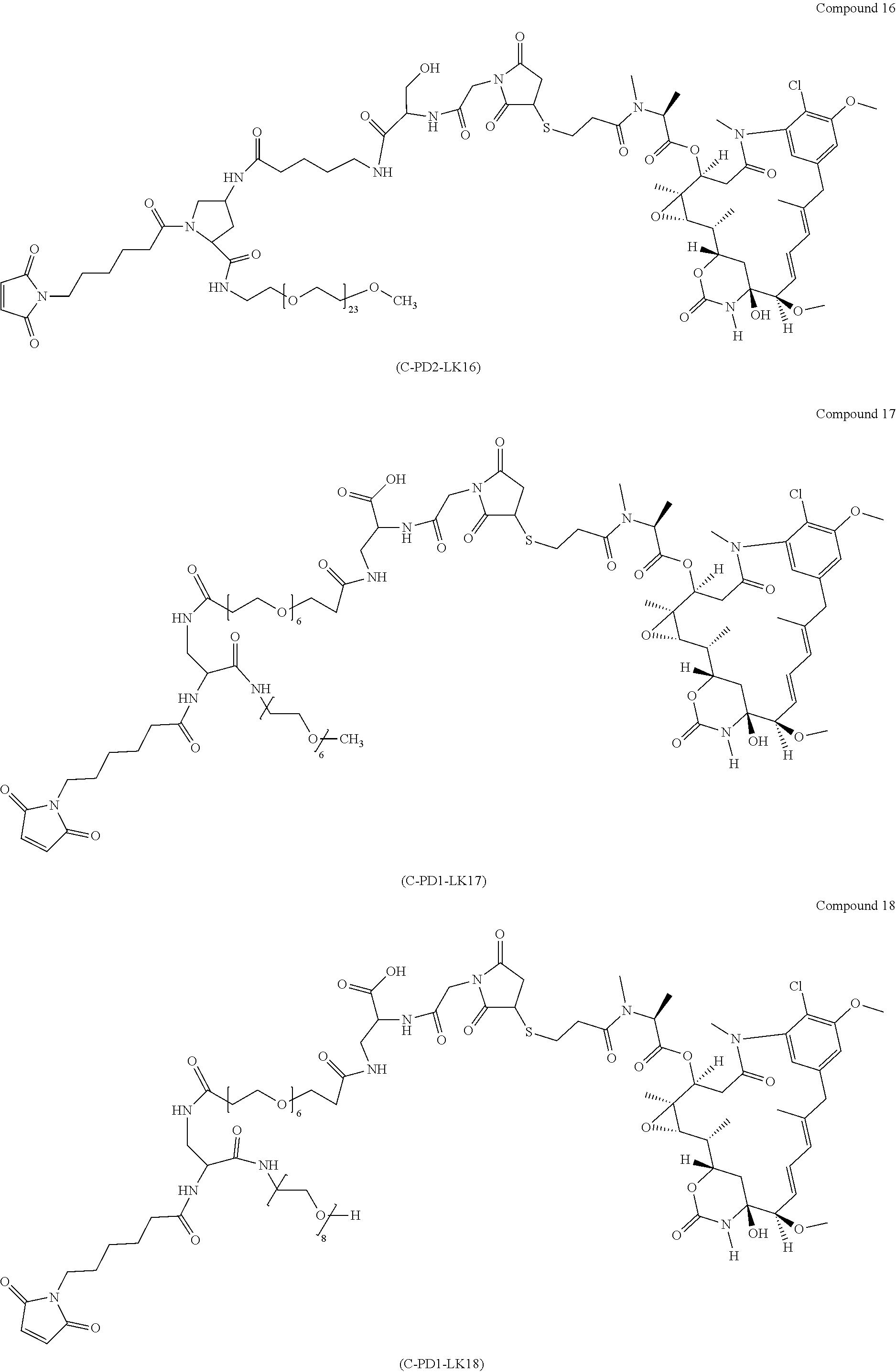

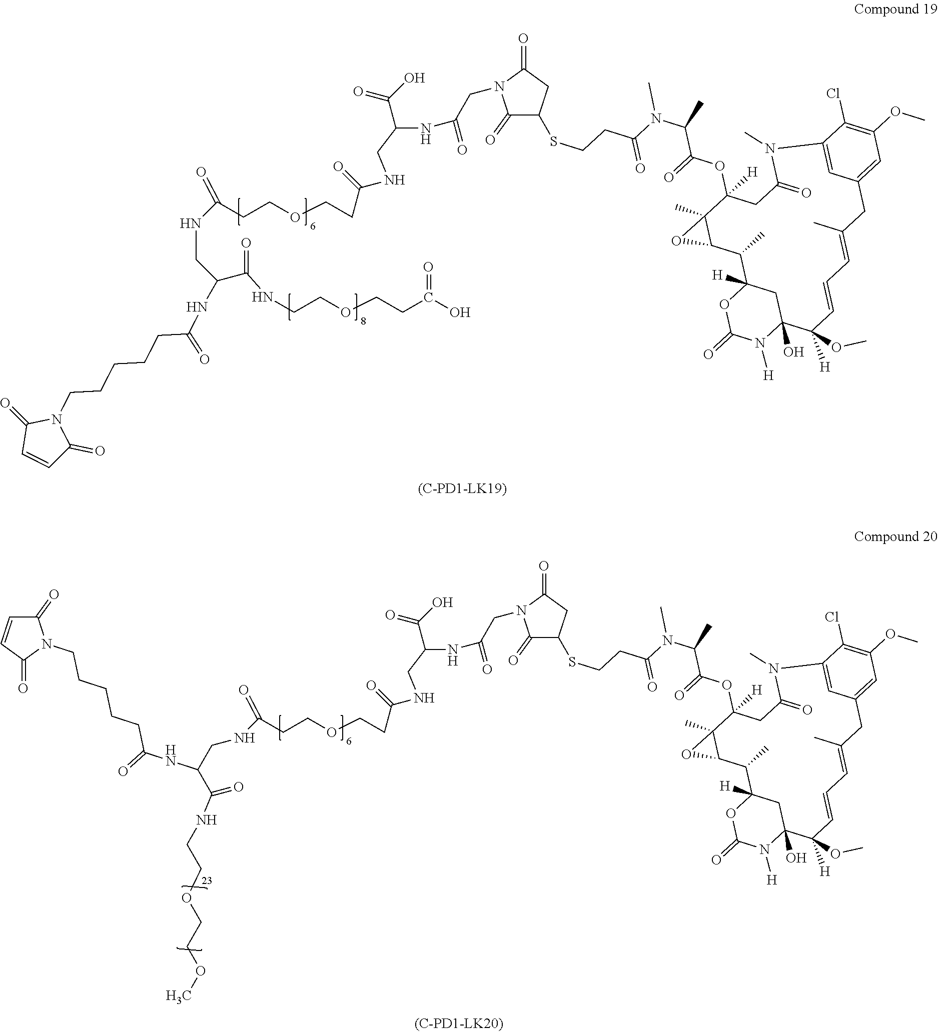

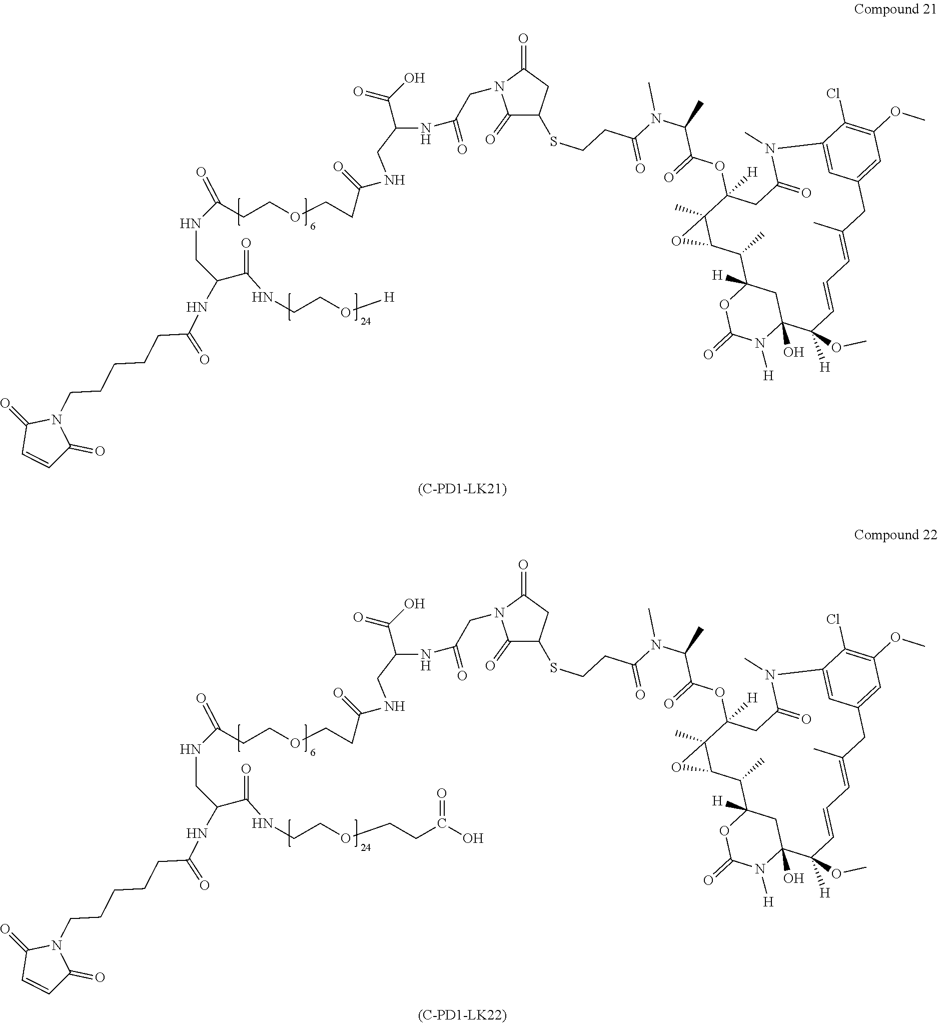

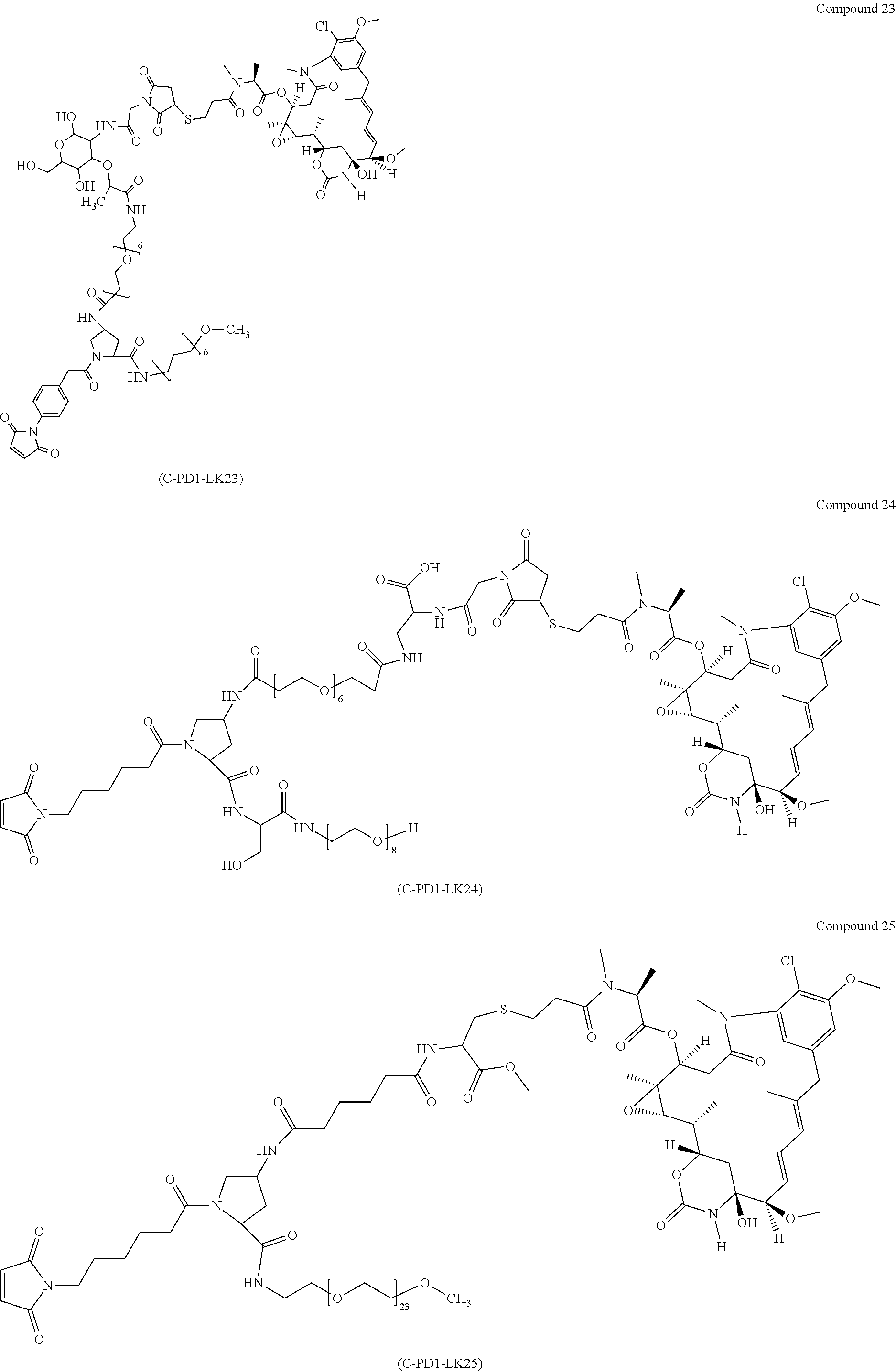

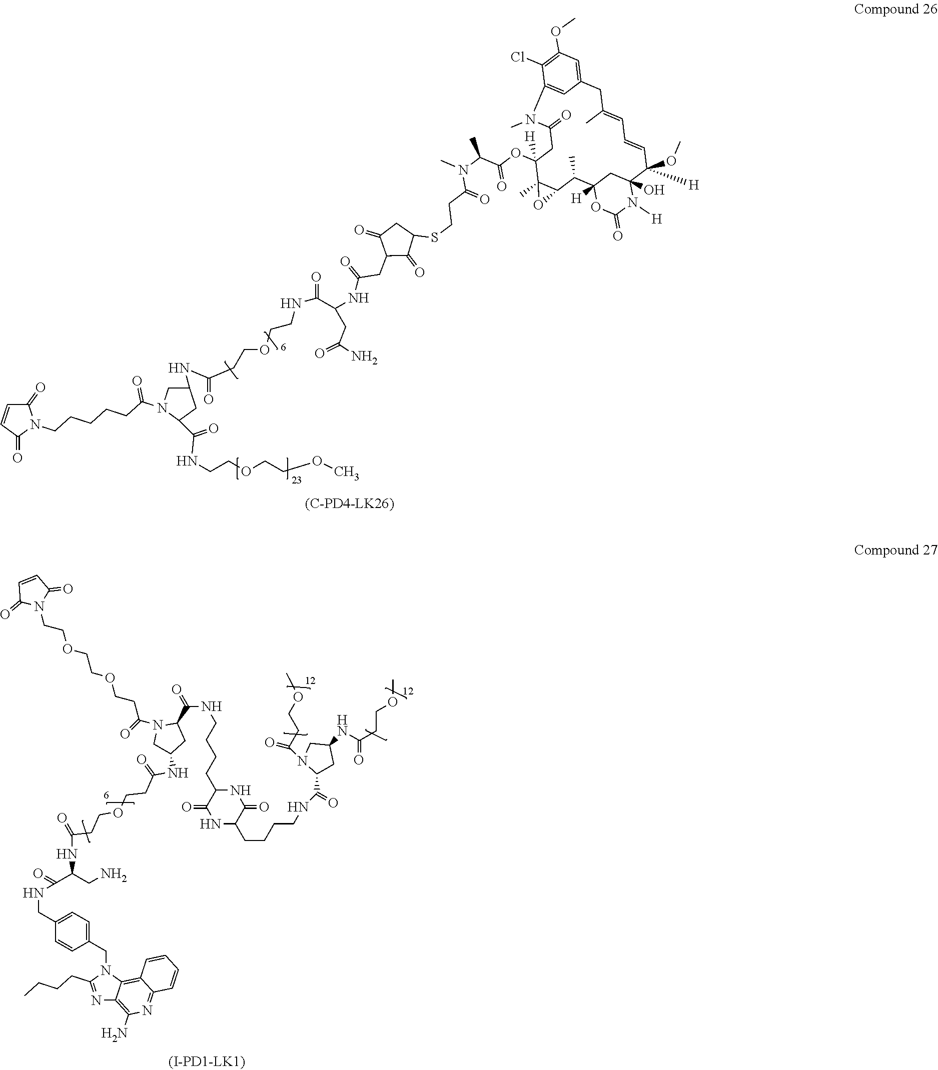

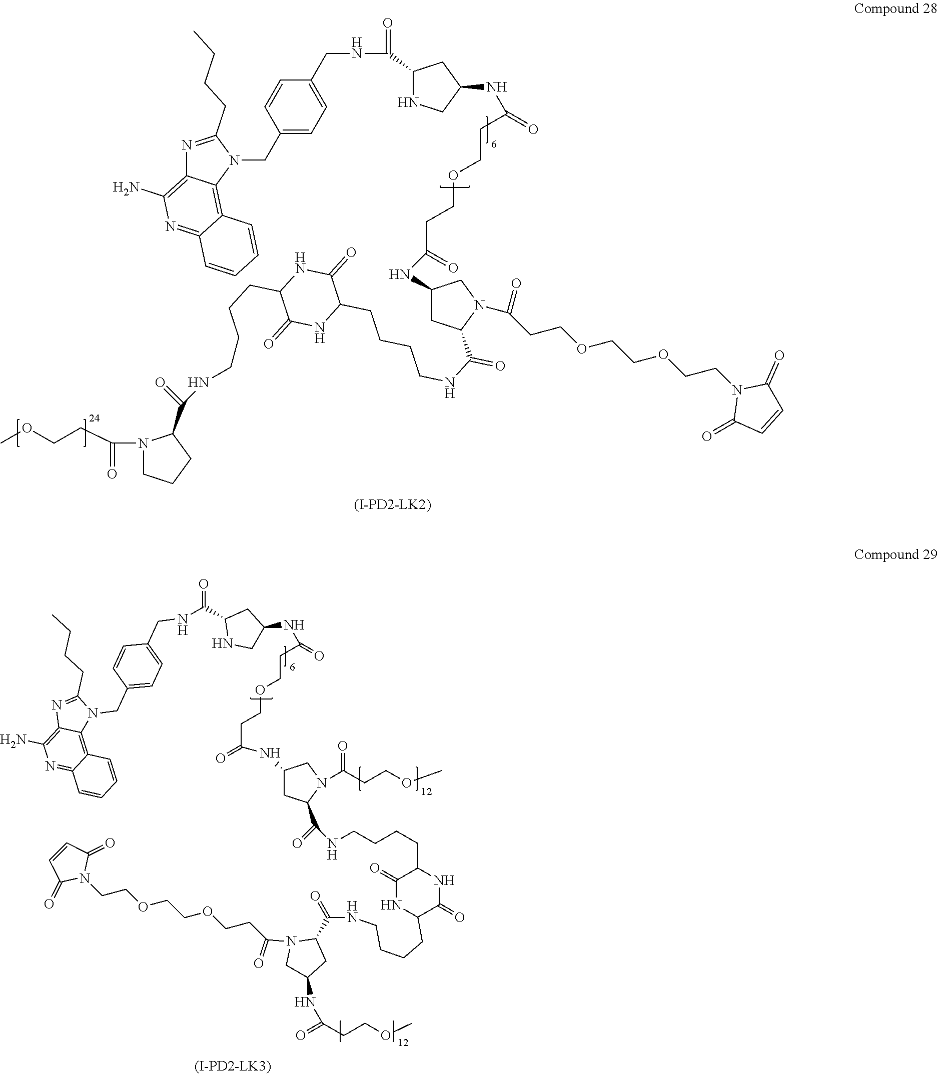

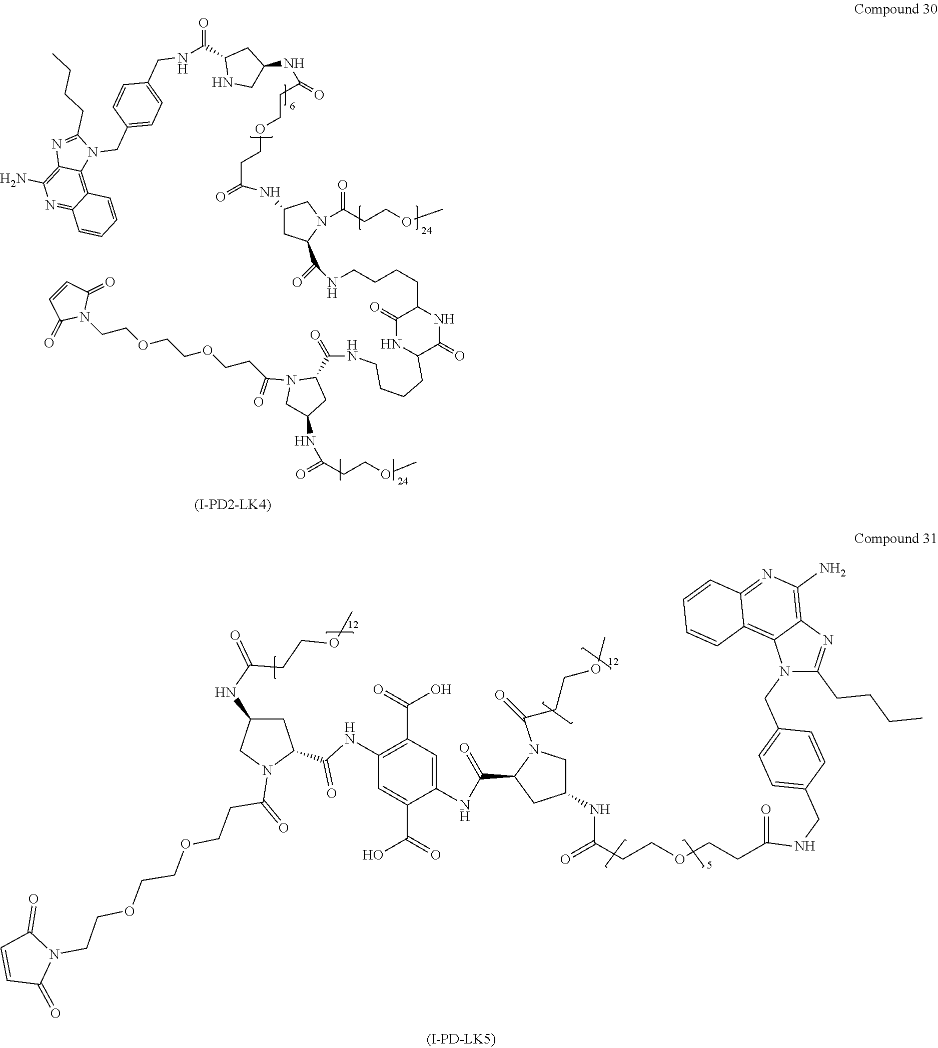

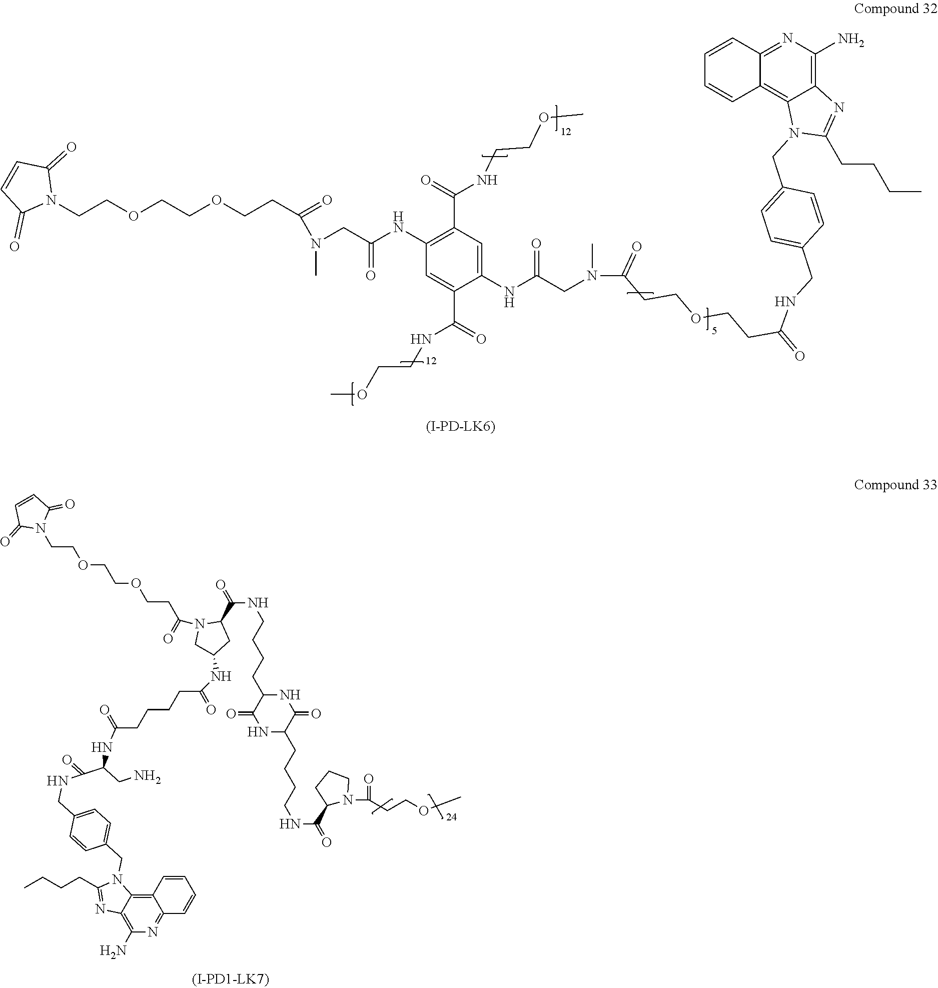

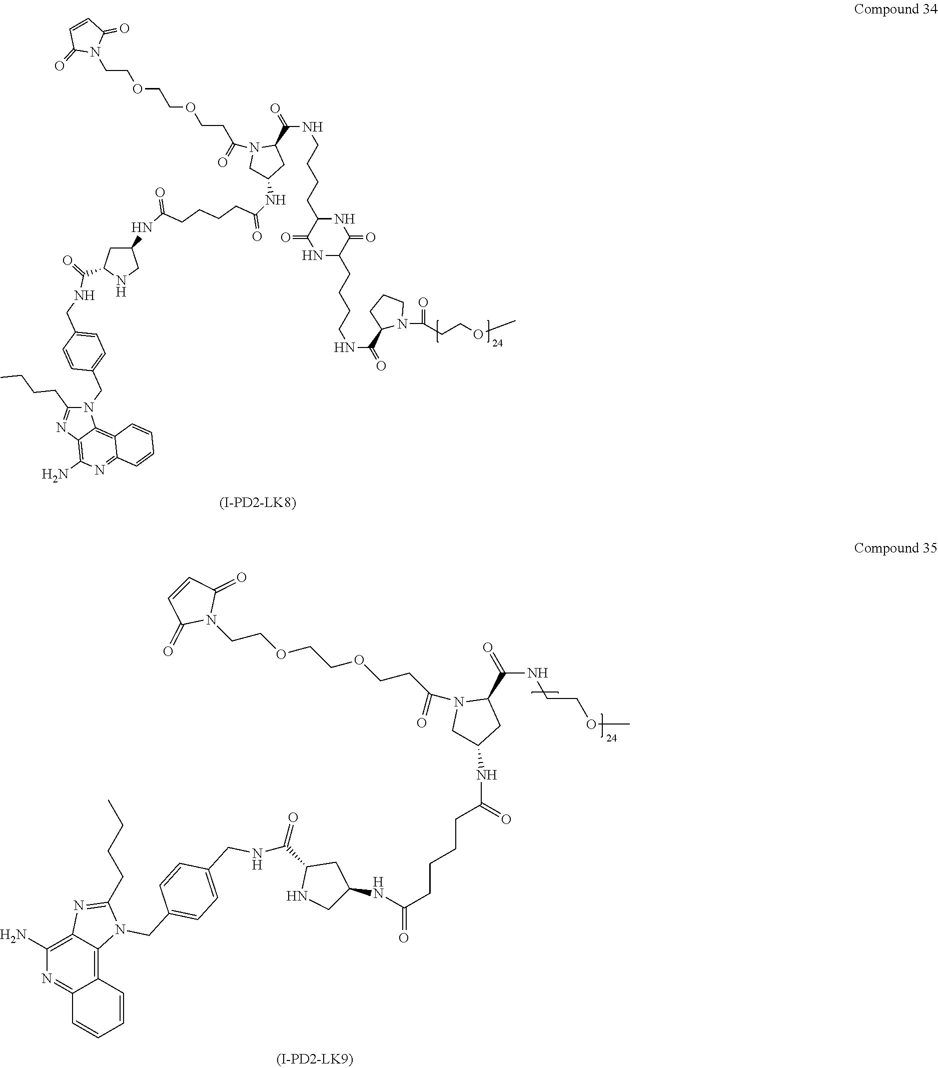

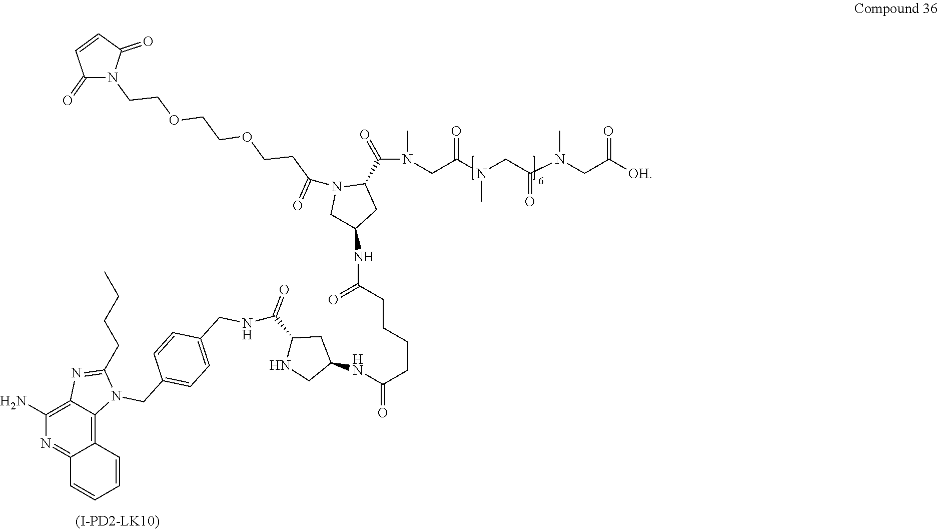

Another aspect of this invention relates to ligand-PD conjugates each containing a ligand moiety and a moiety derived from any one of the compounds described above. The ligand is bonded to the compound via a covalent bond formed between a functional group from the ligand and L1 in formula I, and the functional group is sulfhydryl, amino, glutamine, or formyl.

Exemplary ligands include trastuzumab, albumin, and pertuzumab. The covalent bond is formed between sulfhydryl of the ligand and the maleimide, iodo, or bromo moiety of L1.

Typically, the molar ratio of the ligand to the compound is between 1:1 and 1:20, preferably between 1:2 and 1:8.

Exemplary conjugates of this invention, i.e., Conjugates 1-28, are shown below.

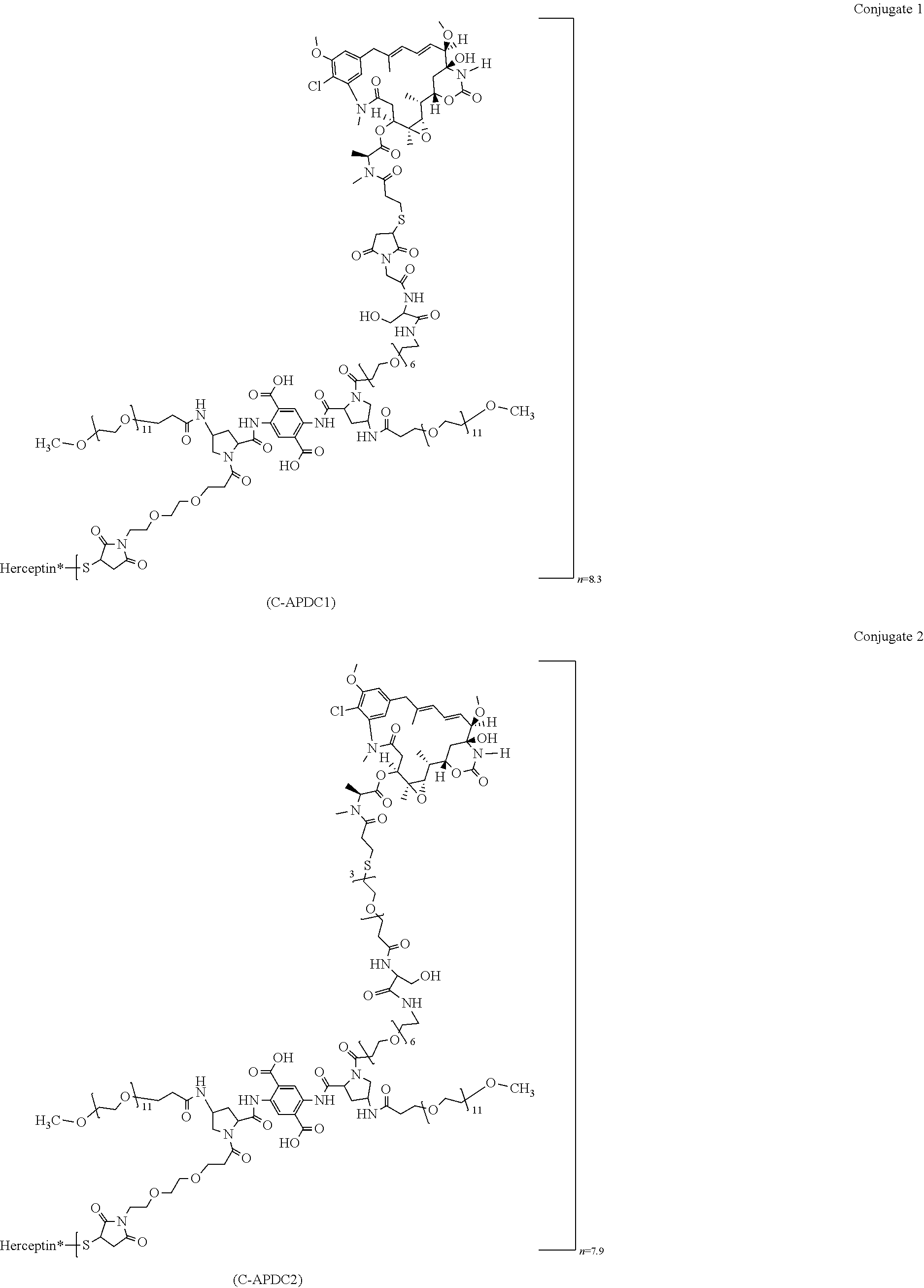

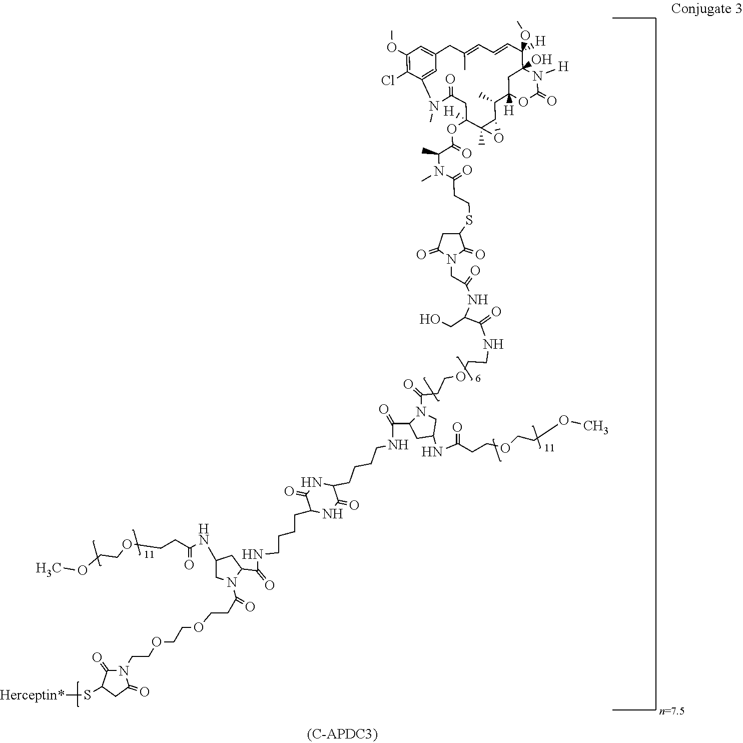

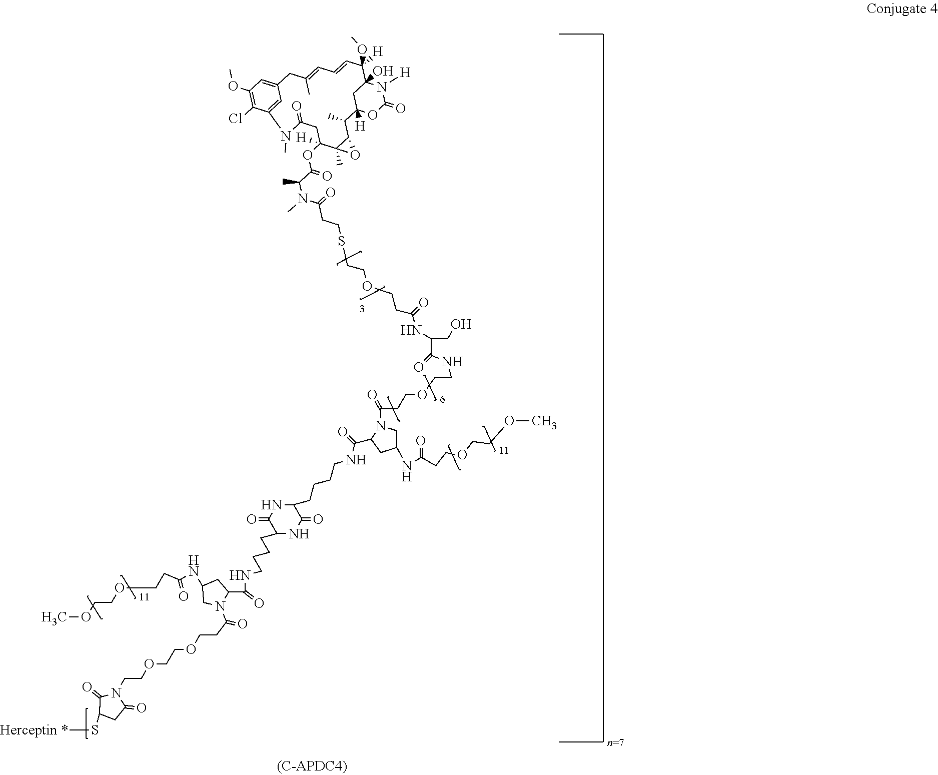

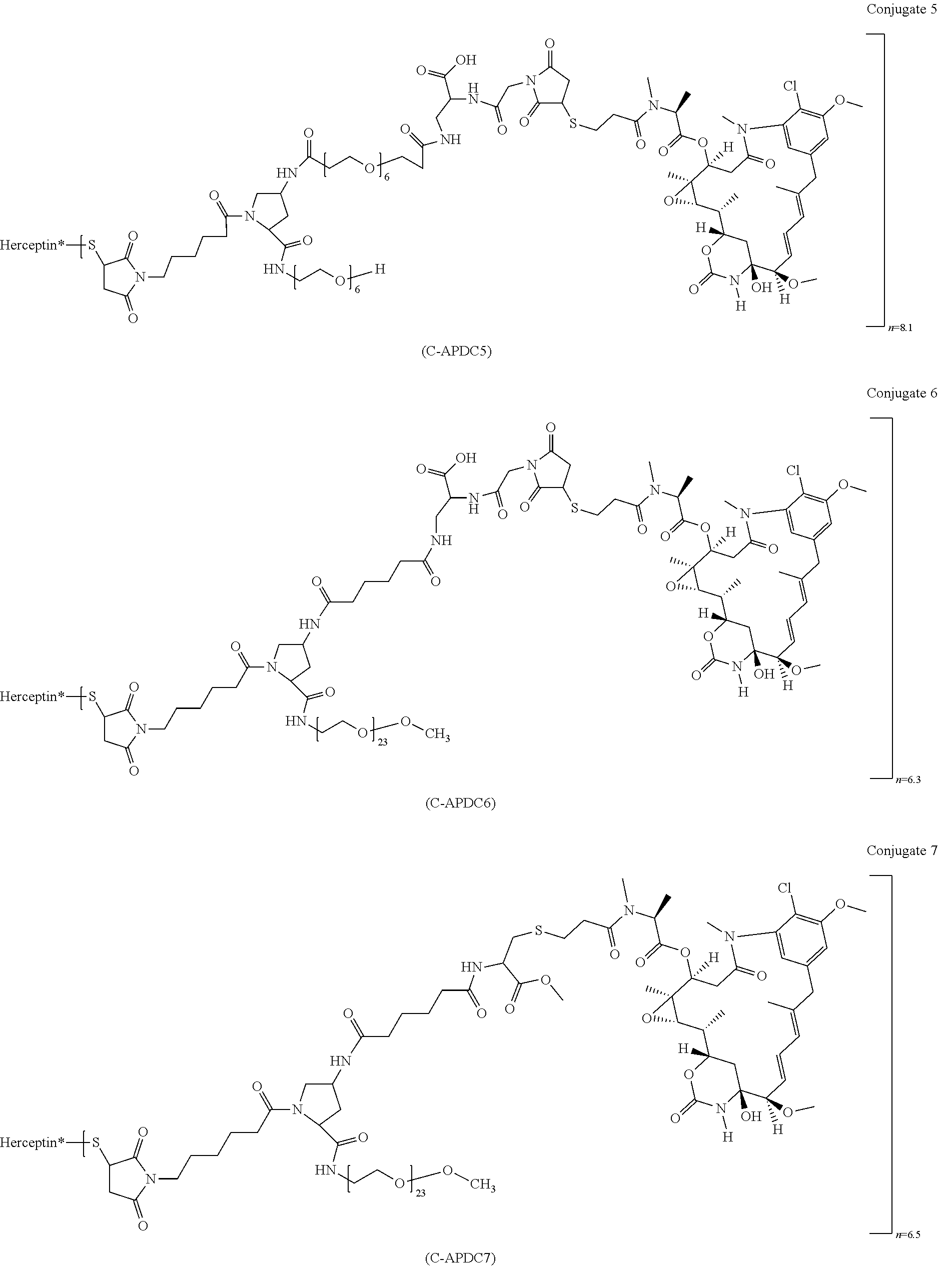

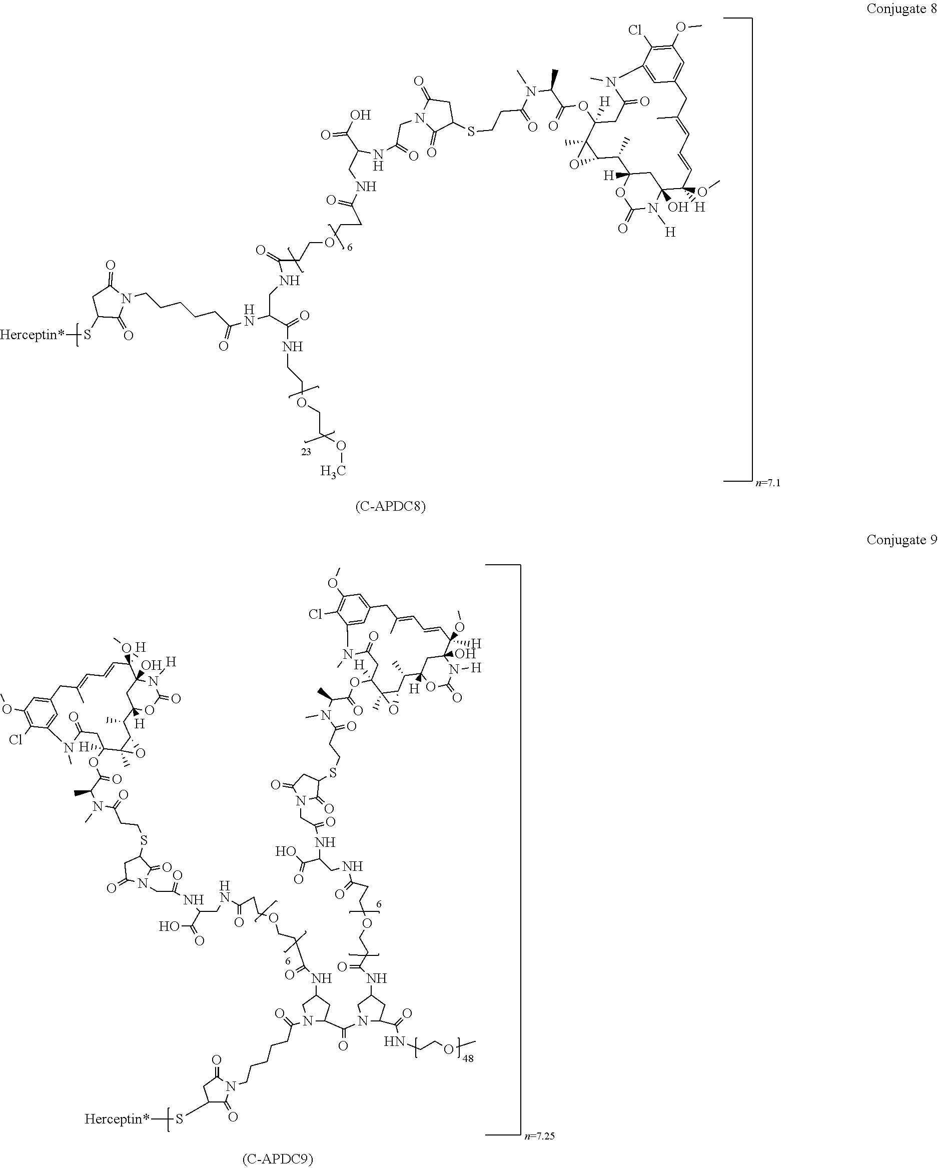

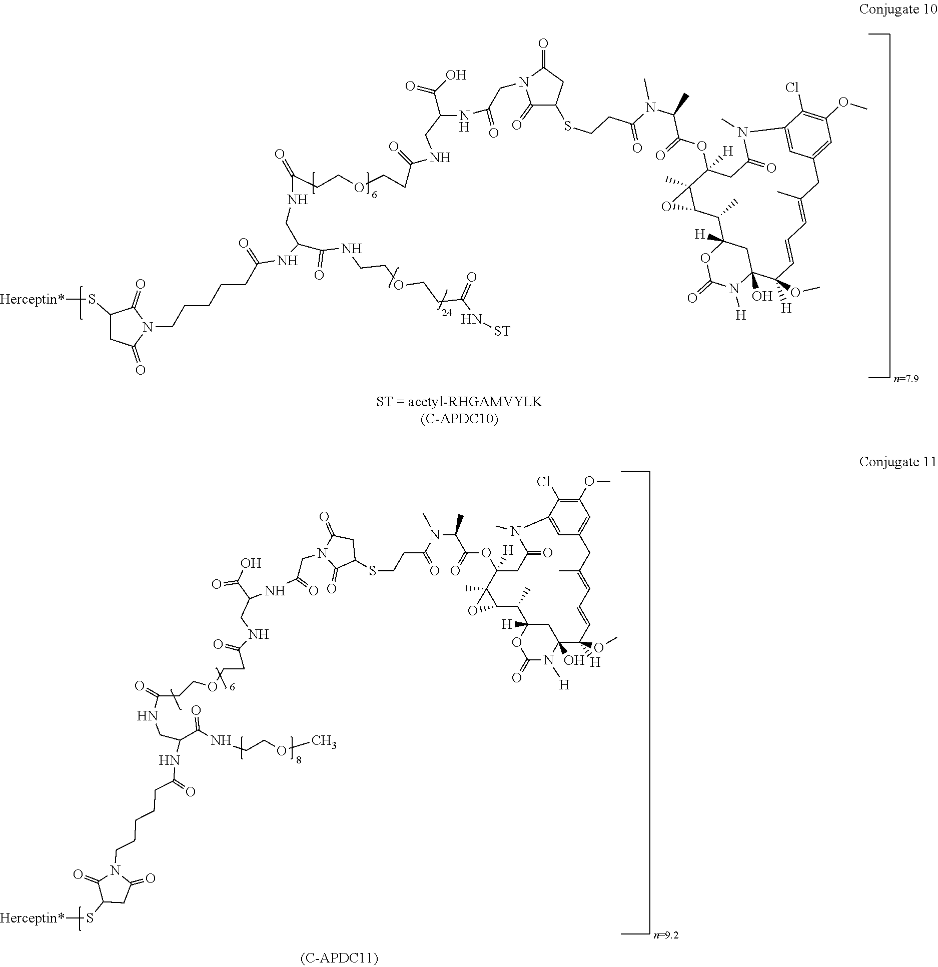

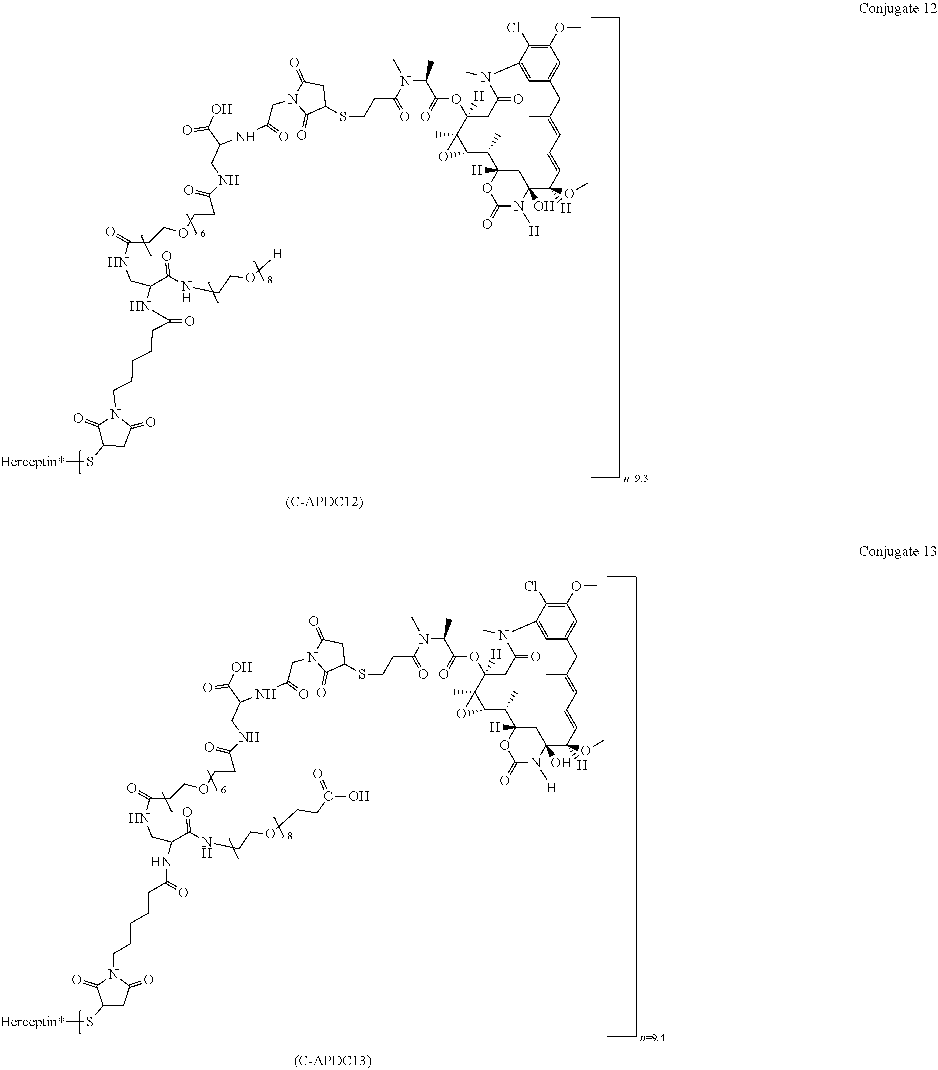

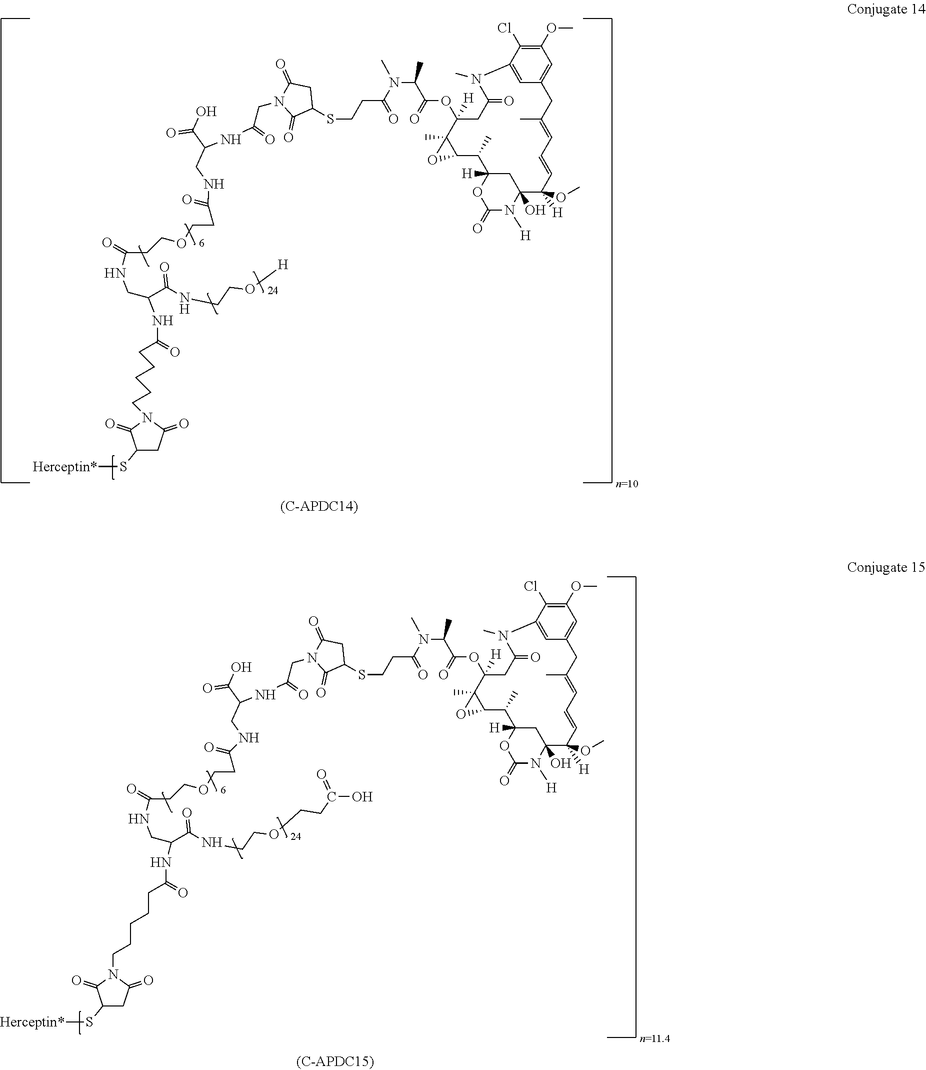

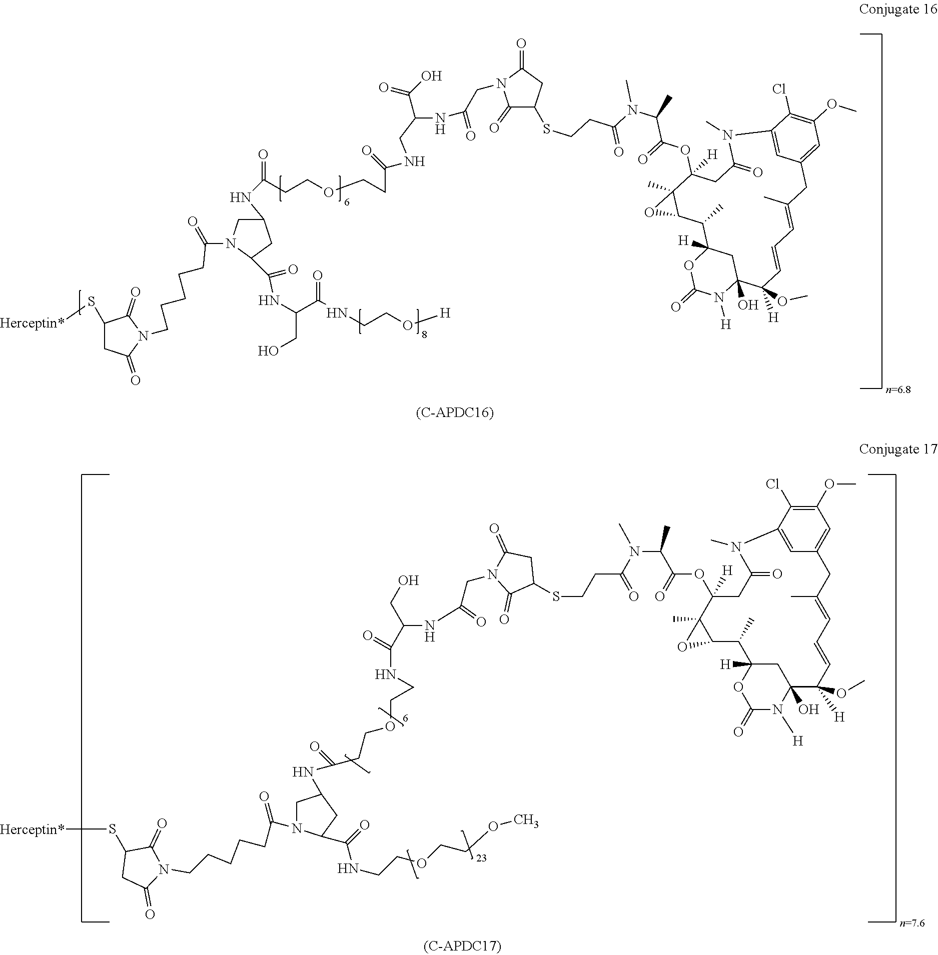

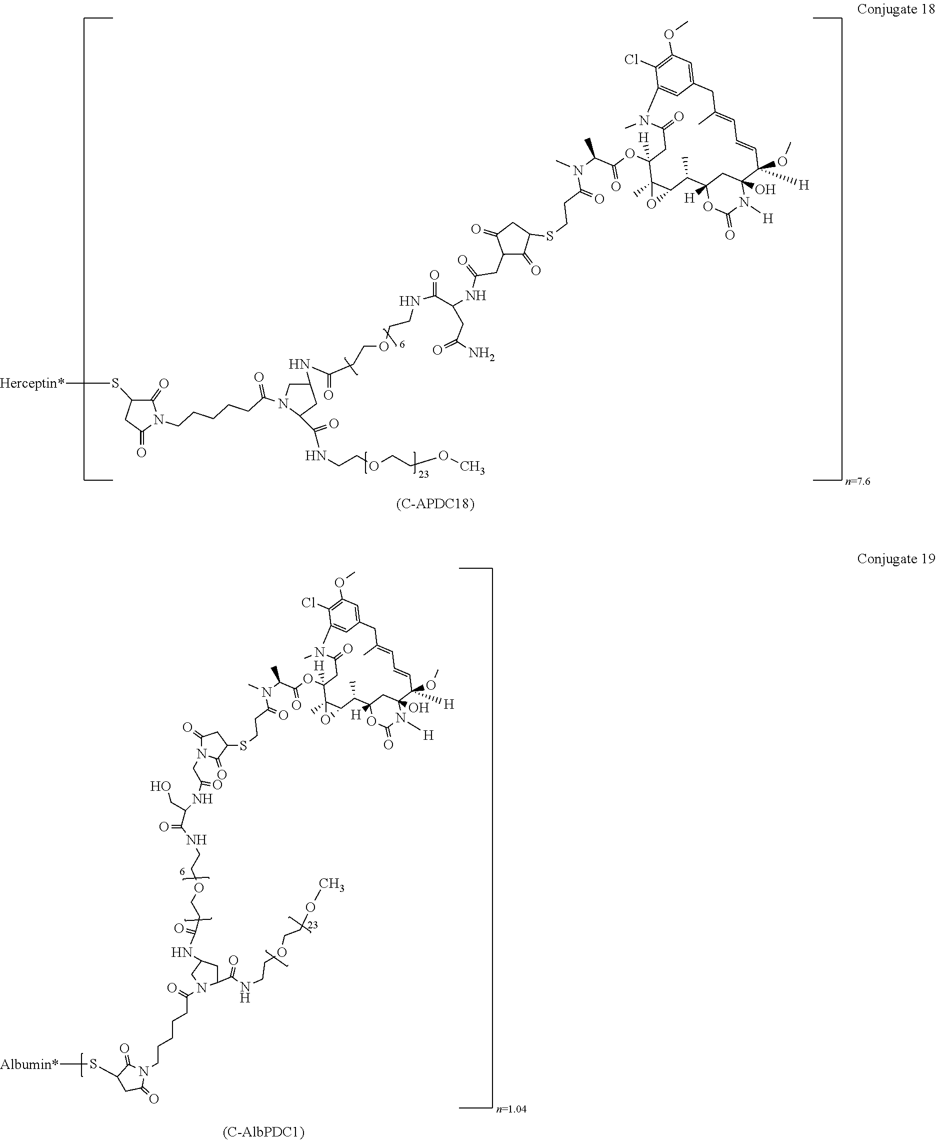

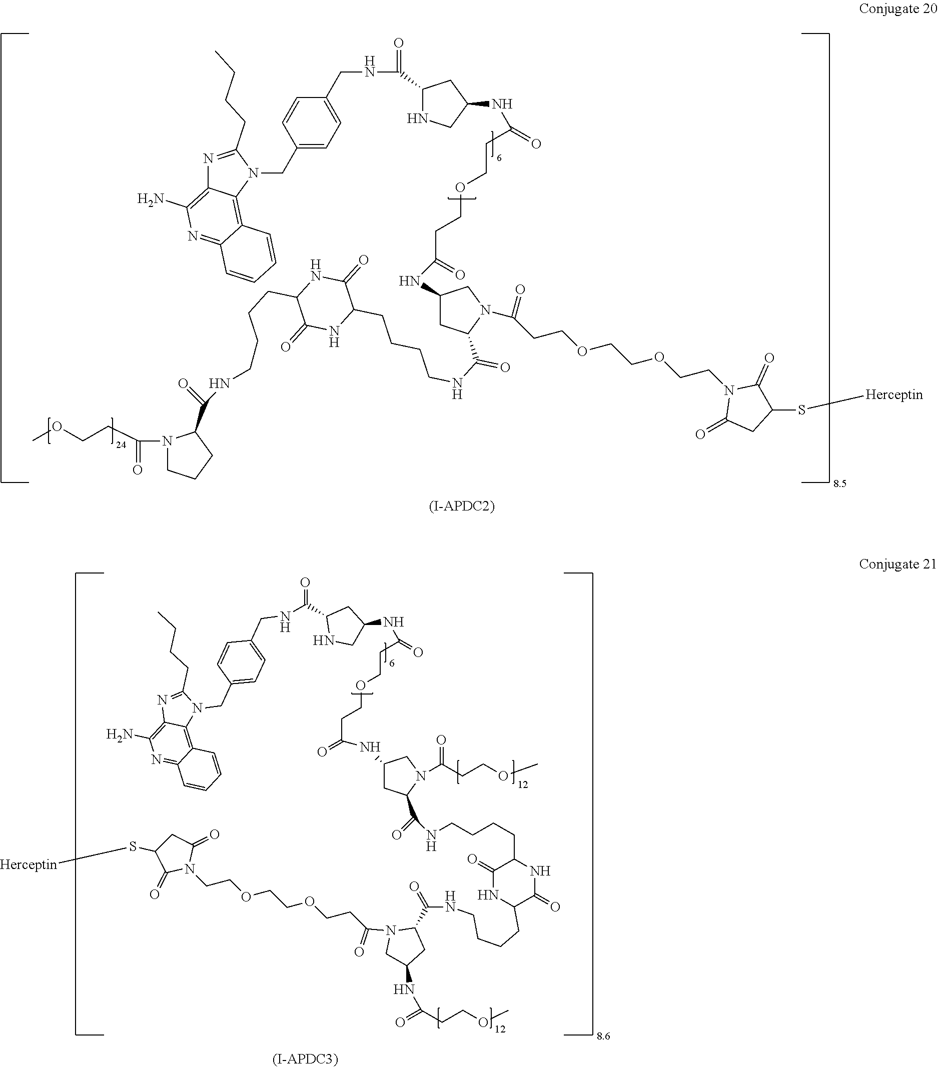

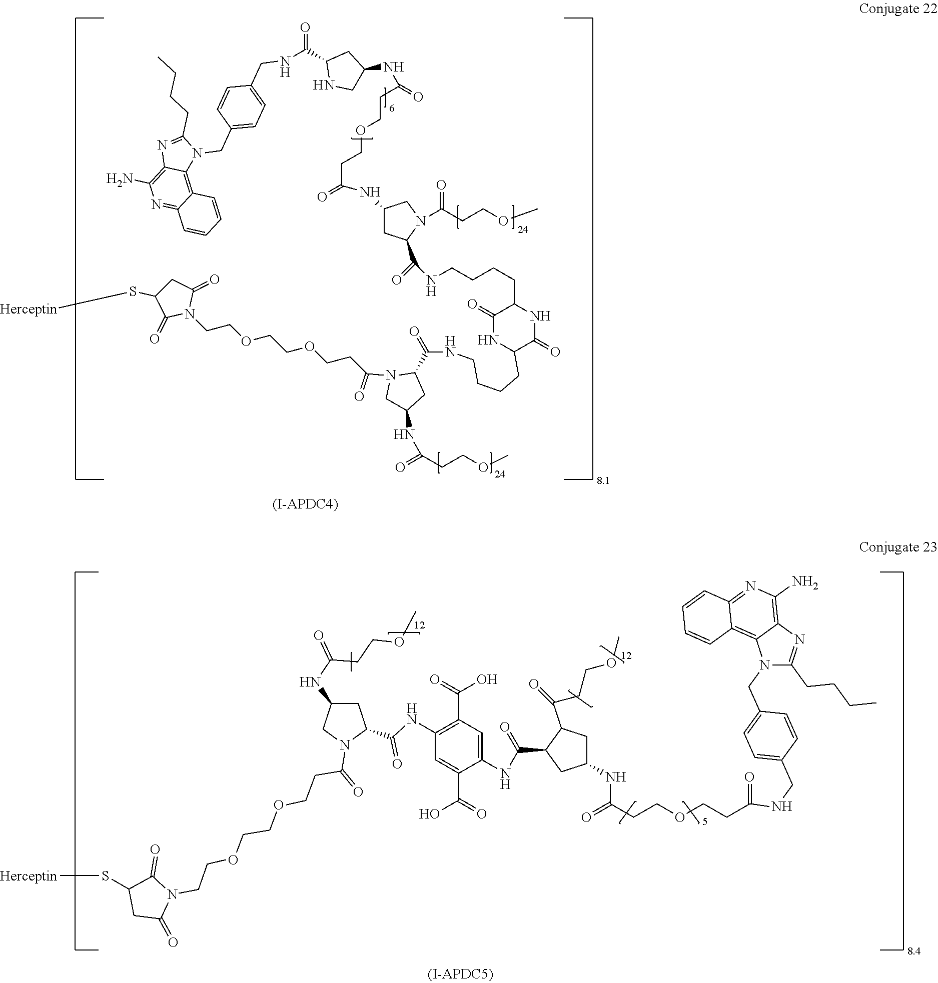

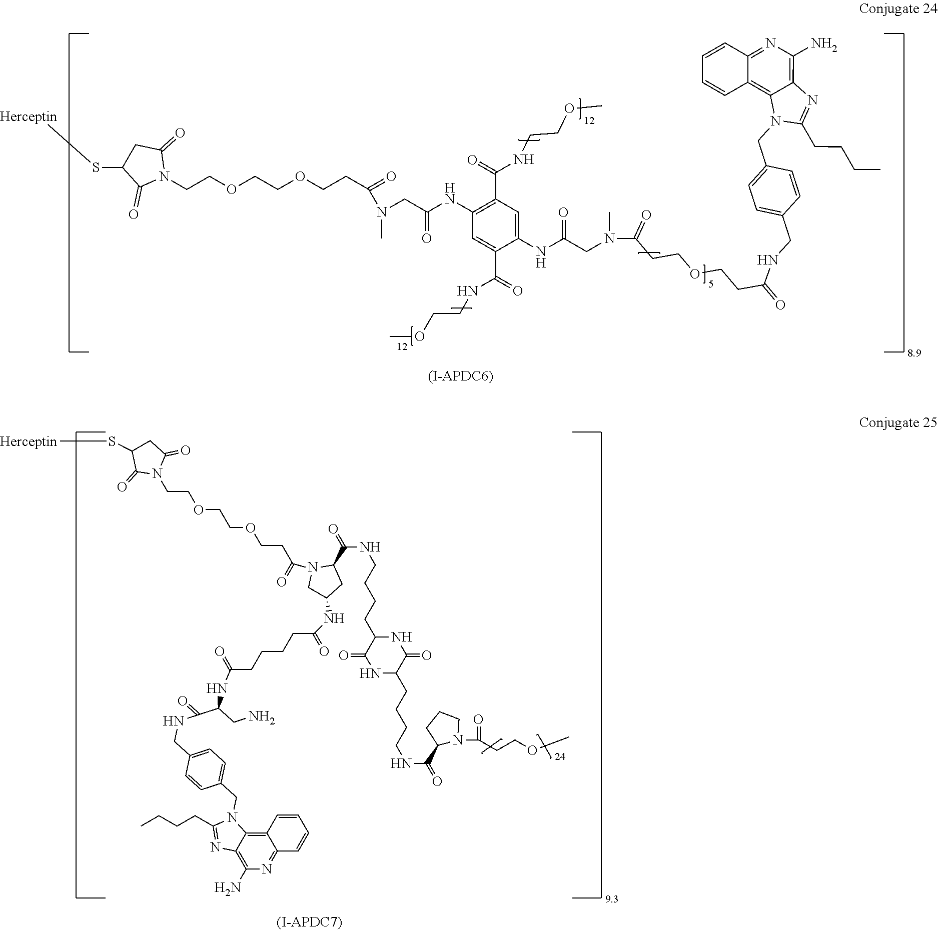

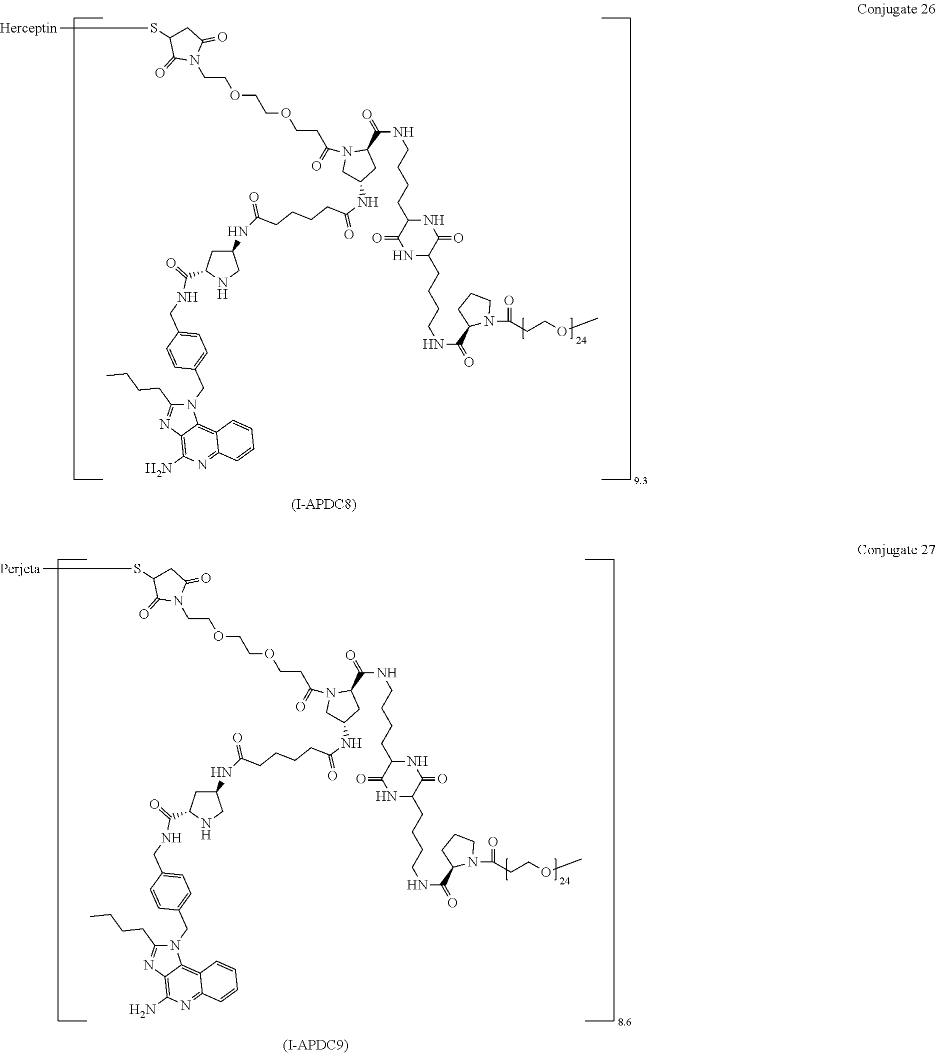

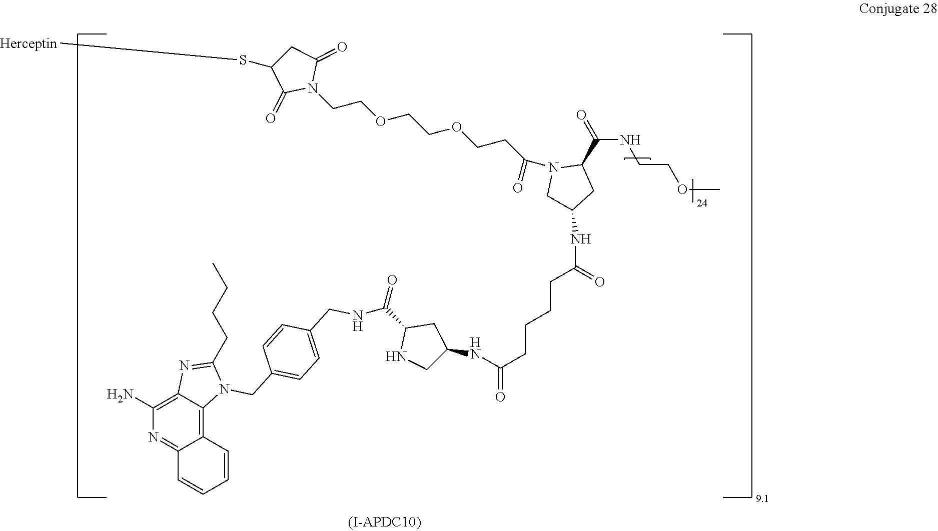

Also within the scope of this invention is a method of treating cancer including the step of administrating to a patient in need thereof an effective amount of any one of the conjugates described above.

The method can be used to treat any cancer. Nonlimiting examples include hematopoietic cancers such as, for example, lymphomas (Hodgkin Lymphoma and Non-Hodgkin Lymphomas) and leukemias and solid tumors. Examples of hematopoietic cancers include, follicular lymphoma, anaplastic large cell lymphoma, mantle cell lymphoma, acute myeloblastic leukemia, chronic myelocytic leukemia, chronic lymphocytic leukemia, diffuse large B cell lymphoma, and multiple myeloma. Examples of solid tumors include fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon cancer, colorectal cancer, kidney cancer, pancreatic cancer, bone cancer, breast cancer, ovarian cancer, prostate cancer, esophageal cancer, stomach cancer, oral cancer, nasal cancer, throat cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, uterine cancer, testicular cancer, small cell lung carcinoma, bladder carcinoma, lung cancer, epithelial carcinoma, glioma, glioblastoma multiforme, astrocytoma, medulloblastoma, craniopharyngioma, ependymoma, pinealoma, hemangioblastoma, acoustic neuroma, oligodendroglioma, meningioma, skin cancer, melanoma, neuroblastoma, and retinoblastoma.

Still within the scope of this invention is a pharmaceutical composition containing any one of the conjugates of this invention, and a pharmaceutically acceptable carrier, diluent, or excipient.

Also provided is any one of the conjugates of this invention for the manufacture of a medicament for treating cancer.

A method of preparing any one of the conjugates described above is also envisioned. The method involves reacting a ligand with any compound described above, in which the ligand contains one or more of functional groups that are, independently, sulfhydryl, amino, glutamine, or formyl.

The term “alkyl” refers to a straight or branched hydrocarbon group, containing 1-20 carbon atoms (e.g., C₁₋₆) and a monovalent radical center derived by the removal of a hydrogen atom from a carbon atom of a parent alkane. Exemplary alkyl groups are methyl, ethyl, n-propyl, isopropyl, n-butyl, iso-butyl, tert-butyl, n-pentyl, and n-hexyl. The term “alkylcarbonyl” or “carbonyl” refers to alkyl-C(O)—. The term “haloalkyl” refers to alkyl substituted with one or more halo atoms. Examples include fluoromethyl, difluoromethyl, trifluoromethyl, fluoroethyl (e.g., 1-fluoroetyl and 2-fluoroethyl), difluoroethyl (e.g., 1,1-, 1,2-, and 2,2-difluoroethyl), and trifluoroethyl (e.g., 2,2,2-trifluoroethyl). The term “heteroalkyl” refers to alkyl having one or more heteroatoms (e.g., O, N, P, and S) that each replaces a carbon atom therein. The term “alkylene” refers to bivalent alkyl containing two monovalent radical centers derived by the removal of two hydrogen atoms from the same carbon atom or two different carbon atoms of a parent alkane. The term “heteroalkylene” refers to bivalent heteroalkyl containing two monovalent radical centers.

The term “halogen” herein refers to a fluoro, chloro, bromo, or iodo radical. Examples include a fluoro radical (F) and a bromo radical (Br).

The term “carboxamide” refers to —C(O)NH₂.

The term “amino” refers to a radical derived from amine, which is unsubstituted or substituted with alkyl, aryl, cycloalkyl, heterocycloalkyl, or heteroaryl. The term “aminoalkyl” refers to NH₂-alkyl, i.e., an alkyl that is substituted with at least one amino group. The term “alkylamino” refers to alkyl-NH—. Examples of aminoalkyl include aminomethyl and 2-aminoethyl. The term “acylamino” refers to —C(O)—NH—.

The term “aryl” refers to monovalent carbocyclic group containing one or more fused or non-fused phenyl rings. It is understood when multiple rings are employed, the term includes partially unsaturated ring systems. Typical aryl groups include phenyl, biphenyl, 1 or 2-naphthyl, 1,2-dihydronaphthyl, 1,2,3,4-tetrahydronaphthyl, indenyl, indanyl and the like. The term “arylene” refers to bivalent aryl containing two monovalent radical centers.

The term “heteroaryl” refers to an aromatic 5-8 membered monocyclic, 8-12 membered bicyclic, or 11-14 membered tricyclic ring system having one or more heteroatoms (e.g., O, N, P, and S). Examples include pyridinyl, pyrimidinyl, quinolinyl, isoquinolinyl, indolyl, benzimidazolyl, benzoxazolyl, benzothiophenyl, benzofuranyl, pyrazolyl, triazolyl, oxazolyl, thiadiazolyl, tetrazolyl, oxazolyl, isoxazolyl, carbazolyl, furyl, imidazolyl, thienyl, thiazolyl, and benzothiazolyl.

The term “cycloalkyl” refers to a nonaromatic, saturated or unsaturated monocyclic, bicyclic, tricyclic, or tetracyclic hydrocarbon group containing 3 to 12 carbons (e.g., C3-6 and C3-10). It is understood when multiple rings are employed, the term includes fused, bridged and spiro ring systems. Typical cycloalkyl groups include monocyclic, bicyclic, and spiro rings such as cyclopropyl, cyclobutyl, cyclopentyl, cyclopentenyl, cyclohexyl, cyclohexenyl, cycloheptyl, cyclooctyl, bicyclo[1.1.1]pentyl, bicyclo[2.1.1]hexyl, decahydronaphthalene and the like. The term “carbocyclo” refers to bivalent cycloalkyl containing two monovalent radical centers.

The term “heterocycloalkyl” refers to a nonaromatic, saturated or unsaturated, 3-8 membered monocyclic, 8-12 membered bicyclic, or 11-14 membered tricyclic ring system having one or more heteroatoms (e.g., 0, N, P, and S). Examples include aziridinyl, azetidinyl, pyrrolidinyl, dihydrofuranyl, tetrahydrofuranyl, tetrahydropyranyl, tetrahydrothiophenyl, tetrahydro-2-H-thiopyran-1,1-dioxidyl, piperazinyl, piperidinyl, morpholinyl, imidazolidinyl, azepanyl, dihydrothiadiazolyl, dioxanyl, quinuclidinyl, 2-azaspiro[3.3]heptanyl, and 8-azabicyclo[3.2.1]octanyl. The term “heterocyclo” refers to bivalent heterocycloalkyl containing two monovalent radical centers.

The term “formyl” refers to a —C(O)H group. The term “carboxyl” refers to a —C(O)—OH group. The term “carboxylate” refers to a —O—C(O)-alkyl group. The term “acetyloxy” refers to a —OC(O)CH₃ group. The term “oxo” refers to a ═O group.

The term “amino acid” refers to any monomeric unit that can be incorporated into a peptide, polypeptide, or protein. Amino acids include natural amino acids and their stereoisomers, as well as non-natural amino acids and their stereoisomers. “Stereoisomers” of a given amino acid refer to isomers having the same molecular formula and intramolecular bonds but different three-dimensional arrangements of bonds and atoms (e.g., an L-amino acid and the corresponding D-amino acid).

Natural amino acids are naturally-occurring α-amino acids encoded by the genetic code, as well as those amino acids that are later modified, e.g., hydroxyproline, α-carboxyglutamate, and O-phosphoserine. Naturally-occurring α-amino acids include, without limitation, alanine (Ala), cysteine (Cys), aspartic acid (Asp), glutamic acid (Glu), phenylalanine (Phe), glycine (Gly), histidine (His), isoleucine (Ile), arginine (Arg), lysine (Lys), leucine (Leu), methionine (Met), asparagine (Asn), proline (Pro), glutamine (Gln), serine (Ser), threonine (Thr), valine (Val), tryptophan (Trp), tyrosine (Tyr), and combinations thereof. Stereoisomers of a natural amino acids include, without limitation, D-alanine (D-Ala), D-cysteine (D-Cys), D-aspartic acid (D-Asp), D-glutamic acid (D-Glu), D-phenylalanine (D-Phe), D-histidine (D-His), D-isoleucine (D-Ile), D-arginine (D-Arg), D-lysine (D-Lys), D-leucine (D-Leu), D-methionine (D-Met), D-asparagine (D-Asn), D-proline (D-Pro), D-glutamine (D-Gln), D-serine (D-Ser), D-threonine (D-Thr), D-valine (D-Val), D-tryptophan (D-Trp), D-tyrosine (D-Tyr), and combinations thereof.

Non-natural (non-naturally occurring) amino acids include, without limitation, amino acid analogs, amino acid mimetics, synthetic amino acids, N-substituted glycines, and N-methyl amino acids in either the L- or D-configuration that function in a manner similar to the natural amino acids. For example, “amino acid analogs” can be non-natural amino acids that have the same basic chemical structure as natural amino acids (i.e., a carbon that is bonded to a hydrogen, a carboxyl group, an amino group) but have modified side-chain groups or modified peptide backbones, e.g., homoserine, norleucine, methionine sulfoxide, methionine methyl sulfonium. “Amino acid mimetics” refer to chemical compounds that have a structure that is different from the general chemical structure of an amino acid, but that functions in a manner similar to a natural amino acid. Amino acids may be referred to herein by either the commonly known three letter symbols or by the one-letter symbols recommended by the IUPAC-IUB Biochemical Nomenclature Commission.

The term “diamino dicarboxylic acid” refers to an aromatic or nonaromatic, saturated or unsaturated, cyclic or non-cyclic hydrocarbon having two or more (e.g., two, three, and four) amino groups, two or more (e.g., two, three, and four) carboxyl groups, and at least six heteroatoms (e.g., O, N, P, and S). Examples include 2,5-diaminobenzene-1,4-dicarboxylic acid, 3,6-diaminopyrazine-2,5-dicarboxylic acid, 2,2′-diamino-[1,1′-Biphenyl]-4,4′-dicarboxylic acid, 1,4-diamino-cyclohexane-1,4-dicarboxylic acid, 4,4′-Methylene bisanthranilic acid, 2,5-bis(methylamino)terephthalic acid, 3,6-diamino-phthalic acid, 2,5-diaminocyclohexa-1,4-diene-1,4-dicarboxylic acid, 2,3-diaminoterephthalic acid, 2,5-diaminocyclohexane-1,4-dicarboxylic acid, 2,5-diaminoterephthalic acid, 2,5-diaminobenzene-1,3-dicarboxylic acid, 2,5-dihydroxycyclohexa-1,4-diene-1,4-dicarboxylic acid, 2,5-dihydroxycyclohexa-1,3-diene-1,4-dicarboxylic acid, 4,6-diaminobenzene-1,3-dicarboxylic acid, 3,6-diaminopyrazine-2,5-dicarboxylic acid, 3,3′-diamino-[1,1′-biphenyl]-4,4′-dicarboxylic acid, 2,2′-diamino-4,4′-biphenyldicarboxylic acid, 2,2′-diamino-4,4′-stilbenedicarboxylic acid, 2,3-diaminosuccinic acid, 2,4-diamino-pentanedioic acid, 2,5-diaminoadipic acid, 2,6-diaminopimelic acid, 2,7-diaminosuberic acid, 2,8-diaminoazelaic acid, 2,9-diaminodecanedioic acid, 2,10-diaminoundecanedioic acid, di-O-methylated ristomycinic acid, cystathionine, lanthionine, and djenkolic acid.

A stable (or enzymatically resistant) amide bond refers to either (1) a peptide bond formed between two amino acids, natural or non-natural where said peptide bond is formed between a secondary amine group from one amino acid and/or a carboxylic acid group from the other amino acid independently selected from cyclic □-amino acids, cyclic O-amino acids, or cyclic secondary amino acids (see below); (2) a peptide bond found in a cyclic peptide; (3) an isopeptide bond which few enzymes are capable of hydrolyzing (see KW-1017, UniProtKB entries), or (4) an amide bond formed between an amino acid and a non-amino acid compound (such as a bifunctional crosslinker, a polyamine, a monosaccharide, or a diamino dicarboxylic acid). Actually, amide bonds found in the active metabolites released from conventional ADCs with a non-cleavable linker are stable ones (Erickson et al., 2006; Phillips et al., 2008; Leal et al., 2015).

A simple amide bond refers to a peptide bond formed between two natural amino acids excluding proline.

An amide bond refers to either a simple amide bond or a stable amide bond.

A stable glycoside bond refers to one formed between a hemiacetal and a hydroxyl group where at least one of them is a secondary hemiacetal, a tertiary hemiacetal, a secondary hydroxyl group, or a tertiary hydroxyl group.

A simple glycoside bond refers to one formed between a primary hemiacetal and a primary hydroxyl group.

A glycoside bond refers to either a simple glycoside bond or a stable glycoside bond.

A stable ester bond refers to one formed between a hydroxyl group and a carboxylic acid group where at least one of them is a secondary hydroxyl group, a tertiary hydroxyl group, a secondary carboxylic acid group or a tertiary carboxylic acid group. And the stable ester bond derived from cyclopropanecarboxylic acid is most known (Bender et al., 2008).

A simple ester bond refers to one derived from a primary hydroxyl group and a primary carboxylic acid group.

An ester bond refers to either a simple ester bond or a stable ester bond.

A stable disulfide bond refers to one formed between two thiol groups, at least one of which is a secondary thiol or a tertiary thiol.

A simple disulfide bond refers to one formed between two primary thiol groups.

A disulfide bond refers to either a simple disulfide bond or a stable disulfide bond.

Compared to their simple counterparts, stable amide bonds, stable glycoside bonds, stable ester bonds and stable disulfide bonds are more resistant to chemical or enzymatic hydrolysis.

The term “enzymatically cleavable peptide (or amino acid)” refers to a peptide chain (or an amino acid) linking a drug linker and a polar drug moiety via a cleavable bond for the polar drug release, for example upon enzymatic treatment. Examples of a cleavable bond include disulfide bonds, acid-labile bonds that are cleavable at acidic pH, and bonds that are cleavable by hydrolases (e.g., peptidases, esterases, and glucuronidases).

The term “self-immolative spacer” refers to a chemical moiety having a first bond linking to an enzymatically cleavable peptide and a second bond linking to a polar drug moiety, in which both the first and second bonds are capable of spontaneous degradation in response to a specific stimulus.

An antigen is an entity to which a ligand or a secondary targeting moiety specifically binds.

The term “ligand” used herein covers monoclonal antibodies, PEGylated monoclonal antibodies (Chapman 2002), Fc-fusion proteins (Czajkowsky et al., 2012; Jafari et al., 2017), albumin, PEGylated albumin (Akbarzadehlaleh et al., 2016), and albumin-fusion proteins (Rogers et al., 2015; Wang et al., 2020b).

Non-limiting examples of a secondary targeting moiety include glucose (Calvaresi and Hergenrother, 2013), AGM-330 (Kim et al., 2020). iRGD peptide (Sugahara et al., 2009), APTEDB (Saw et al., 2021), Centyrin (Goldberg et al., 2016), F3 peptide (Lam et al., 2016). DVN peptide (Zhu et al., 2018), Pasireotide (Wang et al., 2022), Afamelanotide (Wang et al., 2022). Etelcalcetide (Wang et al., 2022). Somatostatin, Folic acid (Dharmatti et al., 2019), BMS-1166 (Pan et al., 2021), aptamer (Jiang et al., 2015; Kuai et al., 2017).

The term monosaccharide as used herein covers monosaccharides and their derivatives. A monosaccharide has the chemical formula: (CH₂O)ₓ, where x≥3. A large number of monosaccharide derivatives are known. Non-limiting examples include glucosamine, sialic acid, galactosamine, ascorbic acid, mannitol, glucuronic acid, muramic acid, and Neuraminic acid.

A hydrophilic polymer (HP) is frequently applied to increase half-life, reduce immunogenicity, and improve solubility and stability of both small and macromolecules (Greenwald et al., 2003; Fishburn 2008; Schlapschy et al., 2013; Lin et al., 2015; Lyon et al., 2015; Podust et al., 2016; Chan et al., 2017; Hu et al., 2018; Gupta et al., 2019; Hou et al., 2019; Viricel et al., 2019; Tian et al., 2021). Non-limiting examples of HP include PEG (—C₀-C₁₀ alkylene-(CH₂—CH₂—O)ₚ—C₀-C₁₀ alkylene-, wherein subscript p is an integer selected from 2-50; see above for the definition of alkylene); polysarcosine (C₀-C₁₀ alkylene-[N(CH₃)—CH₂—(C═O)—C₀-C₁₀ alkylene-, wherein subscript s is an integer selected from 2-500; PAS (poly Pro-Ala-Ser) [C₀-C₁₀ alkyl-(Pro-Ala-Ser)ₐ-C₀-C₁₀ alkyl, wherein subscript a is an integer selected from 2-501; polypeptoid polymer, zwitterionic polymers, and hydroxyproline polymers.

A functional group refers to either an inert group or a reactive group.

An inert group refers to any chemical non-reactive group. Non-limiting examples of inert groups includes: C₁-C₁₀alkyl, C₁-C₁₀ alkylene, aryl, arylene, C₃-C₈ heterocycle, C₃-C₈ heterocyclo, C₃-C₈ carbocycle, C₃-C₈ carbocyclo, C₁-C₁₀ heteroalkyl, and C₁-C₁₀ heteroalkylene. Said inert group may also be selected from the list of reactive groups.

A reactive group refers to any chemical moiety that is reactive for covalently binding a bindable group. Non-limiting examples of reactive groups include carboxylic acid; amine; aminooxy; hydroxyl; halogen; activated ester such as N-hydroxysuccinimide ester, alkynyl; alkenyl; azide; isocyanate; aldehyde; keto; maleimide, pyridyl disulfide; and thiol.

A bifunctional or multifunctional crosslinker is a PEG-containing or non-PEG-containing chemical reagent that has two or more reactive groups at its ends. These reagents can be used to crosslink two or more molecules together using suitable reactions. Bifunctional or multifunctional crosslinkers include homobifunctional crosslinkers, heterobifunctional crosslinkers, and photoreactive crosslinkers. Homobifunctional (such as glutaraldehyde) crosslinkers are crosslinking agents that have the same functional chemistry at each end of the structure. Heterobifunctional crosslinkers, (such as SMCC (N-Succininidyl 4-(Maleimidomethyl)cyclohexane-1-carboxylate) and Sulfo-SMCC) are crosslinking agents that have different reactive groups at each end of the structure. Photoreactive crosslinkers (such as ABH (p-azidobenzoyl hydrazide)) have photoactivatable reactive groups that only become reactive when exposed to UV or visible light. Examples of reactive groups found in a crosslinker include, but are not limited to, amine for conjugation with the carboxylate group of target molecule; NHS-Ester for the amine group of target molecule; maleimide for the thiol group of target molecule; isocyanate for the hydroxyl group of target molecule; alkyne (—C≡CH) for the azide group (—N₃) of target molecule; hydrazine or aminooxy for the aldehyde or keto group of target molecule, respectively.

The term “compound”, when referring to a compound of this invention, also includes its salts, solvates, and prodrugs. The pharmaceutically acceptable salts include those listed in Handbook of Pharmaceutical Salts: Properties, Selection and Use, 2^{nd} Revised Edition, P. H. Stahl and C. G. Wermuth (Eds.), Wiley-VCH, New York, (2011). In addition to pharmaceutically acceptable salts, other salts are contemplated in the invention. They may serve as intermediates in the purification of compounds or in the preparation of other pharmaceutically acceptable salts, or are useful for identification, characterization or purification of compounds of the invention. A solvate refers to a complex formed between an active compound and a pharmaceutically acceptable solvent. Examples of a pharmaceutically acceptable solvent include water, ethanol, isopropanol, ethyl acetate, acetic acid, and ethanolamine. A prodrug refers to a compound that, after administration, is metabolized into a pharmaceutically active drug. Examples of a prodrug include esters and other pharmaceutically acceptable derivatives.

The compounds of the present invention may contain one or more non-aromatic double bonds or asymmetric centers. Each of them occurs as a racemate or a racemic mixture, a single R enantiomer, a single S enantiomer, an individual diastereomer, a diastereometric mixture, a cis-isomer, or a trans-isomer. Compounds of such isomeric forms are within the scope of this invention. They can be present as a mixture or can be isolated using chiral synthesis or chiral separation technologies.

It is understood that compounds of the present invention may exist as stereoisomers. It is further understood that compounds of the present invention include all forms of stereoisomers including enantiomers, diastereomers, and mixtures thereof. Preferred stereoisomers are predominantly one diastereomer. More preferred stereoisomers are predominantly one enantiomer.

As used herein, the depiction of an asterisk (*) in a chemical formula represents the point of attachment of the group to the corresponding parent formula.

It is recognized that one skilled in the art may treat cancer by administering to a patient presently displaying symptoms an effective amount of a conjugate of this invention. Thus, the terms “treatment” and “treating” are intended to refer to therapeutic treatment and prophylactic measures to prevent relapse, wherein the object is to inhibit or slow down (lessen) an undesired physiological change or disorder, such as, for example, the development or spread of cancer. For purposes of this invention, beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. “Treatment” can also mean prolonging survival as compared to expected survival if not receiving treatment. Those in need of treatment include those already with the condition or disorder as well as those prone to have the condition or disorder. In the context of cancer, the term “treating” includes any or all of: inhibiting growth of tumor cells, cancer cells, or of a tumor; inhibiting replication of tumor cells or cancer cells, lessening of overall tumor burden or decreasing the number of cancerous cells, and ameliorating one or more symptoms associated with the disease.

Further, it is recognized that one skilled in the art may treat cancer by administering to a patient at risk of future symptoms an effective amount of the conjugate of this invention and is intended to include prophylactic treatment of such.

As used herein, the term “effective amount” of a compound of formula I refers to an amount, that is a dosage, which is effective in treating a disorder, such as the diseases described herein. The attending diagnostician, as one skilled in the art, can readily determine an effective amount by the use of conventional techniques and by observing results obtained under analogous circumstances. In determining an effective amount or dose of a conjugate of this invention, a number of factors are considered, including, but not limited to the conjugate to be administered; the co-administration of other agents, if used; the species of mammal; its size, age, and general health; the degree of involvement or the severity of the disorder, such as cancer, the response of the individual patient; the mode of administration; the bioavailability characteristics of the preparation administered; the dose regimen selected; the use of other concomitant medication; and other relevant circumstances. The combinations of this invention may be administered at therapeutically effective single or divided daily doses. The active components of the combination may be administered in such doses which are therapeutically effective in monotherapy, or in such doses which are lower than the doses used in monotherapy, but when combined result in a desired (joint) therapeutically effective amount.

A conjugate of this invention may be administered alone or in the form of a pharmaceutical composition with pharmaceutically acceptable carriers, diluents or excipients. Such pharmaceutical compositions and processes for making the same are known in the art (See, e.g., Remington: The Science and Practice of Pharmacy, A. Adejare, Editor, 23rd Edition, Academic Press, 2020).

Within this invention it is to be understood that the combinations, compositions, kits, methods, uses or compounds for use according to this invention may envisage the simultaneous, concurrent, sequential, successive, alternate, or separate administration of the active ingredients or components.

The administration of a conjugate of this invention and the at least one other pharmacologically active substance may take place by co-administering the active components or ingredients, such as e.g., by administering them simultaneously, concurrently, sequentially, successively, alternately, or separately, in one single or in two or more separate formulations or dosage forms.

For example, simultaneous administration includes administration at substantially the same time. This form of administration may also be referred to as “concomitant” administration. Concurrent administration includes administering the active agents within the same general time period, for example on the same day(s) but not necessarily at the same time. Alternate administration includes administration of one agent during a time period, for example over the course of a few days or a week, followed by administration of the other agent(s) during a subsequent period of time, for example over the course of a few days or a week, and then repeating the pattern for one or more cycles. Sequential or successive administration includes administration of one agent during a first time period (for example over the course of a few days or a week) using one or more doses, followed by administration of the other agent(s) during a second and/or additional time period (for example over the course of a few days or a week) using one or more doses. An overlapping schedule may also be employed, which includes administration of the active agents on different days over the treatment period, not necessarily according to a regular sequence. Variations on these general guidelines may also be employed, e.g., according to the agents used and the condition of the subject.

The elements of the combinations of this invention may be administered (whether dependently or independently) by methods customary to the skilled person, e.g. by oral, enteral, parenteral (e.g., intramuscular, intraperitoneal, intravenous, transdermal or subcutaneous injection, or implant), nasal, vaginal, rectal, or topical routes of administration and may be formulated, alone or together, in suitable dosage unit formulations containing conventional non-toxic pharmaceutically acceptable carriers, excipients and/or vehicles appropriate for each route of administration.

Without further elaboration, it is believed that one skilled in the art can, based on the above description, utilize the present invention to its fullest extent. The following examples are to be construed as merely illustrative and not limitative of the remainder of the disclosure in any way whatsoever.

All publications cited herein are hereby incorporated by reference in their entirety.

### BRIEF DESCRIPTION OF DRAWINGS

FIG. 1 shows tumor volume (mm³) as a function of time (day) in a JIMT-1 xenograft model, comparing tumor growth inhibition by conjugate C-APDC17 of this invention with Kadcyla and Enhertu, two commercial ADCs.

FIG. 2 shows a single intravenous dose (10 mg/kg at day zero) of one of four conjugates of this invention (i.e., C-APDC5, C-APDC-8, C-APDC9, and C-APDC16) each was more efficacious than two doses (10 mg/kg at day zero and day 7, respectively) of Kadcyla and Enhertu. Tumor volume as a function of time was determined using the formula (L×W2)/2.

FIG. 3 demonstrates that a dual-targeting APDC conjugate, C-APDC10, of this invention was more efficacious than a monotargeting ADC, Kadcyla, or a monotargeting APDC, C-APDC15, in killing cancer cells in a cell-based assay.

FIG. 4 illustrates antitumor activities of conjugates I-APDC2, I-APDC3, I-APDC4, and I-APDC6 of this invention in an N87 xenograft model, performing significantly better than Her-T785, a Bolt's immune-stimulating ADC.

FIG. 5 shows in an N87 xenograft model the tolerability of conjugates I-APDC2, I-APDC3, I-APDC4, and I-APDC6 of this invention, similar to that of Her-T785.

FIG. 6 demonstrates in a JIMT-1 xenograft model that conjugates I-APDC7, I-APDC8, I-APDC9, and I-APDC10 of this invention each effectively eliminate tumor cells in Kadcyla-resistant tumor xenograft models in the absence of B, T or NK cell activities, performing significantly better than Her-T785.

### DETAILED DESCRIPTION

The conjugates of this invention are stable in circulation as well as in endosome to prolong their pharmacokinetics as the PD linker contains no cutting sites of cathepsins. The conjugates can be delivered into tumor interstitium via endosomal recycling to effectively kill interstitial tumor cells. In addition, the conjugates typically have a high DLR (drug/ligand ratio) value of about 7-8 and are versatile in the incorporation of a variety of polar drugs as payloads. And in contrast to conventional ADCs with a non-cleavable linker, the PD payloads of the conjugates are quickly liberated via the cleavage of an amide bond or a glycoside bond in lysosome to achieve an improved bioavailability and pharmacodynamics. One or more optional secondary targeting moieties can be included in the PD linker of the conjugates to improve the latter's activity against cancer cells lacking the antigen recognized by the ligand. Also, one or more optional hydrophilic polymer moieties can be included in the PD linker of the conjugates to improve the latter's solubility and pharmacokinetics.

The linker complex in the conjugates of this invention is stable in circulation to limit damages caused by the agent to healthy tissues before the conjugate is delivered to tumor cells. Once the conjugate enters tumor cells, the linker complex prevents wasteful dumping of a precious payload in endosome to ensure delivery of the conjugate to tumor interstitium via endosomal recycling. Further, it facilitates optimal payload release in lysosome.

A majority of IgGs and IgG-based conjugates can be delivered to the tumor interstitium via the intrinsic mechanism of endosomal recycling.

Not to be bounded by any theory, a conjugate of this invention is passively delivered to the tumor tissue via enhanced permeability and retention (EPR), followed by the specific interaction between the antibody ligand and the tumor antigen of interest. After internalization and transportation to endosome, the conjugate exploits the intrinsic mechanism of endosomal recycling to bypass the barrier posed by TIFP and help drug delivery into the tumor interstitium. As such, the conjugate is stable in endosome and effective in killing interstitial tumor cells.

The conjugates of this invention each contain a ligand moiety and a moiety derived from a compound of formula I described above. The ligand is bonded to the compound via a covalent bond formed between a functional group from the ligand and L1 in the compound of formula I. The functional group is sulfhydryl, amino, glutamine, or formyl. Exemplary bonds are a thioether bond (C—S, e.g., via SH in a cysteine residue of the ligand and a maleimide moiety of L1 in the compound of formula I) and an amide bond (CONH, e.g., via a lysine or glutamine residue of the ligand). Thiol groups in a ligand can be obtained from reduction of interchain disulfide residues and/or thiol-containing residues.

Exemplary ligands include monoclonal antibodies, PEGylated monoclonal antibodies, Fc-fusion proteins albumin, PEGylated albumin, and albumin-fusion proteins.

The antibodies can be autologous or allogeneic antibodies. The terms “allogeneic antibody” or “alloantibody” refer to an antibody that is not from the individual in question (e.g., a cancer patient), but is from the same or different species, and has been engineered to reduce, mitigate, or avoid recognition as a xeno-antibody (e.g., non-self). For example, the “allogeneic antibody” can be a humanized antibody including immunoglobulin G (IgG) or immunoglobulin A (IgA).

In preferred embodiments, the antibody is a monoclonal antibody of a defined sub-class (e.g., IgG₁, IgG₂, IgG₃, IgG₄, IgA₁, or IgA₂). If combinations of antibodies are used, the antibodies can be from the same subclass or from different subclasses. For example, the antibodies can be IgG₁ antibodies. Various combinations of different subclasses, in different relative proportions, can be obtained by those of skill in the art. In some cases, a specific subclass, or a specific combination of different subclasses can be particularly effective at cancer treatment or tumor size reduction.

As an illustration, the antibody binds to an antigen of a cancer cell. For example, the antibody can bind to a target antigen that is present at an amount of at least 10; 100; 1,000; 10,000; 100,000; 1,000,000; 2.5×10⁶; 5×10⁶; or 1×10⁷ copies or more on the surface of a cancer cell. Further, the antibody can bind to an antigen on a cancer or immune cell at a higher affinity than a corresponding antigen on a non-cancer cell. For example, the antibody may preferentially recognize an antigen containing a polymorphism that is found on a cancer or immune cell as compared to recognition of a corresponding wild-type antigen on the non-cancer or non-immune cell. In some cases, the antibody binds a cancer or immune cell with greater avidity than a non-cancer or non-immune cell. For example, the cancer or immune cell can express a higher density of an antigen, thus providing for a higher affinity binding of a multivalent antibody to the cancer or immune cell.

Suitable ligands include abagovomab, abatacept (also known as ORENCIA™), abciximab (also known as REOPRO™, c7E3 Fab), adalimumab (also known as HUMIRA™), adecatumumab, alemtuzumab (also known as CAMPATH™, MabCampath or Campath-1H), altumomah, afelimomab, anatumomab mafenatox, anetumumab, anrukizumab, apolizumab, arcitumomab, aselizumab, atlizumab, atorolimumab, Avelumab, bapineuzumab, basiliximab (also known as SIMULECT™), bavituxinab, bectumomab (also known as LYMPHOSCAN™), belimumab (also known as LYMPHO-STAT-B™), bertilimumab, besilesomab, bevacizumab (also known as AVASTIN™), biciromab brallobarbital, bivatuzumab mertansine, campath, canakinumab (also known as ACZ885), cantuzumab mertansine, capromah (also known as PROSTASCINT™), catumaxonab (also known as REMOVAB™), cedelizumab (also known as CIMZIA™), certolizumab pegol, cetuximah (also known as ERBTTUX™), clenoliximah, dacetuzumab, dacliximab, daclizumab (also known as ZENAPAX™), denosumab (also known as AMG 162), detumomah, dorlimonab aritox, dorlixizumab, duntumumab, durimulunab, durmulumab, ecromeximab, eculizumab (also known as SOLIRIS™), edobacomab, edrecolonab (also known as Mab17-1A, PANOREX™), efalizumab (also known as RAPTIVA™), efungumab (also known as MYCOGRAB™), elsilimomab, enlimomab pegol, epitumomab cituxetan, efalizumab, epitumomab, epratuzumab, erlizumab, ertuinaxomab (also known as REXOMUN™), etanercept (also known as ENBREL™), etaracizumab (also known as etaratuzumab, VITAXIN™ ABEGRIN™), exbivirumab, fanolesomab (also known as NEUTROSPEC™), faralimomab, felvizumab, fontolizumab (also known as HUZAF™), galiximab, gantenerumab, gavilimomab (also known as ABXCBL™), gemtuzumab ozogamicin (also known as MYLOTARG™), golimumab (also known as CNTO 148), gomiliximab, ibalizumab (also known as TNX-355), ibritumomab tiuxetan (also known as ZEVALIN™), igovomab, imeiromab, infliximab (also known as REMICADE™), inolimomab, inotuzumab ozogamicin, ipilimumab (also known as MDX-010, MDX-101), iratumumab, keliximab, labetuzunab, lemalesomab, lebrilizumab, lerdelimumab, lexatumunab (also known as, HGS-ETR2. ETR2-STO 1), lexitumumab, libivirumab, lintuzumab, lucatumumab, lumiliximab, mapatumumab (also known as HGSETR1, TRM-1), maslimomab, matuzumab (also known as EMD72000), mepolizumab (also known as BOSATRIA™), metelimumab, milatuzumab, minretumomab, mitumomab, morolimunab, motavizumab (also known as NUMAX™), muromonab (also known as OKT3), nacolomab tafenatox, naptumomab estafenatox, natalizumab (also known as TYSABRI™, ANTEGREN™), nebacumab, nerelimomab, nimrotuzumab (also known as THERACIM hR3™, THERA-CIM-hR3™, THERALOC™), nofetumomah merpentan (also known as VERLUMA™), ocrelizumab, odulimomab, ofatumumab, ormalizumab (also known as XOLAIR™), oregovomab (also known as OVAREX™), otelixizumab, pagibaximah, palivizumab (also known as SYNAGIS™), panitumumab (also known as ABX-EGF, VECTIBIX™), pascolizumab, pemtumomab (also known as THERAGYN™), pertuzumab (also known as 2C4, OMNITARG™), pexelizumab, pintumomab, priliximab, pritumumab, ranibizumab (also known as LUCENTIS™), raxibacumab, regavirumab, reslizumab, rituximab (also known as RITUXAN™, MabTHERA™), rovelizumab, ruplizumah, satumomah, sevirumab, sibrotzurmab, siplizumab (also known as MEDI-507), sontuzumab, stamulumab (also known as MYO-029), sulesomab (also known as LEUKOSCAN™), tacatuzumab tetraxetan, tadocizumab, talizumab, taplitumormab paptox, tefibazumab (also known as AUREXIS™), telimomab aritox, teneliximab, teplizumab, ticilimumab, tocilizunmab (also known as ACTEMRA™), toralizumab, tositumomab, trastuzumab (also known as HERCEPTIN™), tremelimumab (also known as CP-675,206), tucotuzumab celmoleukin, tuvirumab, urtoxazumab, ustekinumab (also known as CNTO 1275), vapaliximab, veltuzumab, vepalimomab, visilizumab (also known as NUVION™), volocixinab (also known as M200), votumumab (also known as HUMASPECT™), zalutumumab, zanolimumah (also known as HuMAX-CD4), ziralimumab, zolimomab aritox, daratumumab, elotuxumab, obintunzumab, olaratumab, brentuximab vedotin, afibercept, abatacept, belatacept, afibercept, etanercept, romiplostim, SBT-040 (sequences listed in US 2017/0158772. Preferably, the ligand is Herceptin, Albumin, Perjeta, or any combination thereof.

In preferred embodiments, the molar ratio of the ligand and the compound of formula I in the conjugate is between 1:1 and 1:20 (e.g., 1:1 to 1:16, 1:1 to 1:12, 1:2 to 1:12, 1:2 to 1:10, 1:4 to 1:10, 1:2 to 1:8; 1:6 to 1:8; and 1:5 to 1:12).

Also within the scope of the invention is a compound of formula I:

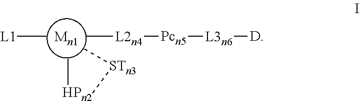

The compound has four components: a ligand linker L1, an Mₙ₁ linker complex, a drug linker L2, and a first polar drug moiety PD1. Optionally, the compound has one to five hydrophilic polymer moieties HP, one to five secondary targeting moieties ST, or any combinations thereof.

The ligand linker contains a functional group capable of reacting with a ligand. In a conjugate of this invention, the ligand linker connects the ligand and the Mₙ₁ linker complex.

The Mₙ₁ linker complex contains 1 to 15 monomeric units represented by M. Each monomeric unit M in the Mₙ₁ linker complex can be the same or different from each other. Each M covalently bonds to its adjacent M via stable amide bond, a stable glycoside bond, a stable ester bond, a stable disulfide bond, a C—S bond, a C—O bond, a C—N bond, a urea moiety (—NH—C(O)—NH—) or a triazole moiety so that the Mₙ₁ complex is stable, namely resistant to enzymatical cleavage. Mₙ₁ can be a linear oligomer, a branched oligomer having four or more monomeric units, a cyclic oligomer, or a mosaic oligomer which is a cyclic oligomer with one or more ring members having one or more substituents such as a monomeric unit M or a linear, branched, cyclic, or mosaic oligomer containing two to ten monomeric units M. Each monomer of the Mₙ₁ complex exists in residual form.

M, in each occurrence, can be independently selected from:
(1) Natural or non-natural amino acids, with a preference for those with a secondary amine groups (such as N-methyl-L-alanine; N-methyl-L-serine; N2-methyl-L-asparagine, N-(4-hydroxyphenyl)glycine, iminodiacetic acid, N-(2-aminoethyl)glycine, sarcosine) or those from the following groups of amino acids: (a) cyclic α-amino acids (e.g., 1-aminocyclopropane-1-carboxylic acid, cycloleucine, 3-aminotetrahydrofuran-3-carboxylic acid, 4-aminotetrahydro-furan-3-carboxylic acid, 4-amino-tetrahydro-pyran-4-carboxylic acid); (b) cyclic O-amino acids (i.e., 2-amino-cyclopropanecarboxylic acid, and 4-aminotetrahydro-3-thiophenecarboxylic acid); and (c) cyclic secondary amino acids, such as azetidine-2-carboxylic acid, pipecolic acid, proline, 4-aminoproline, 4-hydroxyproline, 4-oxazolidinecarboxylic acid, 4-azidoproline, L-trans-pyrrolidine-2,4-dicarboxylic acid, pyrrolidine-2,5-dicarboxylic acid, piperidine-2,6-dicarboxylic acid, 2,4-cis-piperidine-2,4-dicarboxylic acid, piperidine-2,3-dicarboxylic acid, imidazolidine-2-carboxylic acid, 3-morpholine carboxylic acid, nipecotic acid, 3-(methylamino)tetrahydrofuran-3-carboxylic acid, 3-((methylamino)methyl)azetidine-1-carboxylic acid, 3-((methylamino)methyl)piperidine-1-carboxylic acid, 2-((methylamino)methyl)pyrrolidine-1-carboxylic acid, 3-((methylamino)methyl)pyrrolidine-1-carboxylic acid;
(2) 3,6-bis-(4-amino-butyl)-piperazine-2,5-dione (CDK) and its derivatives. It is noted that 3,6-bis-(4-amino-butyl)-piperazine-2,5-dione can be deemed as a cyclic dipeptide (i.e., cyclic di-lysine);
(3) monosaccharides;
(4) bifunctional crosslinkers or multi-functional crosslinkers
(5) polyamines with at least one amino group being secondary or tertiary; or
(6) diamino-dicarboxylic acids.

In a preferred embodiment, the Mₙ₁ linker complex contains at least a monomeric unit derived from 3,6-bis-(4-amino-butyl)-piperazine-2,5-dione, a cyclic peptide, or a diamino dicarboxylic acid (e.g., 2,5-diaminobenzene-1,4-dicarboxylic acid).

The drug linker L2 connects the Mₙ₁ linker complex to the first polar drug moiety PD1. The bond between L2 and PD1 is an amide bond or a glycoside bond. Not to be bound by any theory, the bond between L2 and PD1 are stable enough in circulation and endosome of a patient but quickly cleaved by lysosomal enzymes to liberate PD1, eliminating the slow process of antibody degradation mandated by conventional ADCs with a non-cleavable linker to release active metabolites.

L2 can be a bifunctional crosslinker containing —C1-C10 alkylene- or -C0-C10 alkylene-(CH2-CH2-O)g-C0-C10 alkylene- where g is an integer of 1-10.

Polar drug PD1 is a biologically active ingredient in a medicament, dissolved in water at 25° C. at a ratio of the polar drug to water (weight to weight) of at least 1 to 10000, 1 to 1000, 1 to 100, 1 to 30, 1 to 10, or 1 to 1. A hydrophobic drug can be modified to be a polar drug by introducing one or more moieties, e.g., —NH₂, —OH, and COOH, that are polar, hydrophilic, or ionic. Non-limiting examples of hydrophobic drugs include maytansinoids, auristatins, calicheamicins, duocarmycins, pyrrolobenzodiazepines, lapatinib, and immune adjuvants (e.g., TLR7/8 agonist 1, CAS No.: 1620278-72-9); T785 (Ackerman et al., 2021), and diABZI-C2-NH2 (a STING agonist, CAS No.: 2137975-93-8).

The compound of this invention can contain a second polar drug moiety (PD2), which is linked to the linker complex Mₐ₁ via L2 or a drug linker similar to L2 as described above.

As indicated above, the compound also optionally contains one or more secondary targeting moieties and one or more hydrophilic polymer moieties.

The details of one or more embodiments of the invention are set forth in the description below. Other features, objects, and advantages of the invention will be apparent from the description and from the claims.

Without further elaboration, it is believed that one skilled in the art can, based on the above description, utilize the present invention to its fullest extent. The following examples are to be construed as merely illustrative and not limitative of the remainder of the disclosure in any way whatsoever.

### EXAMPLES

The following reagents, cell lines and animals were used in (1) preparing compounds of formula (I), and conjugates of this invention, and (2) cell-based assays and animal studies. Their suppliers are provided below, as well as their CAS registry numbers or catalog numbers (Cat No).

TLR7/8 agonist 1 dihydrochloride (Ag), MedChemExpress, CAS No. 1620278-72-9; Na-Fmoc-NO-Boc-L-2,3-diaminopropionic acid (Fmoc-Dap(Boc)-OH), Tokyo Chemical Industry Co., Ltd., CAS No. 162558-25-0; HATU, Combi-Blocks, CAS No. 148893-10-1; N-Ethyldiiso-propylamine (DIPEA or DIEA), Alfa Aesar, CAS No. 7087-68-5; DMSO, Sigma-Aldrich; Acetonitrile (ACN), J. T. Baker; (2R,4S)-1-Fmoc-4-Boc-amino pyrrolidine-2-carboxylic acid (Fmoc-4AP(Boc)-OH), Combi-Blocks, CAS No. 1820570-42-0; Bis-PEG6-NHS ester, Broadpharm, CAS No. 1526718-98-8; HEPES, Sigma-Aldrich, CAS No. 7365-45-9; Sodium hydroxide (NaOH), Sigma-Aldrich; (2R,4S)-1-Boc-4-Aminopyrrolidine-2-carboxylic acid (NH2-4AP(Boc)-OH), AK Scientific, CAS No. 132622-78-7; Trifluoroacetic acid, Sigma-Aldrich, CAS No. 76-05-1; m-PEG12-NHS ester, Broadpharm, CAS No. 174569-25-6; (2R,4S)-1-Fmoc-4-Boc-amino pyrrolidine-2-carboxylic acid (Boc-4AP(Fmoc)-OH), AK Scientific, CAS No. 1253791-18-2; 1-(3-Dimethylaminopropyl)-3-ethylcarbodiimide (EDC) hydrochloride, Sigma-Aldrich, CAS No. 25952-53-8; N-hydroxysuccinimide, Sigma-Aldrich, CAS No. 6066-82-6; 3,6-bis(4-aminobutyl)-2,5-piperazinedione (CDK), Broadpharm, CAS No. 23409-32-7; Morpholine, Alfa Aesar; Mal-PEG2-acid, Broadpharm, CAS No. 1374666-32-6; Herceptin (trastuzumab), Roche, Cat No. 000961-1-CTN-05; Perjeta, Roche, Cat No. H0536B33; DL-Dithiothreitol (DTT), VWR LIFE SCIENCE, CAS No. 3483-12-3; Sephadex™ G25 Fine, Cytiva; Sephadex™ G-100, Cytiva; D-proline, AK Scientific, CAS No. 344-25-2; m-PEG24-NHS ester, Broadpharm, CAS No. 2395839-96-8; 2,5-Diaminoterephthalic acid (H2DATA), Ambeed, CAS No. 945-30-2; Boc-Sar-Osu, Combi-Blocks, CAS No. 80621-90-5; HOBT, Sigma-Aldrich, CAS No. 123333-53-9; mPEG12-amine, Broadpharm, CAS No. 1977493-48-3; Triethylamine, Sigma-Aldrich, CAS No. 121-44-8; Acid-PEG5-NHS, Broadpharm, CAS No. 1343476-41-4; N,N-Dimethylformamide (DMF), Sigma-Aldrich, CAS No. 68-12-2; Di(N-succinimidyl)adipate (DSA), Broadpharm, CAS No. 59156-70-6; Sucrose, VWR LIFE SCIENCE, CAS No. 57-50-1; sodium succinate dibasic hexahydrate, Sigma-Aldrich, CAS No. 6106-21-4; Tween-20, VWR LIFE SCIENCE, CAS No. 9005-64-5; m-PEG24-amine, Broadpharm, CAS No. 32130-27-1; Gel Filtration Standard, Bio-Rad, Cat No. 151-1901; Mertansine (DM1), Cyaman, CAS: 139504-50-0; N-Me-L-Ala-maytansinol, Boc Sciences, CAS: 77668-69-0; Monomethyl auristatin E (MMAE), Long Tail Intl. (Taiwan), CAS: 474645-27-7; DM4, MedChemExpress, CAS: 796073-69-3; SN38, MedChemExpress, CAS: 86639-52-3; Lapatinib, MedChemExpress, CAS: 231277-92-2; Kadcyla, Roche, NDC 50242-088-01; Enhertu, Daiichi-Sankyo AstraZeneca, NDC 65597-406-01; (2S)-2-amino-3-{[(tert-butoxy)carbonyl]amino}propanoic acid [NH2-Dap(Boc)-OH], Combi-Block, CAS: 74536-29-1; (2R,4S)-4-((tert-Butoxycarbonyl)amino)pyrrolidine-2-carboxylic acid (Boc-4AP(NH2)-OH), Ambeed, CAS: 1279030-48-6; L-Asparagine, Combi-Block, CAS: 70-47-3; L-Cysteine hydrochloride monohydrate, Sigma, CAS: 7048-04-6; (S)-3-Amino-2-(tert-butoxycarbonylamino)propionic Acid (Boc-Dap-OH), Tokyo Chemical Industry Co., Ltd., CAS: 73259-81-1; N^{α}-(tert-Butoxycarbonyl)-L-lysine (Boc-Lys-OH), Tokyo Chemical Industry Co., Ltd., CAS: 13734-28-6; Serine, Tokyo Chemical Industry Co., Ltd., CAS: 56-45-1; 3-mercaptolactic acid, Toronto Research Chemicals, CAS: 2614-83-7; H-Lys(Boc)-OH, Combi-Block, CAS: 2418-95-3; L-(+)-Penicillamine, Santa Cruz, CAS: 1113-41-3; L-5-Amino-2-N-Boc-amino-pentanoic acid, Santa Cruz, CAS: 21887-64-9; 1-[(tert-Butoxycarbonyl)-amino]cyclopropanecarboxylic acid, Combi-Block, CAS: 88950-64-5; Maleimidocaproic acid, Combi-Block, 55750-53-3; N-Succinimidyl Maleimidoacetate (AMAS), Tokyo Chemical Industry Co., Ltd., CAS: 55750-61-3; N-Succinimidyl 3-Maleimidopropionate (BMPS), Tokyo Chemical Industry Co., Ltd., CAS: 55750-62-4; 1,2-Bis(maleimido)ethane, Tokyo Chemical Industry Co., Ltd., CAS: 5132-30-9; Di(N-succinimidyl) adipate (DSA), Broadpharm, CAS: 59156-70-6; N-Succinimidyl 4-(N-Maleimidomethyl)cyclohexanecarboxylate (SMCC), Tokyo Chemical Industry Co., Ltd., CAS: 64987-85-5; N-Succinimidyl 3-(2-Pyridyldithio)propionate (SPDP), Tokyo Chemical Industry Co., Ltd., CAS: 68181-17-9; Iodo-PEG3-carboxylic acid, Lumiprobe, Cat. 2063-1g; N-Fmoc-3-iodo-L-alanine methyl [Fmoc-Ala(Iodo)-Ome], Ambeed, CAS: 156017-42-4; 1,2-Bis(2-iodoethoxy)ethane, Combi-Blocks, 36839-55-1; 4-(2-Boc-aminoethyl)piperidine, AK Scientific, Inc., CAS:165528-81-4); t-Boc-N-amido-PEG6-acid, Broadpharm, CAS: 882847-13-4; Amino-PEG8-alcohol, Broadpharm, CAS: 352439-37-3; m-peg8-amine, Broadpharm, CAS: 869718-81-0; Amino-PEG8-acid, Broadpharm, CAS: 756526-04-2; amino-peg24-acid, Broadpharm, CAS: 196936-04-6; Glucose-PEG-NH2, RuixiBiotechCo. Ltd, Cat No R—CP-060; 2-(4-(2,5-dioxo-2H-pyrrol-1(5H)-yl)phenyl)acetic acid (4-Maleimidophenylacetic Acid), Broadpharm, CAS: 91574-45-7; tris(2-carboxyethyl)phosphine (TCEP), GoldBio, CAS: 51805-45-9; Dichloromethane (DCM) Anhydrous, Sigma, CAS: 75-09-2; Pyridine anhydrous, Sigma, CAS: 110-86-1; N,N′-Dicyclohexyl-carbodiimide (DCC), Tokyo Chemical Industry Co., Ltd., CAS: 538-75-0; 4-Dimethyl-aminopyridine (DMAP), Tokyo Chemical Industry Co., Ltd., CAS: 1122-58-3; Human Albumin 20%, CSL Behring, ATC Code: B05A A01; 1-(4-Aminobutyl)-2-butyl-1H-imidazo[4,5-c]quinolin-4-amine (T785), Amadis Chemical, CAS No. 313350-31-1; Muramic acid, Toronto Research Chemicals, CAS: 1114-41-6. 96-well plate, Nunc, Thermo Fisher, Cat No 167008; Sodium Chloride, Sigma, Cat No S3014, Potassium Chloride, Amresco, Cat No 395; Sodium Phosphate Dibasic Anhydrius, Amresco, Cat No 404; Potassium phosphate monobasic, Sigma, Cat No 795488; Sodium carbonate, Sigma, Cat No 497198; Sodium bicarbonate, Sigma, Cat No 144568; Bovine Serum Albumin, Gibco, Cat No 30063721; Ethylenediaminetetraacetic acid disodium salt dehydrate, Sigma, Cat No E4884; Citric acid, Merck, Cat No 77929; DM1 Monoclonal Antibody (A4G2), Invitrogen, Cat No MA5-42527; Her2/ERBB2 Protein, Human, Recombinant (ECD), SinoBiological, Cat No 10004-HCCH; Her2/ERBB2 Protein, Human, Recombinant (ECD), Biotinylated, SinoBiological, Cat No 10004-HCCH—B; Goat F(ab′)2 Anti-Human IgG Fc (HRP), Abcam, Cat No ab98530; Peroxidase-conjugated Streptavidin, Jackson Immunoresearch, Cat No 16030084; 3,3,5,5-Tetramethylbenzidine, Sigma, Cat No 860336-5G; Puromycin, Sigma, Cat No P8833; DMEM (GIBCO, Cat No 12100-061; Heat-inactivated fetal bovine serum (FBS), GIBCO, Cat No 10437-028; RPMI-1640, GIBCO, Cat No 31800-022; McCoy's 5A, Sigma, Cat No M4892; IMDM, GIBCO, Cat No 12200-036; M199, GIBCO, Cat No 31100-035; Heparin, Sigma, Cat No H3149; ECGS, Sigma, Cat No 02-102; and CellTiter-Glo® Luminescent Cell Viability Assay, Promega, Cat No G7571.

Breast cancer cell line MDA-MB-231 and SK-BR-3, gastric cancer cell line NCI-N87, ovarian cancer cell line SK-OV-3, and pancreatic cancer cell line Capan-1 were purchased from ATCC. Breast cancer cell line JIMT-1 was purchased from Deutsche Sammlung von Mikroorganismen und Zellkulturen (DSMZ, Germany). Gastric cancer cell line MKN45 was purchased from RIKEN. Human umbilical vein endothelial cell line (HUVAC) was purchased from BCRC (Bioresource Collection and Research Center, Hsinchu City, Taiwan).

Human MDR1 overexpressed NCI-N87 (NCI-N87-hMDR1) cell line was produced by lentiviral vector-mediated gene transduction. Briefly, the lentiviral transfer vector contains both hEF1α promotor-driven hMDR1 coding sequence (CDS) and hPGK promoter-driven EGFP-IRES-Puro bicistronic cassette. The lentivirus was packaged using second-generation lentiviral system in 293T cell line. After transfection, stable NCI-N87-hMDR1 was selected and maintained in puromycin (0.55 μg/mL) containing RPMI-1640 medium. JIMT-1 and MDA-MB-231 were maintained in DMEM supplemented with 10% heat-inactivated fetal bovine serum (FBS). NCI-N87, NCI-N87-hMDR1, SK—OV-3, and MKN45 were maintained in RPMI-1640 supplemented with 10% heat-inactivated FBS. SK-BR-3 was maintained in McCoy's 5A supplemented with 10% heat-inactivated FBS. Capan-1 was maintained in IMDM supplemented with 20% heat-inactivated FBS. HUVAC was maintained in M199 supplemented with 25 U/mL heparin, 30 μg/mL ECGS and 10% heat-inactivated FBS. All cell lines were cultured at 37° C. under 5% CO2 atmosphere.

BALB/c mice, BALB/c Nude mice and Sprague-Dawley (SD) rats were obtained from BioLASCO Co., Ltd (Taiwan). T-DM1 (Trastuzumab emtansine) as a comparative sample was purchased from Shanghai Union Dispensary Co. (Taipei, Taiwan). All animal studies were conducted in accordance with the Animal Care and Use Committee in a facility accredited by the association for Assessment and Accreditation of laboratory Animal Care.

The following columns were used for separation and analysis: ACE® 5 C18-300, 250×4.6 mm, Advanced Chromatography Technologies Ltd (ACE), Cat No. ACE-221-2546; ACE® 5 phenyl-300 250×4.6 mm, ACE, Cat No. ACE-225-2546; Zorbax® RRHD Eclipse Plus C18, 95 Å, 2.1×50 mm, 1.8 μm, Agilent, Cat No. 959757-902; ENrich™ SEC 650 10×300 Column, Biorad, Cat. #7801650, Serial #100601.

Instruments were used to carried out preparation or analysis: High-performance liquid chromatography (HPLC), Agilent, model: 1260 Infinity® II; 6545 Q-TOF LC/MS, Agilent, model: G6545B; ThermoCell Mixing Block, Bioer, model: MB-101; Manifold Lyophilzer, Uniss, model: FDM-5; ImageXpress® Micro Imaging system, Molecular Devices.

Compounds prepared below were characterized and confirmed using mass spectrometer (Agilent 6545 Q-TOF LC/MS equipped with an EclipsePlusC18 RRHD 1.8 um 2.1×50 mm).

Several procedures were followed to prepare exemplary compounds and conjugates of this invention. They are briefly described below.

### Protocol for Removal of Boc Protection Group (Boc Protocol)

The tert-butoxycarbonyl (Boc) protection group of a compound was removed by incubation with 70% trifluoroacetic acid (TFA) at room temperature for 1 hour (yields about 90-99%). The desired product was purified by HPLC (Agilent® 1260 Infinity® II with ACE C18-300 column), followed by lyophilization with Manifold Lyophilizer (−80° C., 10 mTorr).

### Protocol for Removal of TRT Protection Group (TRT Protocol)

The trityl (TRT) protection group of a compound was removed by incubation with 80-90% trifluoroacetic acid (TFA) at room temperature for 1 hour (yields about 90-99%). The desired product was purified by HPLC (Agilent® 1260 Infinity® II with ACE C18-300 column), followed by lyophilization with Manifold Lyophilizer (−80° C., 10 mTorr).

### Protocol for Removal of Fmoc Protection Group (Fmoc Protocol)

The fluorenylmethyloxycarbonyl (Fmoc) protection group of a compound was removed by incubation with 12.5% morpholine at room temperature for 20 min (yields about 90-99%). The desired product was purified by HPLC (Agilent® 1260 Infinity® II with ACE® C18-300 column), followed by lyophilization with Manifold Lyophilizer (−80° C., 10 mTorr).

### Protocol for Amide Bond Formation Using NHS Reagent (NHS Protocol):

N-Hydroxysuccinimide (NHS, 65 mM) was mixed with a solution containing an amine (130 mM), 80% dimethylsulfoxide (DMSO), and a 20% 50 mM HEPES buffer (50 mM HEPES, 100 mM NaCl, and 1 mM ethylenediaminetetraacetic acid, i.e., EDTA) at pH 8-9 with agitation at room temperature for 2 hours (yields about 60-95%). The desired product was purified by HPLC (Agilent® 1260 Infinity® II with ACE® C18-300 column), followed by lyophilization with Manifold Lyophilizer (−80° C., 10 mTorr).

### Protocol for Amide Bond Formation Using HATU Reagent (HATU Protocol)

An amine (51.8 mM) was stirred with a carboxylic acid (104 mM), HATU (207 mM), and DIEA (518 mM) in DMF at room temperature for 1 hour (yields about 40-90%). The desired product was purified by HPLC (Agilent® 1260 Infinity® II with ACE® C18-300 column), followed by lyophilization with Manifold Lyophilizer (−80° C., 10 mTorr).

### Protocol for C—S Bond Formation Using Cesium Carbonate Reagent (C—S Bond Protocol)

A thiol (6.82 mM) was stirred with an iodo compound (20.5 mM), tris(2-carboxyethyl)-phosphine (TCEP, 3.41 mM) and cesium carbonate (54.6 mM) in a solution containing 60% DMSO and a 40% 50 mM potassium phosphate buffer (30.8 mM K₂HPO₄, 19.2 mM KH2PO4, 100 mM NaCl, and 1 mM EDTA; pH 7) at room temperature for 2 hours (yields about 50-90%). The desired product was purified by HPLC (Agilent® 1260 Infinity II with ACE C18-300 column), followed by lyophilization with Manifold Lyophilizer (−80° C., 10 mTorr).

### Protocol for Maleimide-Thiol Reaction (Maleimide-Thiol Protocol)

Maleimide (35.4 mM) was stirred with a thiol (29.5 mM) in a solution containing 80% DMSO and 20% 50 mM HEPES buffer (pH 6.5) at room temperature for 1 hour (yields about 90-99%). The desired product was purified by HPLC (Agilent® 1260 Infinity® II with ACE® C18-300 column), followed by lyophilization with Manifold Lyophilizer (−80° C., 10 mTorr).

### Protocol for NHS Ester Synthesis (NHS Ester Protocol)

A carboxylic acid (65.4 mM) was stirred with NHS (654 mM) and EDC (654 mM) in DMSO at room temperature for 1 hour (yields about 65-95%). The desired product was purified by HPLC (Agilent® 1260 Infinity® II with ACE® C18-300 column), followed by lyophilization with Manifold Lyophilizer (−80° C., 10 mTorr).

### Protocol for SPDP-Thiol Reaction (SPDP-Thiol Protocol)

SPDP (11.4 mM) was stirred with a thiol (5.69 mM) in a solution containing 80% DMSO and 20% 50 mM HEPES buffer (50 mM HEPES, 100 mM NaCl, and 1 mM EDTA, having a pH value of 8) at room temperature for 1 hour (yields about 90-99%). The desired product was purified by HPLC (Agilent® 1260 Infinity® II with ACE® C18-300 column), followed by lyophilization with Manifold Lyophilizer (−80° C., 10 mTorr).

### Protocol for Boc-Hydroxyl Reaction (Boc-Hydroxyl Protocol)

A hydroxyl (OH)-containing compound (11.9 mM) was stirred with Boc₂O (15.6 mM) and pyridine (35.7 mM) in a solution containing 50% DMSO and 50% DCM at room temperature overnight (yields about 95-99%). The desired product was purified by HPLC (Agilent® 1260 Infinity® II with ACE® C18-300 column), followed by lyophilization with Manifold Lyophilizer (−80° C., 10 mTorr).

### Protocol for Ester Bond Formation Using DCC/DMAP Reagent (DCC/DMAP Protocol)

An OH-containing compound (9.22 mM) was stirred with a carboxylic acid (18.4 mM) and DCC (36.9 mM) and DMAP (44.4 mM) in a solution containing 50% DMSO and 50% DCM at room temperature overnight (yields about 55-75%). The desired product was purified by HPLC (Agilent® 1260 Infinity® II with ACE® C18-300 column), followed by lyophilization with Manifold Lyophilizer (−80° C., 10 mTorr).

### Protocol for Synthesis of Polysarcosine (SPPS Protocol)

Fmoc-Sar-OH (1.5 eq.) was added to a swelled 2-chlorotrityl chloride (CTC) resin and stirred at room temperature overnight. The CTC resin was washed three times with DMF and again three times with DCM. Subsequently, the CTC resin was capped by a capping solution (DCM:MeOH:DIEA=17:2:1). Fmoc was removed by an Fmoc deprotection solution (DCM:DMF:morpholine=1:1:2) at room temperature for 1 hour. For peptide coupling, 1 eq Fmoc-Sar-OH was activated using 1 eq. HATU, followed by addition of 4 eq. DIEA. The coupling reaction was incubated at room temperature for 4 hours. The coupling step was repeated until a desired polypeptide was prepared, which was eluted from the resin after cleavage with a solution containing 20% hexafluoroisopropanol in DCM.

### Preparation and Characterization of Antibody-Polar Drug Conjugates (APDCs)

IgG1 antibody (Ab) molecules (10 mg) were reduced in 0.5 mL of a buffer containing α,α-trehalose dihydrate (9.09) mg, L-histidine HCl (0.225 mg), L-histidine (0.145 mg), polysorbate 20 (0.04 mg), and DTT (0.83 mg). After all accessible disulfides of the antibody were reduced, the buffer was changed to a 50 mM HEPES buffer (50 mM HEPES at pH 8 with 100 mM NaCl and 1 mM EDTA) using G-25 Gel Filtration Columns to remove excess DTT (yields about 80-90%). A Linker-PD (5-10 molar eq. relative to that of the antibody) was dissolved in DMSO and added to the antibody solution prepared above thereby obtaining a solution containing 2 mg/mL of Ab, 70% 50 mM HEPES buffer (50 mM HEPES pH 7.4, 100 mM NaCl and 1 mM EDTA) and 30% DMSO. The conjugation reaction was performed at room temperature for 1-2 hours. The buffer was subsequently replaced by a solution containing 6% sucrose, 10 mM sodium succinate dibasic hexahydrate, and 0.02% Tween 20 at pH 5 through G-100 Gel Filtration Columns. The protein concentration in the resulting conjugate solution was determined by the BCA protein assay using Herceptin as a standard (yields about 80-90%). The drug/antibody ratio (DAR) was determined with HPLC. Briefly, the weight of the antibody was determined by a fluorescence detector (FLD; excitation at 274 nm and emission at 310 nm), while the weight of the drug was determined by a specific UV absorbance wavelength e.g., 254 nm for DM1. The FLD and UV absorbance of a control (i.e., an antibody solution) was used as a reference. Whole-antibody mass spectrometry provides a second methodology for assessing drug loading homogeneity and DAR of APDCs. Antibody aggregates were analyzed using a size exclusion column (SEC). The APDC thus purified was lyophilized and stored at −20° C. until use. Bolt's immune-stimulating ADC, Her-T785, as a comparative sample was prepared as described (Ackerman et al., 2021).

### Preparation and Characterization of AbPDCs

In a similar manner as described above, albumin (0.74 mM) was incubated with TCEP (0.74 mM) for 10 min at 37° C. and the buffer was exchanged to 50 mM HEPES buffer (50 mM HEPES at pH 8 with 100 mM NaCl and 1 mM EDTA) by G-25 Gel Filtration Columns to remove excess DTT (yields around 80-90%). A Linker-PD (2 molar eq. relative to the albumin) of this invention was dissolved in DMSO and added to the albumin buffer to obtain a solution containing 2 mg/mL of albumin, 70% HEPES buffer (50 mM HEPES pH 7.4, 100 mM NaCl and 1 mM EDTA) and 30% DMSO. Conjugation was performed at room temperature for 1-2 hours. The buffer was replaced by another buffer containing 6% sucrose, 10 mM sodium succinate dibasic hexahydrate, and 0.02% Tween 20 (pH 5) through G-100 Gel Filtration Columns. The protein concentration in the conjugate solution thus prepared was determined by the BCA protein assay using human serum albumin as a standard (yields around 80-90%). The drug/albumin ratio was determined by HPLC. Briefly, the quantity of albumin was determined by FLD (excitation at 274 nm and emission at 310 nm) and the weight of the drug was determined at 254 using a UV spectrometer. Human serum albumin was used as a standard in both FLD and UV analysis.

### Preparation of Polar Drugs or Polar Drug Intermediates

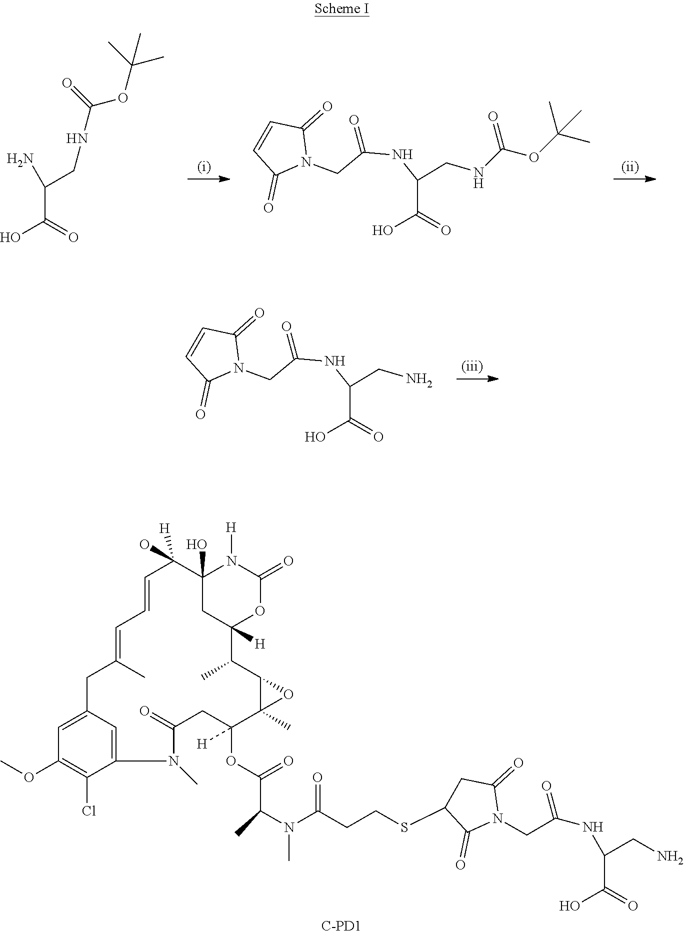 [SMILES: CC(C)(C)OC(=O)NCC(N)C(=O)O ; CC(C)(C)OC(=O)NCC(NC(=O)CN1C(=O)C=CC1=O)C(=O)O ; NCC(NC(=O)CN1C(=O)C=CC1=O)C(=O)O ; COc1cc2cc(c1Cl)N(C)C(=O)C[C@H](OC(=O)[C@H](C)N(C)C(=O)CCSC1CC(=O)N(CC(=O)NC(CN)C(=O)O)C1=O)[C@]1(C)O[C@H]1[C@H](C)[C@@H]1C[C@@](O)(NC(=O)O1)[C@H](O)/C=C/C=C(\C)C2 ; "C-PD1"]

Polar drug C-PD1, i.e., DM1-AMAS-Dap(NH2)-OH, was prepared as depicted in Scheme I above having three steps: (i) amide bond formation between (2S)-2-amino-3-{[(tert-butoxy)carbonyl]amino}propanoic acid [NH2-Dap(Boc)-OH] and N-Succinimidyl Maleimidoacetate (AMAS) following the NHS protocol described above, (ii) removal of the Boc protection group of AMAS-Dap(Boc)-OH following the Boc protocol also described above, and (iii) maleimide-S bond formation between AMAS-Dap(Boc)-OH and DM1 following the maleimide-thiol protocol again described above.

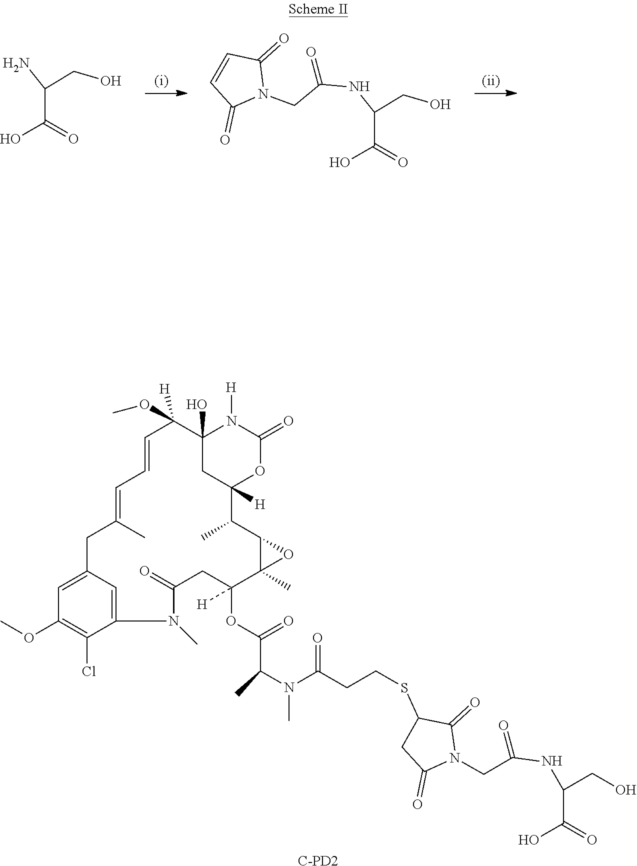 [SMILES: NC(CO)C(=O)O ; O=C(CN1C(=O)C=CC1=O)NC(CO)C(=O)O ; COc1cc2cc(c1Cl)N(C)C(=O)C[C@H](OC(=O)[C@H](C)N(C)C(=O)CCSC1CC(=O)N(CC(=O)NC(CO)C(=O)O)C1=O)[C@]1(C)O[C@H]1[C@H](C)[C@@H]1C[C@@](O)(NC(=O)O1)[C@H](OC)/C=C/C=C(\C)C2 ; "C-PD2"]

Similarly, polar drug C-PD2, i.e., DM1-AMAS-Ser-OH, was prepared as depicted in Scheme II above: (i) amide bond formation between Serine and N-Succinimidyl Maleimidoacetate (AMAS) using the NHS protocol and (ii) maleimide-S bond formation between AMAS-Ser-OH and DM1 using the maleimide-thiol protocol.

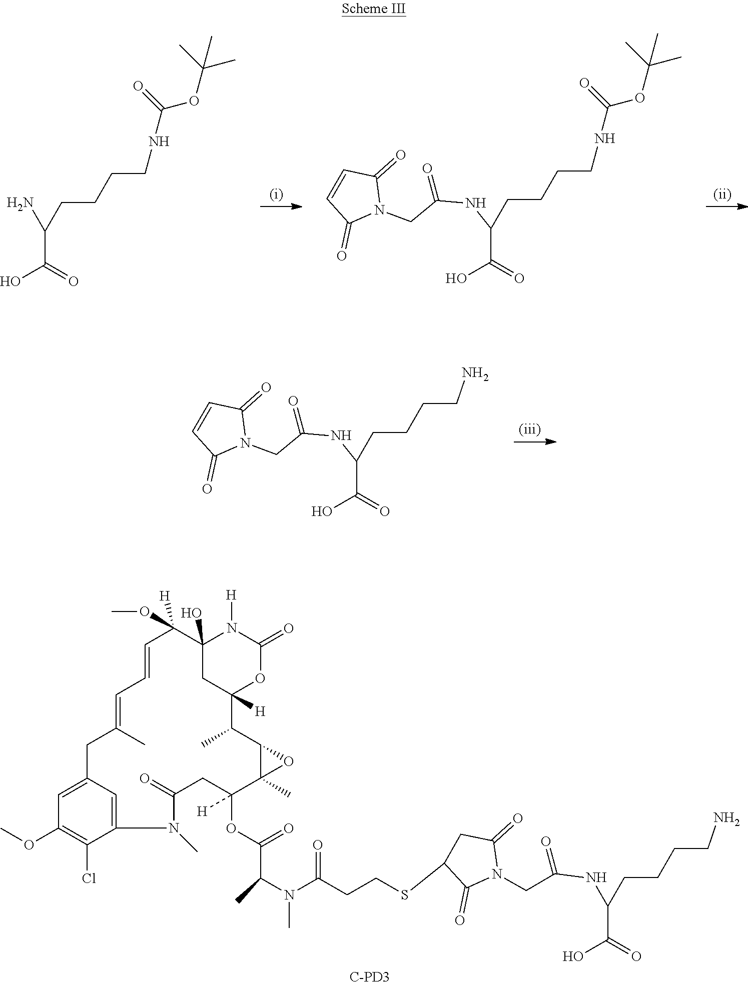

Polar drug C-PD3, i.e., DM1-AMAS-Lys(NH2)-OH, was prepared as depicted in Scheme III above: (i) amide bond formation between H-Lys(Boc)-OH and N-succinimidyl maleimidoacetate (AMAS) using the NHS protocol, (ii) removal of the Boc protection group of AMAS-Lys(Boc)-OH using the Boc protocol, and (iii) maleimide-S bond formation AMAS-Lys(NH2)—OH and DM1 using the maleimide-thiol protocol.

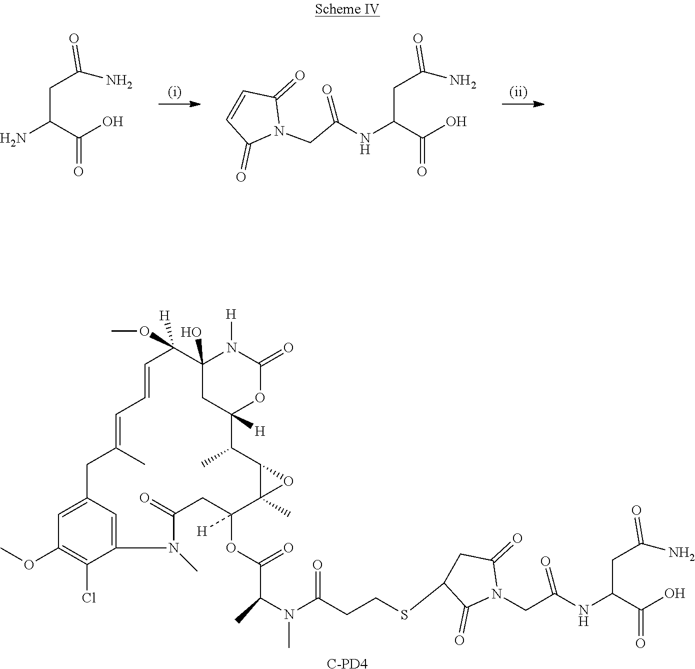

Polar drug C-PD4, i.e., DM1-AMAS-Asn-OH, was prepared as depicted in Scheme IV above: (i) L-Asparagine and N-succinimidyl maleimidoacetate (AMAS) using the NHS protocol and (ii) maleimide-S bond formation between AMAS-Asn-OH and DM1 using the maleimide-thiol protocol.

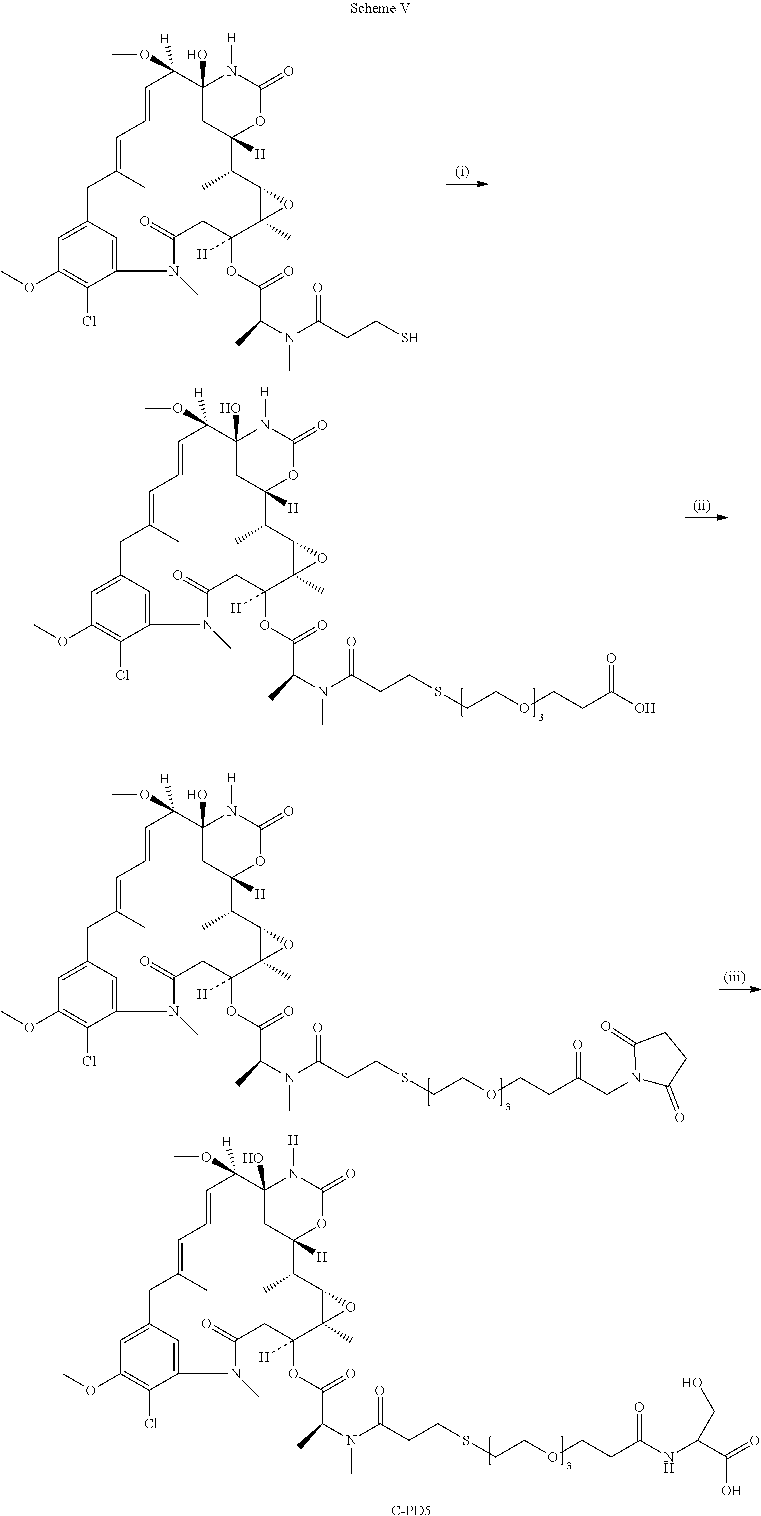

Polar drug C-PD5, i.e., DM1-PEG3-Serine, was prepared as depicted in Scheme V above: (i) C—S bond formation between DM1 and iodo-PEG3-carboxylic acid using the cesium carbonate protocol, (ii) preparing the NHS ester of DM1-PEG3-carboxylic acid using the NHS ester protocol, and (iii) amide bond formation between DM1-PEG3-NHS and serine following the NHS protocol.

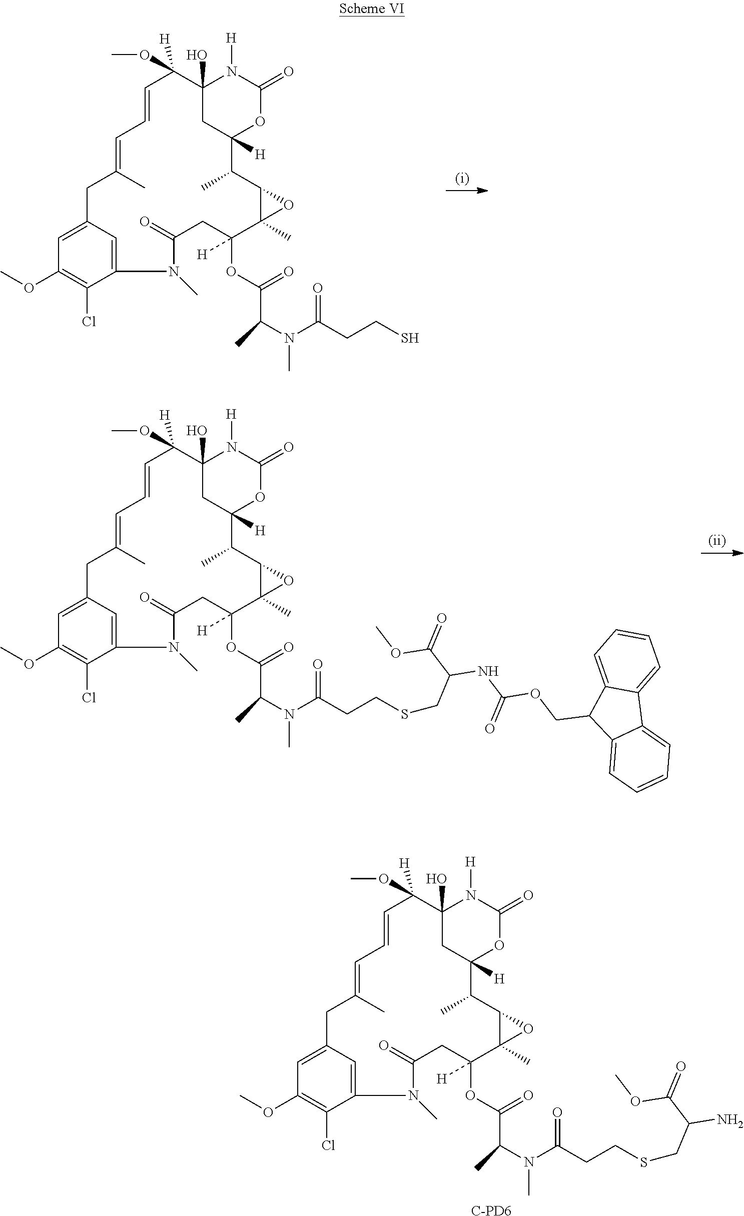

Polar drug C-PD6, i.e., Ala(DM1)-Ome, was prepared as depicted in Scheme VI above: (i) C—S bond formation between DM1 and N-Fmoc-3-iodo-L-alanine methyl (Fmoc-Ala(Iodo)-Ome) using the cesium carbonate protocol and (ii) removing the Fmoc protection group of Fmoc-Ala(DM1)-Ome following the Fmoc protocol.

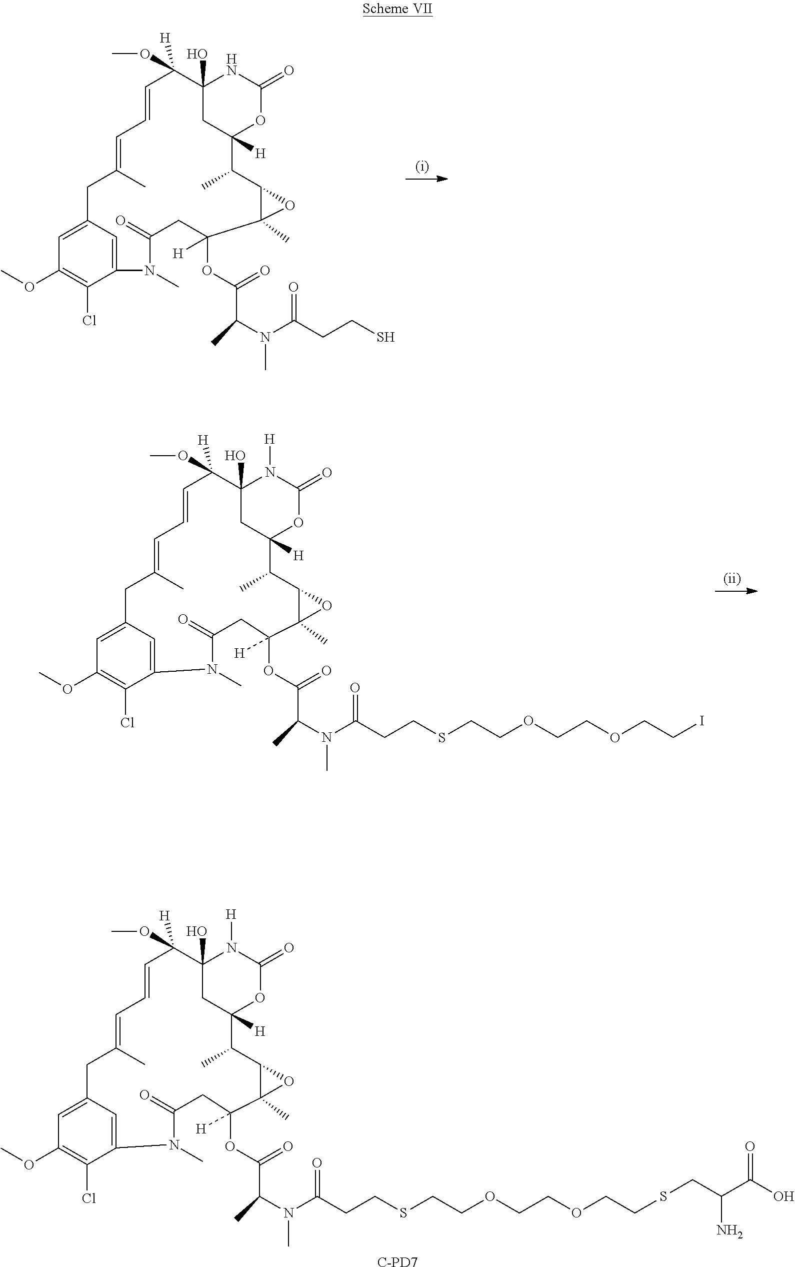

Polar drug C-PD7, i.e., Cysteine-PEG2-DM1, was prepared as depicted in Scheme VII above: (i) C—S bond formation between DM1 and 1,2-bis(2-iodoethoxy)ethane using the cesium carbonate protocol and (ii) C—S bond formation between DM1-[1,2-Bis(2-iodoethoxy)ethane]and cysteine also using the cesium carbonate protocol.

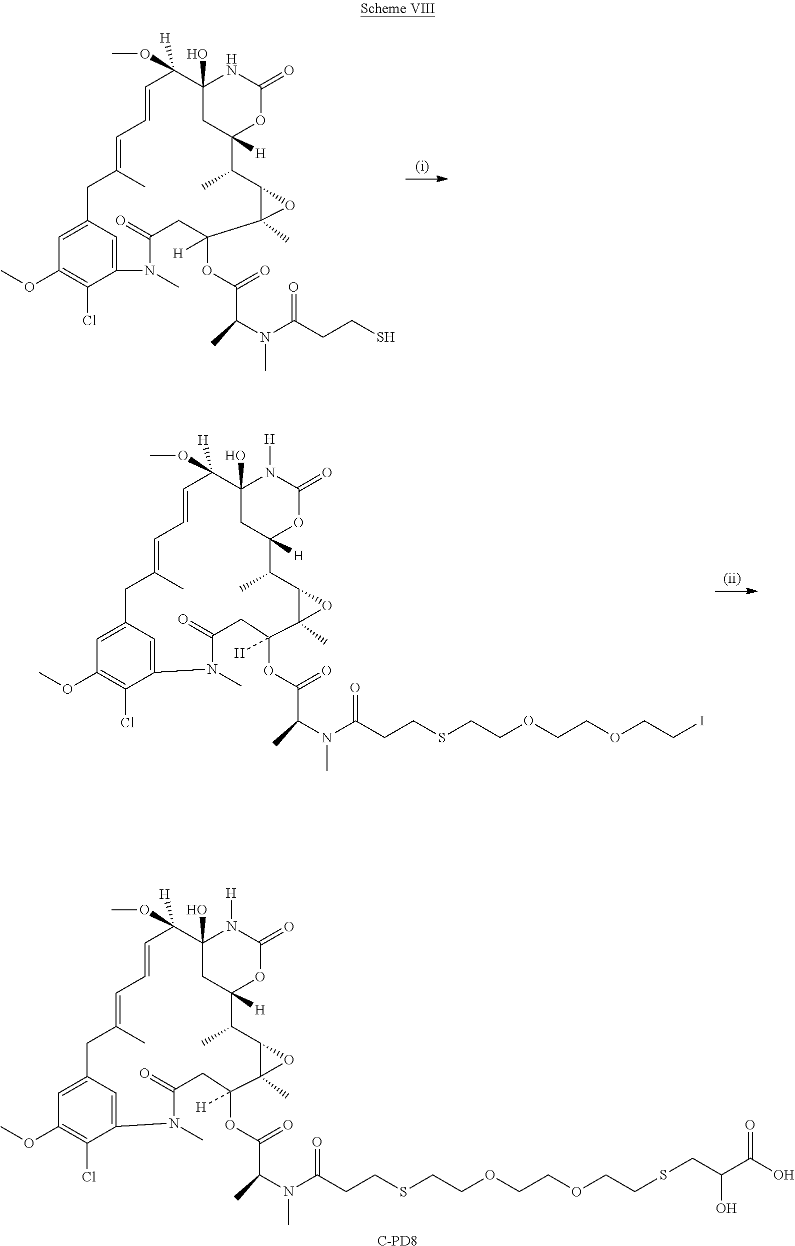

Polar drug C-PD8, i.e., 3-mercaptolactic acid-PEG2-DM1, was prepared as depicted in Scheme VIII above: (i) C—S bond formation between DM1 and 1,2-bis(2-iodoethoxy)ethane using the cesium carbonate protocol and (ii) C—S bond formation between DM1-[1,2-bis(2-iodoethoxy)ethane] and 3-mercaptolactic acid also using the cesium carbonate protocol.

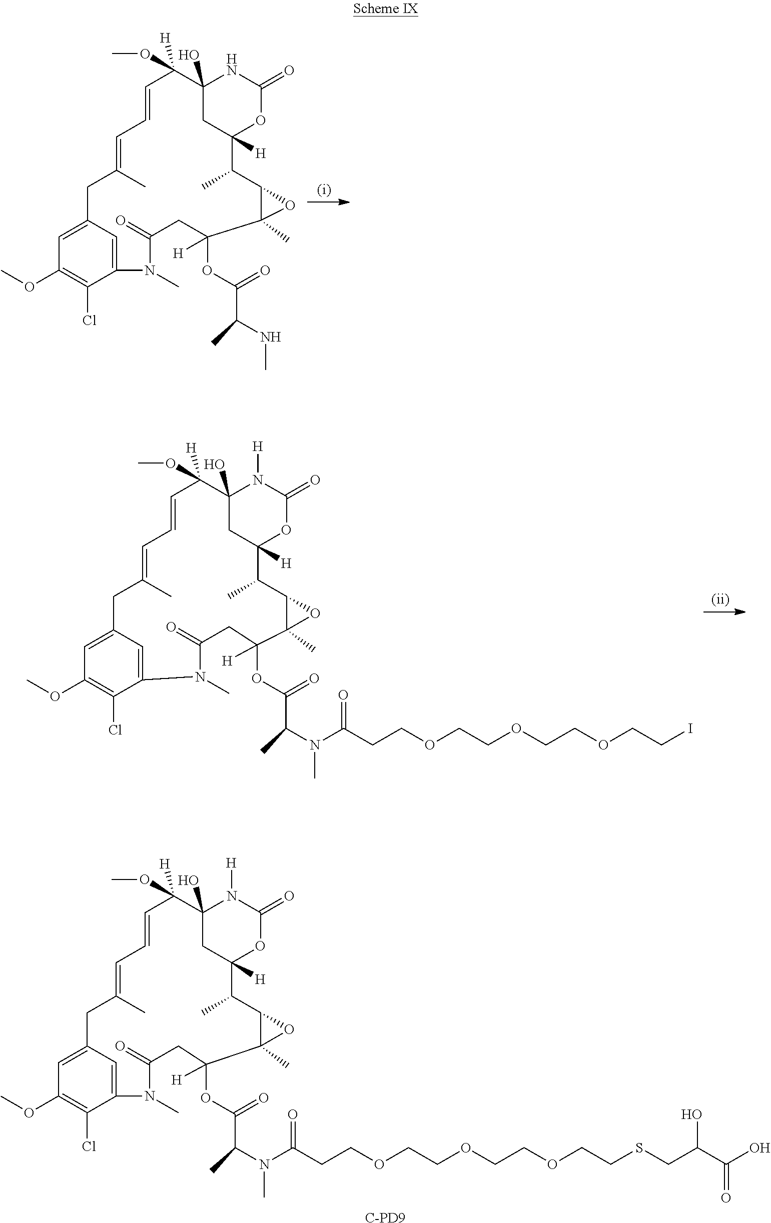

Polar drug C-PD9, i.e., N-Me-L-Ala-maytansinol -PEG3-3-mercaptolactic acid, was prepared as depicted in Scheme IX above: (i) amide bond formation between N-Me-L-Ala-maytansinol and iodo-PEG3-acid following the HATU protocol and (ii) C—S bond formation between N-Me-L-Ala-maytansinol -PEG3-iodo and 3-mercaptolactic acid using the cesium carbonate protocol.

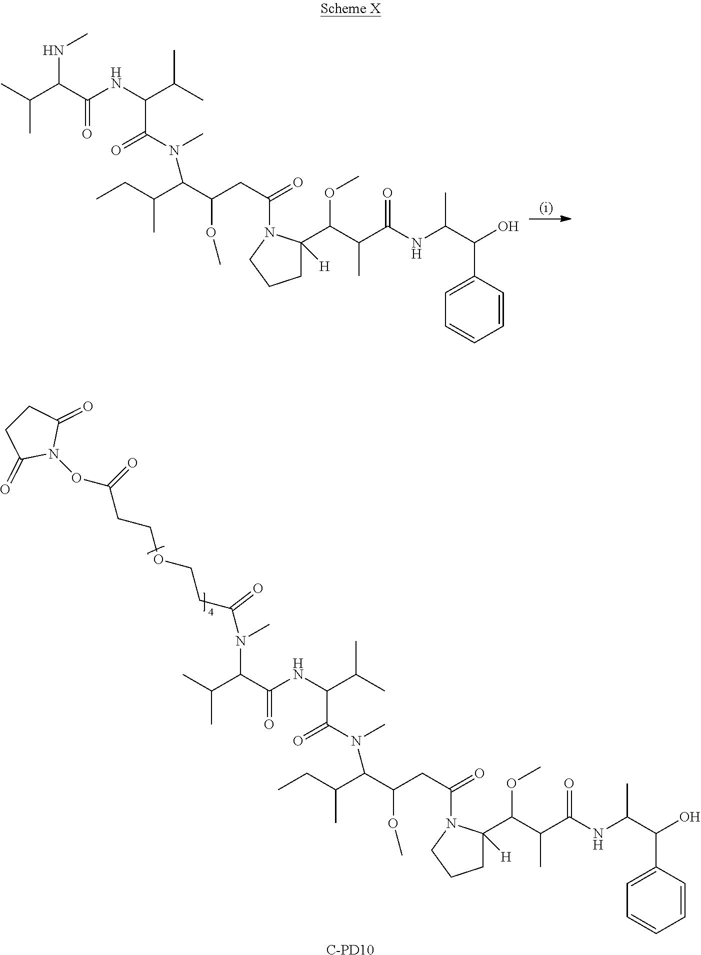

Polar drug C-PD10, i.e., MMAE-PEG4-NHS, was prepared as depicted in Scheme X above following the HATU protocol to form an amide bond between monomethyl auristatin E (MMAE) and Acid-PEG4-NHS ester.

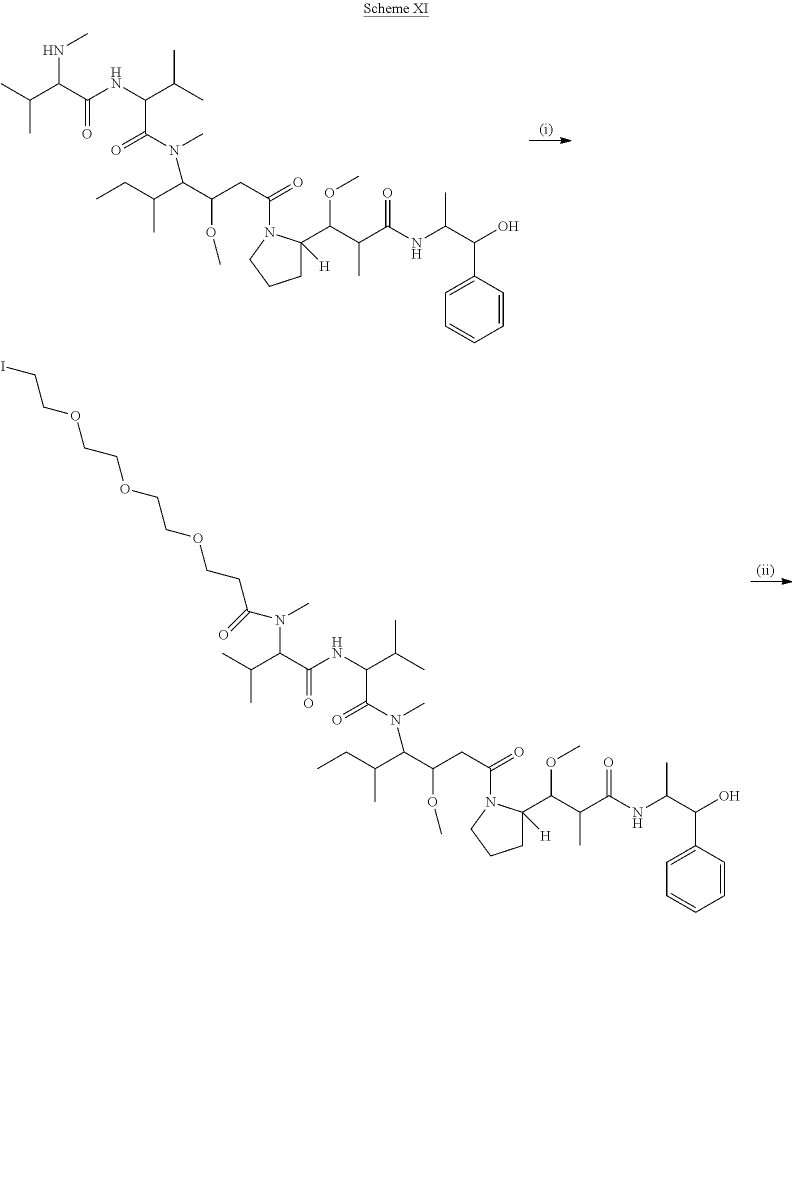

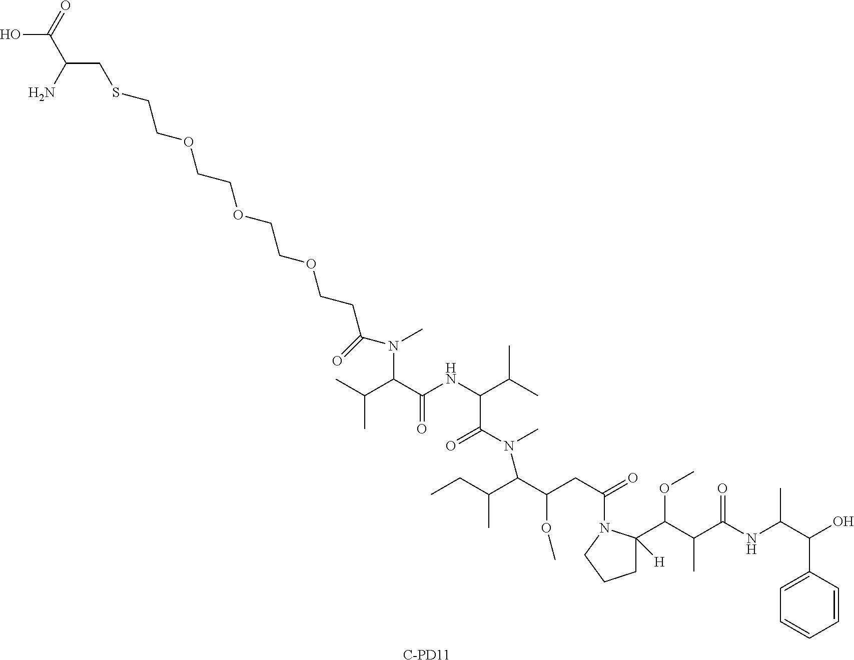 [SMILES: CCC(C)C(C(CC(=O)N1CCCC1C(OC)C(C)C(=O)NC(C)C(O)c1ccccc1)OC)N(C)C(=O)C(NC(=O)C(C(C)C)N(C)C(=O)CCOCCOCCOCCSCC(N)C(=O)O)C(C)C ; "C-PD11"]

Polar drug C-PD11, i.e., MMAE-PEG3-Cysteine, was prepared as depicted in Scheme XI above: (i) amide bond formation between monomethyl auristatin E (MMAE) and Iodo-PEG3-Acid following the HATU protocol and (ii) C—S bond formation between MMAE-PEG3-iodo and cysteine using the cesium carbonate protocol.

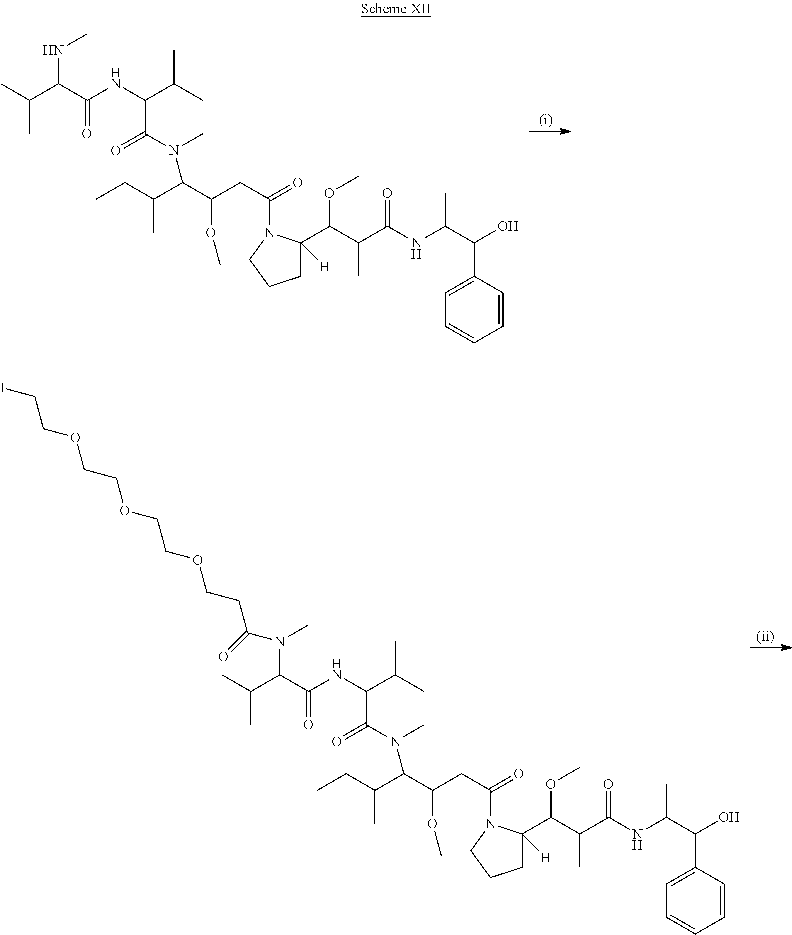 [SMILES: CCC(C)C(C(CC(=O)N1CCCC1C(OC)C(C)C(=O)NC(C)C(O)c1ccccc1)OC)N(C)C(=O)C(NC(=O)C(NC)C(C)C)C(C)C ; CCC(C)C(C(CC(=O)N1CCCC1C(OC)C(C)C(=O)NC(C)C(O)c1ccccc1)OC)N(C)C(=O)C(NC(=O)C(C(C)C)N(C)C(=O)CCOCCOCCOCCI)C(C)C]

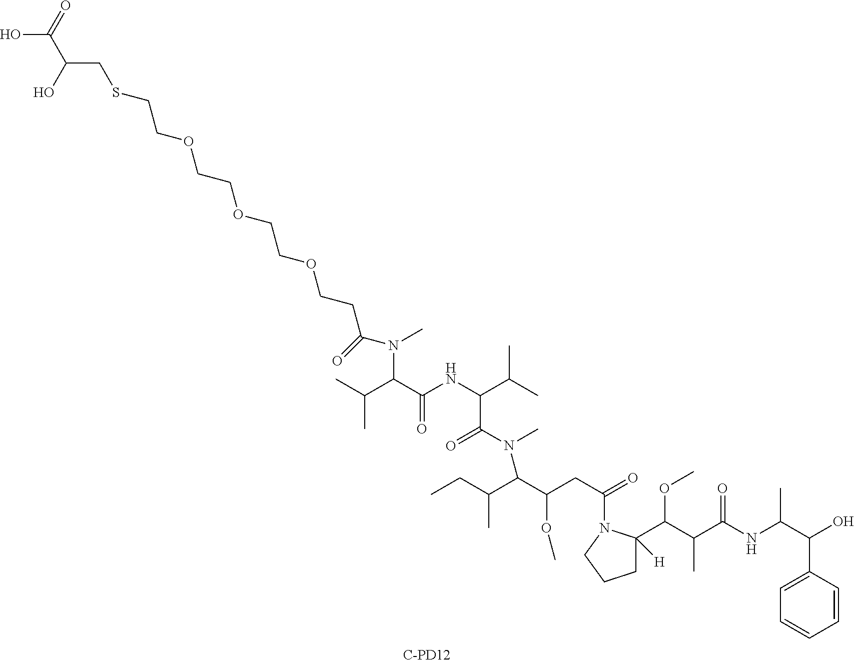 [SMILES: CCC(C)C(C(CC(=O)N1CCCC1C(OC)C(C)C(=O)NC(C)C(O)c1ccccc1)OC)N(C)C(=O)C(NC(=O)C(C(C)C)N(C)C(=O)CCOCCOCCOCCSCC(O)C(=O)O)C(C)C ; "C-PD12"]

Polar drug C-PD12, i.e., MMAE-PEG3-3-mercaptolactic acid, was prepared as depicted in Scheme XII above: (i) amide bond formation between monomethyl auristatin E (MMAE) and Iodo-PEG3-Acid following the HATU protocol and (ii) C—S bond formation between MMAE-PEG3-iodo and 3-mercaptolactic acid using the cesium carbonate protocol.

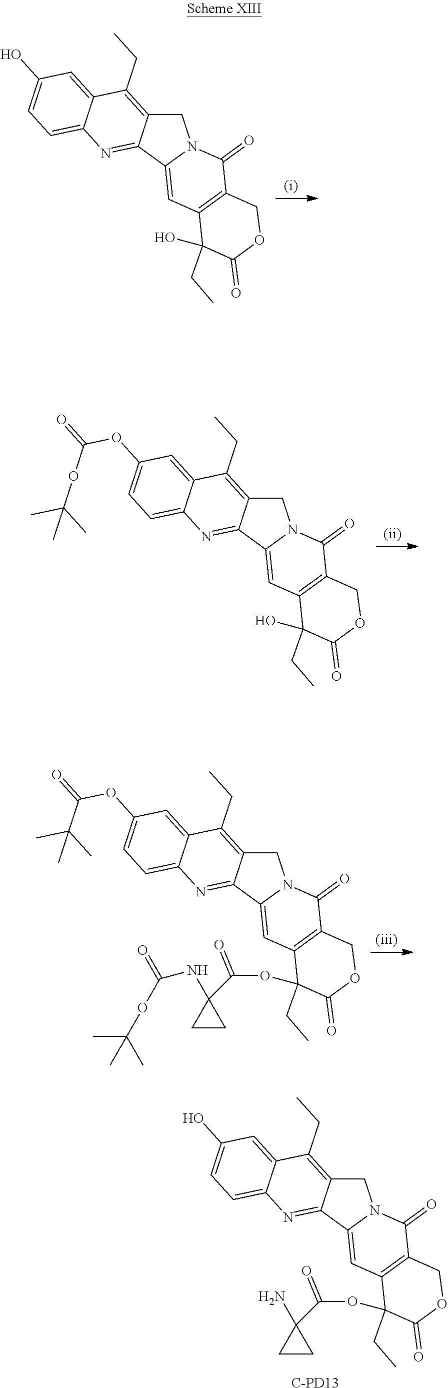

Polar drug C-PD13, i.e., CPCA-SN38, was prepared in three steps as depicted in Scheme XIII above: (i) Boc protection of the 10-hydroxy group of SN-38 following the Boc protection protocol, (ii) ester bond formation between Boc-10-hydroxy-SN38 and 1-[(tert-butoxycarbonyl)-amino]cyclopropanecarboxylic acid (Boc-CPCA-OH) following the carbonic ester protocol, and (iii) removal of the Boc protection group of Boc-CPCA-Boc-10-hydroxy-SN38 using the Boc protocol.

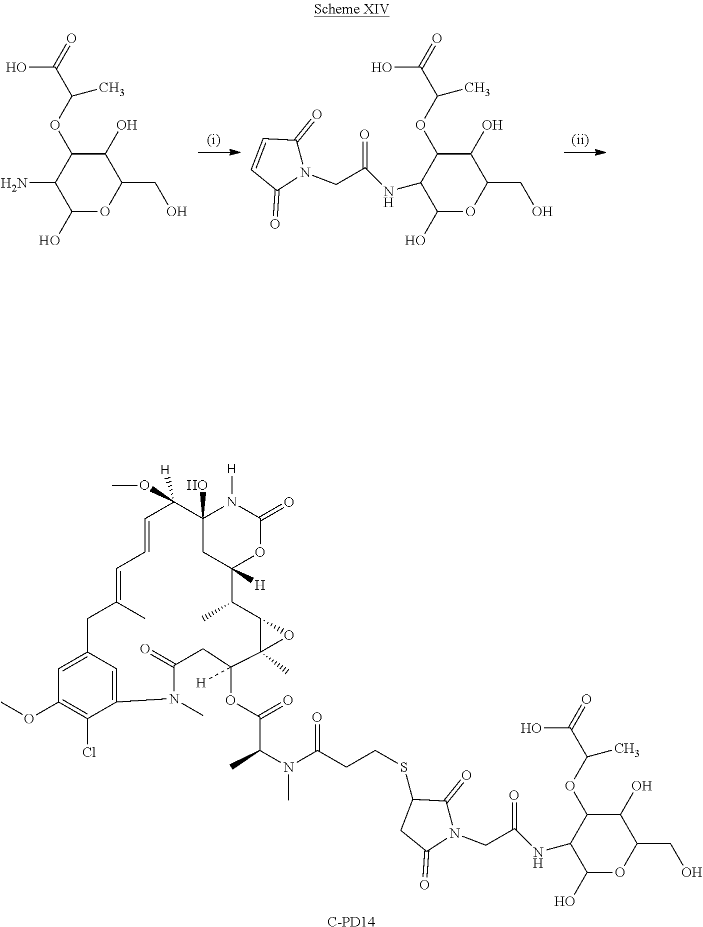

Polar drug C-PD14, i.e., DM1-AMAS-Muramic acid, was prepared as depicted in Scheme XIV above: (i) amide bond formation between muramic acid and N-succinimidyl maleimidoacetate (AMAS) following the NHS protocol and (ii) maleimide-S bond formation between AMAS-Muramic acid and DM1 following the maleimide-thiol protocol.

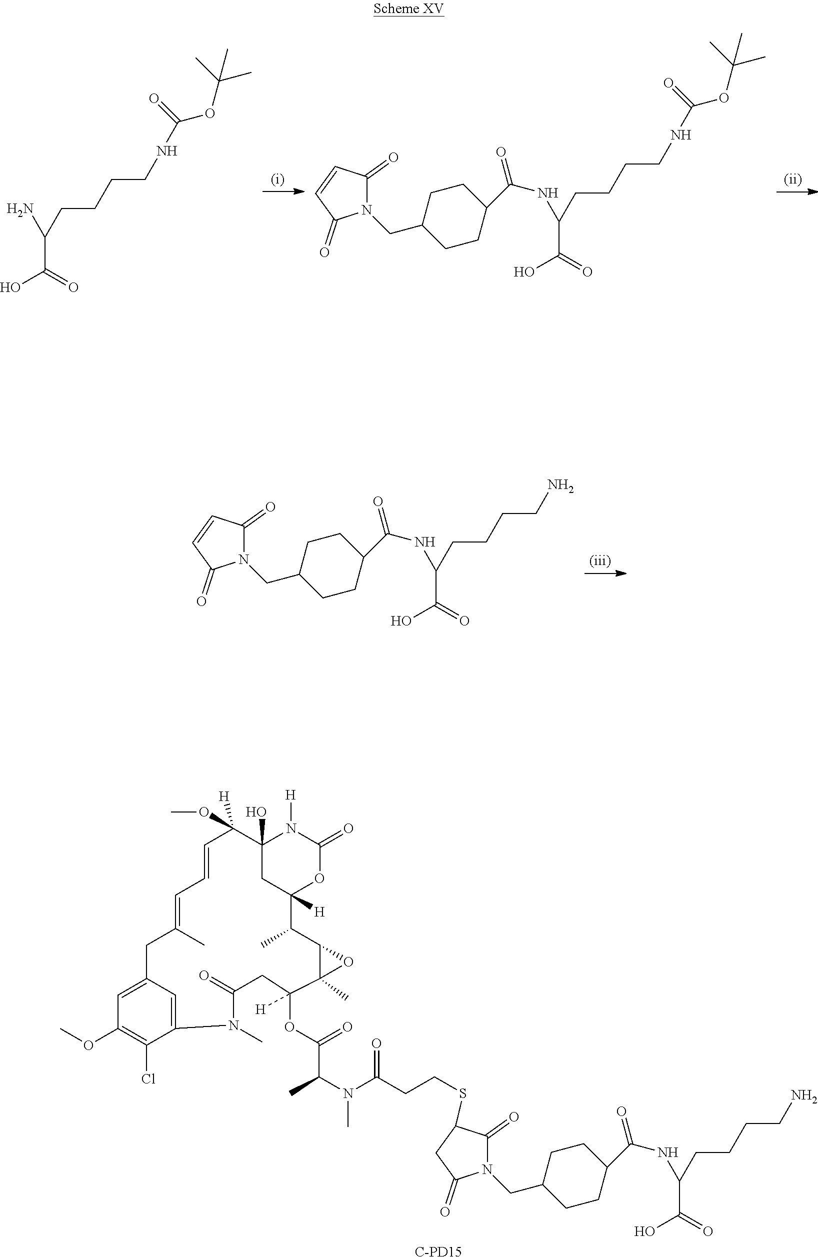

Polar drug C-PD15, i.e., DM1-SMCC -Lys(NH2)-OH, was prepared as depicted in Scheme XV above: (i) amide bond formation between H-Lys(Boc)-OH and n-succinimidyl 4-(N-maleimidomethyl)cyclohexanecarboxylate (SMCC) following the NHS protocol, (ii) removal of the Boc protection group of SMCC-Lys(Boc)-OH followed the Boc protocol, and (iii) maleimide-S bond formation between SMCC-Lys(NH2)-OH and DM1 following the maleimide-thiol protocol.

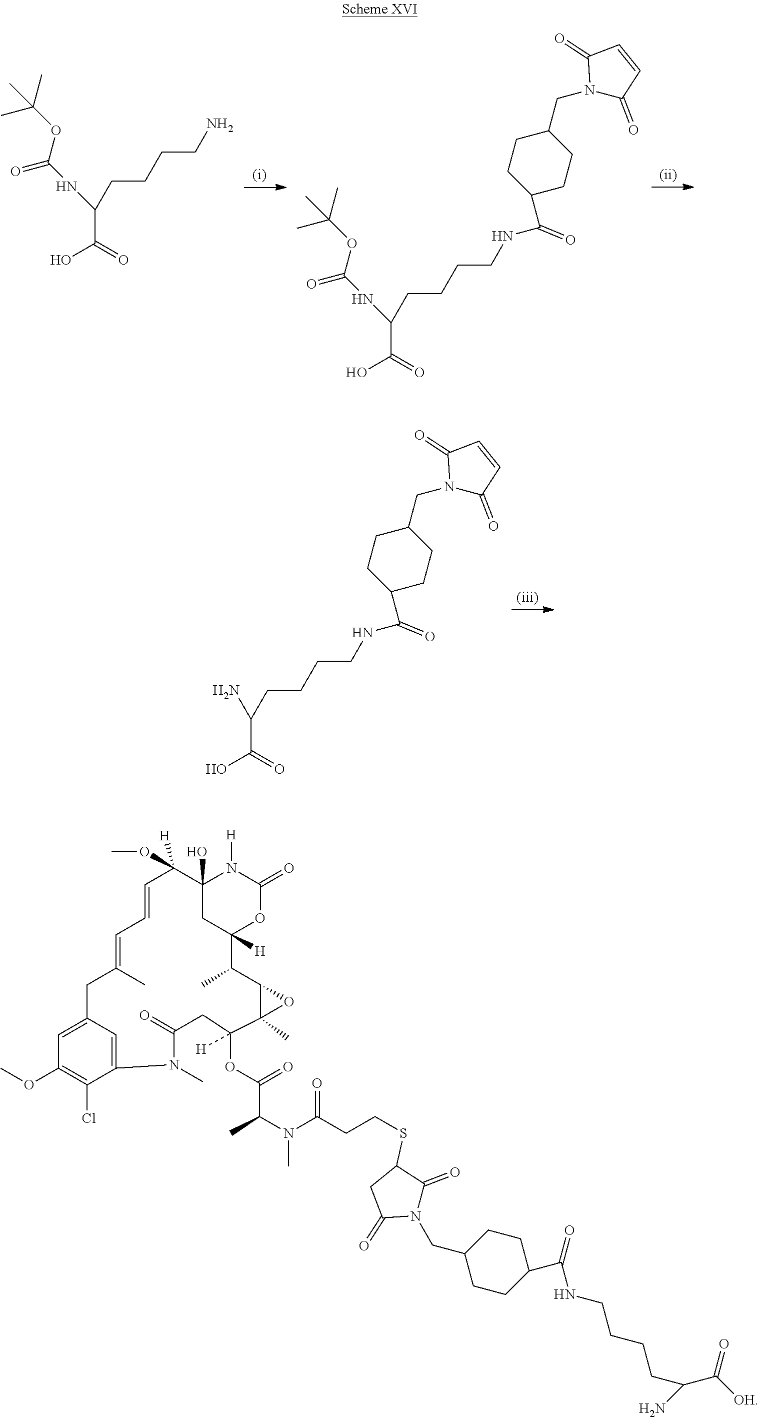

Polar drug C-PD16, i.e., Lys(DM1-SMCC)—OH, was prepared as depicted in Scheme XVI above: (i) amide bond formation between Boc-Lys-OH and n-succinimidyl 4-(N-male-imidomethyl)cyclohexanecarboxylate (SMCC) following the NHS protocol, (ii) removal of the Boc protection group of Boc-Lys(SMCC)—OH following the Boc protocol, and (iii) maleimide-S bond formation between Lys(SMCC)—OH and DM1 following the maleimide-thiol protocol.

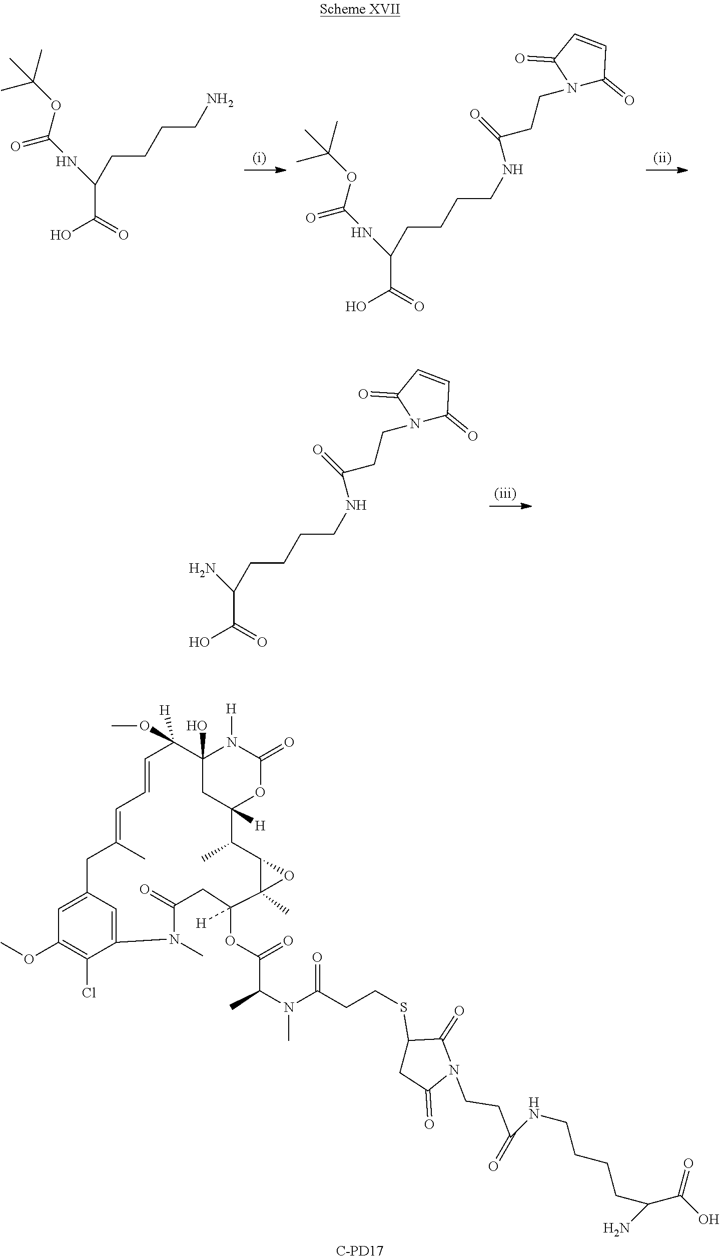

Polar drug C-PD17, i.e., Lys(DM1-BMPS)—OH, was prepared as depicted in Scheme XVII above: (i) amide bond formation between Boc-Lys-OH and N-succinimidyl 3-maleimidopropionate (BMPS) following the NHS protocol, (ii) removal of the Boc protection group of Boc-Lys(BMPS)—OH following the Boc protocol, and (iii) maleimide-S bond formation between Lys(BMPS)—OH and DM1 following the maleimide-thiol protocol.

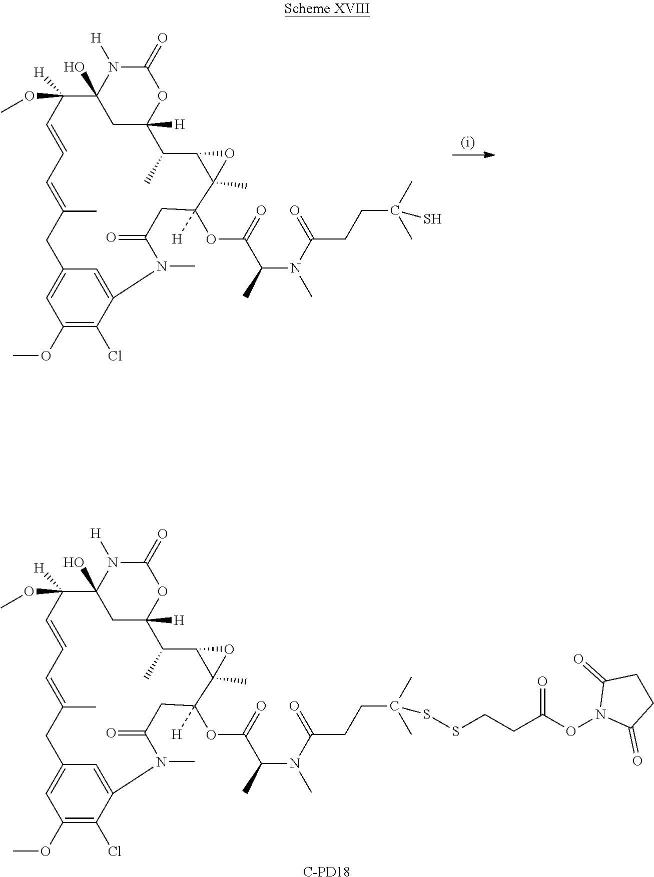 [SMILES: COc1cc2cc(c1Cl)N(C)C(=O)C[C@H](OC(=O)[C@H](C)N(C)C(=O)CCC(C)(C)S)[C@]1(C)O[C@H]1[C@H](C)[C@@H]1C[C@@](O)(NC(=O)O1)[C@H](OC)/C=C/C=C(\C)C2 ; COc1cc2cc(c1Cl)N(C)C(=O)C[C@H](OC(=O)[C@H](C)N(C)C(=O)CCC(C)(C)SSCCC(=O)ON1C(=O)CCC1=O)[C@]1(C)O[C@H]1[C@H](C)[C@@H]1C[C@@](O)(NC(=O)O1)[C@H](OC)/C=C/C=C(\C)C2 ; "C-PD18"]

Polar drug C-PD18, i.e., DM4-SPDP, was prepared as depicted in Scheme XVIII above following the SPDP protocol to form a disulfide bond DM4 and SPDP.

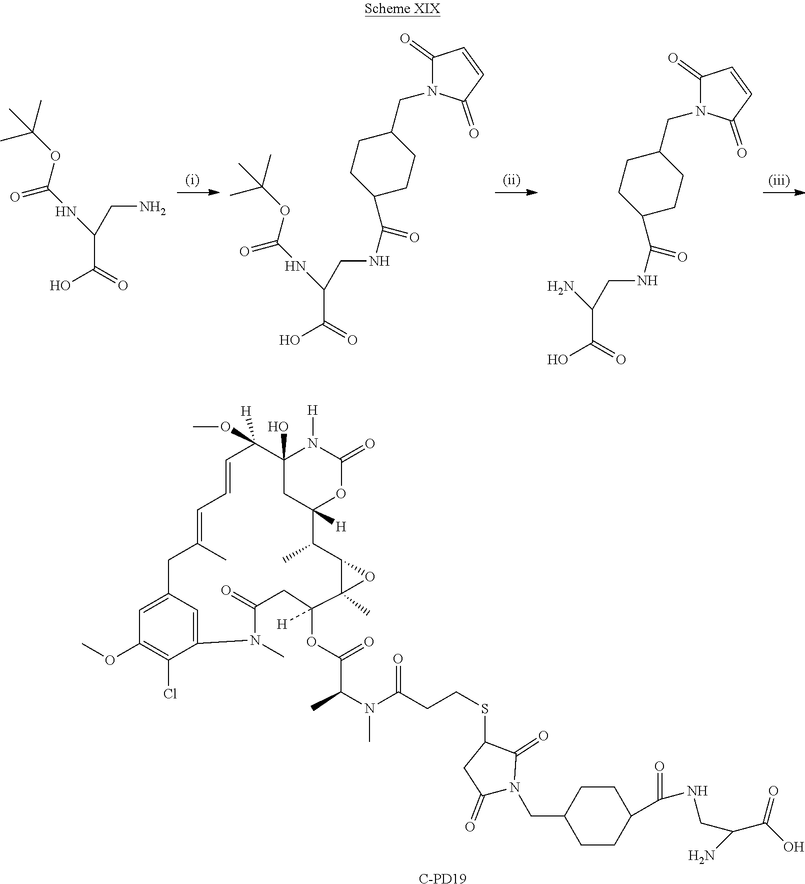 [SMILES: CC(C)(C)OC(=O)NC(CN)C(=O)O ; CC(C)(C)OC(=O)NC(CNC(=O)C1CCC(CN2C(=O)C=CC2=O)CC1)C(=O)O ; NC(CNC(=O)C1CCC(CN2C(=O)C=CC2=O)CC1)C(=O)O ; COc1cc2cc(c1Cl)N(C)C(=O)C[C@H](OC(=O)[C@H](C)N(C)C(=O)CCSC1CC(=O)N(CC3CCC(C(=O)NCC(N)C(=O)O)CC3)C1=O)[C@]1(C)O[C@H]1[C@H](C)[C@@H]1C[C@@](O)(NC(=O)O1)[C@H](OC)/C=C/C=C(\C)C2 ; "C-PD19"]

Polar drug C-PD19, i.e., Dap(SMCC-DM1)—OH, was prepared as depicted in Scheme XIX above: (i) amide bond formation between Boc-Dap-OH and and n-succinimidyl 4-(N-maleimidomethyl)cyclohexanecarboxylate (SMCC) following the NHS protocol, (ii) removal of the Boc protection group of Boc-Dap(SMCC)—OH following the Boc protocol, and (iii) maleimide-S bond formation between Dap(SMCC)—OH and DM1 following the maleimide-thiol protocol.

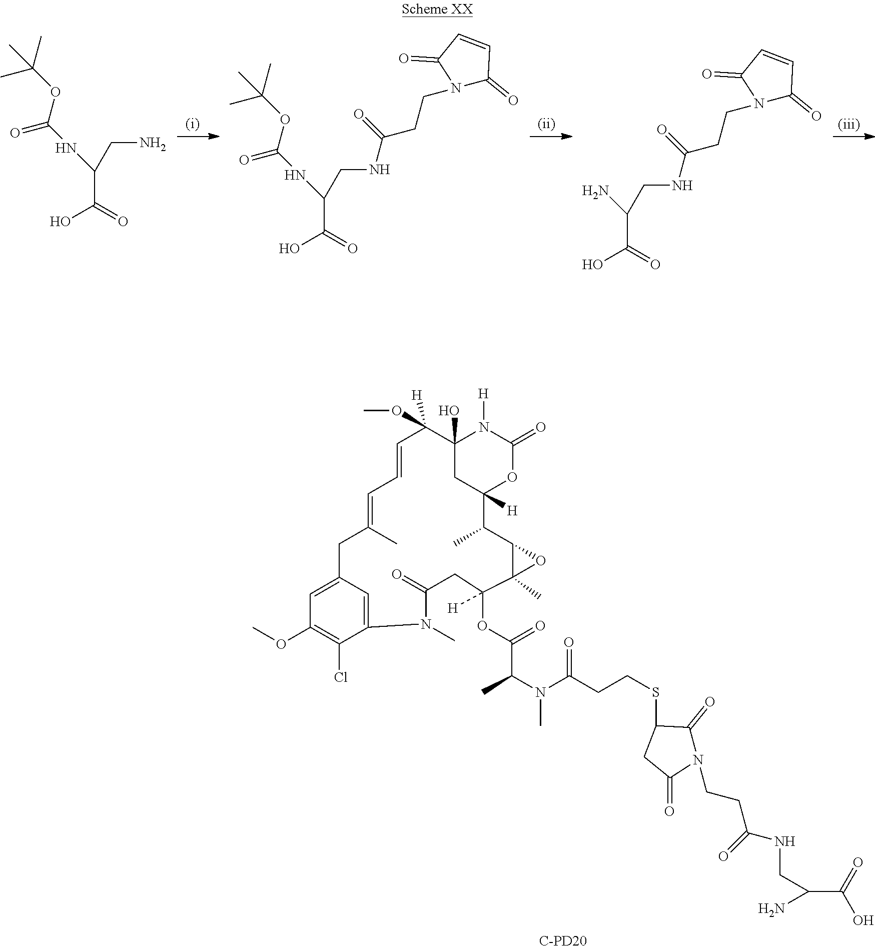 [SMILES: CC(C)(C)OC(=O)NC(CN)C(=O)O ; CC(C)(C)OC(=O)NC(CNC(=O)CCN1C(=O)C=CC1=O)C(=O)O ; NC(CNC(=O)CCN1C(=O)C=CC1=O)C(=O)O ; COc1cc2cc(c1Cl)N(C)C(=O)C[C@H](OC(=O)[C@H](C)N(C)C(=O)CCSC1CC(=O)N(CCC(=O)NCC(N)C(=O)O)C1=O)[C@]1(C)O[C@H]1[C@H](C)[C@@H]1C[C@@](O)(NC(=O)O1)[C@H](OC)/C=C/C=C(\C)C2 ; "C-PD20"]

Polar drug C-PD20, i.e., Dap(BMPS-DM1)—OH, was prepared as depicted in Scheme XX above: (i) amide bond formation between Boc-Dap-OH and N-succinimidyl 3-maleimidopropionate (BMPS) following the NHS protocol, (ii) removal of the Boc protection group of Boc-Dap(BMPS)—OH following the Boc protocol, and (iii) maleimide-S bond formation between Dap(BMPS)—OH and DM1 following the maleimide-thiol protocol.

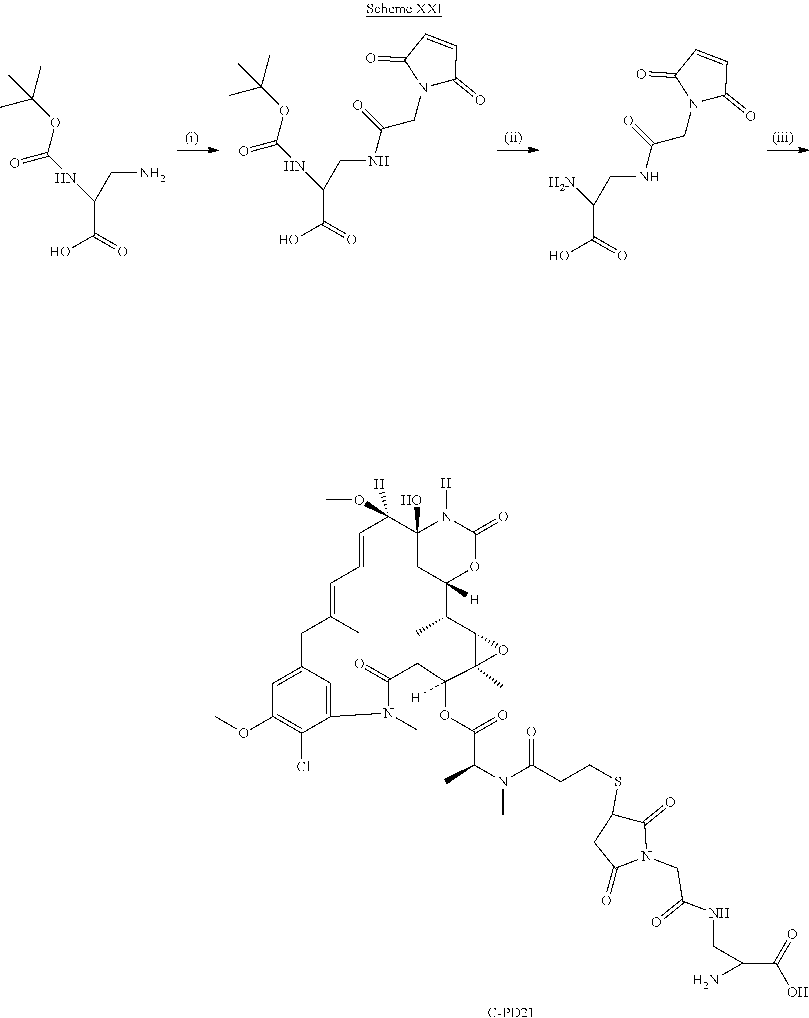 [SMILES: CC(C)(C)OC(=O)NC(CN)C(=O)O ; CC(C)(C)OC(=O)NC(CNC(=O)CN1C(=O)C=CC1=O)C(=O)O ; NC(CNC(=O)CN1C(=O)C=CC1=O)C(=O)O ; COc1cc2cc(c1Cl)N(C)C(=O)C[C@H](OC(=O)[C@H](C)N(C)C(=O)CCSC1CC(=O)N(CC(=O)NCC(N)C(=O)O)C1=O)[C@]1(C)O[C@H]1[C@H](C)[C@@H]1C[C@@](O)(NC(=O)O1)[C@H](OC)/C=C/C=C(\C)C2 ; "C-PD21"]

Polar drug C-PD21, i.e., Dap(AMAS-DM1)—OH, was prepared as depicted in Scheme XXI above: (i) amide bond formation between Boc-Dap-OH and N-succinimidyl maleimidoacetate (AMAS) following the NHS protocol, (ii) removal of the Boc protection group of Boc-Dap(AMAS)—OH following the Boc protocol, and (iii) maleimide-S bond formation between Dap(AMAS)—OH and DM1 following the maleimide-thiol protocol.

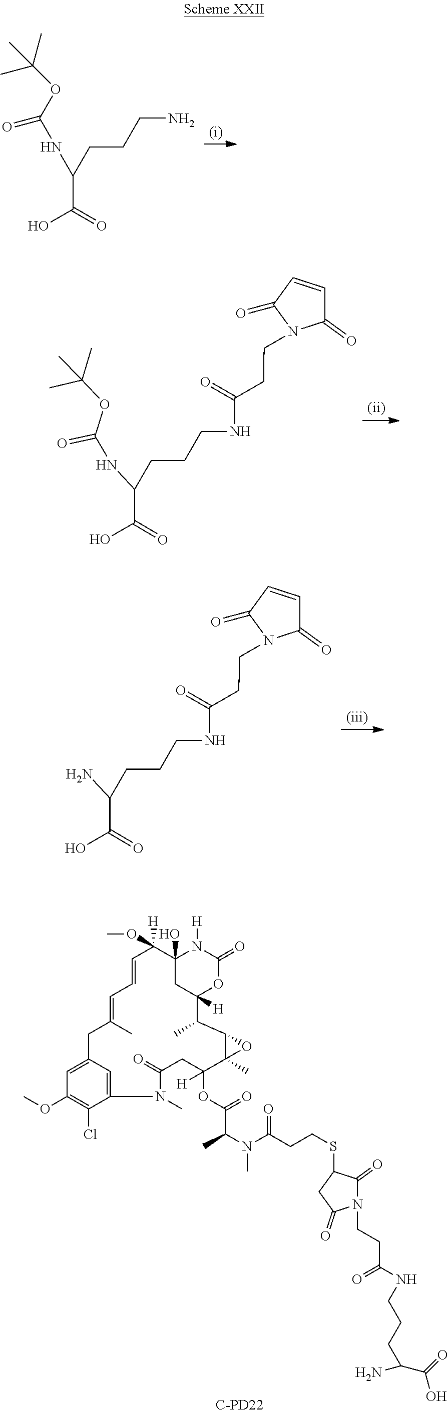 [SMILES: CC(C)(C)OC(=O)NC(CCCN)C(=O)O ; CC(C)(C)OC(=O)NC(CCCNC(=O)CCN1C(=O)C=CC1=O)C(=O)O ; NC(CCCNC(=O)CCN1C(=O)C=CC1=O)C(=O)O ; COc1cc2cc(c1Cl)CN(C)C(=O)CC(OC(=O)[C@H](C)N(C)C(=O)CCSC1CC(=O)N(CCC(=O)NCCCC(N)C(=O)O)C1=O)[C@]1(C)O[C@H]1[C@H](C)[C@@H]1C[C@@](O)(NC(=O)O1)[C@H](OC)/C=C/C=C(\C)C2 ; "C-PD22"]

Polar drug C-PD22, i.e., Om(BMPS-DM1)—OH, was prepared as depicted in Scheme XXII above: (i) amide bond formation between Boc-Orn-OH and N-succinimidyl 3-maleimidopropionate (BMPS) following the NHS protocol, (ii) removal of the Boc protection group of Boc-Orn(BMPS)—OH following the Boc protocol, and (iii) maleimide-S bond formation between Orn(BMPS)—OH and DM1 following the maleimide-thiol protocol.

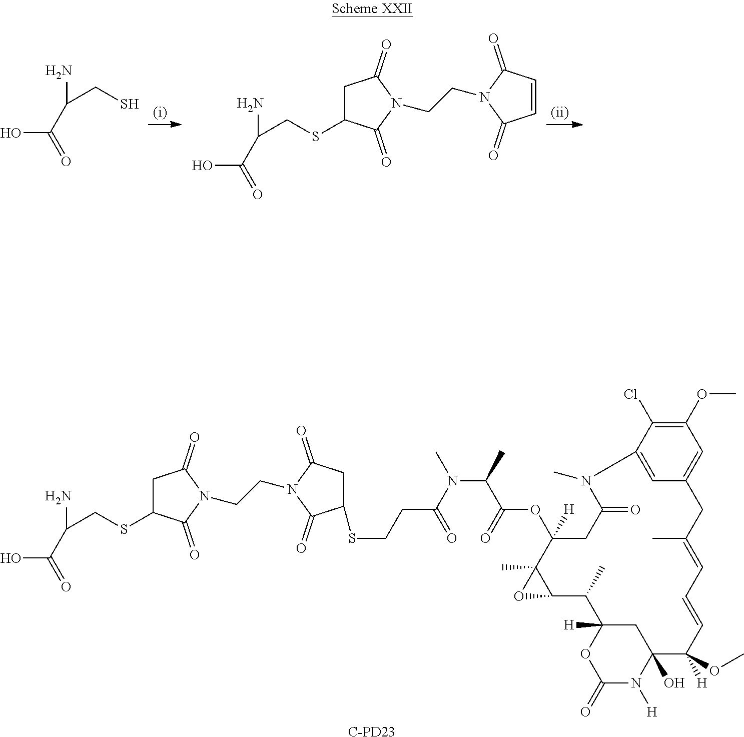 [SMILES: NC(CS)C(=O)O ; NC(CSC1CC(=O)N(CCN2C(=O)C=CC2=O)C1=O)C(=O)O ; COc1cc2cc(c1Cl)N(C)C(=O)C[C@H](OC(=O)[C@H](C)N(C)C(=O)CCSC1CC(=O)N(CCN3C(=O)CC(SCC(N)C(=O)O)C3=O)C1=O)[C@]1(C)O[C@H]1[C@H](C)[C@@H]1C[C@@](O)(NC(=O)O1)[C@H](OC)/C=C/C=C(\C)C2 ; "C-PD23"]

Polar drug C-PD23, i.e., Cys-1,2-Bis(maleimido)ethane-DM1, was prepared as depicted in Scheme XXIII above: (i) maleimide-S bond formation between cysteine and 1,2-bis(male-imido)ethane following the maleimide-thiol protocol and (ii) maleimide-S bond formation between cysteine-1,2-bis(maleimido)ethane and DM1 followed the maleimide-thiol protocol.

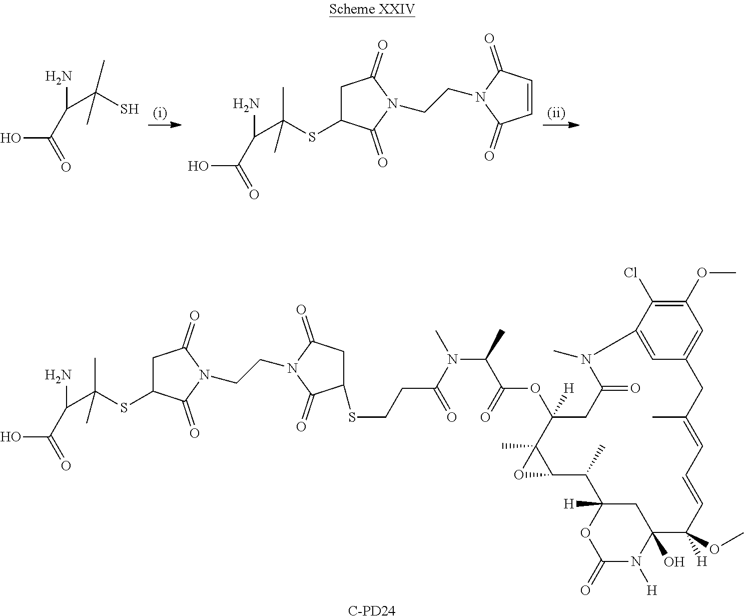

Polar drug C-PD24, i.e., L-(+)-Penicillamine-1,2-Bis(maleimido)ethane-DM1, was prepared as depicted in Scheme XXIV above: (i) maleimide-S bond formation between L-(+)-penicillamine and 1,2-bis(maleimido)ethane following the maleimide-thiol protocol and (ii) maleimide-S bond formation between L-(+)-penicillamine-1,2-bis(maleimido)ethane and DM1 followed the maleimide-thiol protocol.

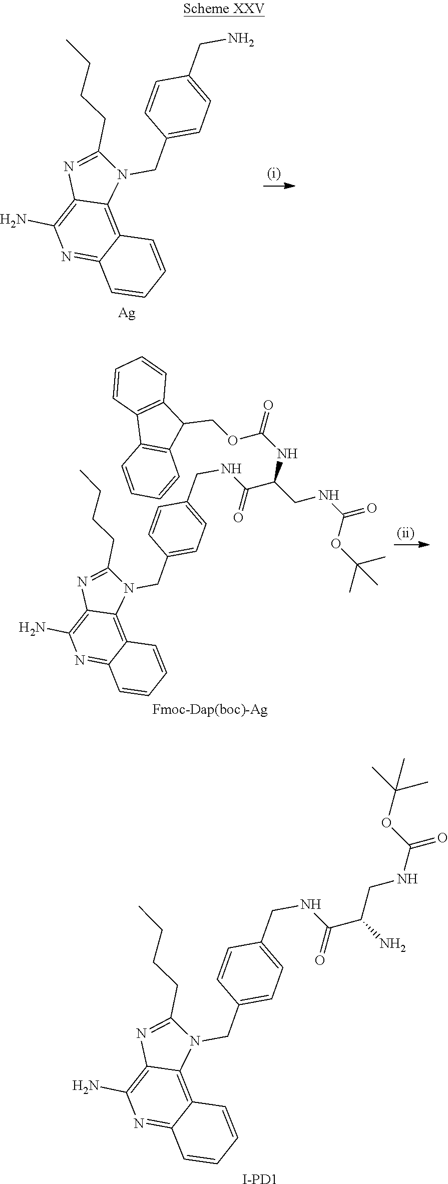

Polar drug I-PD1, i.e., Dap(Boc)-Ag, was prepared as depicted in Scheme XXV above: (i) amide bond formation between TLR7/8 agonist Ag and Na-Fmoc-NO-Boc-L-2,3-diaminopropionic acid (Fmoc-Dap(Boc)-OH) following the HATU protocol and (ii) removal of the Fmoc protection group of Fmoc-Dap(Boc)-Ag followed the Fmoc protocol.

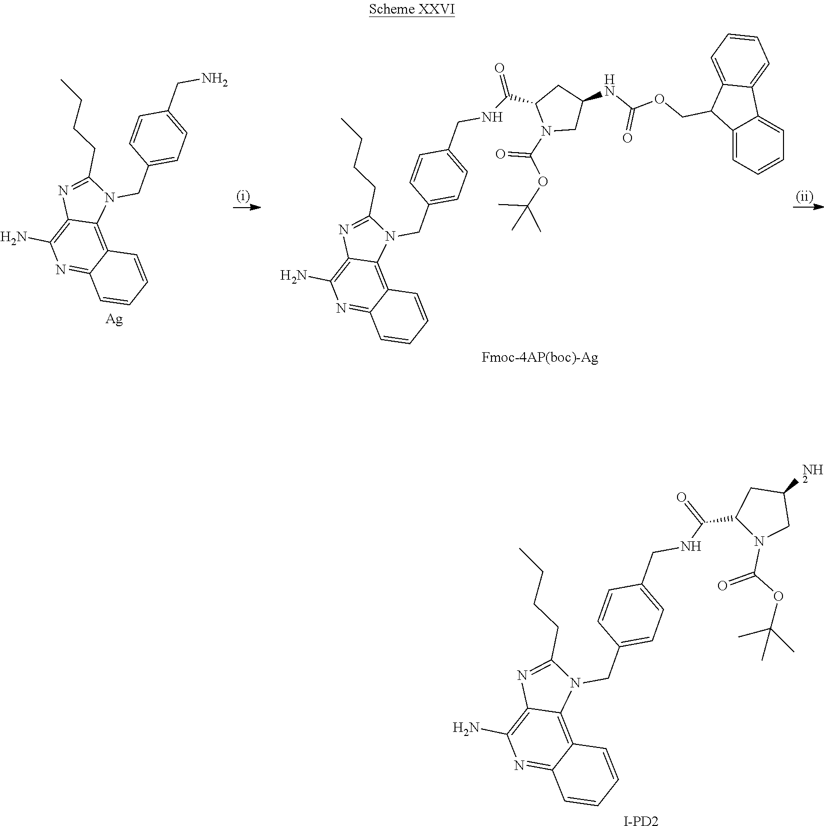

Polar drug I-PD2, i.e., 4AP(Boc)-Ag, was prepared as depicted in Scheme XXVI above: (i) amide bond formation between TLR7/8 agonist Ag and (2S,4R)-Fmoc-4-amino-1-Boc-pyrrolidine-2-carboxylic acid (Fmoc-4AP(Boc)-OH) following the HATU protocol and (ii) removal of the Fmoc protection group of Fmoc-4AP(Boc)-Ag followed the Fmoc protocol.

### Example 1: Compound 1, i.e., C-PD2-LK1 (PD=Ser-AMAS-DM1; HP=2X PEG12m)

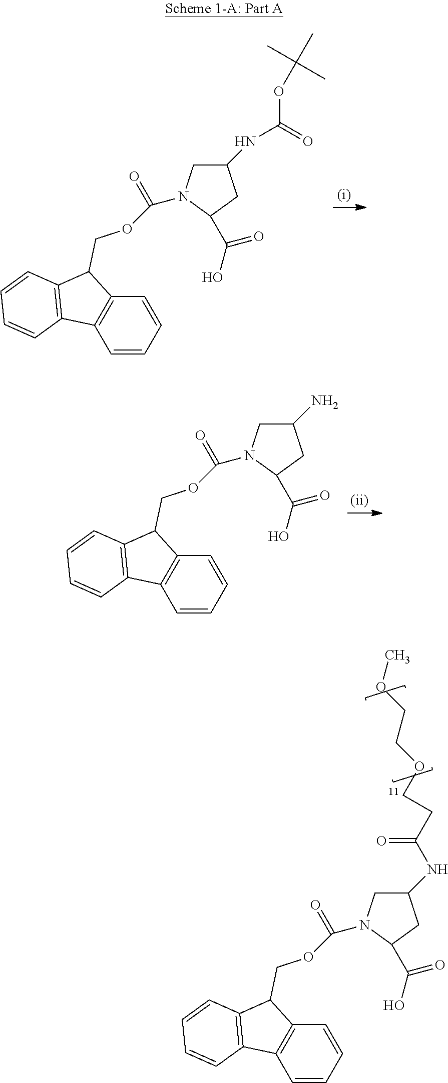

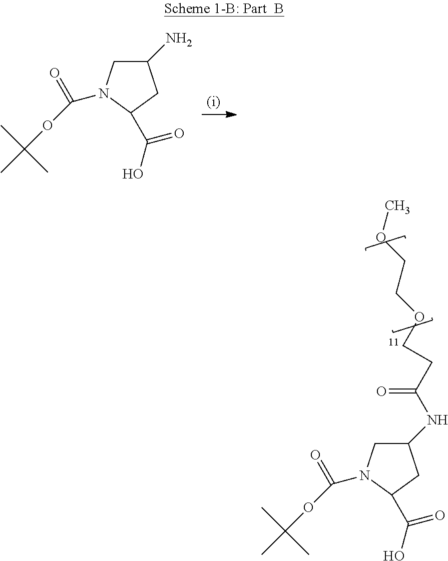

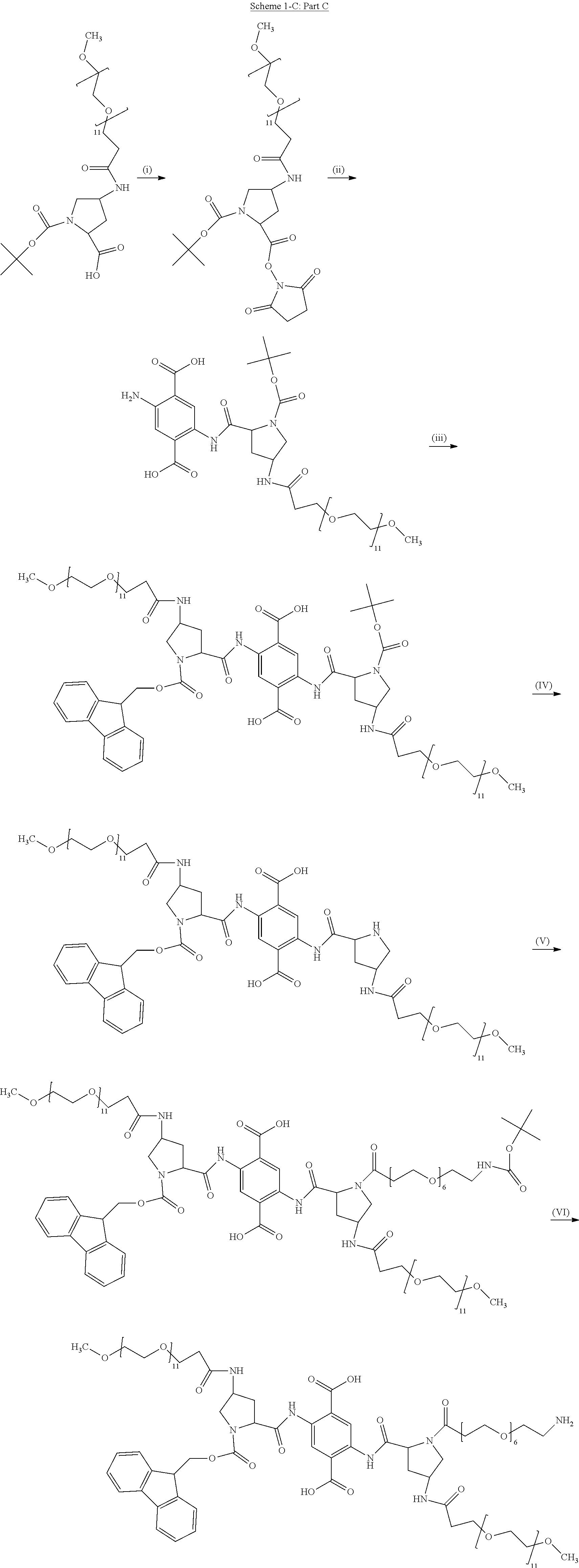

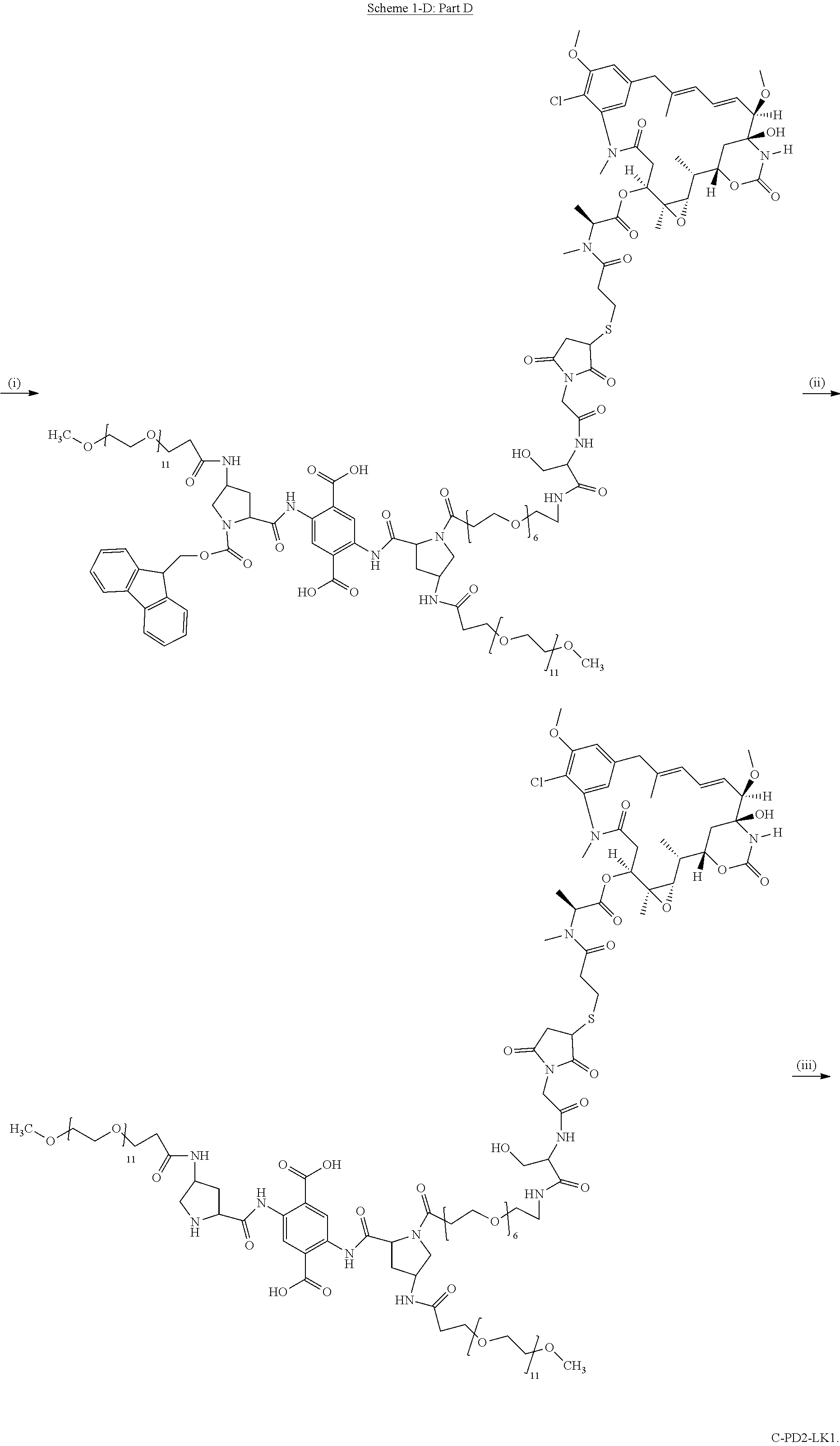

Compound 1 of this invention, i.e., Mal-PEG2-4AP(PEG12m)-H2DATA-4AP(PEG12m)-PEG6-Ser-AMAS-DM1, was prepared as shown in Schemes 1-A to 1-D above. The protocols used in each step are indicated below.

### Part A.

(i) Removal of the Boc protection group of (2R,4S)-1-Fmoc-4-Boc-amino pyrrolidine-2-carboxylic acid (Fmoc-4AP(Boc)-OH) followed the Boc protocol.
(ii) The amide bond formation between Fmoc-4AP(NH2)-OH and m-PEG12-NHS followed the NHS protocol.

### Part B.

(i) The amide bond formation between (2R,4S)-4-((tert-butoxycarbonyl)amino)-pyrrolidine-2-carboxylic acid (Boc-4AP(NH2)-OH) and m-PEG12-NHS followed the NHS protocol.

### Part C.

(i) Synthesis of NHS ester of Boc-4AP(PEG12m)—OH followed the protocol for NHS ester protocol.
(ii) The amide bond formation between Boc-4AP(PEG12m)—NHS and 2,5-Diaminoterephthalic acid (H2DATA) followed the NHS protocol.
(iii) The amide bond formation between Fmoc-4AP(PEG12m)—OH and H2DATA-4AP(PEG12m)-Boc followed the HATU protocol.
(iv) Removal of the Boc protection group of Fmoc-4AP(PEG12m)-H2DATA-4AP(PEG12m)-Boc followed the Boc protocol.
(v) The amide bond formation between Fmoc-4AP(PEG12m)-H2DATA-4AP(PEG12m)—NH2 and t-Boc-N-amido-PEG6-acid followed the HATU protocol.
(vi) Removal of the Boc protection group of Fmoc-4AP(PEG12m)-H2DATA-4AP(PEG12m)-PEG6-N-amido-Boc-t followed the Boc protocol.

### Part D.

(i) The amide bond formation between Fmoc-4AP(PEG12m)-H2DATA-4AP(PEG12m)-PEG6-NH2 and DM1-AMAS-Serine followed the HATU protocol.
(ii) Removal of the Fmoc protection group of Fmoc-4AP(PEG12m)-H2DATA-4AP(PEG12m)-PEG6-Ser-AMAS-DM1 followed the Fmoc protocol.
(iii) The amide bond formation between NH2-4AP(PEG12m)-H2DATA-4AP(PEG12m)-PEG6-Ser-AMAS-DM1 and Mal-PEG2-acid followed the HATU protocol.

### Example 2: Compound 2, i.e., C-PD5-LK2 (PD=Ser-PEG3-DM1; HP=2X PEG12m)

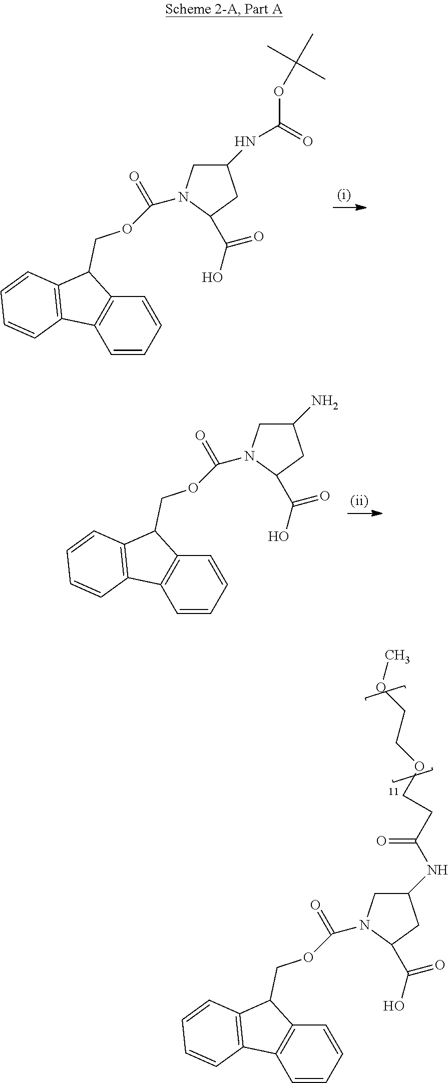

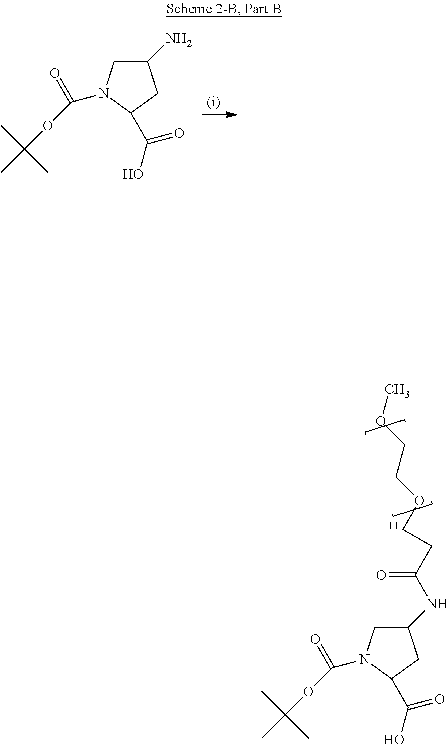

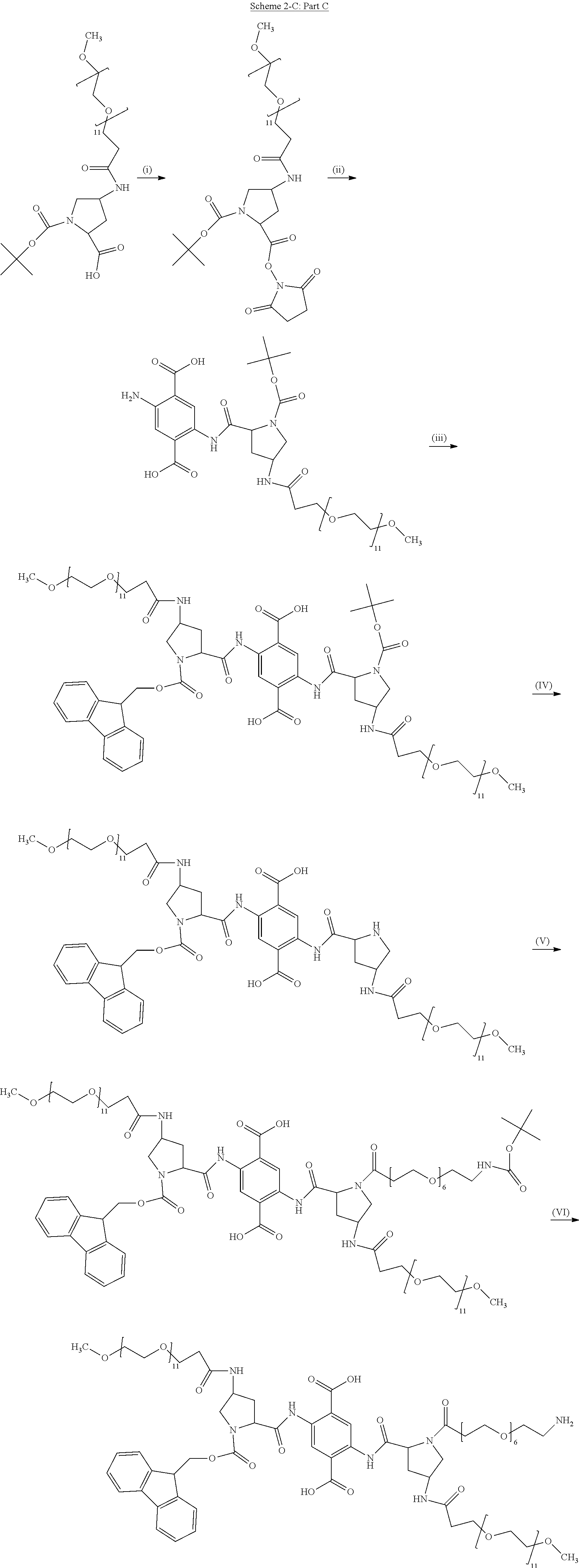

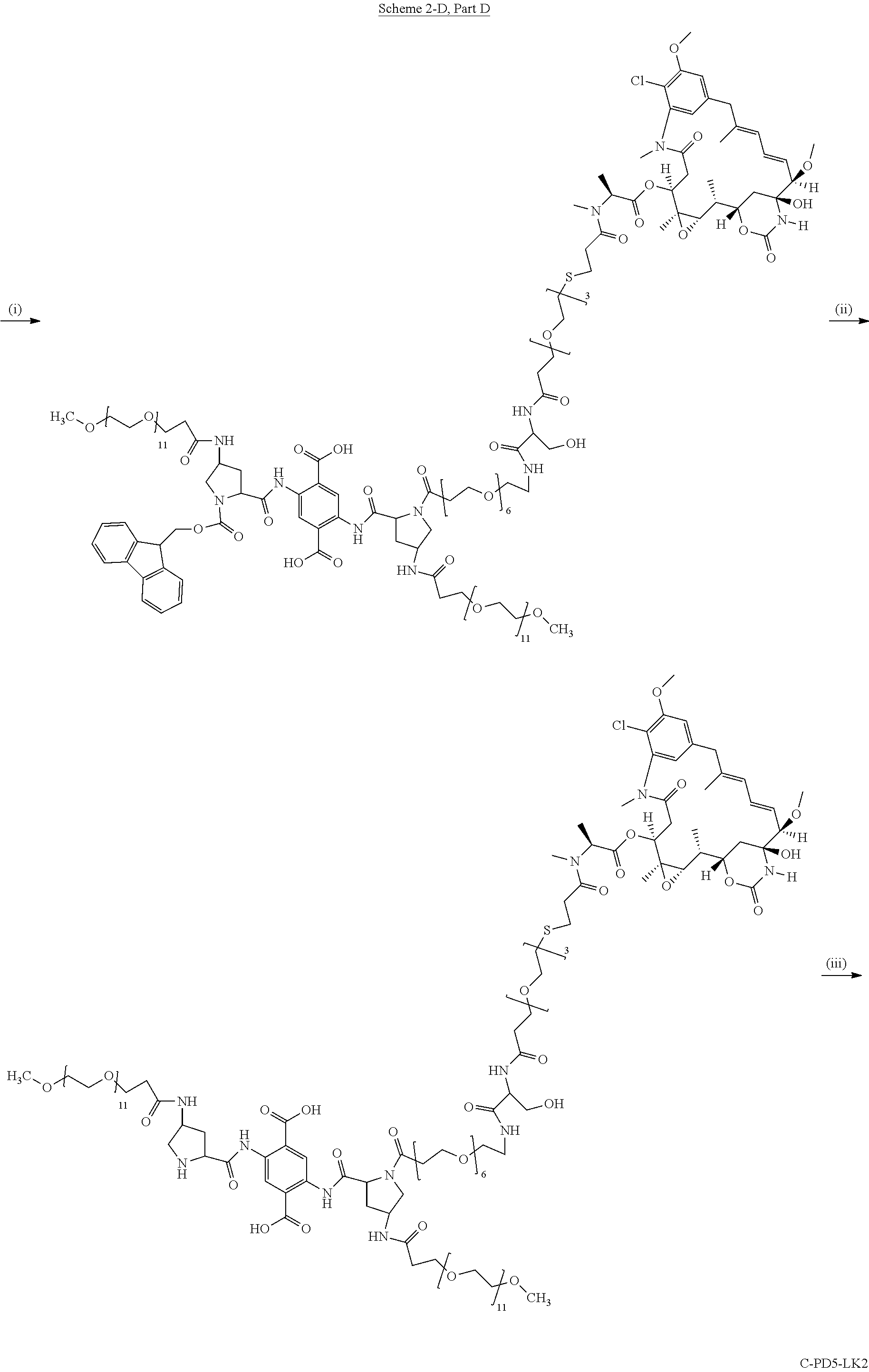

Compound 2 of this invention, i.e., Mal-PEG2-4AP(PEG12m)-H2DATA-4AP(PEG12m)-PEG6-Ser-PEG3-DM1 was prepared following the protocols as indicated below.

### Part A.

(i) Removal of the Boc protection group of (2R,4S)-1-Fmoc-4-Boc-amino pyrrolidine-2-carboxylic acid (Fmoc-4AP(Boc)-OH) followed the Boc protocol.
(ii) The amide bond formation between Fmoc-4AP(NH2)-OH and m-PEG12-NHS followed the NHS protocol.

### Part B.

(i) The amide bond formation between (2R,4S)-4-((tert-Butoxycarbonyl)amino)pyrrolidine-2-carboxylic acid (Boc-4AP(NH2)-OH) and m-PEG12-NHS followed the NHS protocol.

### Part C.

(i) Synthesis of NHS ester of Boc-4AP(PEG12m)—OH followed the NHS ester protocol.
(ii) The amide bond formation between Boc-4AP(PEG12m)—NHS and 2,5-Diaminoterephthalic acid (H2DATA) followed the NHS protocol.
(iii) The amide bond formation between Fmoc-4AP(PEG12m)—OH and H2DATA-4AP(PEG12m)-Boc followed the HATU protocol.
(iv) Removal of the Boc protection group of Fmoc-4AP(PEG12m)-H2DATA-4AP(PEG12m)-Boc followed the Boc protocol.
(v) The amide bond formation between Fmoc-4AP(PEG12m)-H2DATA-4AP(PEG12m)—NH2 and t-Boc-N-amido-PEG6-acid followed the HATU protocol.
(vi) Removal of the Boc protection group of Fmoc-4AP(PEG12m)-H2DATA-4AP(PEG12m)-PEG6-N-amido-Boc-t followed the Boc protocol.

### Part D.

(i) The amide bond formation between Fmoc-4AP(PEG12m)-H2DATA-4AP(PEG12m)-PEG6-NH2 and DM1-PEG3-Serine followed the HATU protocol.
(ii) Removal of the Fmoc protection group of Fmoc-4AP(PEG12m)-H2DATA-4AP(PEG12m)-PEG6-Ser-PEG3-DM1 followed the Fmoc protocol.
(iii) The amide bond formation between NH2-4AP(PEG12m)-H2DATA-4AP(PEG12m)-PEG6-Ser-PEG3-DM1 and Mal-PEG2-acid followed the HATU protocol.

### Example 3: Compound 3, i.e., C-PD18-LK3 (PD=SPDP-DM4; HP=2X PEG24m)

### Part A

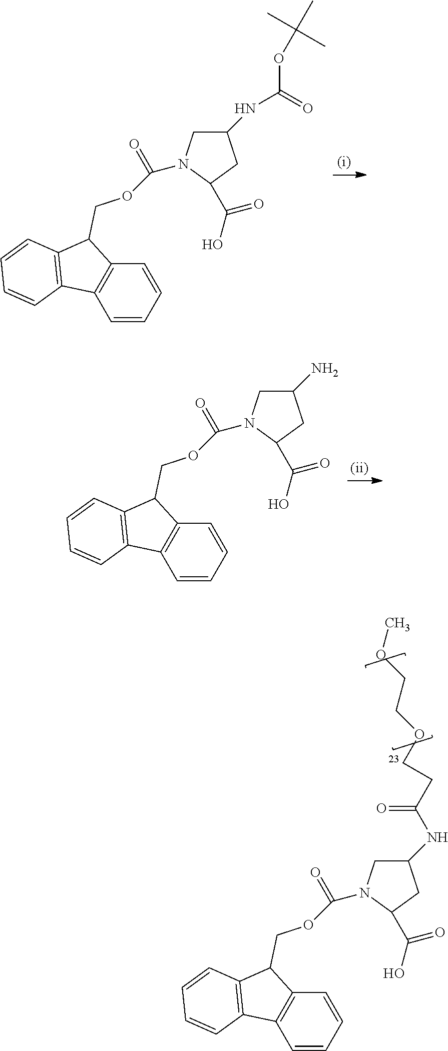

### Part B.

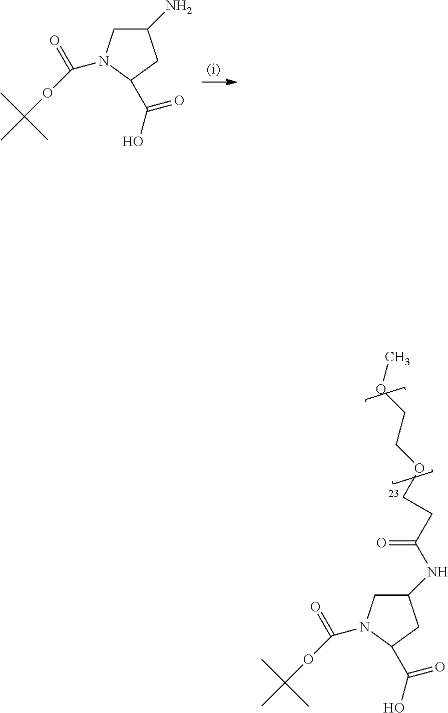

### Part C

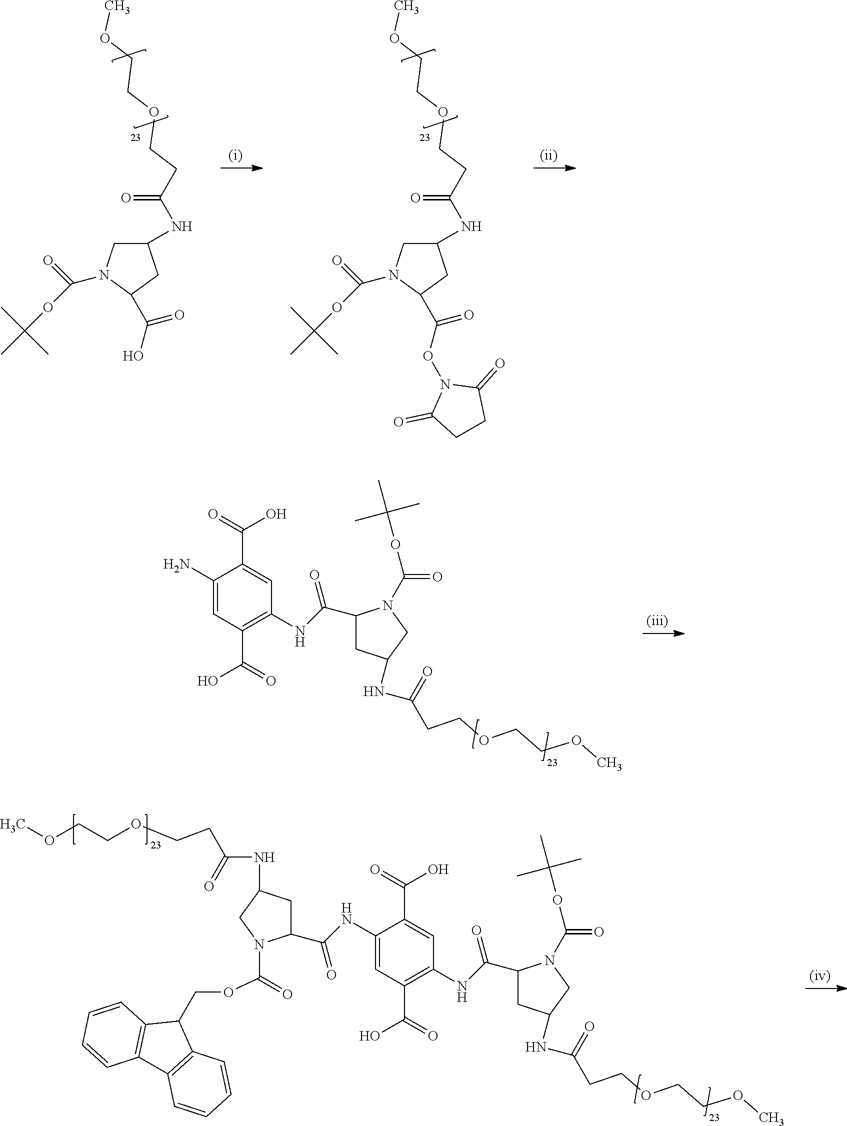

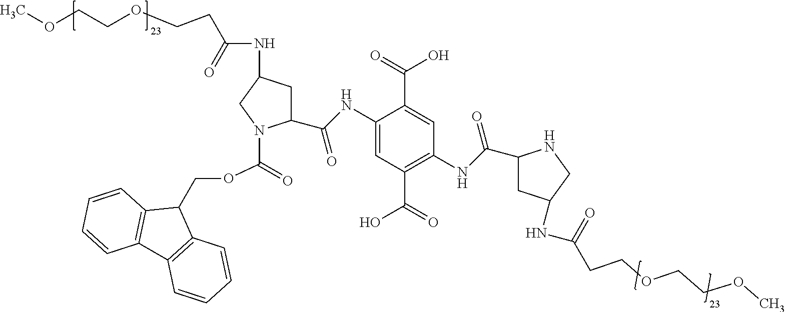

### Part D

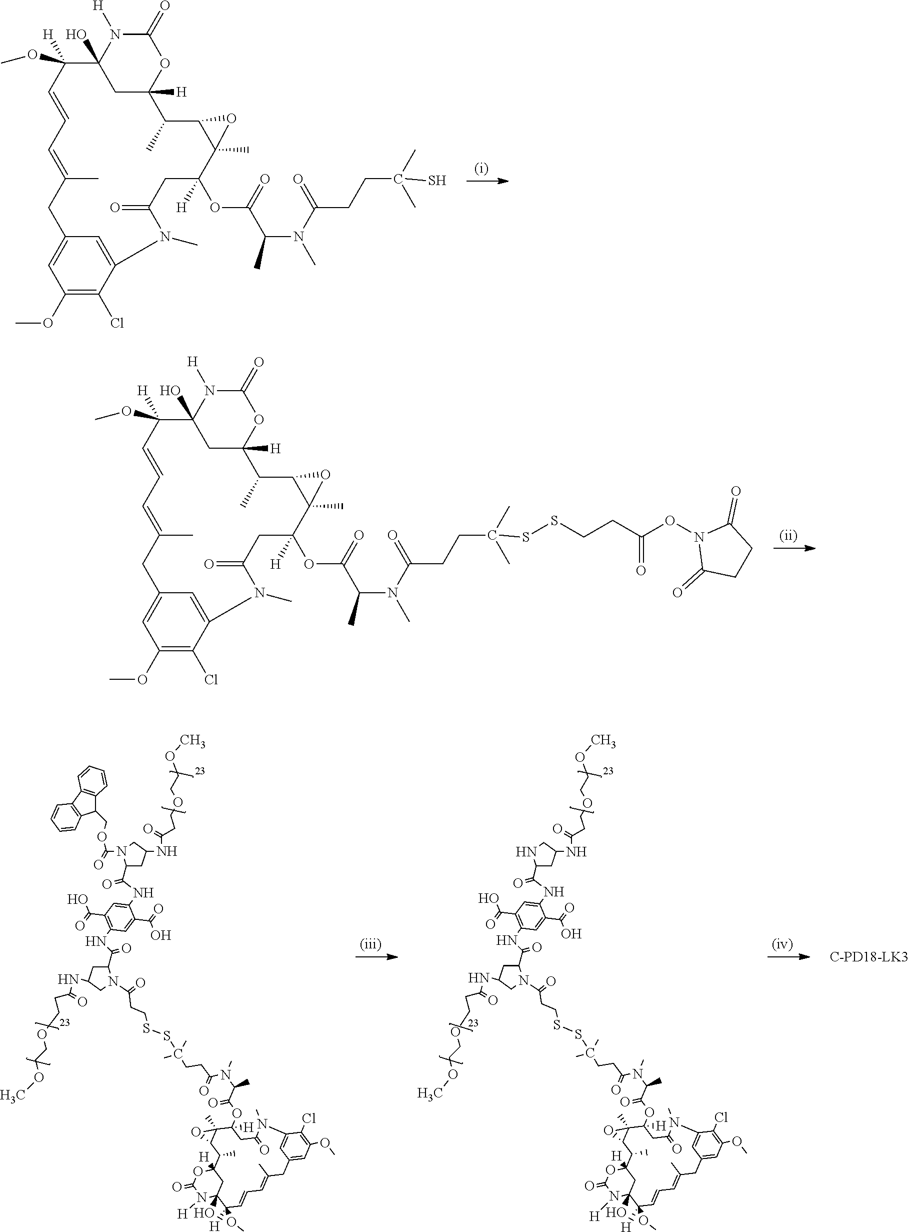

Compound 3 of this invention, i.e., 4-Maleimidophenylacetic Acid-4AP(PEG24m)-H2DATA-4AP(PEG24m)-SPDP-DM4, was prepared following the protocols as indicated below:

### Part A.

(i) Removal of the Boc protection group of (2R,4S)-1-Fmoc-4-Boc-amino pyrrolidine-2-carboxylic acid (Fmoc-4AP(Boc)-OH) followed the Boc protocol.
(ii) The amide bond formation between Fmoc-4AP(NH2)-OH and m-PEG24-NHS followed the NHS protocol.

### Part B.

(i) The amide bond formation between (2R,4S)-4-((tert-butoxycarbonyl)-amino)pyro-lidine-2-carboxylic acid (Boc-4AP(NH2)-OH) and m-PEG24-NHS followed the NHS protocol.

### Part C.

(i) Synthesis of NHS ester of Boc-4AP(PEG24m)—OH followed the NHS ester protocol.
(ii) The amide bond formation between Boc-4AP(PEG24m)—NHS and 2,5-Diaminoterephthalic acid (H2DATA) followed the NHS protocol.
(iii) The amide bond formation between Fmoc-4AP(PEG24m)—OH and H2DATA-4AP(PEG24m)-Boc followed the HATU protocol.
(iv) Removal of the Boc protection group of Fmoc-4AP(PEG24m)-H2DATA-4AP(PEG24m)-Boc followed the Boc protocol.

### Part D.

(i) The disulfide bond formation between DM4 and SPDP followed the SPDP protocol.
(ii) The amide bond formation between Fmoc-4AP(PEG24m)-H2DATA-4AP(PEG24m)—NH2 and DM4-SPDP followed the NHS protocol.
(iii) Removal of the Fmoc protection group of Fmoc-4AP(PEG24m)-H2DATA-4AP(PEG24m)-SPDP-DM4 followed the Fmoc protocol.
(iv) The amide bond formation between NH2-4AP(PEG24m)-H2DATA-4AP(PEG24m)-SPDP-DM4 and 4-Maleimidophenylacetic Acid followed the HATU protocol.

### Example 4: Compound 4, i.e., C-PD18-LK4 (PD=SPDP-DM4; HP=PEG24)

### Part A

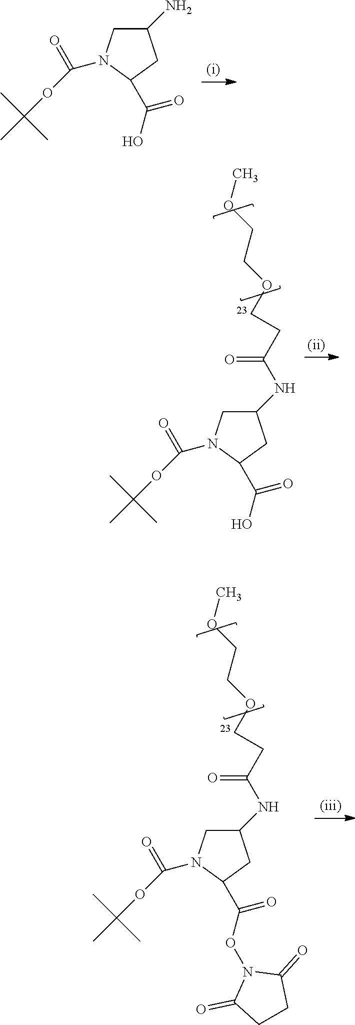

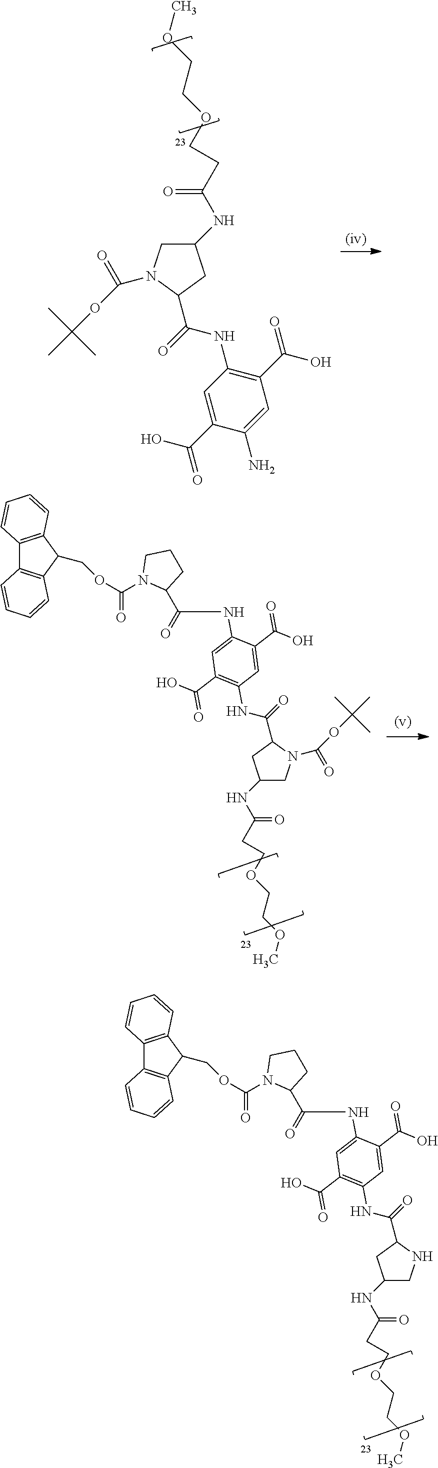

### Part B.

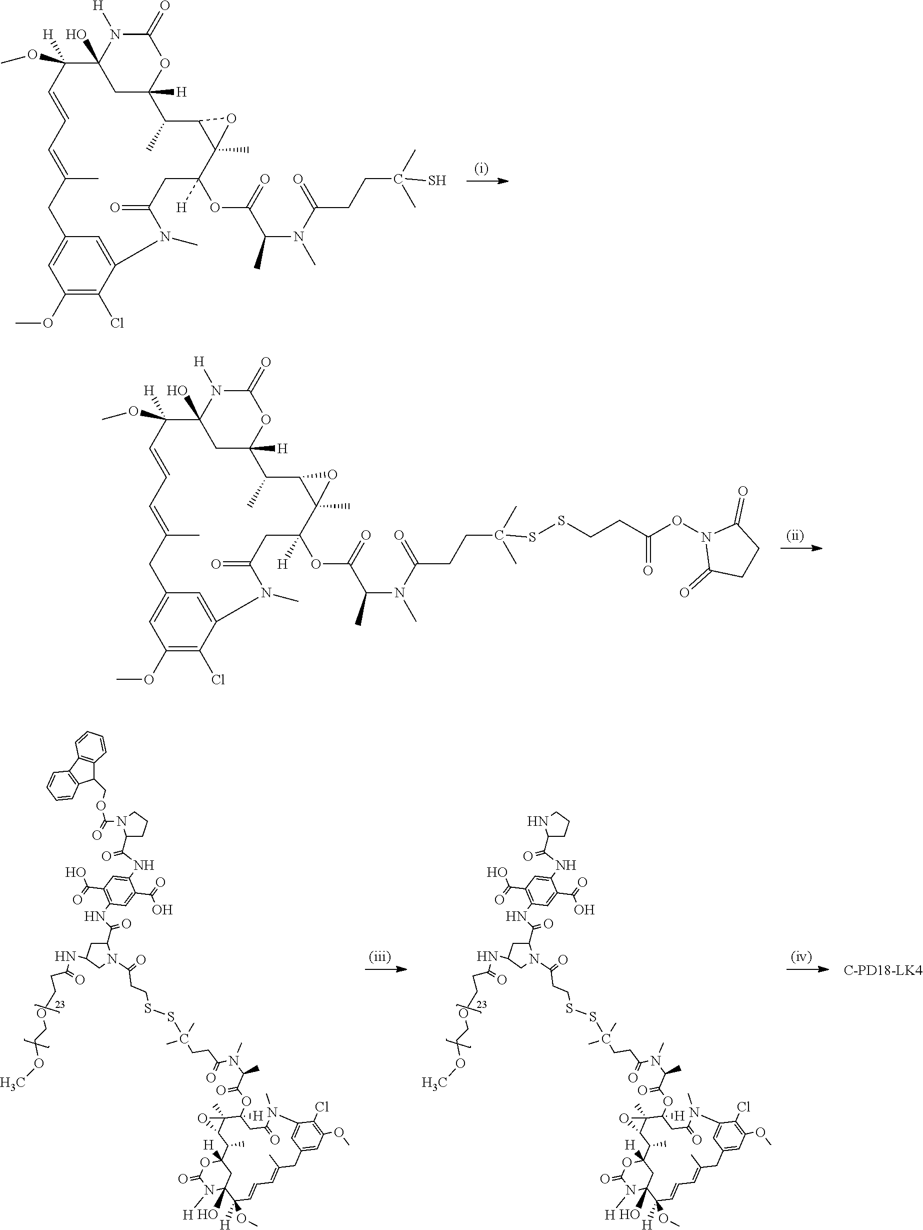

Compound 4 of this invention, i.e., 4-Maleimidophenylacetic Acid-Pro-H2DATA-4AP(PEG24m)-SPDP-DM4, was synthesized following the protocols as indicated below.

### Part A.

(i) The amide bond formation between (2R,4S)-4-((tert-butoxycarbonyl)amino)-pyrrolidine-2-carboxylic acid (Boc-4AP(NH2)-OH) and m-PEG24-NHS followed the NHS protocol.
(ii) Synthesis of NHS ester of Boc-4AP(PEG24m)—OH followed the NHS ester protocol.
(iii) The amide bond formation between Boc-4AP(PEG24m)—NHS and 2,5-Diaminoterephthalic acid (H2DATA) followed the NHS protocol.
(iv) The amide bond formation between Fmoc-Pro-OH and H2DATA-4AP(PEG24m)-Boc followed the HATU protocol.
(v) Removal of the Boc protection group of Fmoc-Pro-H2DATA-4AP(PEG24m)-Boc followed the Boc protocol.

### Part B

(i) The disulfide bond formation between DM4 and SPDP followed the SPDP protocol.
(ii) The amide bond formation between Fmoc-Pro-H2DATA-4AP(PEG24m)—NH2 and DM4-SPDP followed the NHS protocol.
(iii) Removal of the Fmoc protection group of Fmoc-Pro-H2DATA-4AP(PEG24m)-SPDP-DM4 followed the Fmoc protocol.
(iv) The amide bond formation between NH2-Pro-H2DATA-4AP(PEG24m)-SPDP-DM4 and 4-Maleimidophenylacetic Acid followed the HATU protocol.

### Example 5: Compound 5, i.e., C-PD2-LK5 (PD=Ser-AMAS-DM1; HP=2X PEG12)

### Part A

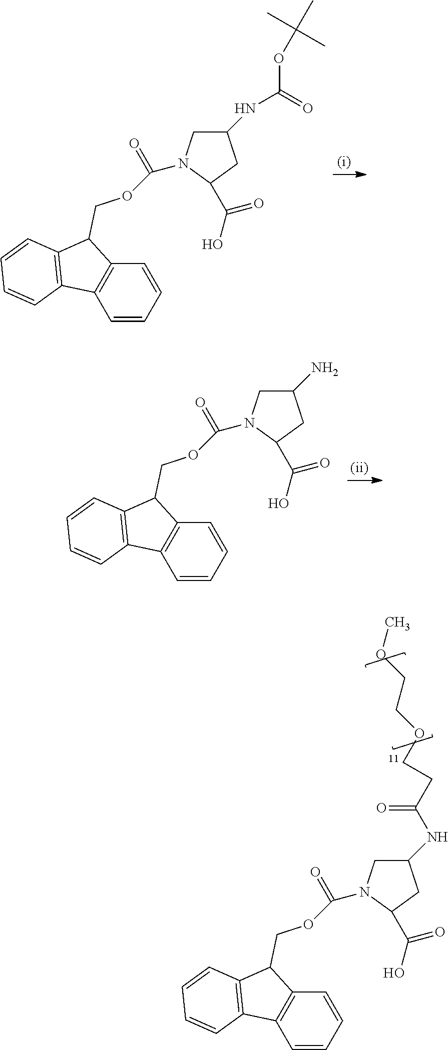

### Part B

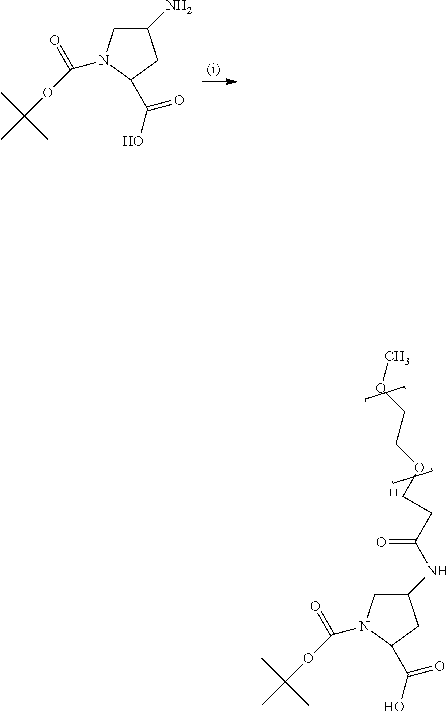

### Part C

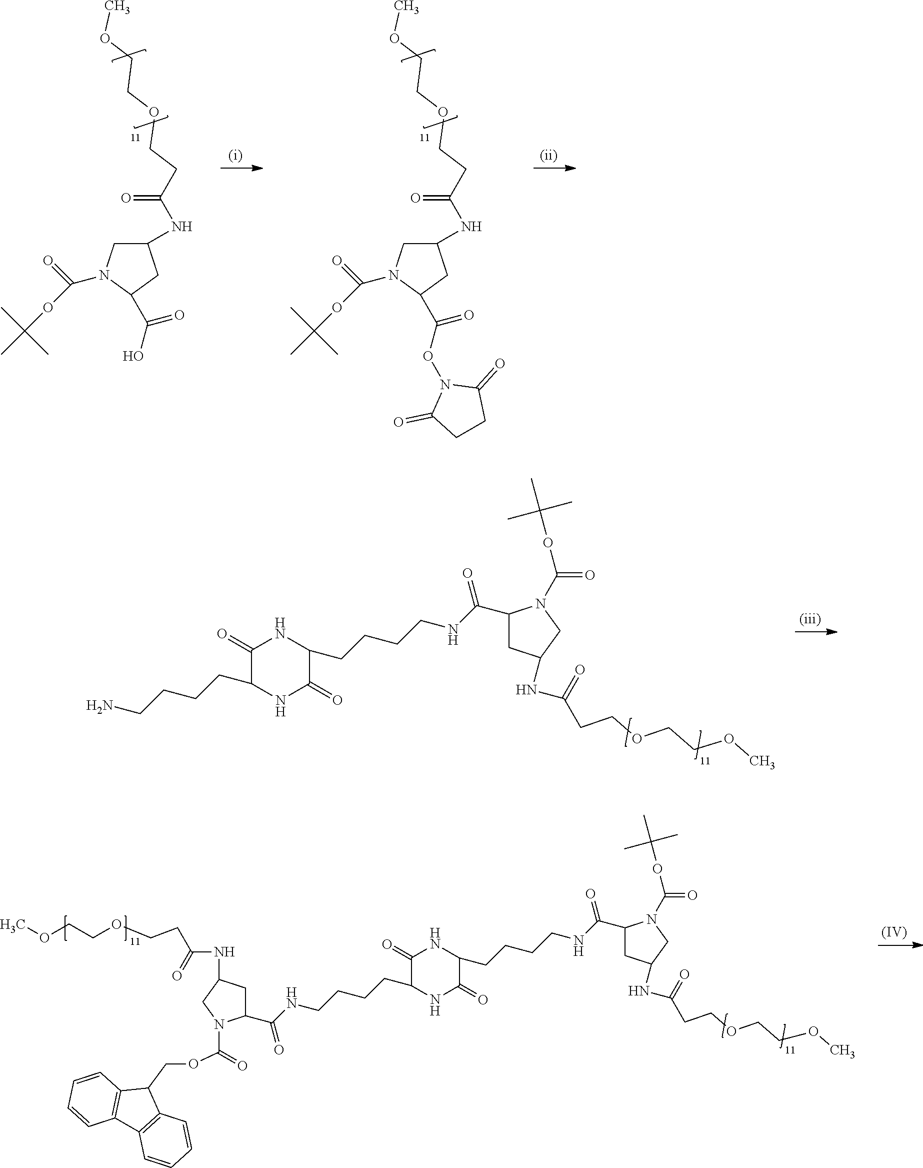

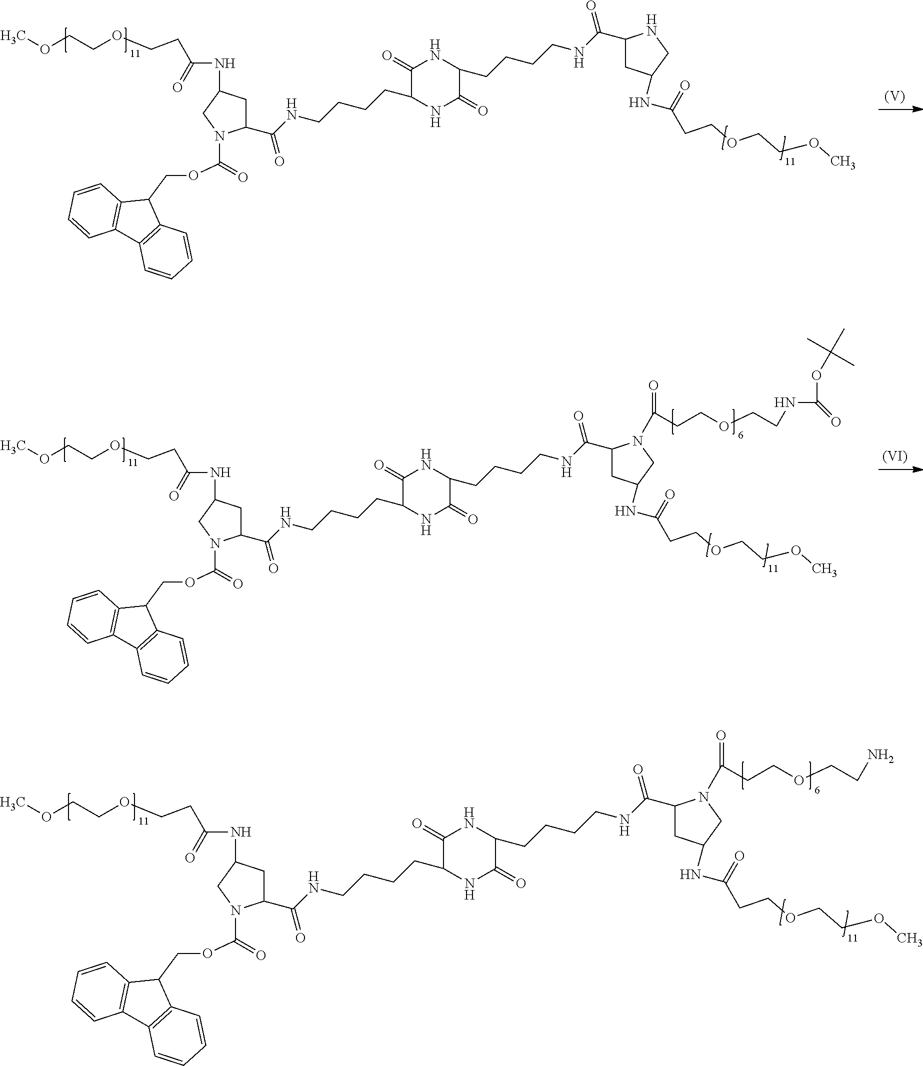

### Part D

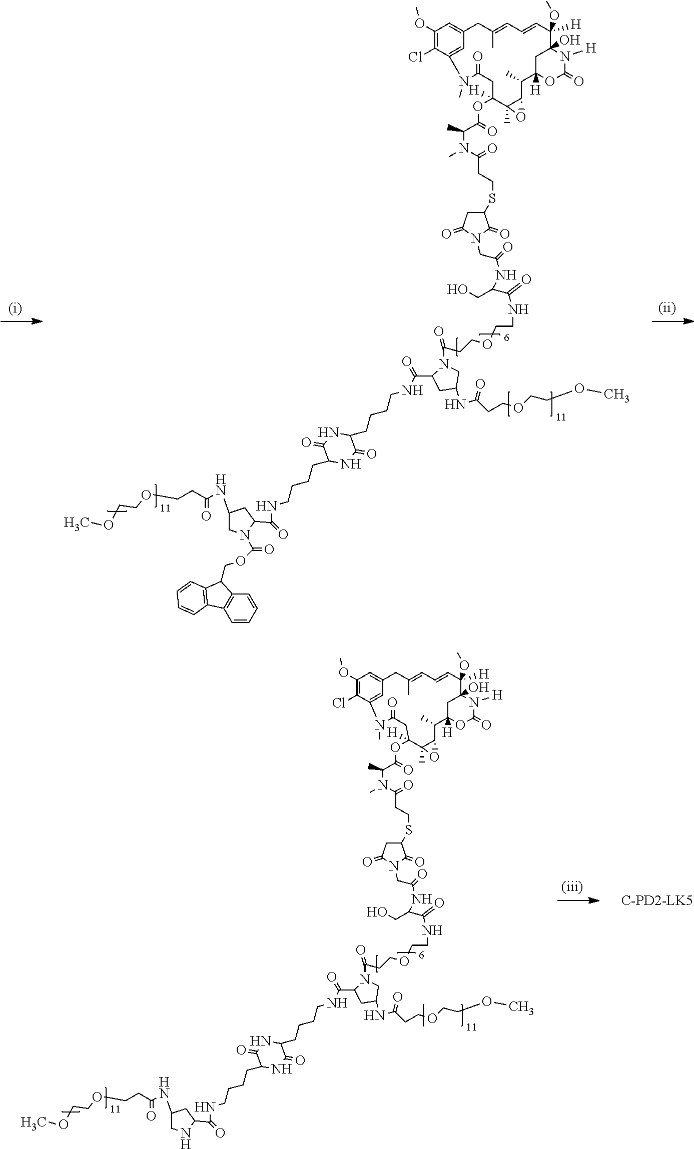

Compound 5 of this invention, i.e., Mal-PEG2-4AP(PEG12m)-CDK-4AP(PEG12m)-PEG6-Ser-AMAS-DM1, was synthesized following the protocols as indicated below.

### Part A.

(i) Removal of the Boc protection group of (2R,4S)-1-Fmoc-4-Boc-amino pyrrolidine-2-carboxylic acid (Fmoc-4AP(Boc)-OH) followed the Boc protocol.
(ii) The amide bond formation between Fmoc-4AP(NH2)-OH and m-PEG12-NHS followed the NHS protocol.

### Part B.

(i) The amide bond formation between (2R,4S)-4-((tert-Butoxycarbonyl)amino)-pyrrolidine-2-carboxylic acid (Boc-4AP(NH2)-OH) and m-PEG12-NHS followed the NHS protocol.

### Part C.

(i) Synthesis of NHS ester of Boc-4AP(PEG12m)—OH followed the NHS ester protocol.
(ii) The amide bond formation between Boc-4AP(PEG12m)—NHS and 3,6-bis(4-aminobutyl)-2,5-piperazinedione (CDK) followed the NHS protocol.
(iii) The amide bond formation between Fmoc-4AP(PEG12m)—OH and CDK-4AP(PEG12m)-Boc followed the HATU protocol.
(iv) Removal of the Boc protection group of Fmoc-4AP(PEG12m)-CDK-4AP(PEG12m)-Boc followed the Boc protocol.
(v) The amide bond formation between Fmoc-4AP(PEG12m)-CDK-4AP(PEG12m)—NH2 and t-Boc-N-amido-PEG6-acid followed the HATU protocol.
(vi) Removal of the Boc protection group of Fmoc-4AP(PEG12m)-CDK-4AP(PEG12m)-PEG6-N-amido-Boc-t followed the Boc protocol.

### Part D.

(i) The amide bond formation between Fmoc-4AP(PEG12m)-CDK-4AP(PEG12m)-PEG6-NH2 and DM1-AMAS-Serine followed the HATU protocol.
(ii) Removal of the Fmoc protection group of Fmoc-4AP(PEG12m)-CDK-4AP(PEG12m)-PEG6-Ser-AMAS-DM1 followed the Fmoc protocol.
(iii) The amide bond formation between NH2-4AP(PEG12m)-CDK-4AP(PEG12m)-PEG6-Ser-AMAS-DM1 and Mal-PEG2-acid followed the HATU protocol.

### Example 6: Compound 6, i.e., C-PD5-LK6 (PD=Ser-PEG3-DM1; HP=2X PEG12)

### Part A

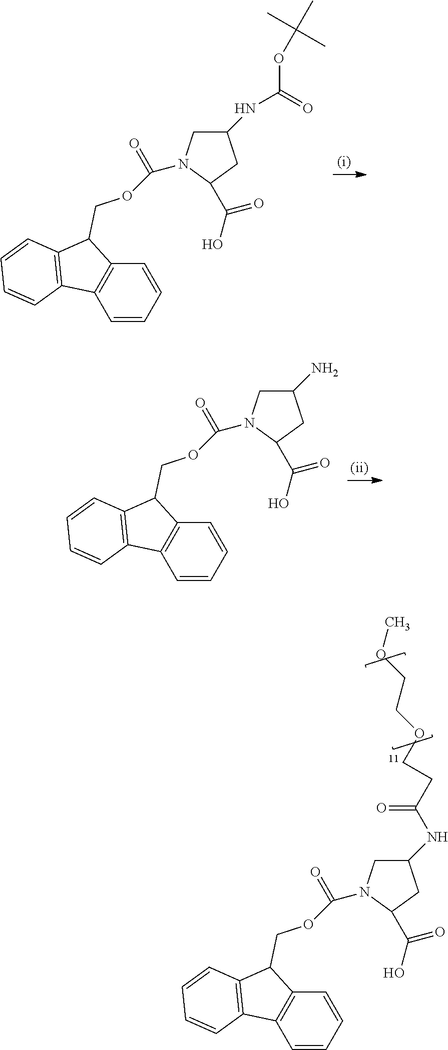

### Part B

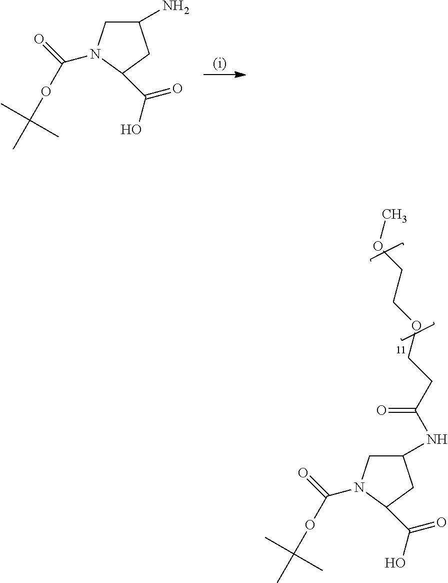

### Part C

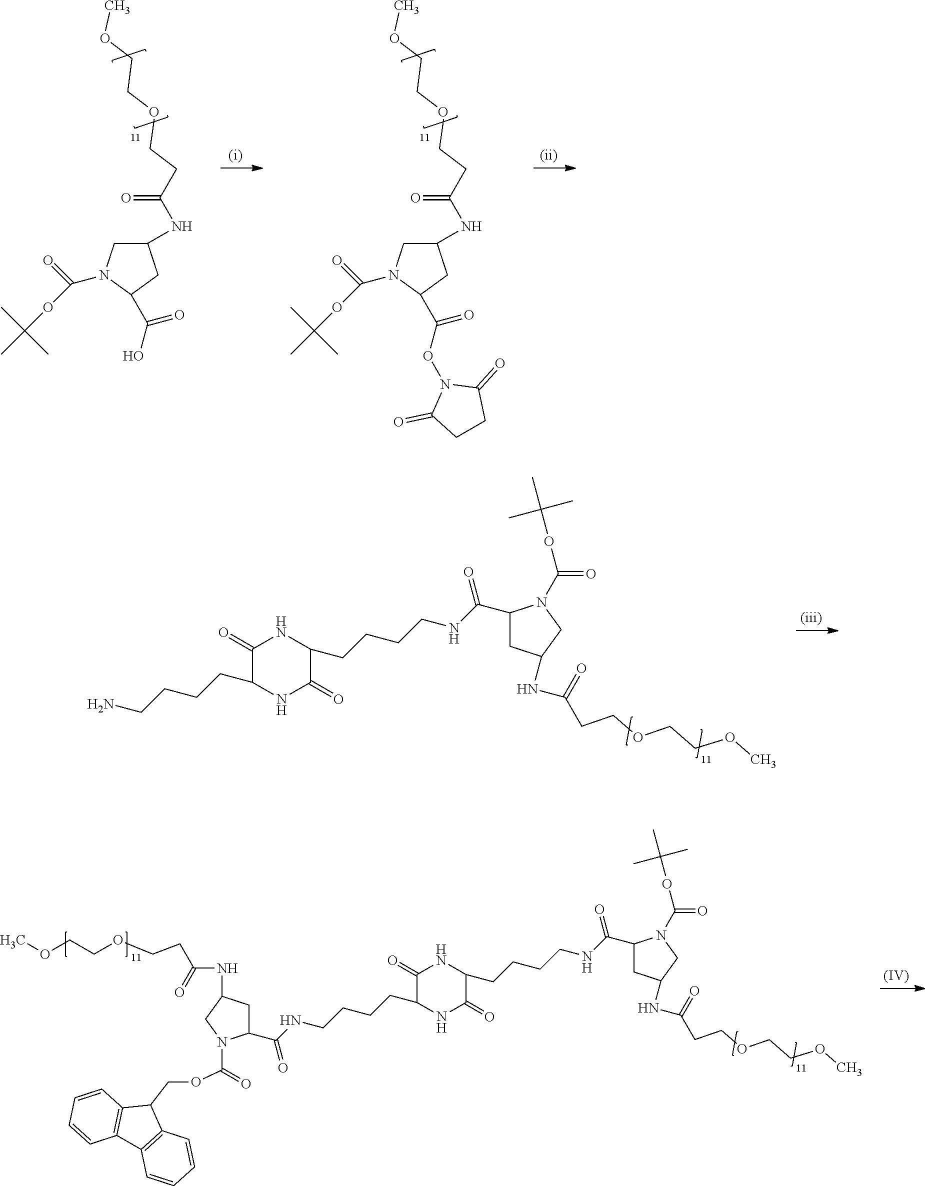

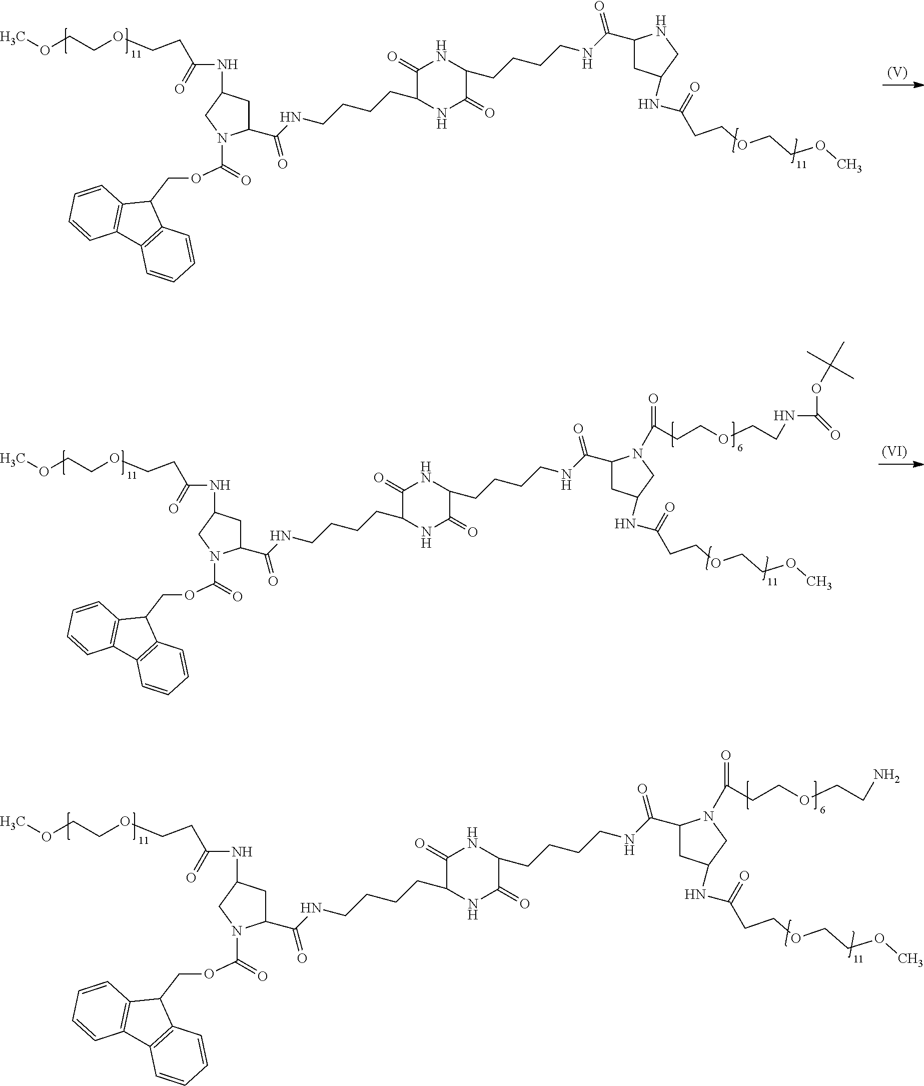

### Part D

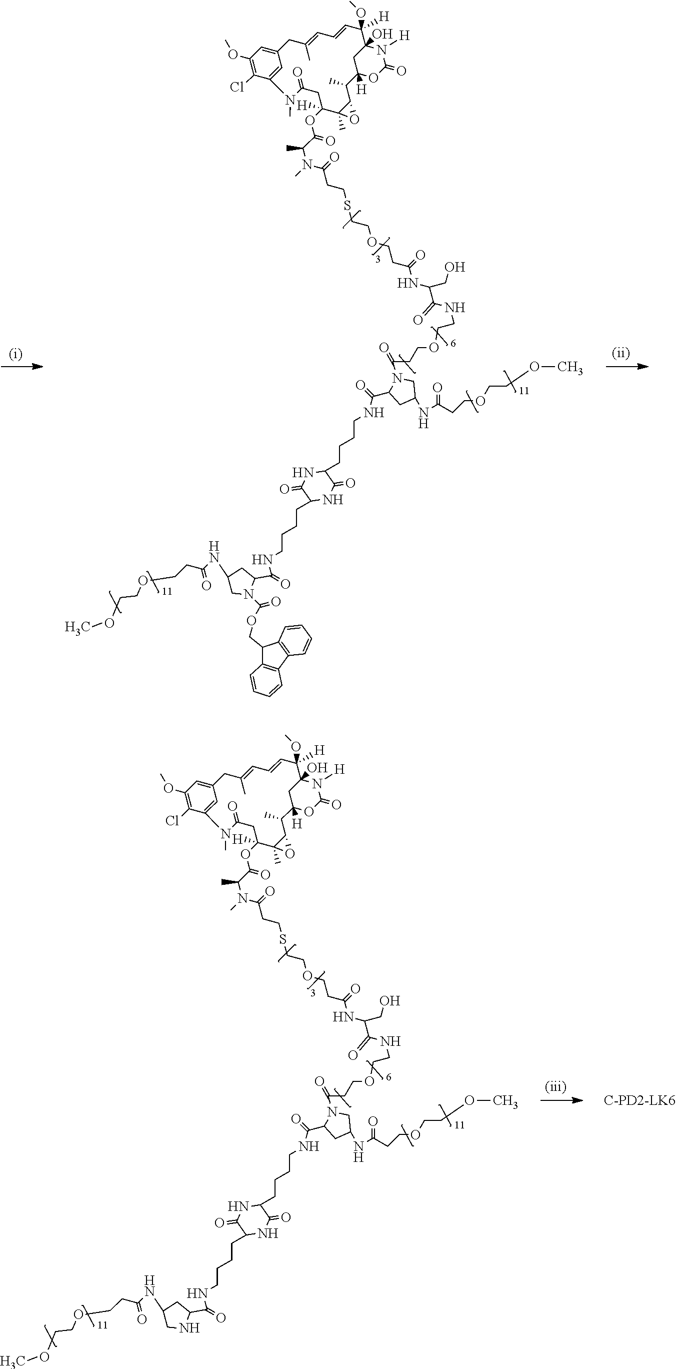

Compound 6 of this invention, i.e., Mal-PEG2-4AP(PEG12m)-CDK-4AP(PEG12m)-PEG6-Ser-PEG3-DM1, was synthesized following the protocols as indicated below.

### Part A.

(i) Removal of the Boc protection group of (2R,4S)-1-Fmoc-4-Boc-amino pyrrolidine-2-carboxylic acid (Fmoc-4AP(Boc)-OH) followed the Boc protocol.
(ii) The amide bond formation between Fmoc-4AP(NH2)-OH and m-PEG12-NHS followed the NHS protocol.

### Part B.

(i) The amide bond formation between (2R,4S)-4-((tert-Butoxycarbonyl)amino)-pyrrolidine-2-carboxylic acid (Boc-4AP(NH2)-OH) and m-PEG12-NHS followed the NHS protocol.

### Part C.

(i) Synthesis of NHS ester of Boc-4AP(PEG12m)—OH followed the NHS ester protocol.
(ii) The amide bond formation between Boc-4AP(PEG12m)—NHS and 3,6-bis(4-aminobutyl)-2,5-piperazinedione (CDK) followed the NHS protocol.
(iii) The amide bond formation between Fmoc-4AP(PEG12m)—OH and CDK-4AP(PEG12m)-Boc followed the HATU protocol.
(iv) Removal of the Boc protection group of Fmoc-4AP(PEG12m)-CDK-4AP(PEG12m)-Boc followed the Boc protocol.
(v) The amide bond formation between Fmoc-4AP(PEG12m)-CDK-4AP(PEG12m)—NH2 and t-Boc-N-amido-PEG6-acid followed the HATU protocol.
(vi) Removal of the Boc protection group of Fmoc-4AP(PEG12m)-CDK-4AP(PEG12m)-PEG6-N-amido-Boc-t followed the Boc protocol.

### Part D.

(i) The amide bond formation between Fmoc-4AP(PEG12m)-CDK-4AP(PEG12m)-PEG6-NH2 and DM1-PEG3-Serine followed the HATU protocol.
(ii) Removal of the Fmoc protection group of Fmoc-4AP(PEG12m)-CDK-4AP(PEG12m)-PEG6-Ser-PEG3-DM1 the Fmoc protocol.
(iii) The amide bond formation between NH2-4AP(PEG12m)-CDK-4AP(PEG12m)-PEG6-Ser-PEG3-DM1 and Mal-PEG2-acid followed the HATU protocol.

### Example 7: Compound 7, i.e., C-PD1-LK7 (PD=2X Dap-AMAS-DM1; HP=PEG48m)

### Part A

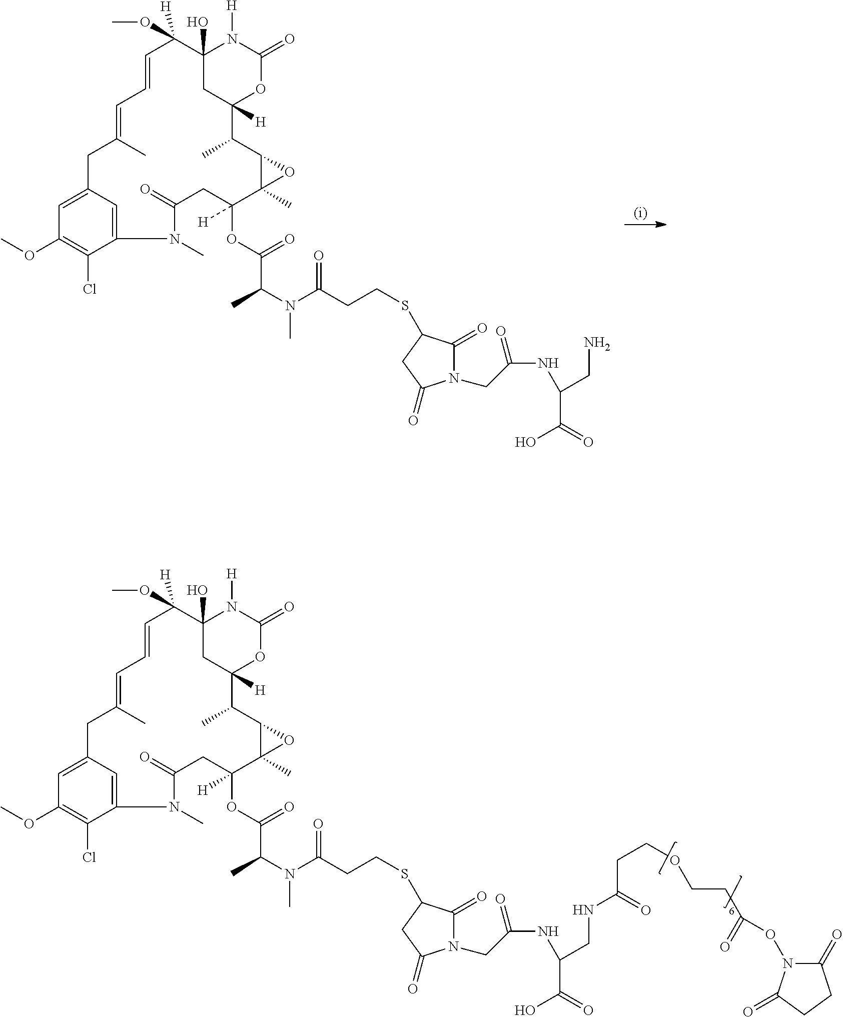

### Part B

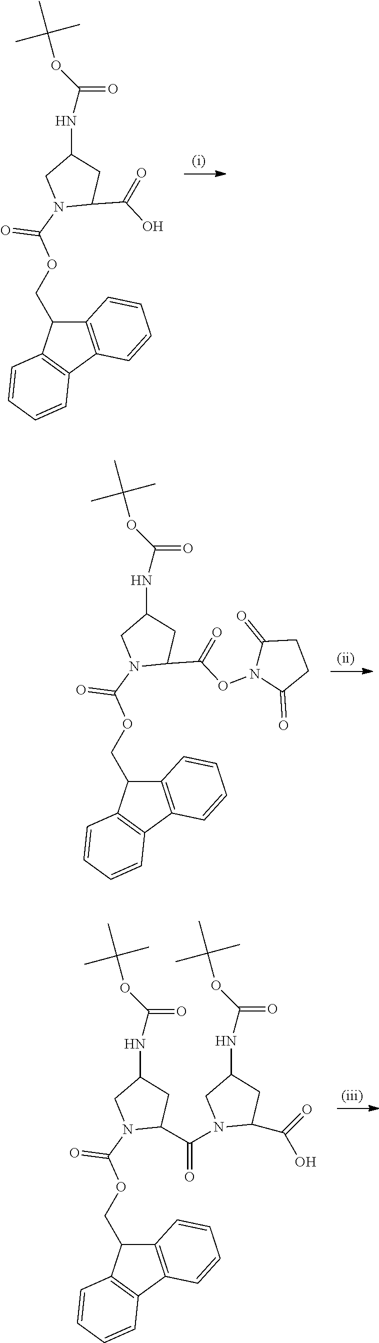

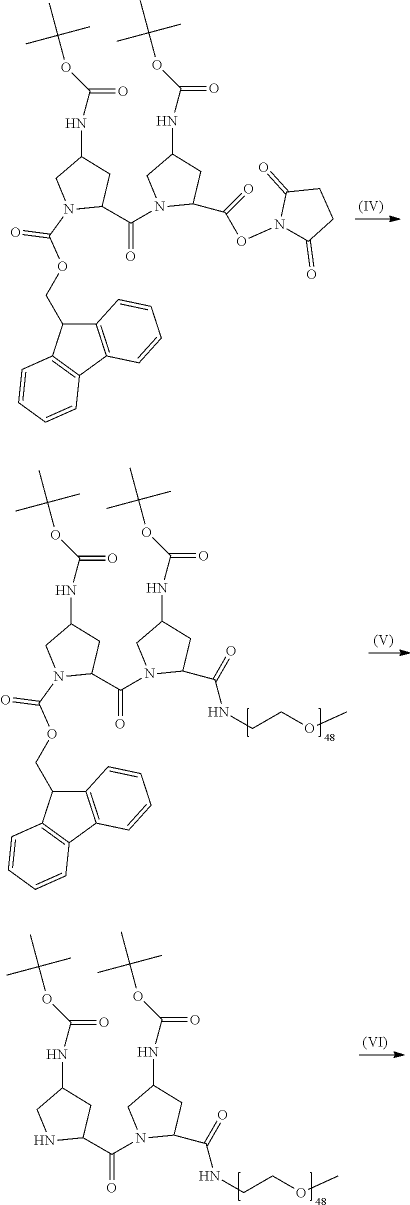

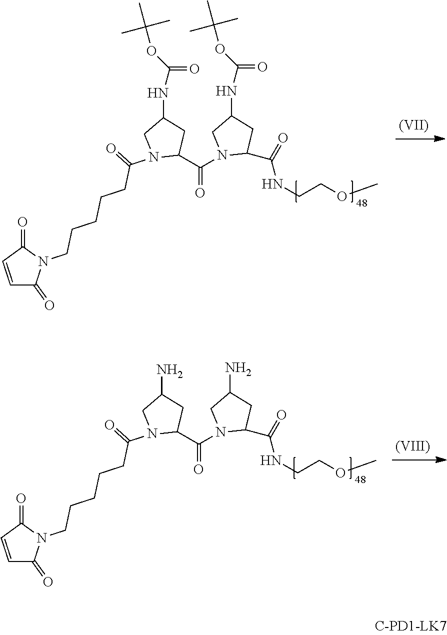

Compound 7 of this invention, i.e., MC-4AP(PEG6-Dap-AMAS-DM1)-4AP(PEG6-Dap-AMAS-DM1)-PEG48-m, was synthesized following the protocols as indicated below.

### Part A.

(i) The amide bond formation between DM1-AMAS-Dap(NH2)-OH and bis-PEG6-NHS followed the NHS protocol.

### Part B.

(i) Synthesis of NHS ester of Fmoc-4AP(Boc)-OH followed the NHS ester protocol.
(ii) The amide bond formation between Fmoc-4AP(Boc)-NHS and Fmoc-4AP(Boc)-OH followed the NHS protocol.
(iii) Synthesis of NHS ester of Fmoc-4AP(Boc)-4AP(Boc)-OH followed the NHS ester protocol.
(iv) The amide bond formation between Fmoc-4AP(Boc)-4AP(Boc)-NHS m-PEG48-amine followed the NHS protocol.
(v) Removal of the Fmoc protection group of Fmoc-4AP(Boc)-4AP(Boc)-PEG48-m followed the Fmoc protocol.
(vi) The amide bond formation between NH2-4AP(Boc)-4AP(Boc)-PEG48-m and 6-Maleimidohexanoic acid N-hydroxysuccinimide ester (MC-NHS) followed the NHS protocol.
(vii) Removal of the Boc protection group of MC-4AP(Boc)-4AP(Boc)-PEG48-m followed the Boc protocol.
(viii) The amide bond formation between Mc-4AP(NH2)—4AP(NH2)-PEG48-m and DM1-AMAS-Dap(PEG6-NHS)—OH followed the NHS protocol.

### Example 8: Compound 8, i.e., C-PD1-LK8 (PD=Dap-AMAS-DM1; HP+2^{nd} D=PEG24-lapatinib)

### Part A

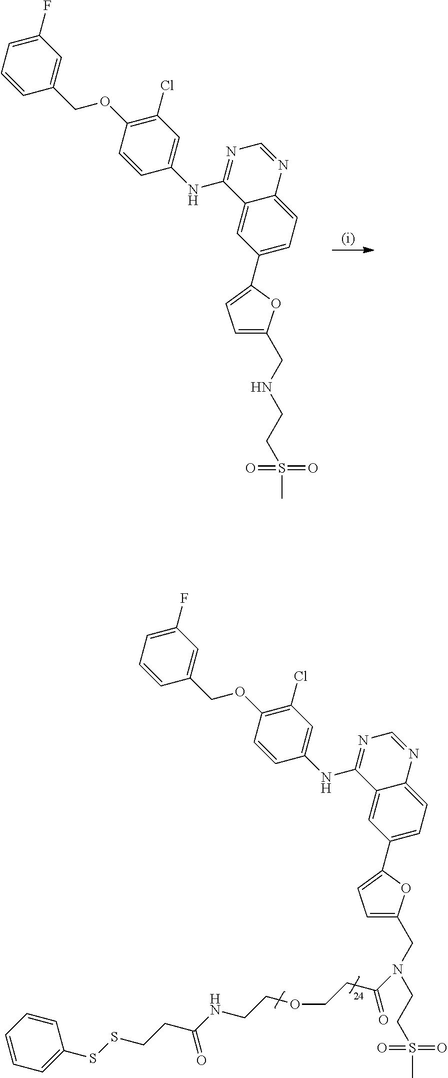

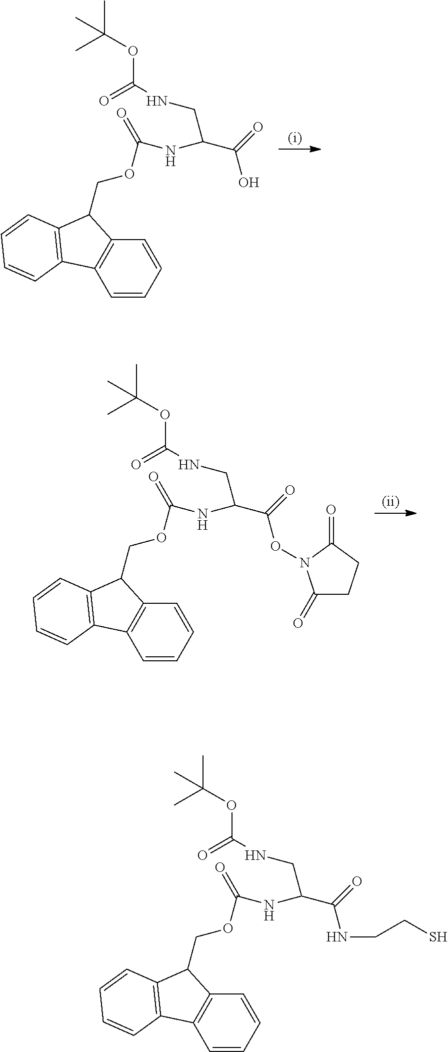 [SMILES: CC(C)(C)OC(=O)NCC(NC(=O)OCC1c2ccccc2-c2ccccc21)C(=O)O ; CC(C)(C)OC(=O)NCC(NC(=O)OCC1c2ccccc2-c2ccccc21)C(=O)ON1C(=O)CCC1=O ; CC(C)(C)OC(=O)NCC(NC(=O)OCC1c2ccccc2-c2ccccc21)C(=O)NCCS]

### Part C

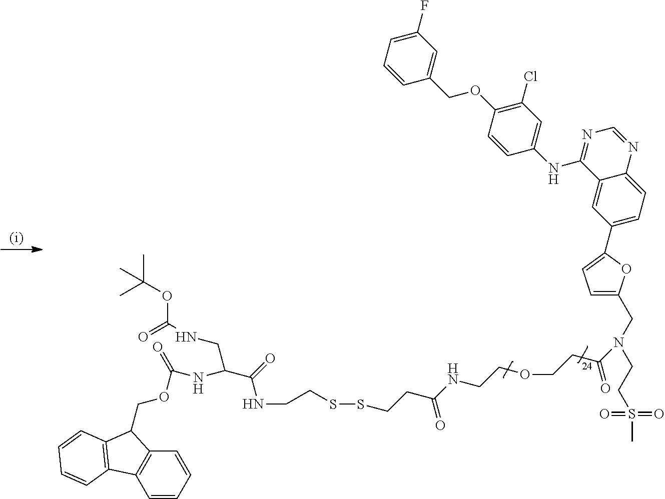

### Part D

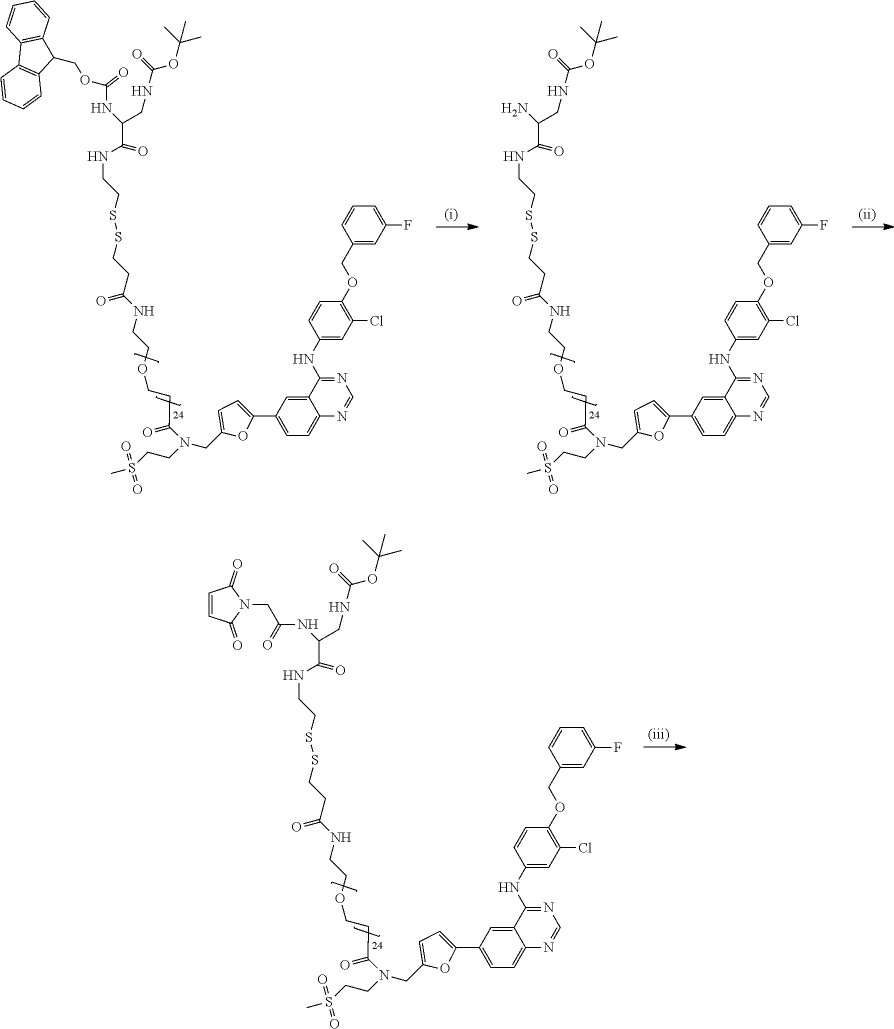

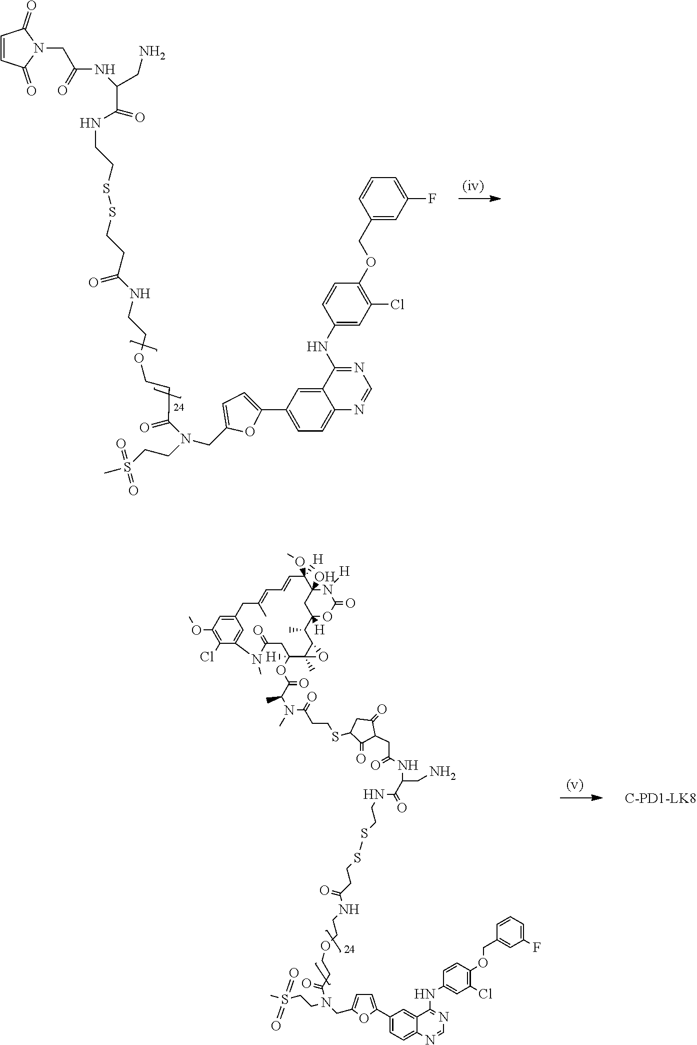

Compound 8 of this invention, i.e., DM1-AMAS-Dap(MC)—(CH2)2-S—S-PEG24-lapatinib, was synthesized following the protocols as indicated below.

### Part A.

(i) The amide bond formation between Lapatinib and SPDP-PEG-24-NHS ester followed the NHS protocol.

### Part B.

(i) Synthesis of NHS ester of Fmoc-Dap(Boc)-OH followed the NHS ester protocol.
(ii) The amide bond formation between Fmoc-Dap(Boc)-NHS and cysteamine followed the NHS protocol.

### Part C.

(i) The disulfide bond formation between Fmoc-Dap(Boc)-cysteamine and SPDP-PEG24-lapatinib followed the SPDP protocol.

### Part D.

(i) Removal of the Fmoc protection group of Fmoc-Dap(Boc)-(CH2)2-S—S-PEG24-lapatinib followed the Fmoc protocol.
(ii) The amide bond formation between NH2-Dap(Boc)-(CH2)2-S—S-PEG24-lapatinib and AMAS followed the NHS protocol.
(iii) Removal of the Boc protection group of AMAS-Dap(Boc)-(CH2)2-S—S-PEG24-lapatinib followed the Boc protocol.
(iv) The maleimide-thiol bond formation between AMAS-Dap(NH2)—(CH2)2-S—S-PEG24-lapatinib and DM1 followed the maleimide-thiol protocol.
(v) The amide bond formation between DM1-AMAS-Dap(NH2)—(CH2)2-S—S-PEG24-lapatinib and 6-Maleimidohexanoic acid N-hydroxysuccinimide ester (MC-NHS) followed the NHS protocol.

### Example 9: Compound 9, i.e., C-PD1-LK9 (PD=Dap-AMAS-DM1: HP+ST=PEG24-(acetyl-RHGAMVYLK))

### Part A

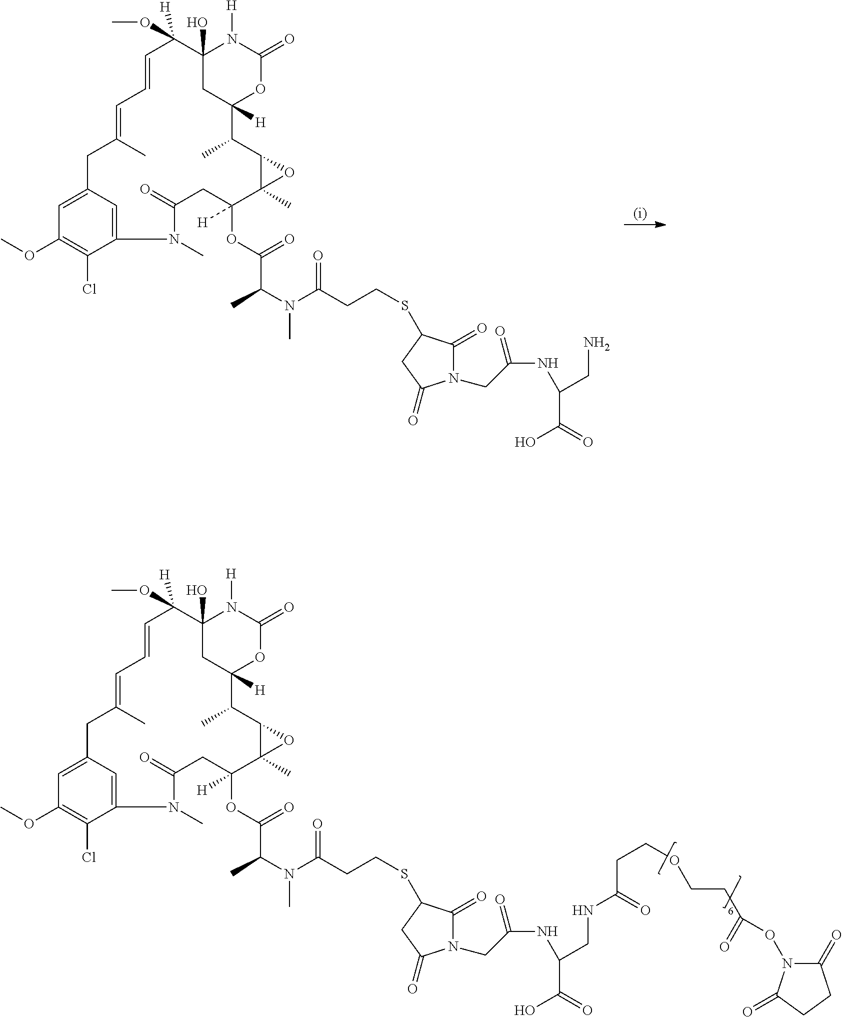

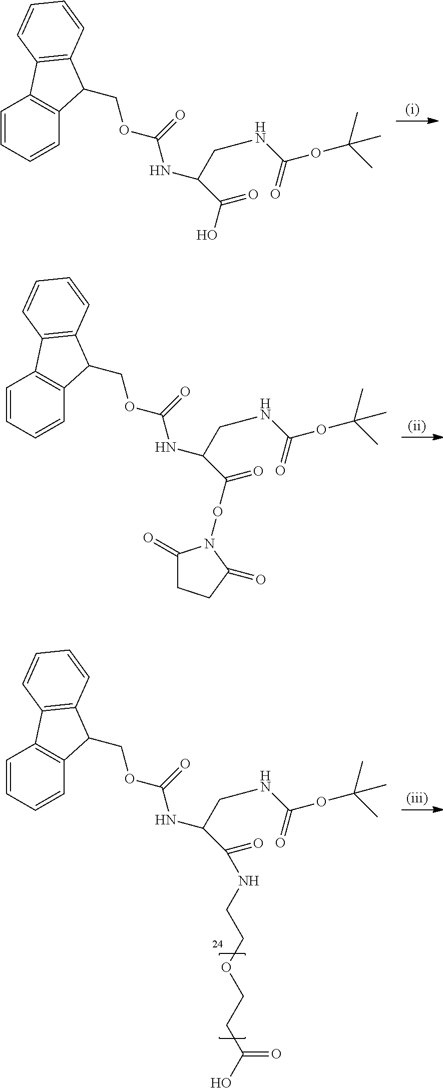

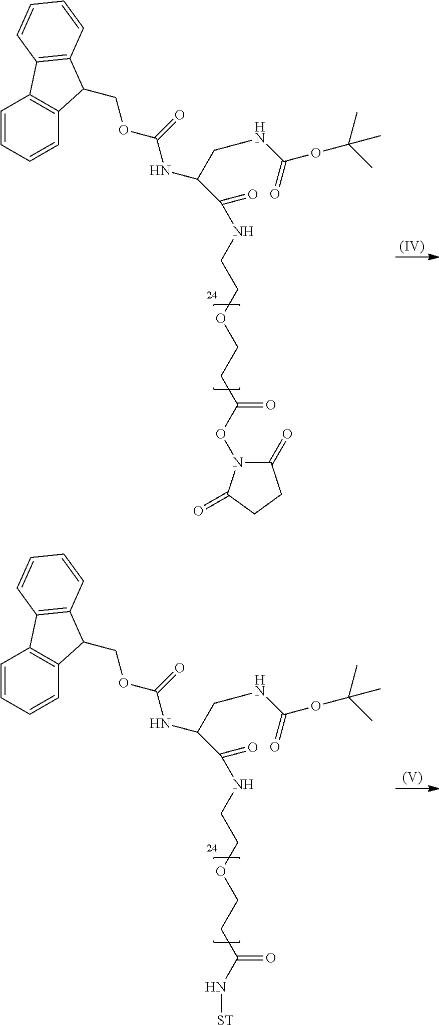

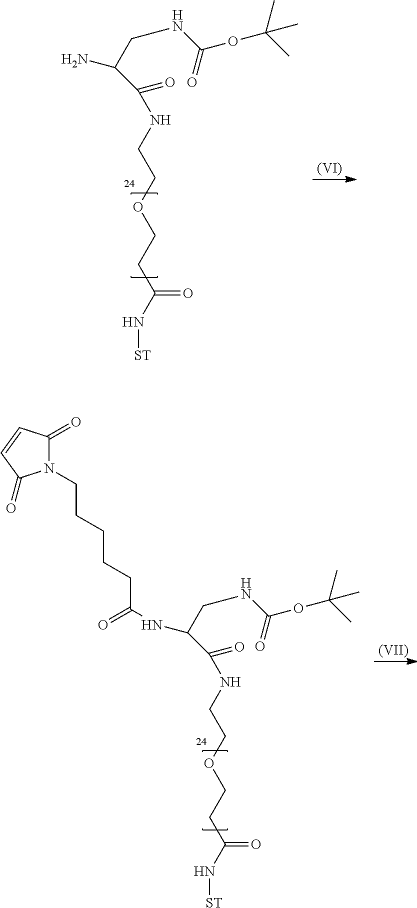

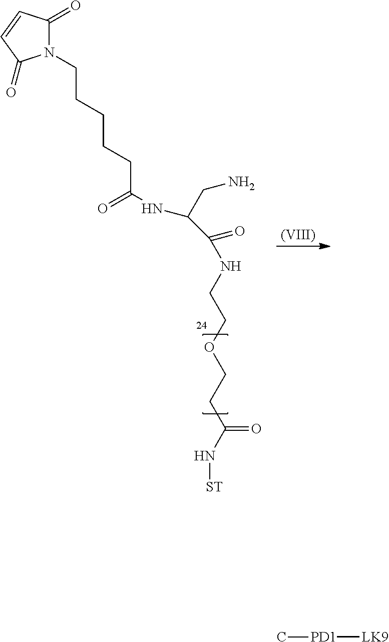

Compound 9 of this invention, i.e., MC-Dap(Dap-AMAS-DM1)-PEG24-(acetyl-RHGAMVYLK), was synthesized following the protocols as indicated below.

### Part A.

(i) The amide bond formation between DM1-AMAS-Dap(NH2)-OH and bis-PEG6-NHS followed the NHS protocol.

### Part B.

(i) Synthesis of NHS ester of Fmoc-Dap(Boc)-OH followed the NHS ester protocol.
(ii) The amide bond formation between Fmoc-Dap(Boc)-NHS and amino-PEG24-acid followed the NHS protocol.
(iii) Synthesis of NHS ester of Fmoc-Dap(Boc)-PEG24-acid followed the NHS ester protocol.
(iv) The amide bond formation between Fmoc-Dap(Boc)-PEG24-NHS and acetyl-RHGAMVYLK followed the NHS protocol.
(v) Removal of the Fmoc protection group of Fmoc-Dap(Boc)-PEG24-(acetyl-RHGAMVYLK) followed the Fmoc protocol.
(vi) The amide bond formation between NH2-Dap(Boc)-PEG24-(acetyl-RHGAMVYLK) and 6-Maleimidohexanoic acid N-hydroxysuccinimide ester (MC-NHS) followed the NHS protocol.
(vii) Removal of the Boc protection group of MC-Dap(Boc)-PEG24-(acetyl-RHGAMVYLK) followed the Boc protocol.
(viii) The amide bond formation between MC-Dap(NH2)-PEG24-(acetyl-RHGAMVYLK) and DM1-AMAS-Dap(PEG6-NHS)—OH followed the NHS protocol.

### Example 10: Compound 10: C-PD1-LK10 (PD=Dap-AMAS-DM1; HP+ST=PEG1K-Folic acid)

### Part A

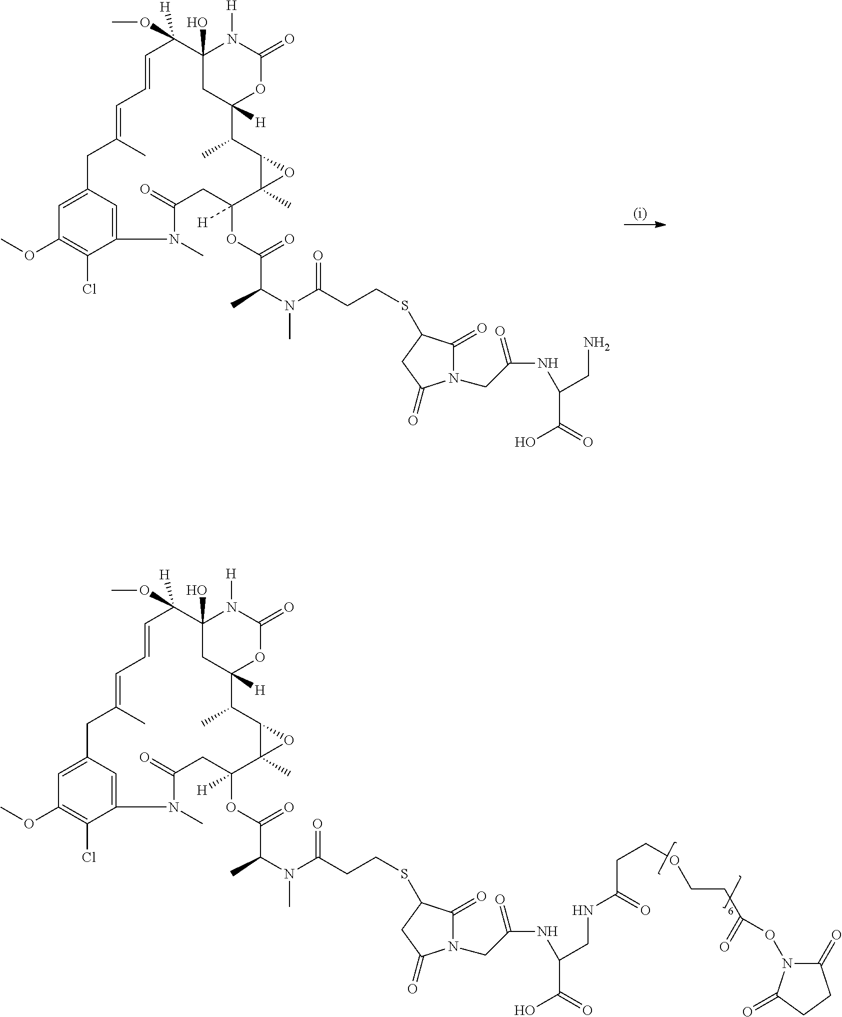

### Part B

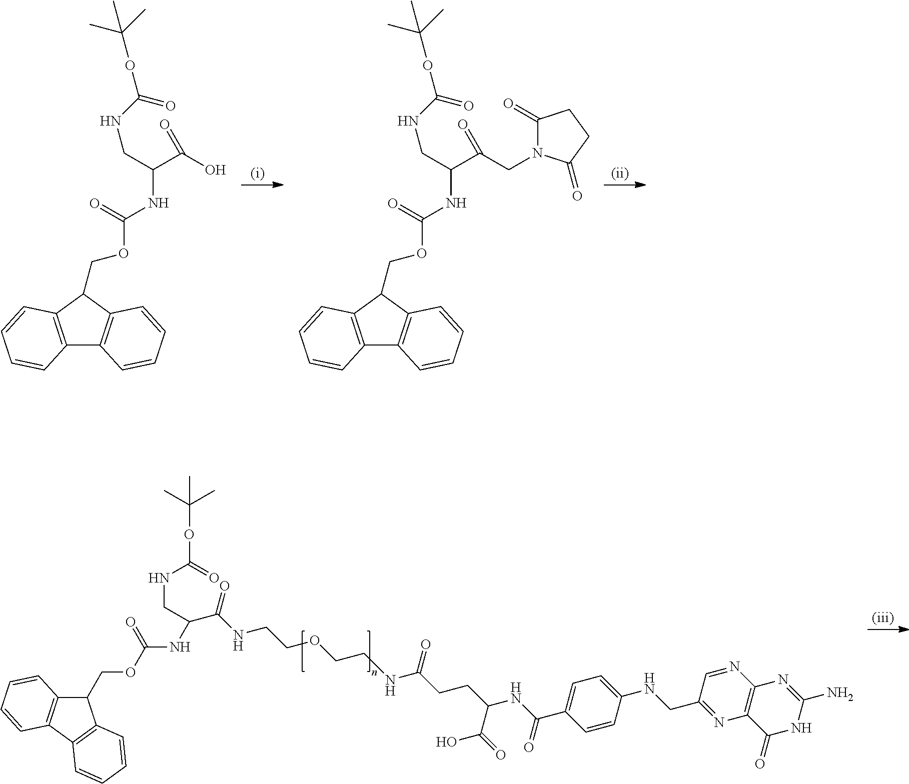

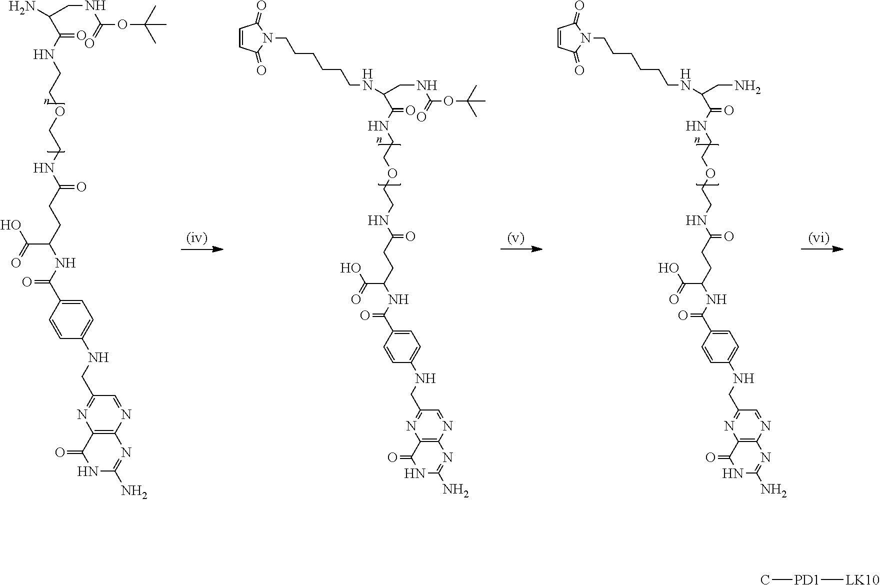

Compound 10 of this invention, i.e., MC-Dap(PEG6-Dap-AMAS-DM1)-PEG1K-Folic acid, was synthesized following the protocols as indicated below.

### Part A.

(i) The amide bond formation between DM1-AMAS-Dap(NH2)-OH and bis-PEG6-NHS followed the NHS protocol.

### Part B.

(i) Synthesis of NHS ester of Fmoc-Dap(Boc)-OH followed the NHS ester protocol.
(ii) The amide bond formation between Fmoc-Dap(Boc)-NHS and amino-PEG1K-Folic acid followed the NHS protocol.
(iii) Removal of the Fmoc protection group of Fmoc-Dap(Boc)-PEG1K-Folic acid followed the Fmoc protocol.
(iv) The amide bond formation between NH2-Dap(Boc)-PEG1K—Folic acid and 6-Maleimidohexanoic acid N-hydroxysuccinimide ester (MC-NHS) followed the NHS protocol.
(v) Removal of the Boc protection group of MC-Dap(Boc)-PEG1K—Folic acid followed the Boc protocol.
(vi) The amide bond formation between MC-Dap(NH2)-PEG1K—Folic acid and DM1-AMAS-Dap(PEG6-NHS)—OH followed the NHS protocol.

### Example 11: Compound 11, i.e., C-PD1-LK11 (PD=Dap-AMAS-DM1; HP+ST=PEG1K-glucose)

### Part A

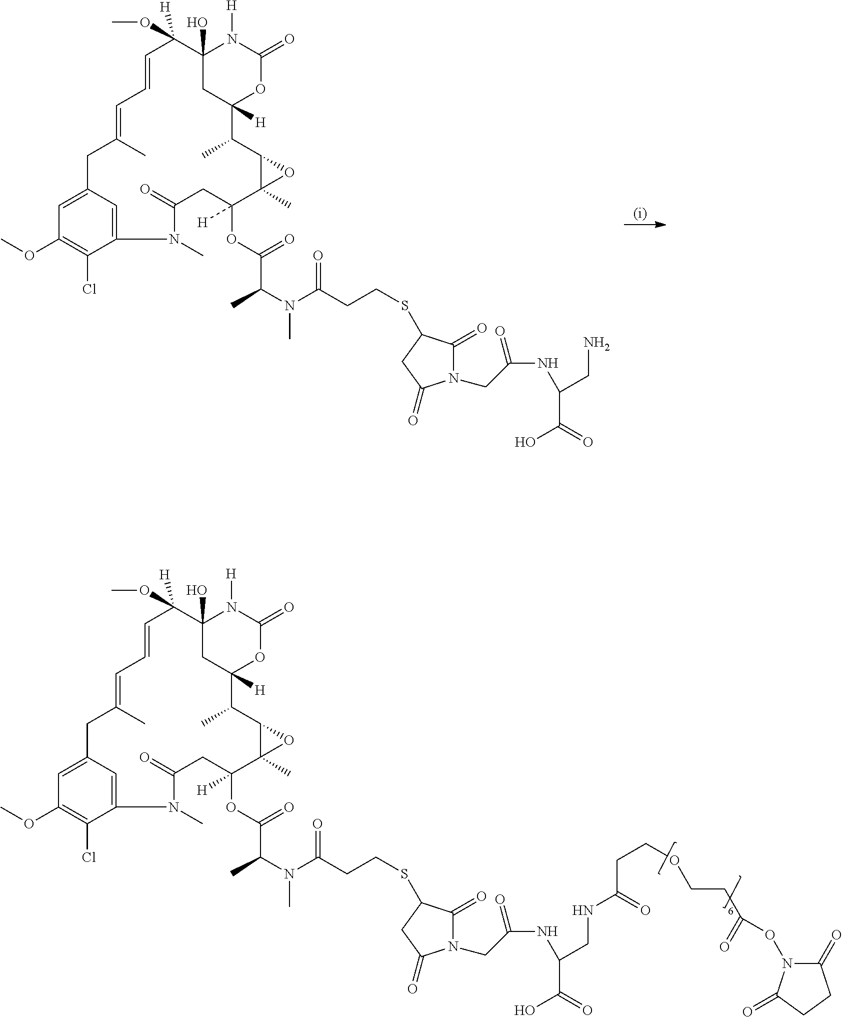

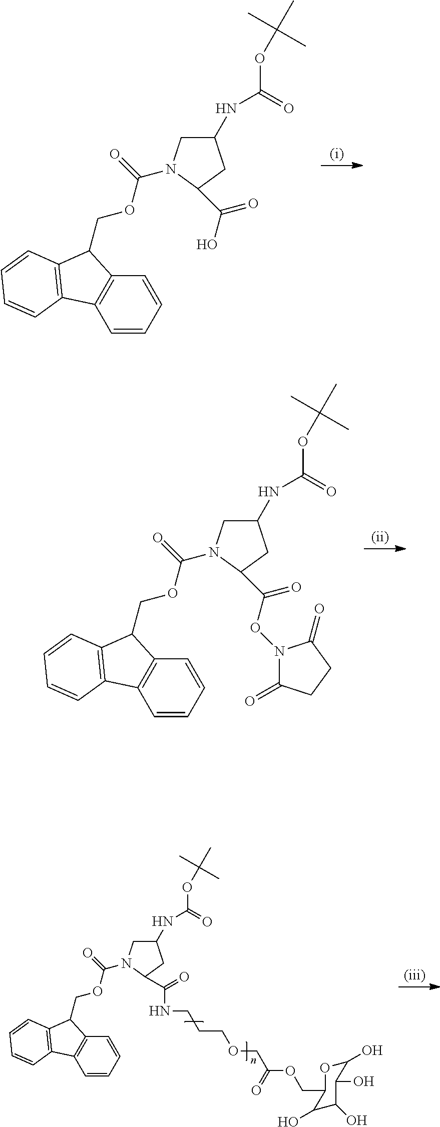

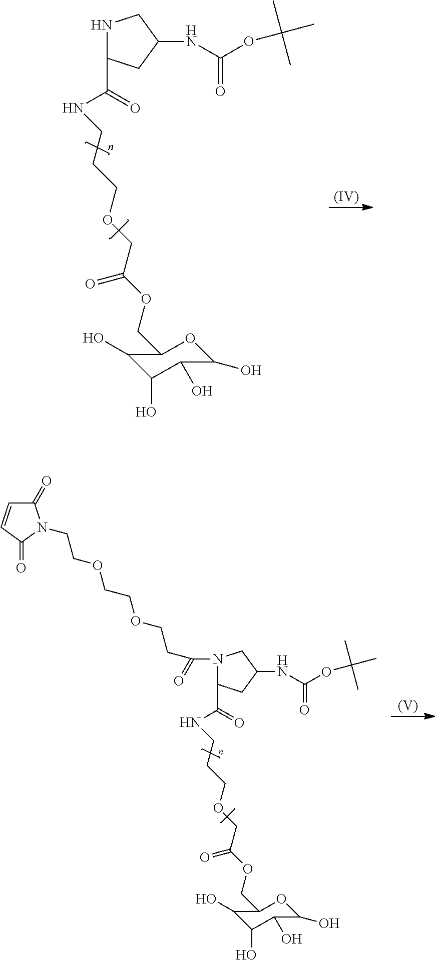

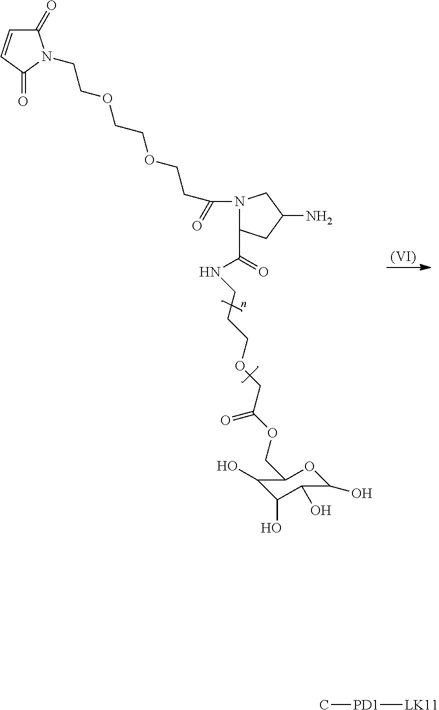

### Part B

Compound 11 of this invention, i.e., Mal-PEG2-4AP(PEG6-Dap-AMAS-DM1)-PEG1K-glucose, was prepared following the protocols as indicated below.

### Part A.

(i) The amide bond formation between DM1-AMAS-Dap(NH2)-OH and bis-PEG6-NHS followed the NHS protocol.

### Part B.

(i) Synthesis of NHS ester of Fmoc-4AP(Boc)-OH followed the NHS ester protocol.
(ii) The amide bond formation between Fmoc-4AP(Boc)-NHS and NH2-PEG1K-glucose followed the NHS protocol.
(iii) Removal of the Fmoc protection group of Fmoc-4Dap(Boc)-PEG1K-glucose followed the Fmoc protocol.
(iv) The amide bond formation between NH2-4AP(Boc)-PEG1K-glucose and Mal-PEG2-acid followed the HATU protocol.
(v) Removal of the Boc protection group of Mal-PEG2-4AP(Boc)-PEG1K-glucose followed the Boc protocol.
(vi) The amide bond formation between Mal-PEG2-4AP(NH2)-PEG1K-PEG1K-glucose and DM1-AMAS-Dap(PEG6-NHS)—OH followed the NHS protocol.

### Example 12: Compound 12, i.e., C-PD1-LK12 (PD=Dap-AMAS-DM1; HP=PEG80H)

### Part A

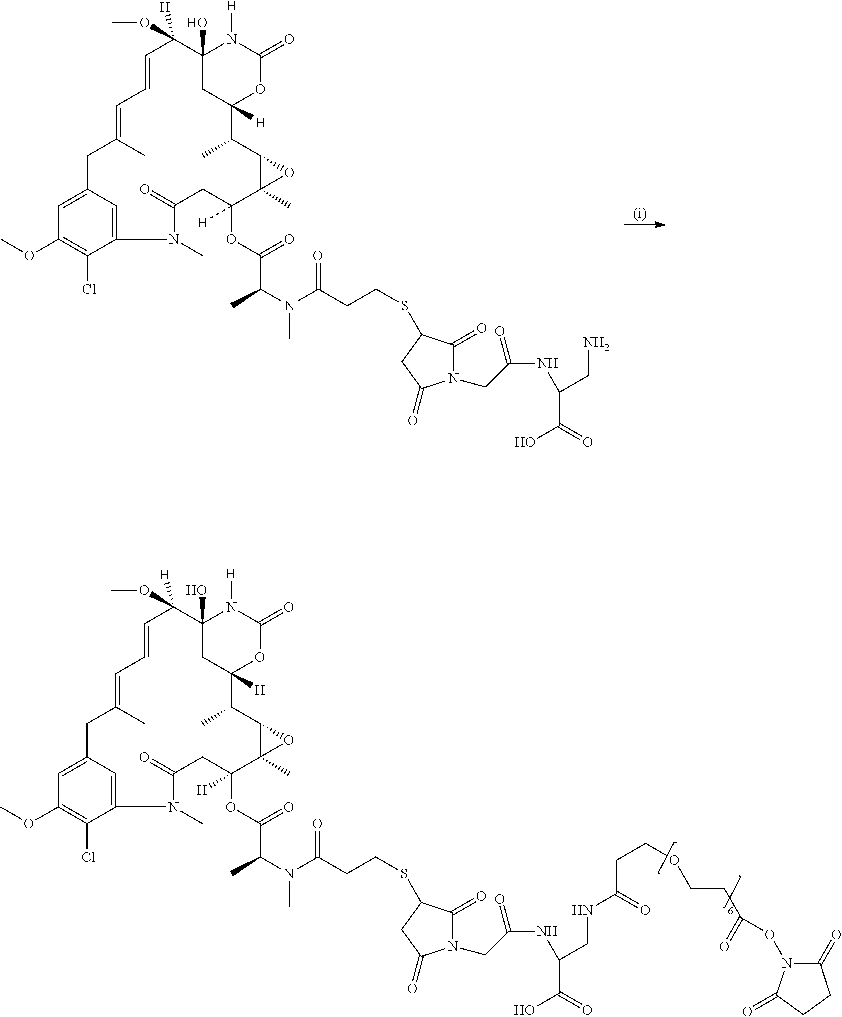

### Part B

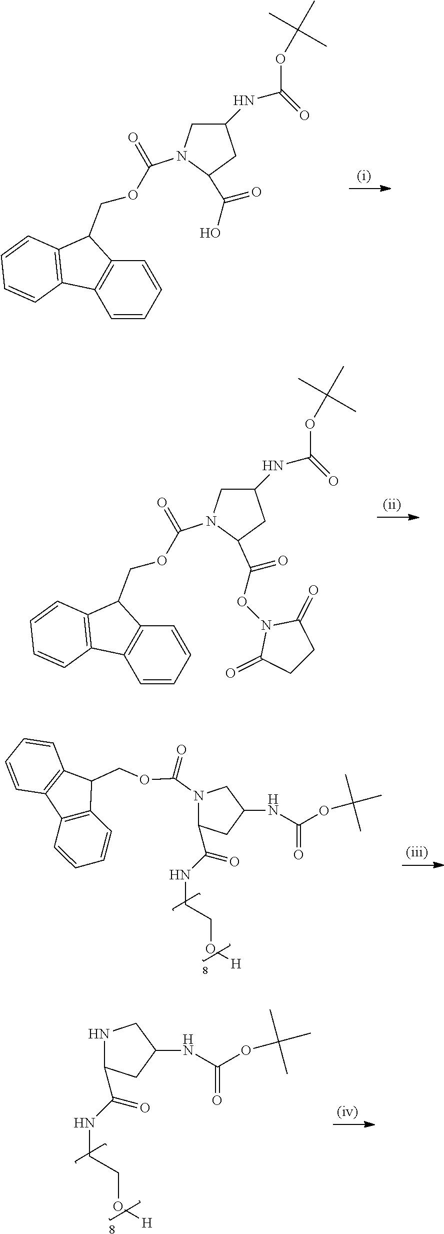

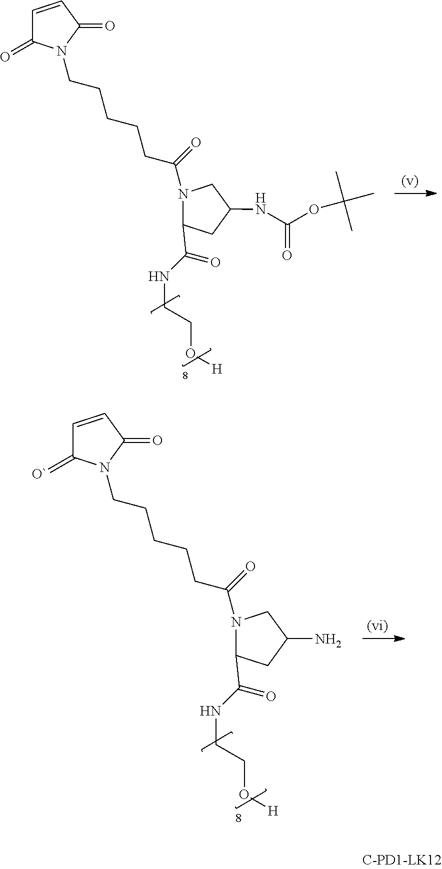

Compound 12 of this invention, i.e., MC-4AP(PEG6-Dap-AMAS-DM1)-PEG80H, was prepared following the protocols as indicated below.

### Part A.

(i) The amide bond formation between DM1-AMAS-Dap(NH2)-OH and bis-PEG6-NHS followed the NHS protocol.

### Part B.

(i) Synthesis of NHS ester of Fmoc-4AP(Boc)-OH followed the NHS ester protocol.
(ii) The amide bond formation between Fmoc-4AP(Boc)-NHS and amino-PEG8-alcohol followed the NHS protocol.
(iii) Removal of the Fmoc protection group of Fmoc-4AP(Boc)-PEG8-OH followed the Fmoc protocol.
(iv) The amide bond formation between NH2-4AP(Boc)-PEG8-OH and 6-Maleimidohexanoic acid N-hydroxysuccinimide ester (MC-NHS) followed the NHS protocol.
(v) Removal of the Boc protection group of MC-4AP(Boc)-PEG8-OH followed the Boc protocol.
(vi) The amide bond formation between MC-4AP(NH2)-PEG8-OH and DM1-AMAS-Dap(PEG6-NHS)—OH followed the NHS protocol.

### Example 13: Compound 13, i.e., C-PD1-LK13 (PD=Dap-AMAS-DM1; HP=PEG24-m)

### Part A

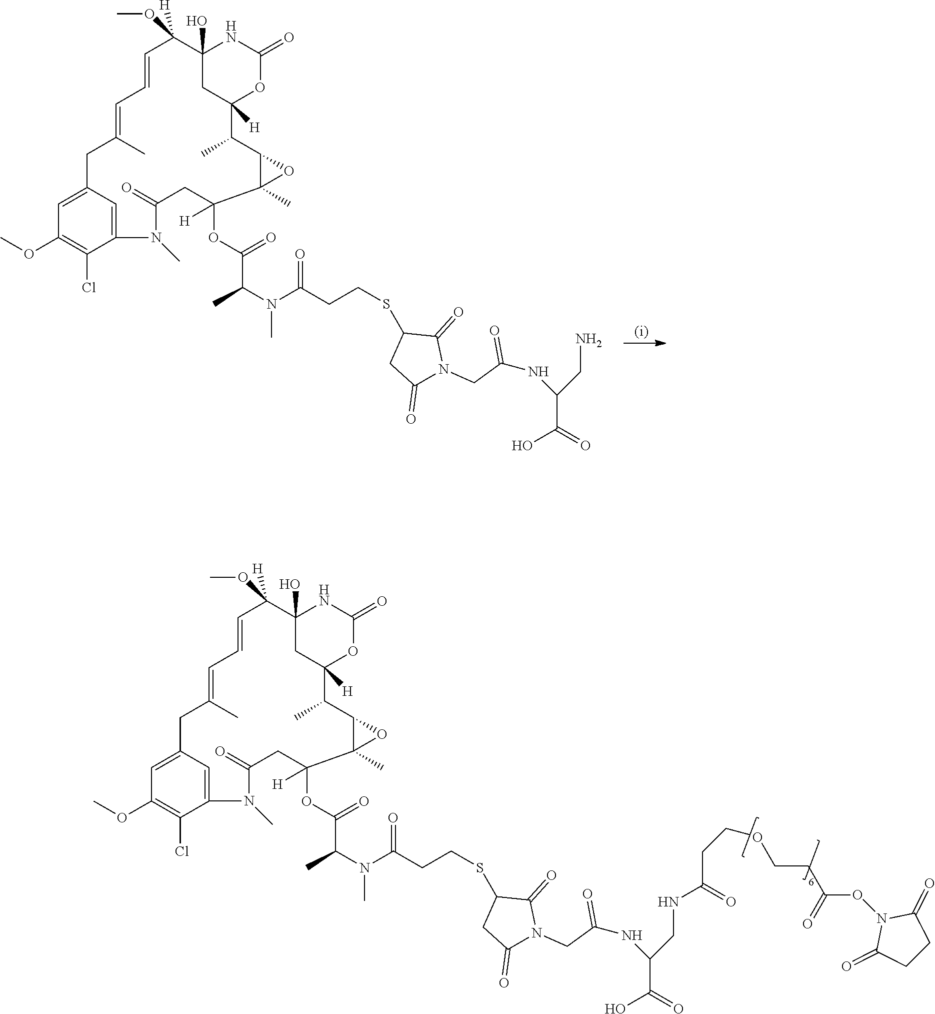

### Part B

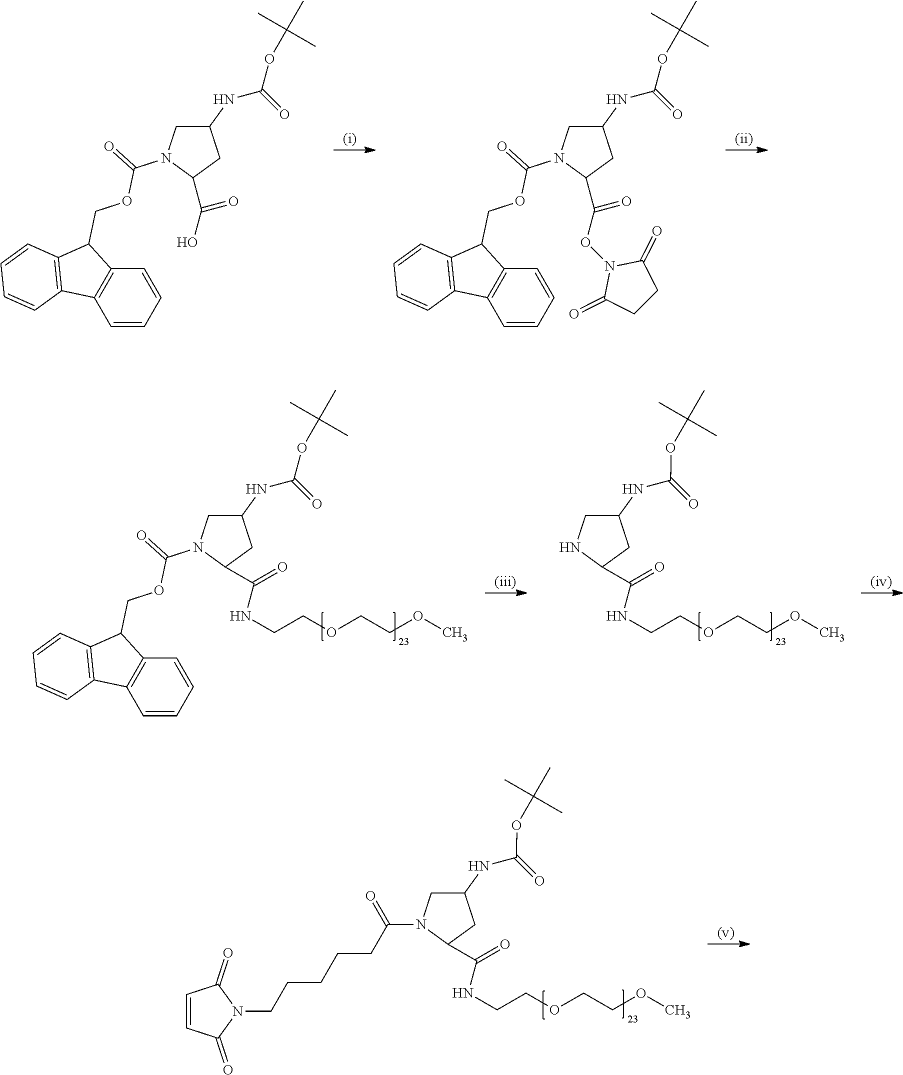

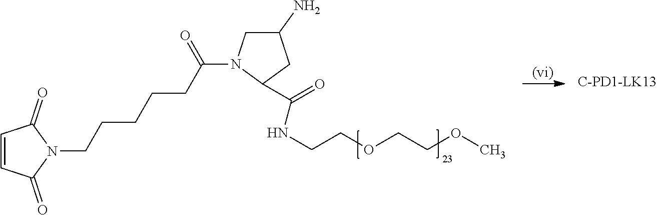

Compound 13 of this invention, i.e., MC-4AP(PEG6-Dap-AMAS-DM1)-PEG24-m, was prepared following the protocols as indicated below.

### Part A.

(i) The amide bond formation between DM1-AMAS-Dap(NH2)-OH and bis-PEG6-NHS followed the NHS protocol.

### Part B.

(i) Synthesis of NHS ester of Fmoc-4AP(Boc)-OH followed the NHS ester protocol.
(ii) The amide bond formation between Fmoc-4AP(Boc)-NHS and amino-PEG24-m followed the NHS protocol.
(iii) Removal of the Fmoc protection group of Fmoc-4AP(Boc)-PEG24-m followed the Fmoc protocol.
(iv) The amide bond formation between NH2-4AP(Boc)-PEG24-m and 6-Maleimidohexanoic acid N-hydroxysuccinimide ester (MC-NHS) followed the NHS protocol.
(v) Removal of the Boc protection group of MC-4AP(Boc)-PEG24-m followed the Boc protocol.
(vi) The amide bond formation between MC-4AP(NH2)-PEG24-m and DM1-AMAS-Dap(PEG6-NHS)—OH followed the NHS protocol.

### Example 14: Compound 14, i.e., C-PD1-LK14 (PD=Dap-AMAS-DM1; HP=PEG24-m)

### Part A

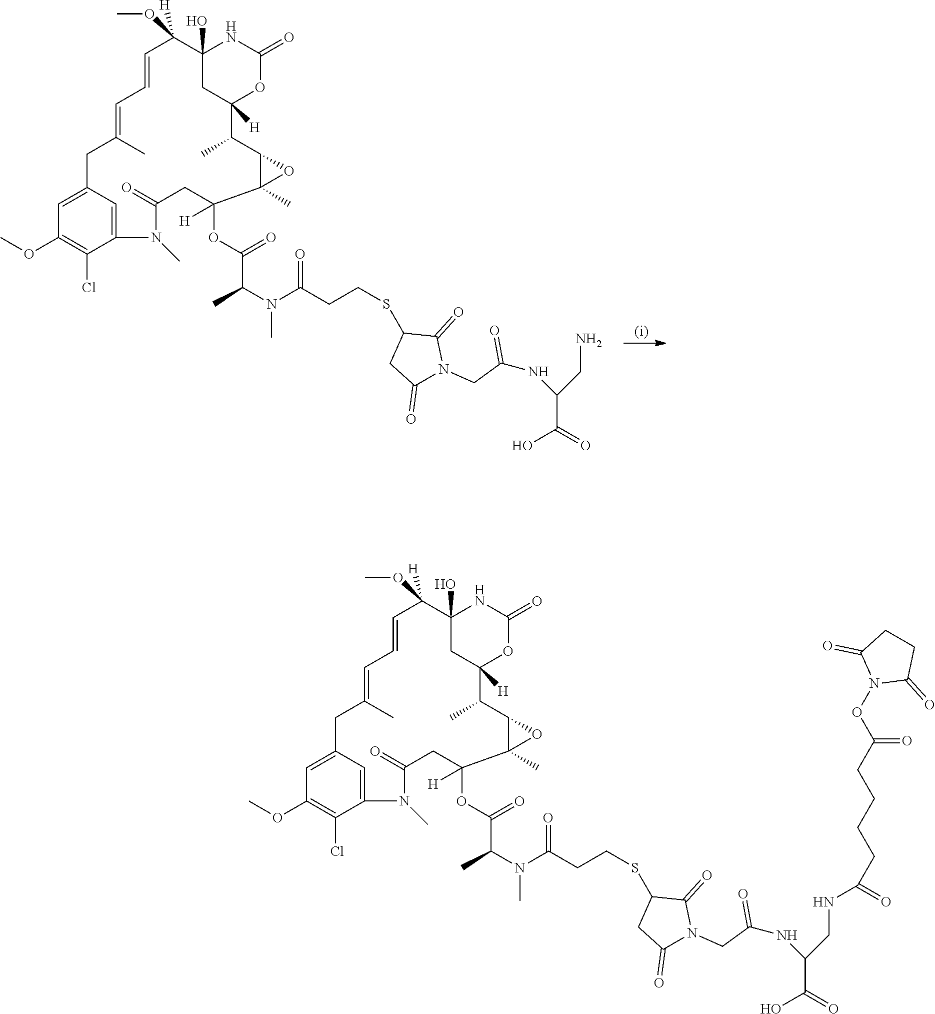 [SMILES: COc1cc2cc(c1Cl)N(C)C(=O)CC(OC(=O)[C@H](C)N(C)C(=O)CCSC1CC(=O)N(CC(=O)NC(CN)C(=O)O)C1=O)[C@]1(C)O[C@H]1[C@H](C)[C@@H]1C[C@@](O)(NC(=O)O1)[C@H](OC)/C=C/C=C(\C)C2 ; COc1cc2cc(c1Cl)N(C)C(=O)CC(OC(=O)[C@H](C)N(C)C(=O)CCSC1CC(=O)N(CC(=O)NC(CNC(=O)CCCCC(=O)ON3C(=O)CCC3=O)C(=O)O)C1=O)[C@]1(C)O[C@H]1[C@H](C)[C@@H]1C[C@@](O)(NC(=O)O1)[C@H](OC)/C=C/C=C(\C)C2]

### Part B

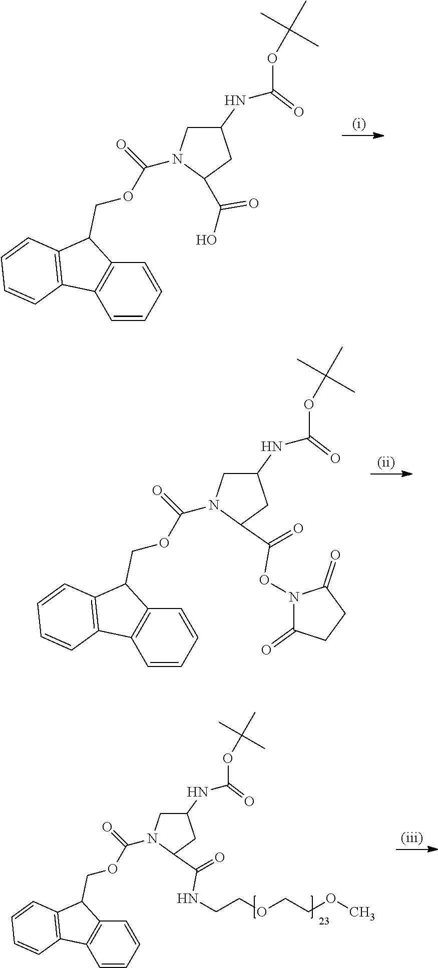

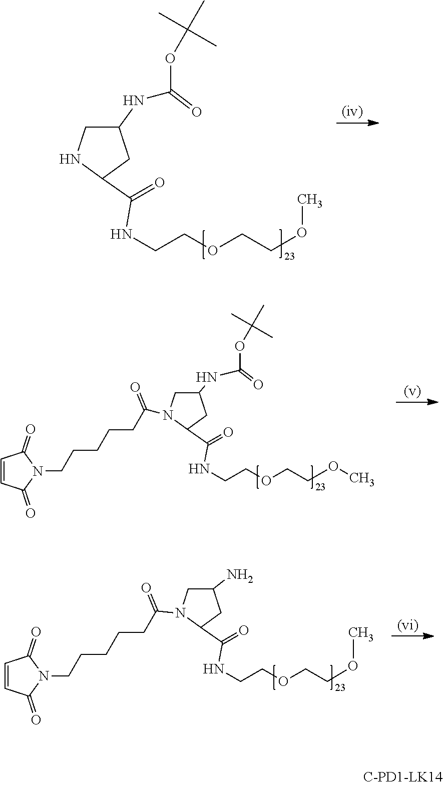

Compound 14 of this invention, i.e., MC-4AP(DSA-Dap-AMAS-DM1)-PEG24-m, was prepared following the protocols as indicated below.

### Part A.

(i) The amide bond formation between DM1-AMAS-Dap(NH2)-OH and DSA followed the NHS protocol.

### Part B.

(i) Synthesis of NHS ester of Fmoc-4AP(Boc)-OH followed the NHS ester protocol.
(ii) The amide bond formation between Fmoc-4AP(Boc)-NHS and amino-PEG24-m followed the NHS protocol.
(iii) Removal of the Fmoc protection group of Fmoc-4AP(Boc)-PEG24-m followed the Fmoc protocol.
(iv) The amide bond formation between NH2-4AP(Boc)-PEG24-m and 6-Maleimidohexanoic acid N-hydroxysuccinimide ester (MC-NHS) followed the NHS protocol.
(v) Removal of the Boc protection group of MC-4AP(Boc)-PEG24-m followed the Boc protocol.
(vi) The amide bond formation between MC-4AP(NH2)-PEG24-m and DM1-AMAS-Dap(DSA)-OH followed the NHS protocol.

### Example 15: Compound 15, i.e., C-PD2-LK15 (PD=Ser-AMAS-DM1; HP=PEG24-m)

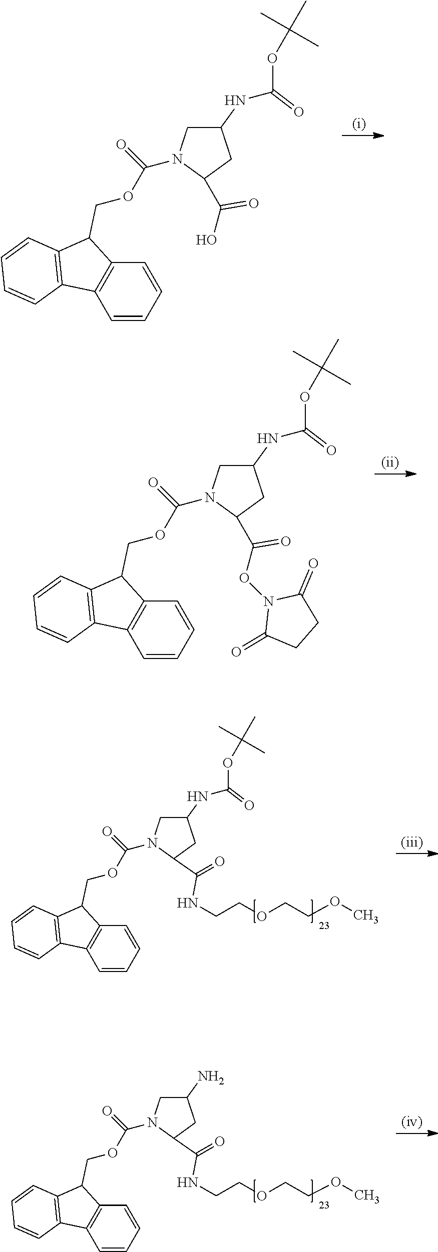

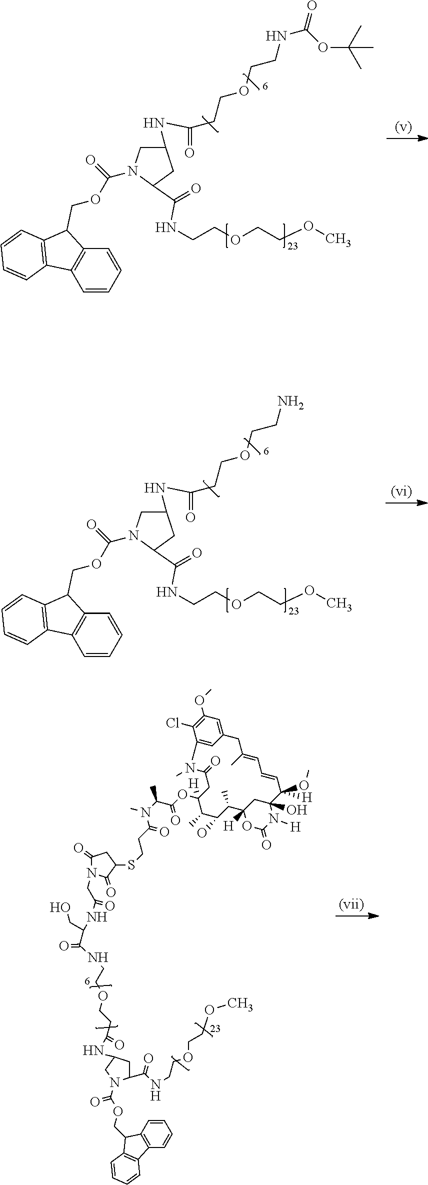

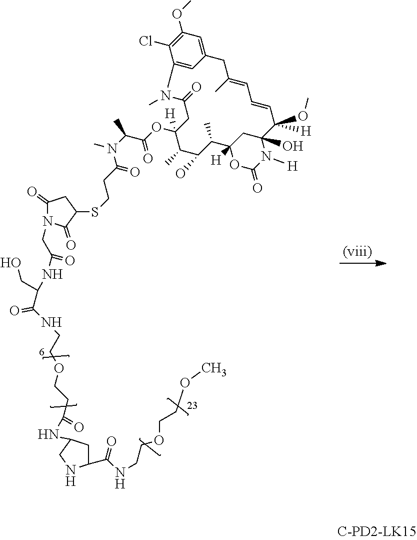

Compound 15 of this invention, i.e., MC-4AP(PEG6-Ser-AMAS-DM1)-PEG24-m, was prepared following the protocols as indicated below.
(i) Synthesis of NHS ester of Fmoc-4AP(Boc)-OH followed the NHS ester protocol.
(ii) The amide bond formation between Fmoc-4AP(Boc)-NHS and amino-PEG24-m followed the NHS protocol.
(iii) Removal of the Boc protection group of Fmoc-4AP(Boc)-PEG24-m followed the Boc protocol.
(iv) The amide bond formation between Fmoc-4AP(NH2)-PEG24-m and t-Boc-N-amido-PEG6-acid followed the HATU protocol.
(v) Removal of the Boc protection group of Fmoc-4AP(t-Boc-N-amido-PEG6)-PEG24-m followed the Boc protocol.
(vi) Amide bond formation between Fmoc-4AP(N-amido-PEG6)-PEG24-m and DM1-AMAS-Ser followed the HATU protocol.
(vii) Removal of the Fmoc protection group of Fmoc-4AP(PEG6-Ser-AMAS-DM1)-PEG24-m followed the Fmoc protocol.
(viii) Amide bond formation between NH2-4AP(PEG6-Ser-AMAS-DM1)-PEG24-m and 6-Maleimidocaproic acid followed the HATU protocol.

### Example 16: Compound 16, i.e., C-PD2-LK16 (PD=Ser-AMAS-DM1; HP=PEG24-m)

### Part A

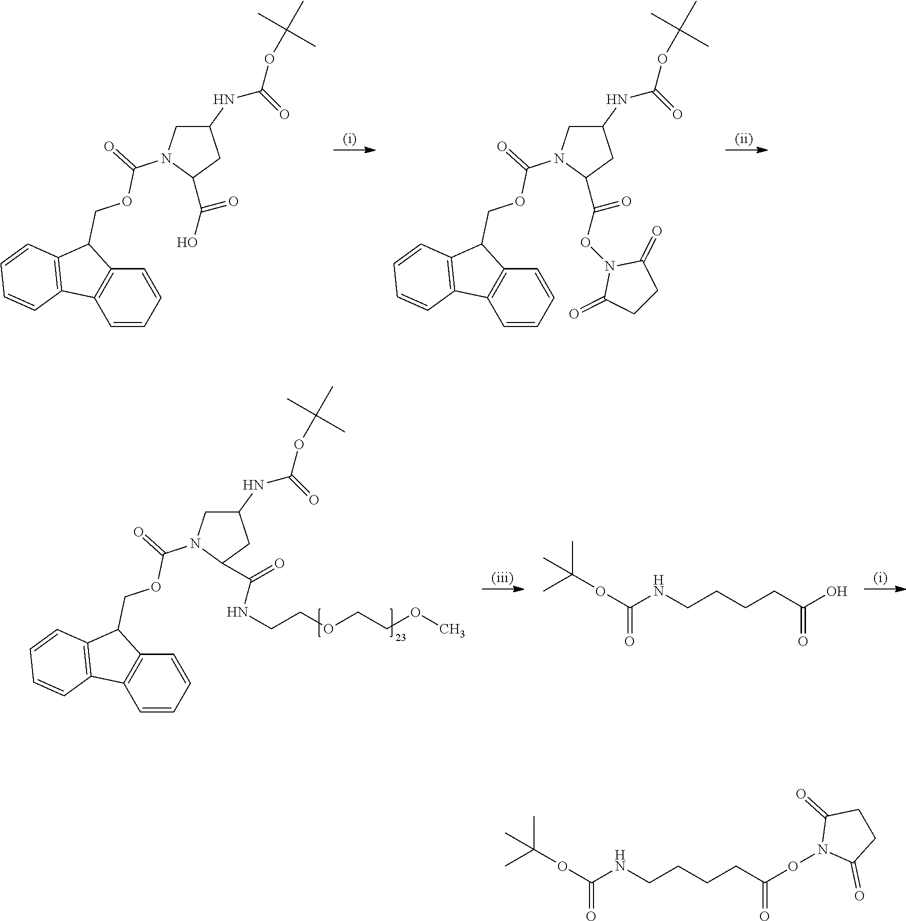

### Part B

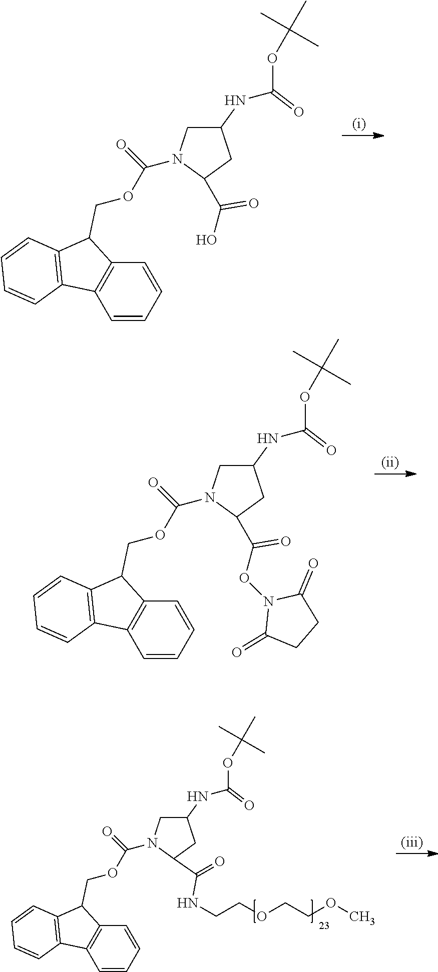

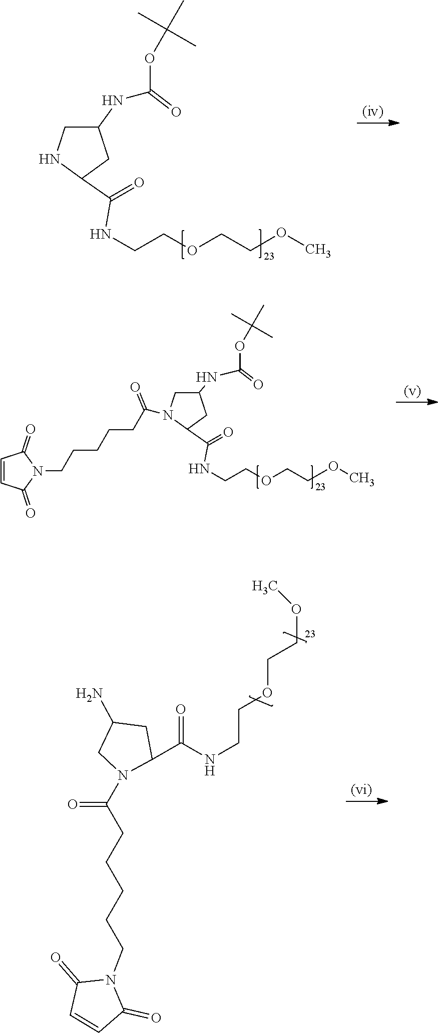

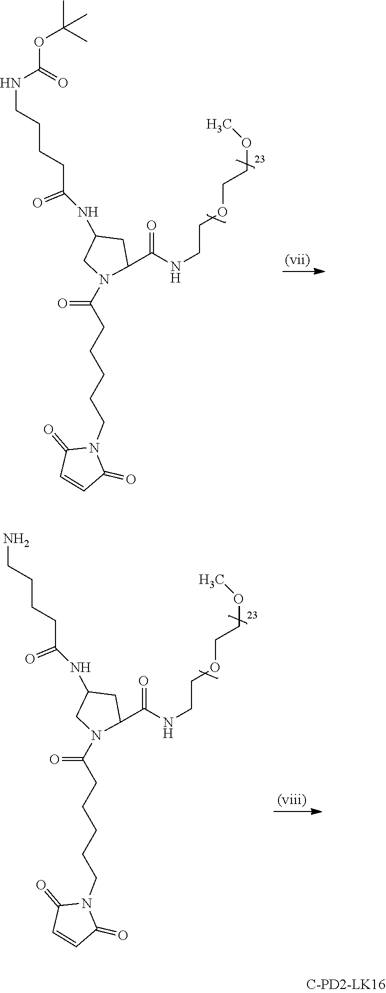

Compound 16 of this invention, i.e., MC-4AP(5-aminopentanoic acid-Ser-AMAS-DM1)-PEG24-m, was prepared following the protocols as indicated below.

### Part A.

(i) Synthesis of NHS ester of Boc-5-aminopentanoic acid followed the NHS ester protocol.

### Part B.

(i) Synthesis of NHS ester of Fmoc-4AP(Boc)-OH followed the NHS ester protocol.
(ii) The amide bond formation between Fmoc-4AP(Boc)-NHS and amino-PEG24-m followed the NHS protocol.
(iii) Removal of the Fmoc protection group of Fmoc-4AP(Boc)-PEG24-m followed the Fmoc protocol.
(iv) The amide bond formation between NH2-4AP(Boc)-PEG24-m and 6-Maleimidohexanoic acid N-hydroxysuccinimide ester (MC-NHS) followed the NHS protocol.
(v) Removal of the Boc protection group of MC-4AP(Boc)-PEG24-m followed the Boc protocol.
(vi) The amide bond formation between MC-4AP(NH2)-PEG24-m and Boc-5-aminopentanoic acid-NHS followed the NHS protocol.
(vii) Removal of the Boc protection group of MC-4AP(Boc-5-aminopentanoic acid)-PEG24-m followed the Boc protocol.
(viii) The amide bond formation between MC-4AP(NH2-5-aminopentanoic acid)-PEG24-m and DM1-AMAS-Ser-OH followed the HATU protocol.

### Example 17: Compound 17, i.e., C-PD1-LK17 (PD=Dap-AMAS-DM1; HP=PEG8-m)

### Part A

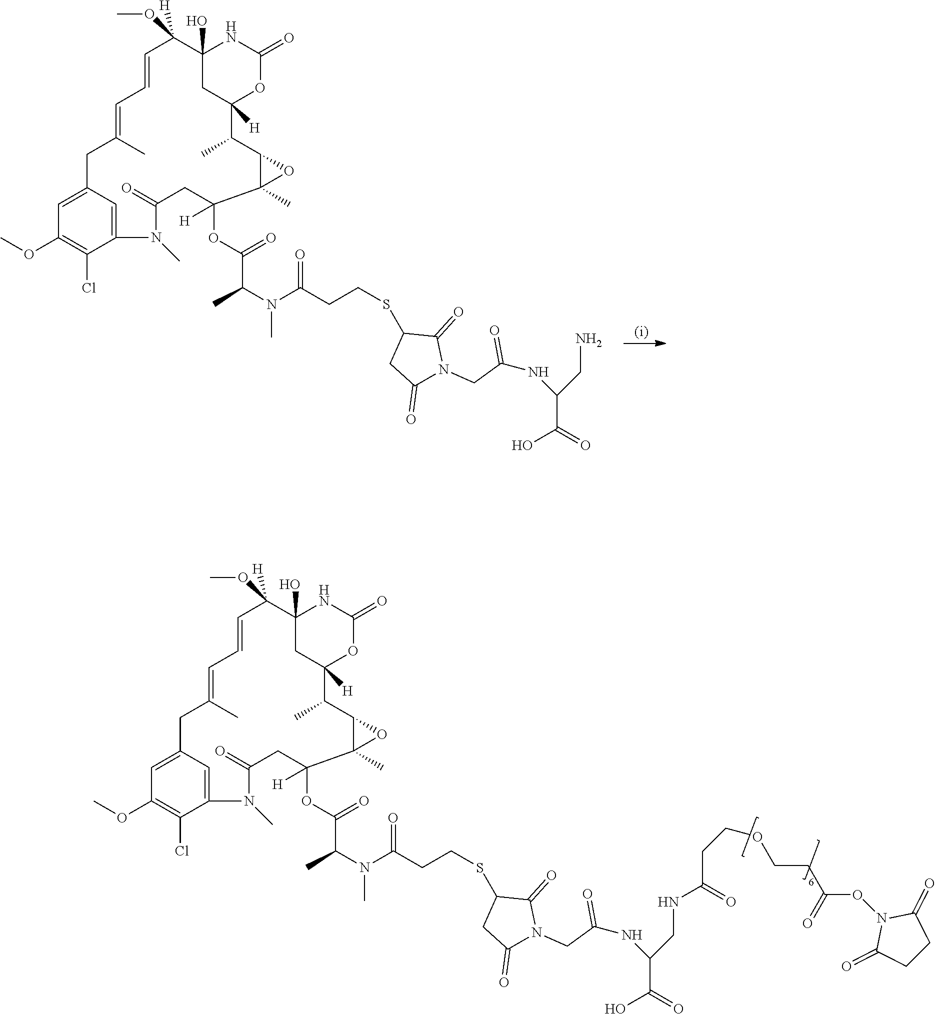

### Part B

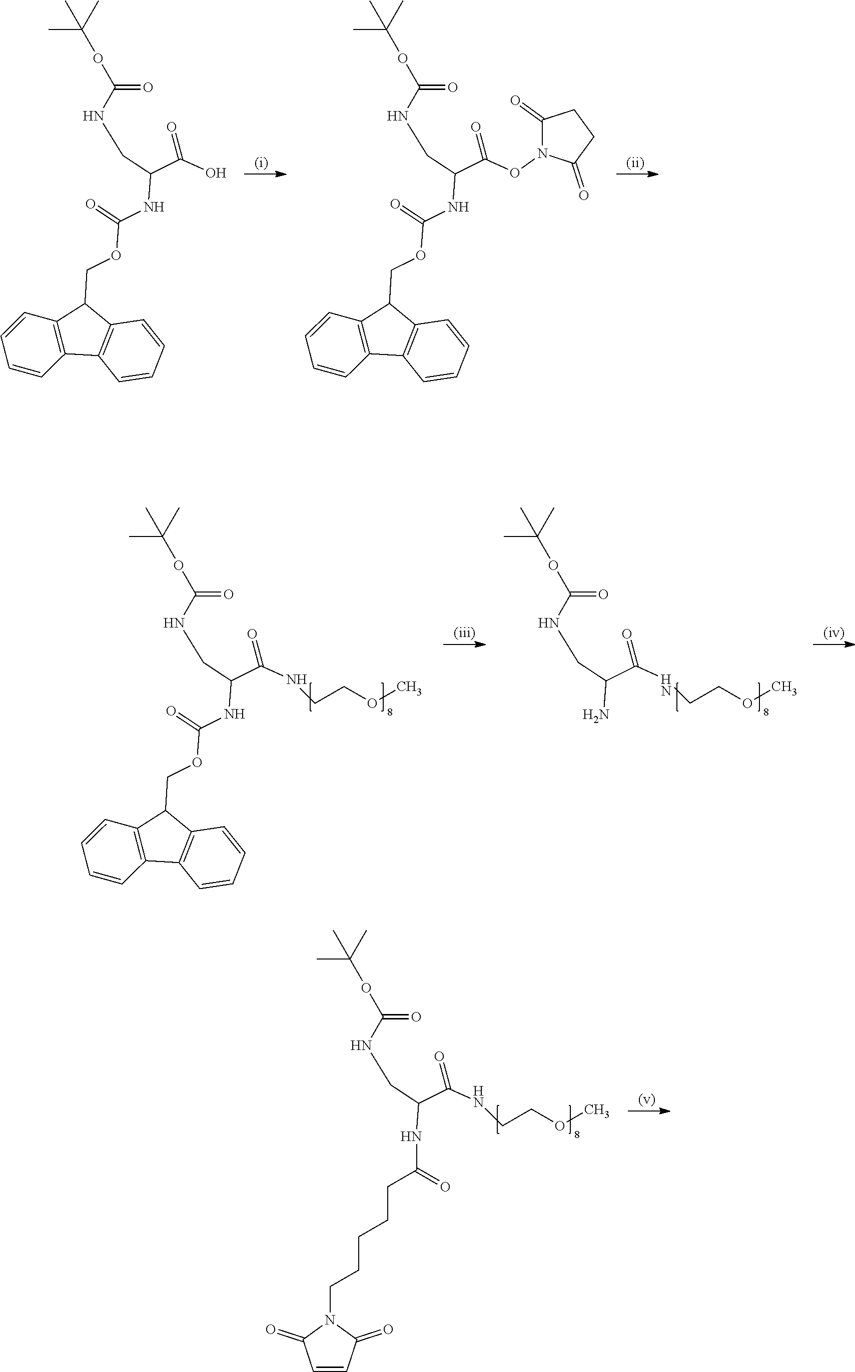

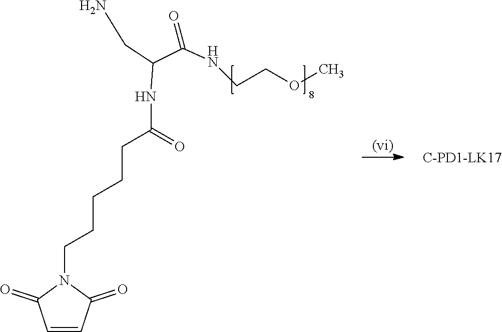

Compound 17 of this invention, i.e., MC-Dap(PEG6-Dap-AMAS-DM1)-PEG8-m, was prepared following the protocols as indicated below.

### Part A.

(i) The amide bond formation between DM1-AMAS-Dap(NH2)-OH and Bis-PEG6-NHS followed the NHS protocol.

### Part B.

(i) Synthesis of NHS ester of Fmoc-Dap(Boc)-OH followed the protocol for NHS ester synthesis.
(ii) The amide bond formation between Fmoc-Dap(Boc)-NHS and amino-PEG8-m followed the NHS protocol.
(iii) Removal of the Fmoc protection group of Fmoc-Dap(Boc)-PEG8-m followed the Fmoc protocol.
(iv) The amide bond formation between NH2-Dap(Boc)-PEG8-m and 6-Maleimidohexanoic acid N-hydroxysuccinimide ester (MC-NHS) followed the NHS protocol.
(v) Removal of the Boc protection group of MC-Dap(Boc)-PEG8-m followed the Boc protocol.
(vi) The amide bond formation between MC-Dap(NH2)-PEG8-m and DM1-AMAS-Dap(PEG6-NHS)—OH followed the NHS protocol.

Formula of C-L-PD=C90H139C1N10035S Formula Weight C-L-PD=1988.63

### Example 18: Compound 18, i.e., C-PD1-LK18 (PD=Dap-AMAS-DM1; HP=PEG8-OH)

### Part A

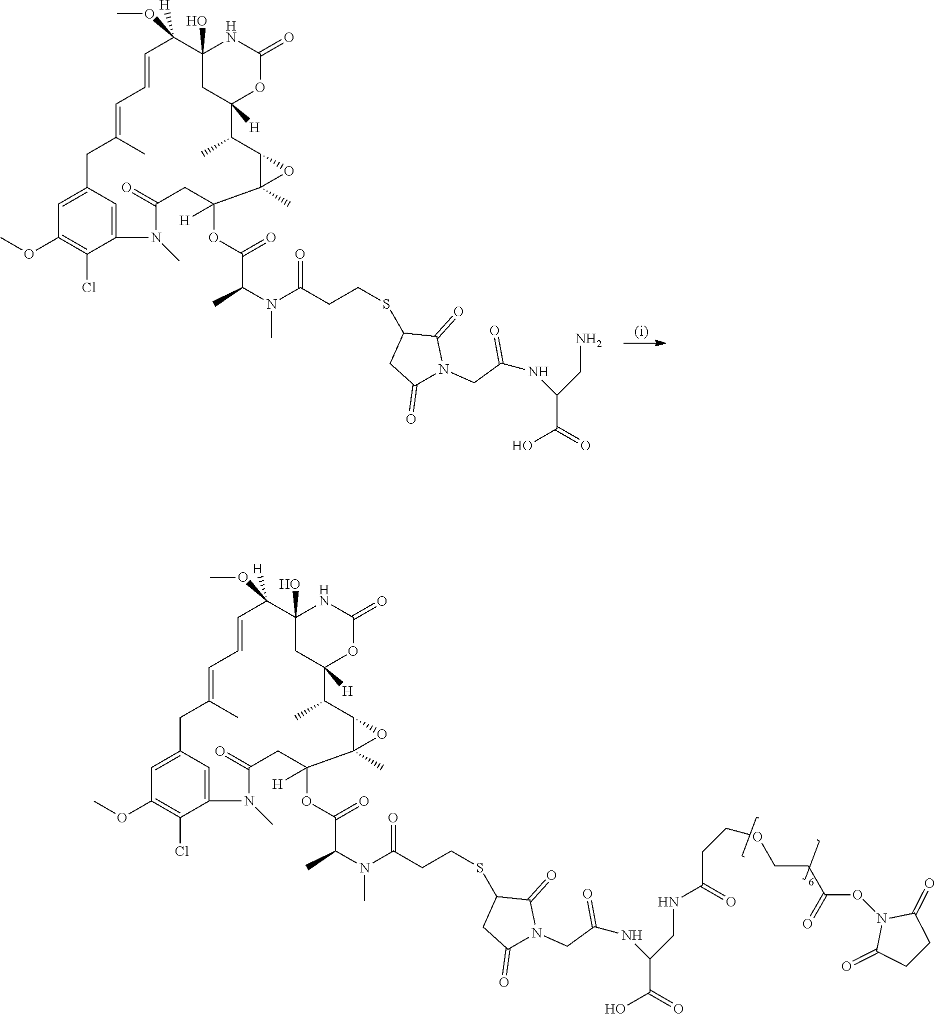

### Part B

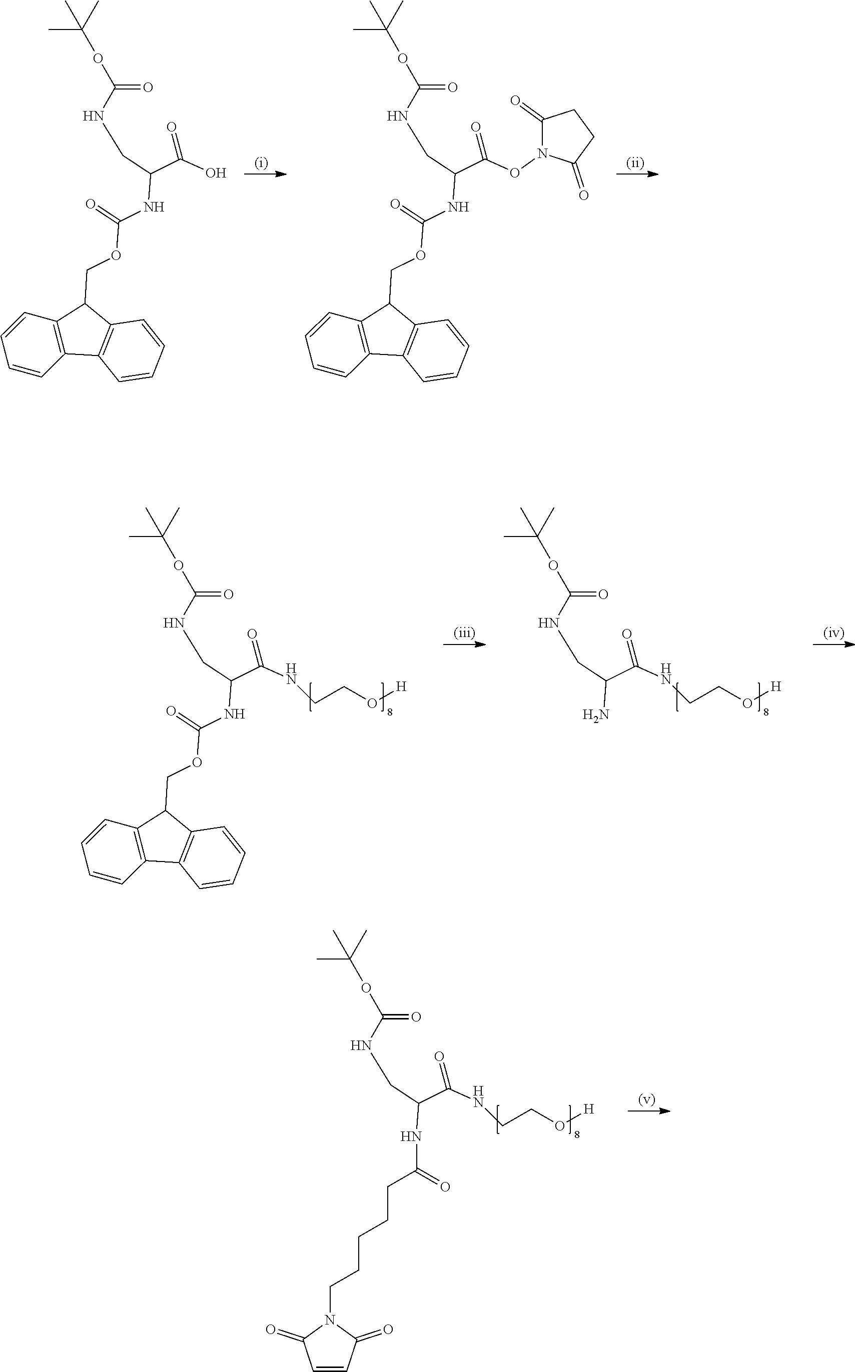

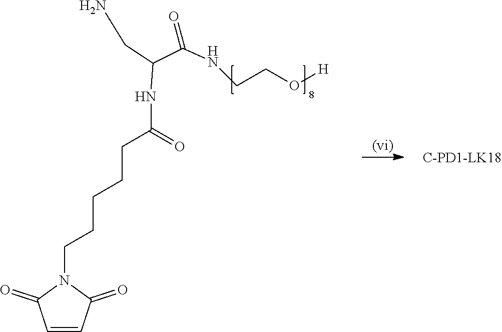

Compound 18 of this invention, i.e., MC-Dap(PEG6-Dap-AMAS-DM1)-PEG8-OH, was prepared following the protocols as indicated below.

### Part A.

(i) The amide bond formation between DM1-AMAS-Dap(NH2)-OH and Bis-PEG6-NHS followed the NHS protocol.

### Part B.

(i) Synthesis of NHS ester of Fmoc-Dap(Boc)-OH followed the protocol for NHS ester synthesis.
(ii) The amide bond formation between Fmoc-Dap(Boc)-NHS and amino-PEG8-OH followed the NHS protocol.
(iii) Removal of the Fmoc protection group of Fmoc-Dap(Boc)-PEG8-OH followed the Fmoc protocol.
(iv) The amide bond formation between NH2-Dap(Boc)-PEG8-OH and 6-Maleimidohexanoic acid N-hydroxysuccinimide ester (MC-NHS) followed the NHS protocol.
(v) Removal of the Boc protection group of MC-Dap(Boc)-PEG8-OH followed the Boc protocol.
(vi) The amide bond formation between MC-Dap(NH2)-PEG8-OH and DM1-AMAS-Dap(PEG6-NHS)—OH followed the NHS protocol.

Formula of C-L-PD=C91H141ClN10O36S Formula Weight C-L-PD=2018.66

### Example 19: Compound 19, i.e., C-PD1-LK19 (PD=Dap-AMAS-DM1; HP=PEG8-Acid)

### Part A

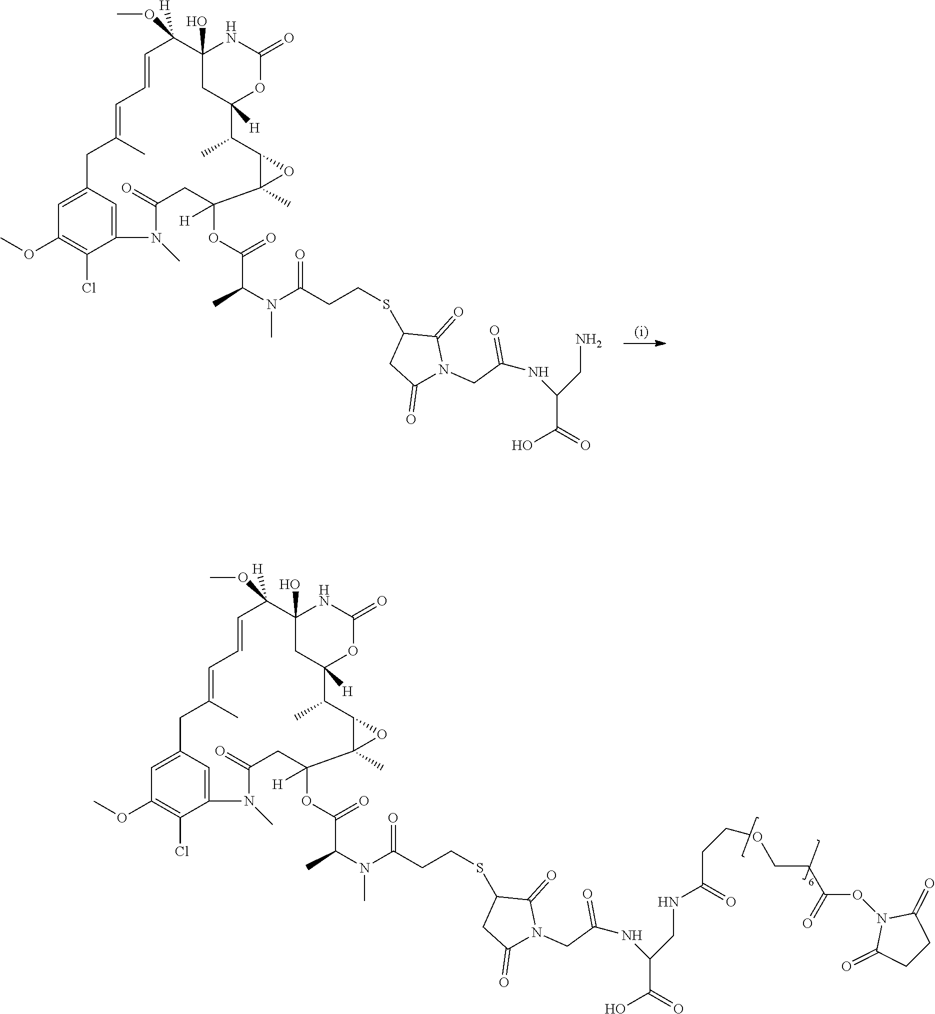

### Part B

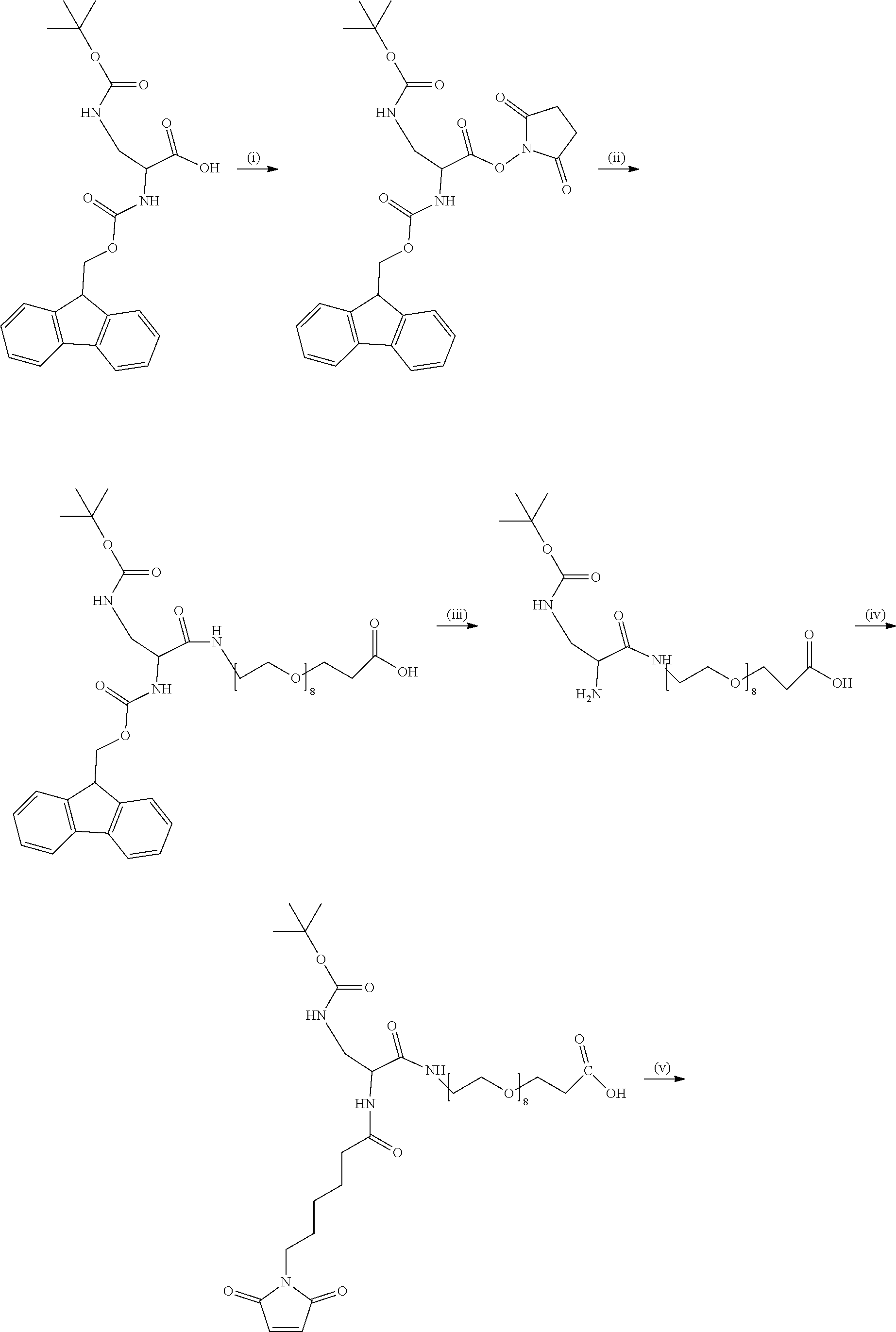

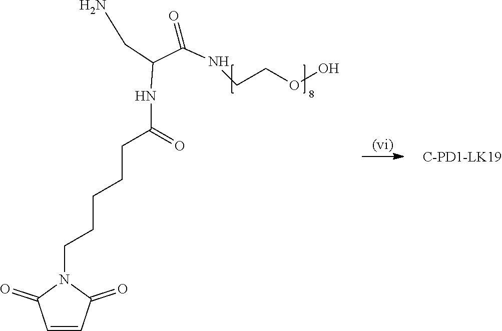

Compound 19 of this invention, i.e., MC-Dap(PEG6-Dap-AMAS-DM1)-PEG8-Acid, was prepared following the protocols as indicated below.

### Part A.

(i) The amide bond formation between DM1-AMAS-Dap(NH2)-OH and Bis-PEG6-NHS followed the NHS protocol.

### Part B.

(i) Synthesis of NHS ester of Fmoc-Dap(Boc)-OH followed the protocol for NHS ester synthesis.
(ii) The amide bond formation between Fmoc-Dap(Boc)-NHS and amino-PEG8-acid followed the NHS protocol.
(iii) Removal of the Fmoc protection group of Fmoc-Dap(Boc)-PEG8-acid followed the Fmoc protocol.
(iv) The amide bond formation between NH2-Dap(Boc)-PEG8-acid and 6-Maleimidohexanoic acid N-hydroxysuccinimide ester (MC-NHS) followed the NHS protocol.
(v) Removal of the Boc protection group of MC-Dap(Boc)-PEG8-acid followed the Boc protocol.
(vi) The amide bond formation between MC-Dap(NH2)-PEG8-acid and DM1-AMAS-Dap(PEG6-NHS)-acid followed the NHS protocol.

Formula of C-L-PD=C92H141C1N10037S Formula Weight C-L-PD=2046.67

### Example 20: Compound 20, i.e., C-PD1-LK20 (PD=Dap-AMAS-DM1; HP=PEG24-m)

### Part A

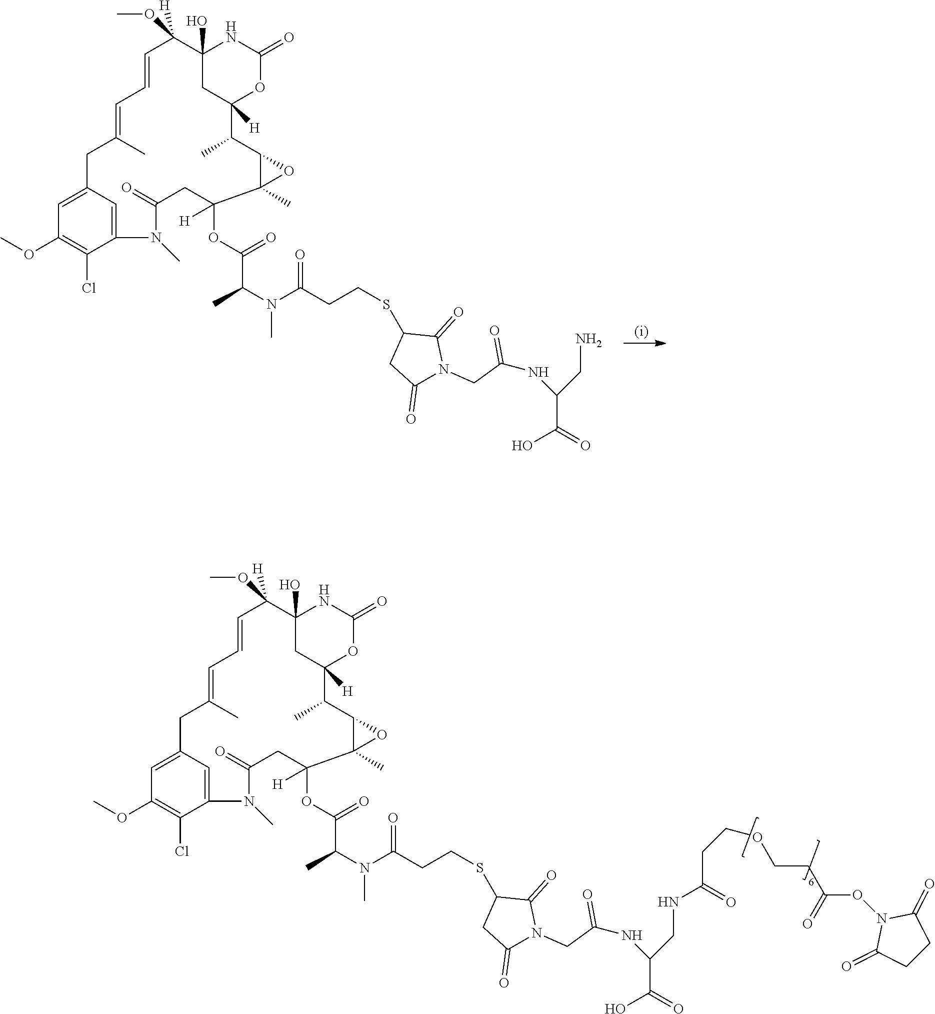

### Part B

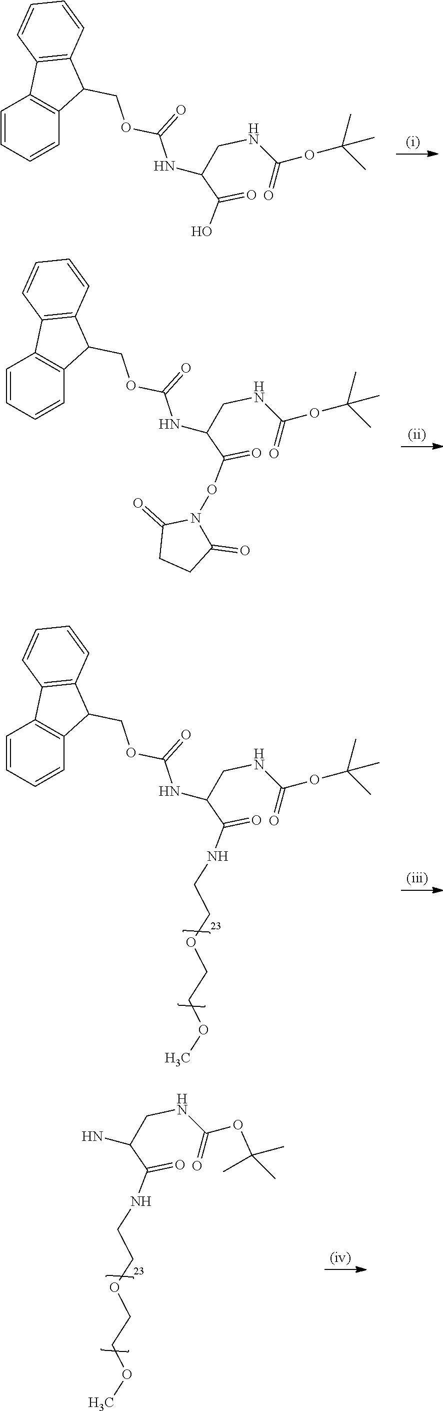

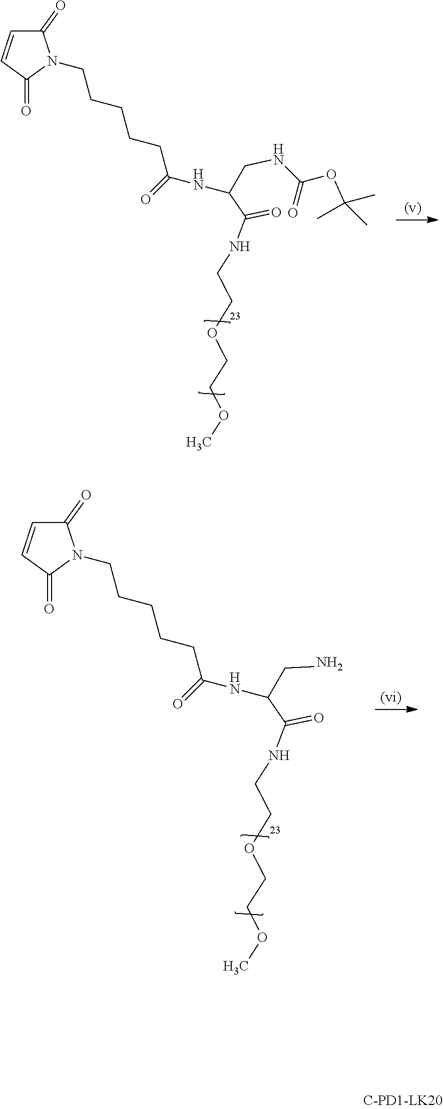

Compound 20 of this invention, i.e., MC-Dap(PEG6-Dap-AMAS-DM1)-PEG24-m, was prepared following the protocols as indicated below.

### Part A.

(i) The amide bond formation between DM1-AMAS-Dap(NH2)-OH and Bis-PEG6-NHS followed the NHS protocol.

### Part B.

(i) Synthesis of NHS ester of Fmoc-Dap(Boc)-OH followed the protocol for NHS ester synthesis.
(ii) The amide bond formation between Fmoc-Dap(Boc)-NHS and amino-PEG24-m followed the NHS protocol.
(iii) Removal of the Fmoc protection group of Fmoc-Dap(Boc)-PEG24-m followed the Fmoc protocol.
(iv) The amide bond formation between NH2-Dap(Boc)-PEG24-m and 6-Maleimidohexanoic acid N-hydroxysuccinimide ester (MC-NHS) followed the NHS protocol.
(v) Removal of the Boc protection group of MC-Dap(Boc)-PEG24-m followed the Boc protocol.
(vi) The amide bond formation between MC-Dap(NH2)-PEG24-m and DM1-AMAS-Dap(PEG6-NHS)—OH followed the NHS protocol.

### Example 21: Compound 21, i.e., C-PD1-LK21 (PD=Dap-AMAS-DM1; HP=PEG24-OH)

### Part A

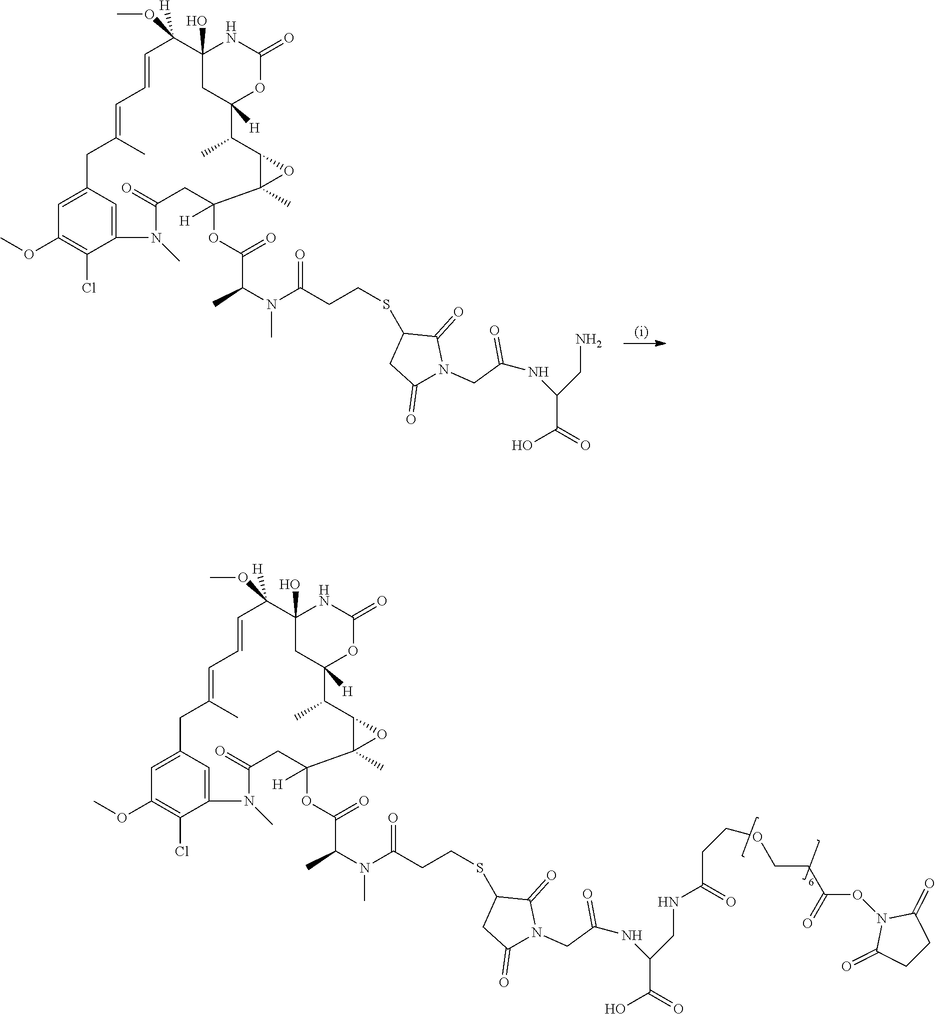

### Part B

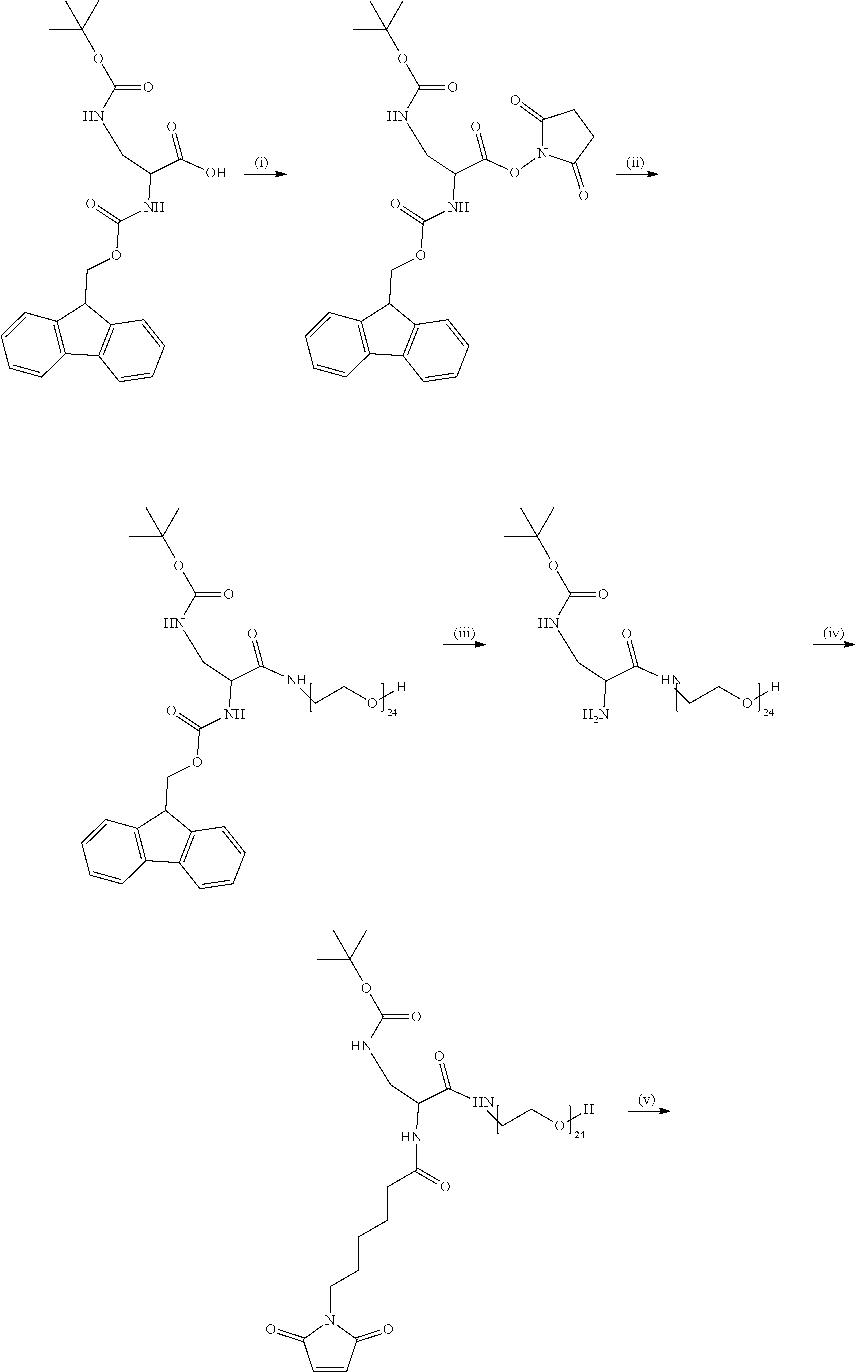

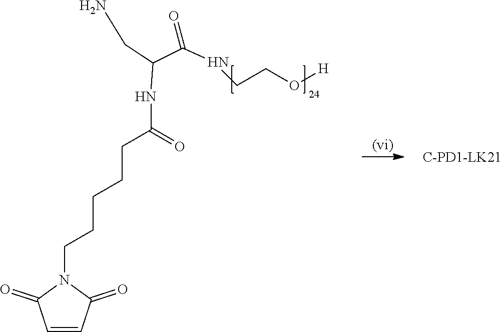

Compound 21 of this invention, i.e., MC-Dap(PEG6-Dap-AMAS-DM1)-PEG24-OH, was prepared following the protocols as indicated below.

### Part A.

(i) The amide bond formation between DM1-AMAS-Dap(NH2)-OH and Bis-PEG6-NHS followed the NHS protocol.

### Part B.

(i) Synthesis of NHS ester of Fmoc-Dap(Boc)-OH followed the protocol for NHS ester synthesis.
(ii) The amide bond formation between Fmoc-Dap(Boc)-NHS and amino-PEG24-OH followed the NHS protocol.
(iii) Removal of the Fmoc protection group of Fmoc-Dap(Boc)-PEG24-OH followed the Fmoc protocol.
(iv) The amide bond formation between NH2-Dap(Boc)-PEG24-OH and 6-Maleimidohexanoic acid N-hydroxysuccinimide ester (MC-NHS) followed the NHS protocol.
(v) Removal of the Boc protection group of MC-Dap(Boc)-PEG24-OH followed the Boc protocol.
(vi) The amide bond formation between MC-Dap(NH2)-PEG24-OH and DM1-AMAS-Dap(PEG6-NHS)-acid followed the NHS protocol.

### Example 22: Compound 22, i.e., C-PD1-LK22 (PD=Dap-AMAS-DM1; HP=PEG24-Acid)

### Part A

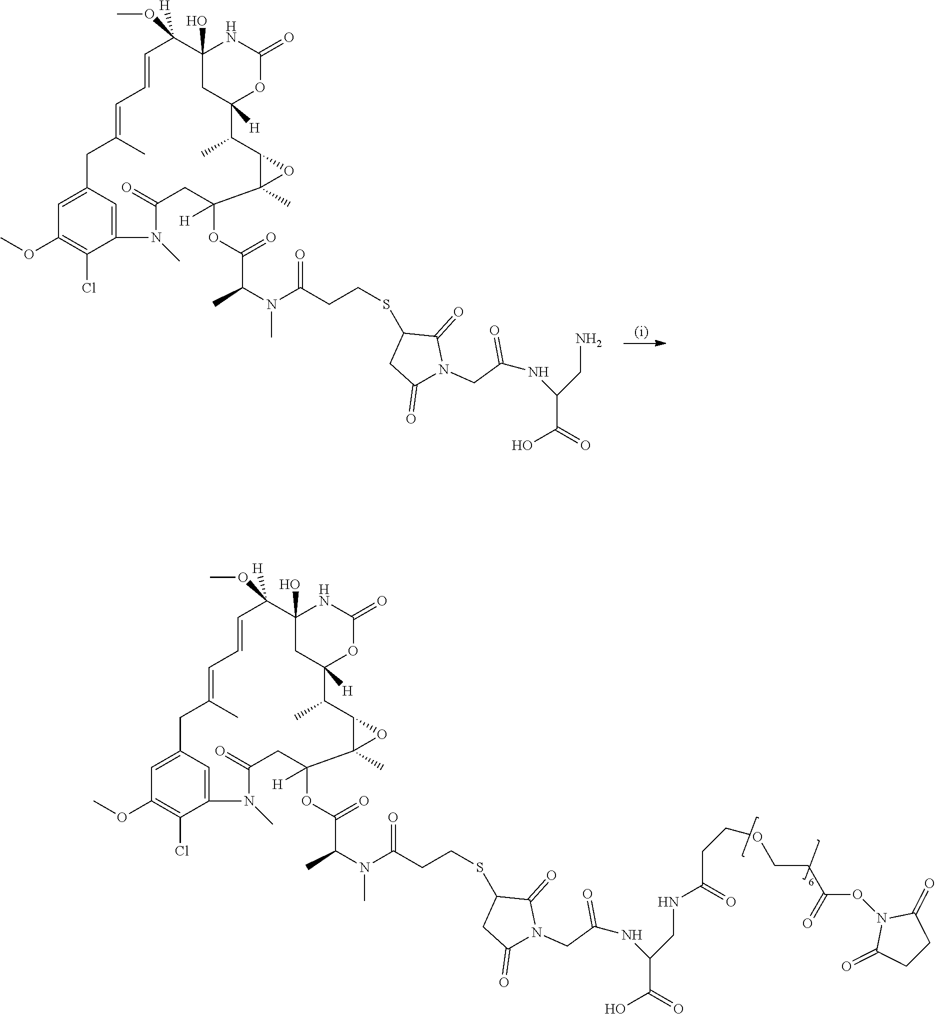

### Part B

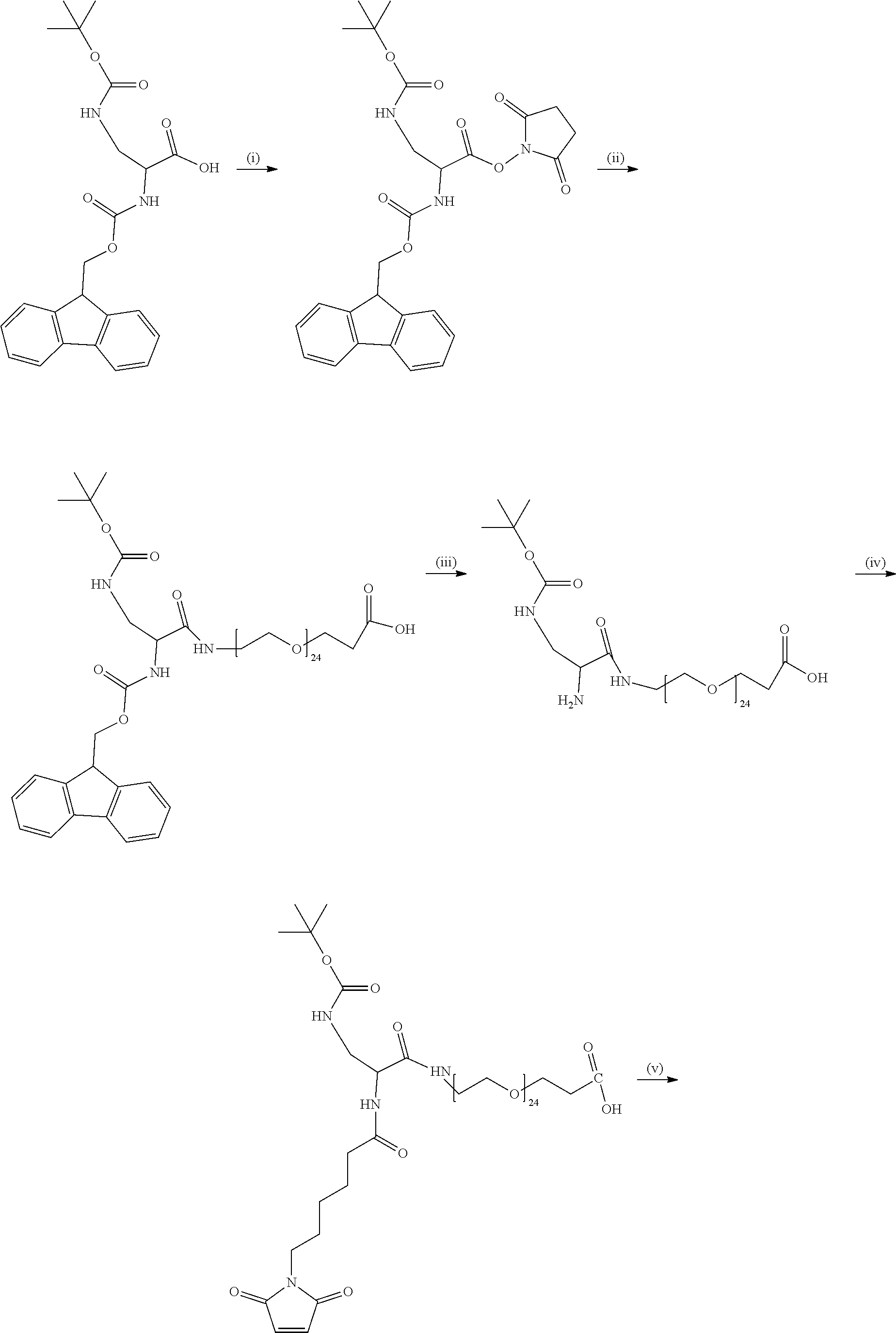

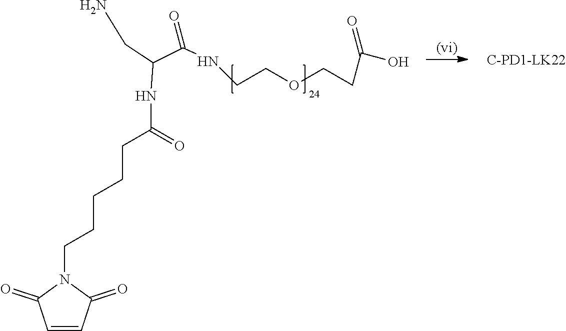

Compound 22 of this invention, i.e., MC-Dap(PEG6-Dap-AMAS-DM1)-PEG24-Acid, was prepared following the protocols as indicated below.

### Part A.

(i) The amide bond formation between DM1-AMAS-Dap(NH2)-OH and Bis-PEG6-NHS followed the NHS protocol.

### Part B.

(i) Synthesis of NHS ester of Fmoc-Dap(Boc)-OH followed the protocol for NHS ester synthesis.
(ii) The amide bond formation between Fmoc-Dap(Boc)-NHS and amino-PEG24-acid followed the NHS protocol.
(iii) Removal of the Fmoc protection group of Fmoc-Dap(Boc)-PEG24-acid followed the Fmoc protocol.
(iv) The amide bond formation between NH2-Dap(Boc)-PEG24-acid and 6-Maleimidohexanoic acid N-hydroxysuccinimide ester (MC-NHS) followed the NHS protocol.
(v) Removal of the Boc protection group of MC-Dap(Boc)-PEG24-acid followed the Boc protocol.
(vi) The amide bond formation between MC-Dap(NH2)-PEG24-acid and DM1-AMAS-Dap(PEG6-NHS)-acid followed the NHS protocol.

### Example 23: Compound 23, i.e., C-PD1-LK23 (PD=muramic acid-AMAS-DM1; HP=PEG24-m)

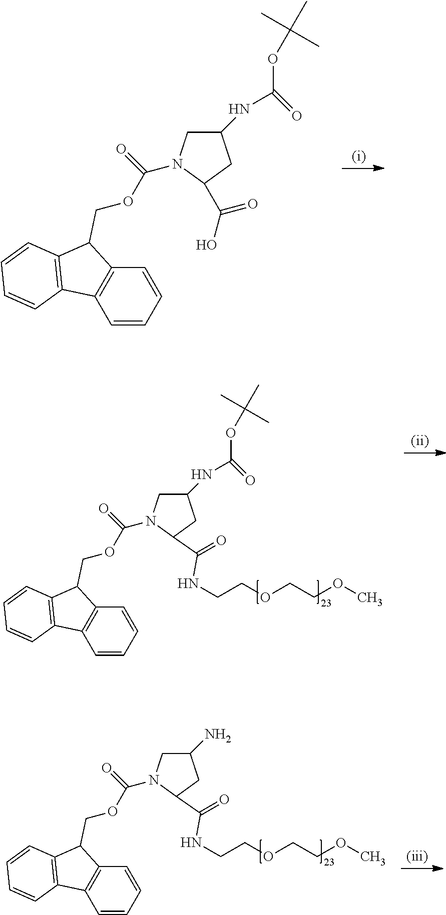

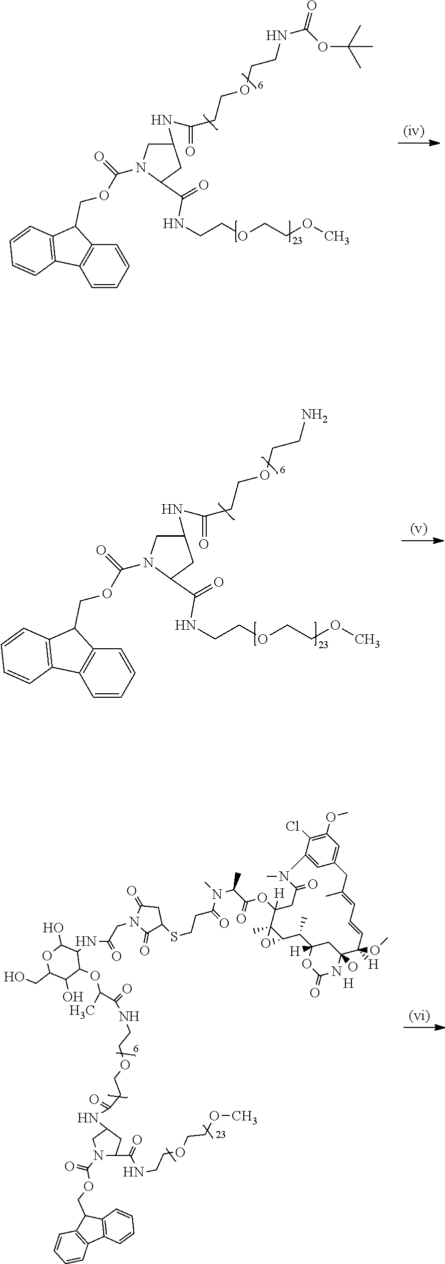

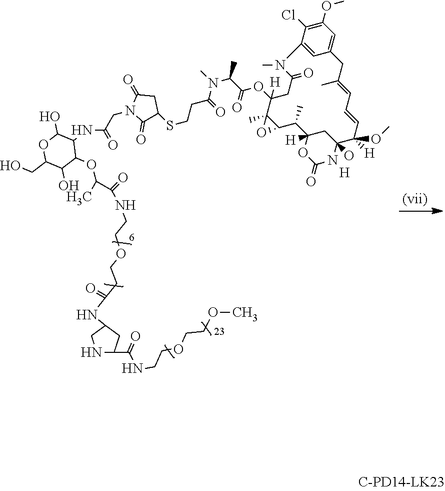

Compound 23 of this invention, i.e., Mal-PEG2-Pro(PEG6-muramic acid-AMAS-DM1)-PEG24-m, was prepared following the protocols as indicated below.
(i) The amide bond formation between Fmoc-4AP(Boc)-OH and amino-PEG24-m followed the HATU protocol.
(ii) Removal of the Boc protection group of Fmoc-4AP(Boc)-PEG24-m followed the Boc protocol.
(iii) The amide bond formation between Fmoc-4AP(NH2)-PEG24-m and t-Boc-N-amido-PEG6-acid followed the HATU protocol.
(iv) Removal of the Boc protection group of Fmoc-4AP(t-Boc-N-amido-PEG6)-PEG24-m followed the Boc protocol.
(v) The amide bond formation between Fmoc-4AP(N-amido-PEG6)-PEG24-m and muramic acid-AMAS-DM1 followed the HATU protocol.
(vi) Removal of the Fmoc protection group of Fmoc-4AP(PEG6-muramic acid-AMAS-DM1)-PEG24-m followed the Fmoc protocol.
(vii) The amide bond formation between NH2-4AP(PEG6-muramic acid-AMAS-DM1)-PEG24-m and 4-maleimidophenylacetic acid followed the HATU protocol.

### Example 24: Compound 24, i.e., C-PD1-LK24 (PD=Dap-AMAS-DM1: HP=PEG80H)

### Part A

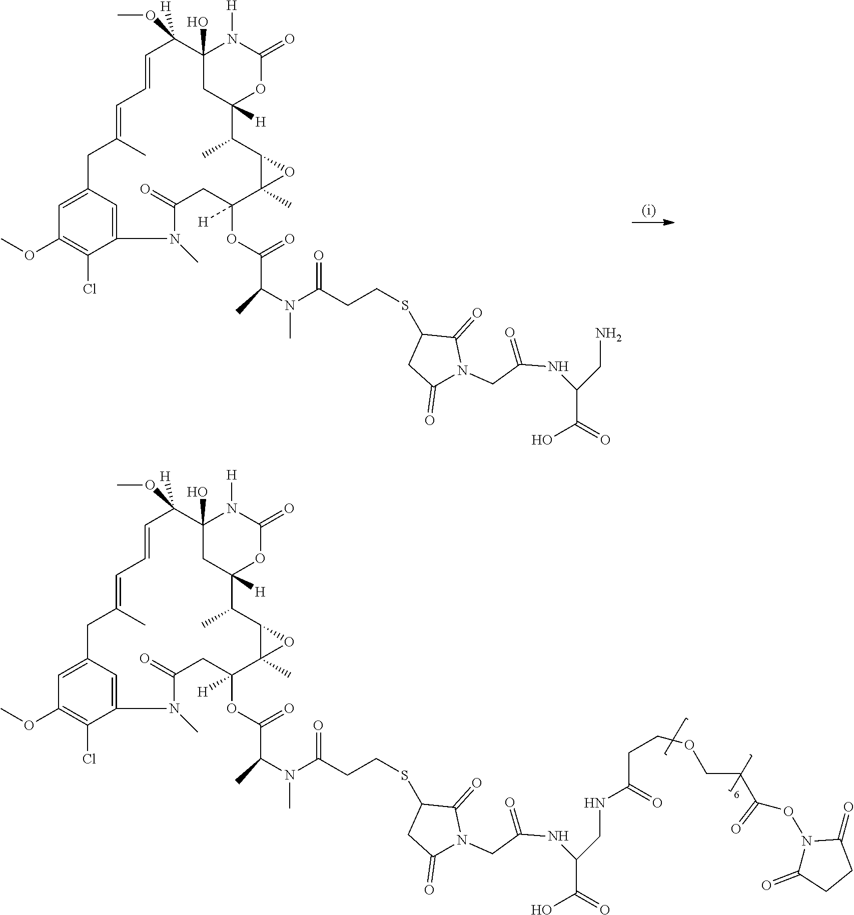

### Part B

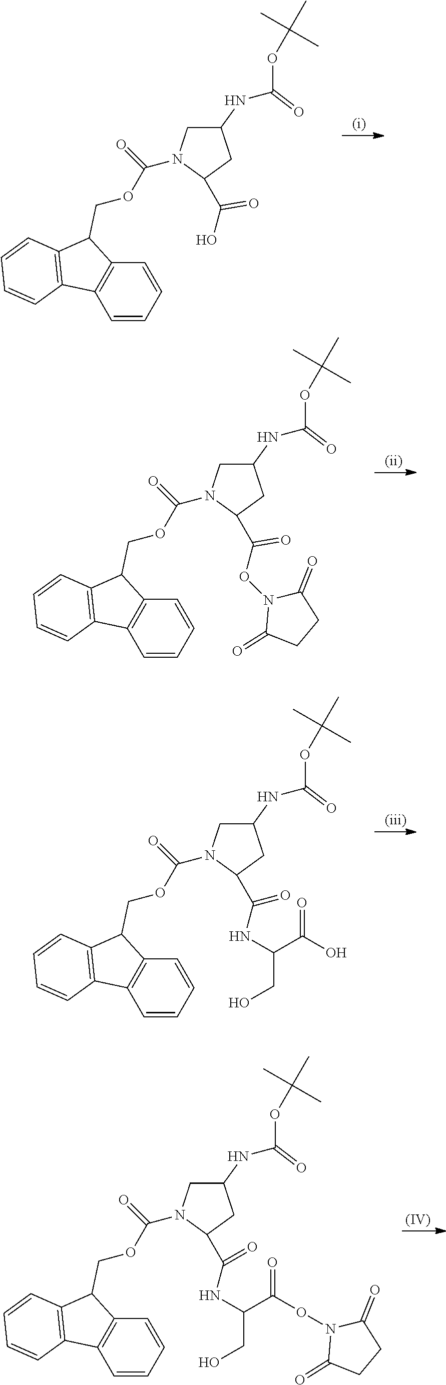 [SMILES: CC(C)(C)OC(=O)NC1CC(C(=O)O)N(C(=O)OCC2c3ccccc3-c3ccccc32)C1 ; CC(C)(C)OC(=O)NC1CC(C(=O)ON2C(=O)CCC2=O)N(C(=O)OCC2c3ccccc3-c3ccccc32)C1 ; CC(C)(C)OC(=O)NC1CC(C(=O)NC(CO)C(=O)O)N(C(=O)OCC2c3ccccc3-c3ccccc32)C1 ; CC(C)(C)OC(=O)NC1CC(C(=O)NC(CO)C(=O)ON2C(=O)CCC2=O)N(C(=O)OCC2c3ccccc3-c3ccccc32)C1 ; "(IV)"]

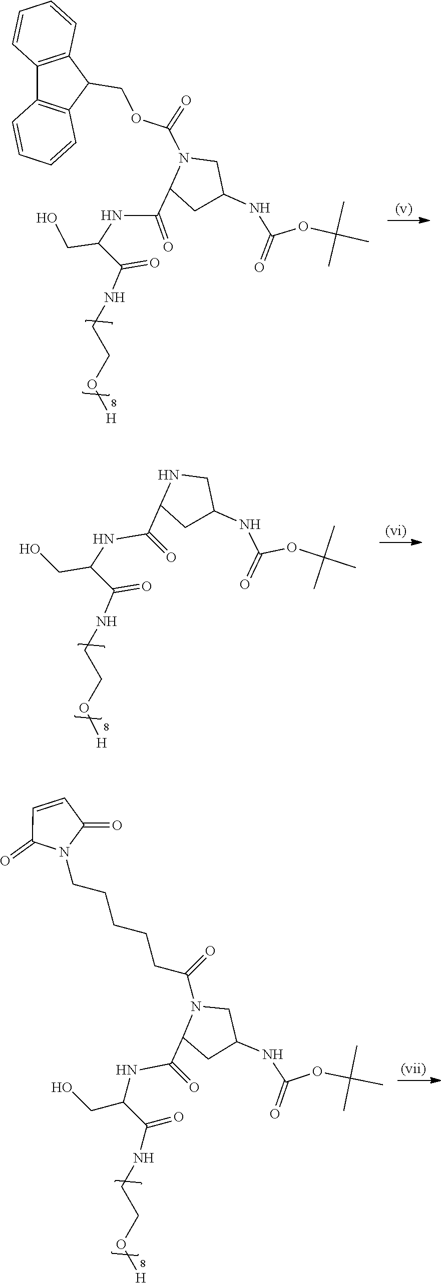

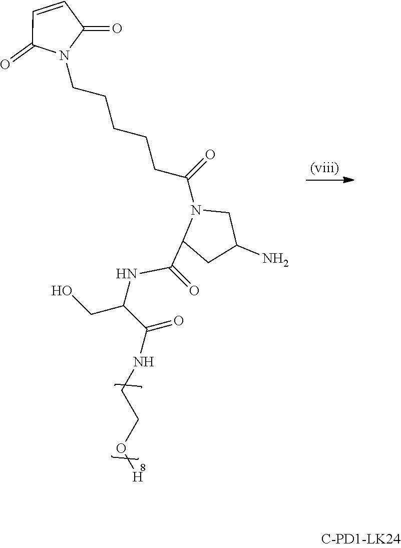

Compound 24 of this invention, i.e., MC-4AP(PEG6-Dap-AMAS-DM1)-Ser-PEG80H, was prepared following the protocols as indicated below.

### Part A.

(i) The amide bond formation between DM1-AMAS-Dap(NH2)-OH and bis-PEG6-NHS followed the NHS protocol.

### Part B.

(i) Synthesis of NHS ester of Fmoc-4AP(Boc)-OH followed the NHS ester protocol.
(ii) The amide bond formation between Fmoc-4AP(Boc)-NHS and serine followed the NHS protocol.
(iii) Synthesis of NHS ester of Fmoc-4AP(Boc)-Ser followed the NHS ester protocol.
(iv) The amide bond formation between Fmoc-4AP(Boc)-Ser-NHS and amino-PEG8-alcohol followed the NHS protocol.
(v) Removal of the Fmoc protection group of Fmoc-4AP(Boc)-Ser-PEG8-OH followed the Fmoc protocol.
(vi) The amide bond formation between NH2-4AP(Boc)-Ser-PEG8-OH and 6-Maleimidohexanoic acid N-hydroxysuccinimide ester (MC-NHS) followed the NHS protocol.
(vii) Removal of the Boc protection group of MC-4AP(Boc)-Ser-PEG8-OH followed the Boc protocol.
(viii) The amide bond formation between MC-4AP(NH2)-Ser-PEG8-OH and DM1-AMAS-Dap(PEG6-NHS)—OH followed the NHS protocol.

### Example 25: Compound 25, i.e., C-PD6-LK25 (PD=Ala(DM1)-Ome; HP=PEG24-m)

### Part A

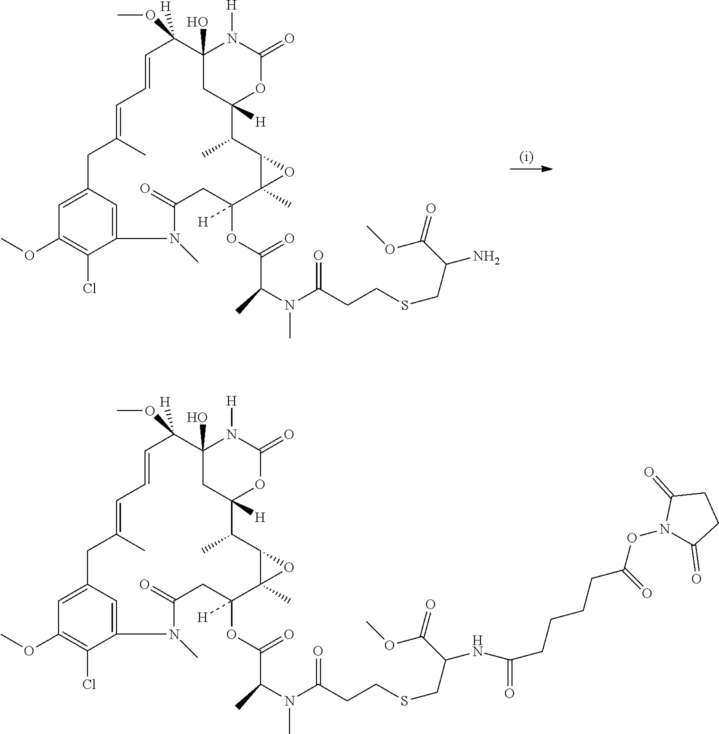 [SMILES: COC(=O)C(N)CSCCC(=O)N(C)[C@@H](C)C(=O)O[C@H]1CC(=O)N(C)c2cc(cc(OC)c2Cl)C/C(C)=C/C=C/[C@@H](OC)[C@@]2(O)C[C@H](OC(=O)N2)[C@@H](C)[C@@H]2O[C@@]12C ; COC(=O)C(CSCCC(=O)N(C)[C@@H](C)C(=O)O[C@H]1CC(=O)N(C)c2cc(cc(OC)c2Cl)C/C(C)=C/C=C/[C@@H](OC)[C@@]2(O)C[C@H](OC(=O)N2)[C@@H](C)[C@@H]2O[C@@]12C)NC(=O)CCCCC(=O)ON1C(=O)CCC1=O]

### Part B

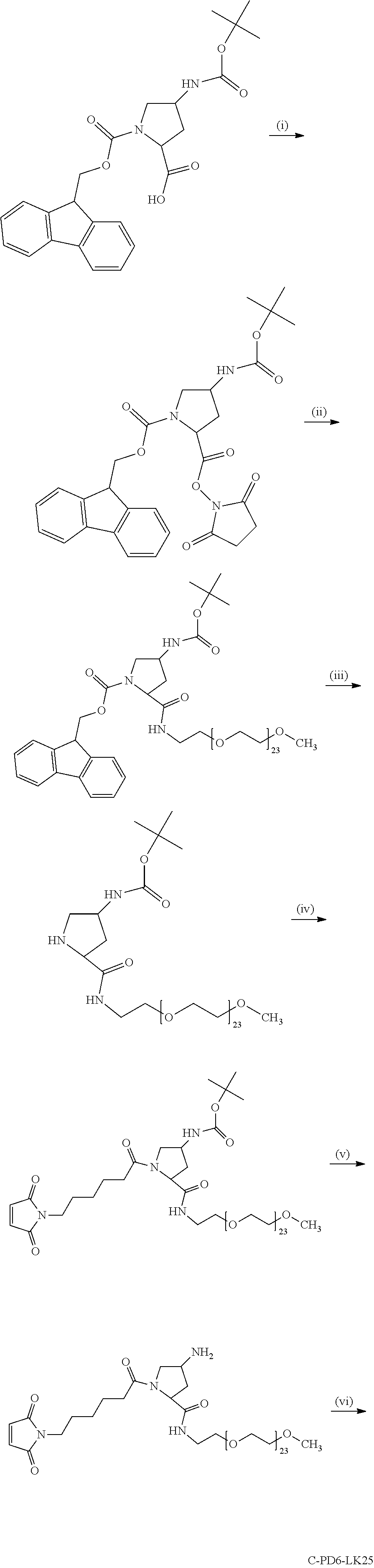

Compound 25 of this invention, i.e., Mc-4AP(DSA-Ala(DM1)-Ome)-PEG24-m, was prepared following the protocols as indicated below.

### Part A.

(i) The amide bond formation between NH2-Ala(DM1)-Ome and Di(N-succinimidyl)adipate (DSA) followed the NHS protocol.

### Part B.

(i) Synthesis of NHS ester of Fmoc-4AP(Boc)-OH followed the NHS ester protocol.
(ii) The amide bond formation between Fmoc-4AP(Boc)-NHS and amino-PEG24-m followed the NHS protocol.
(iii) Removal of the Fmoc protection group of Fmoc-4AP(Boc)-PEG24-m followed the Fmoc protocol.
(iv) The amide bond formation between NH2-4AP(Boc)-PEG24-m and 6-Maleimidohexanoic acid N-hydroxysuccinimide ester (MC-NHS) followed the NHS protocol.
(v) Removal of the Boc protection group of MC-4AP(Boc)-PEG24-m followed the Boc protocol.
(vi) The amide bond formation between MC-4AP(NH2)-PEG24-m and DSA-Ala(DM1)-Ome followed the NHS protocol.

### Example 26: Compound 26, i.e., C-PD4-LK26 (PD=DM1-AMAS-Asn-OH; HP=PEG24-m)

### Part A

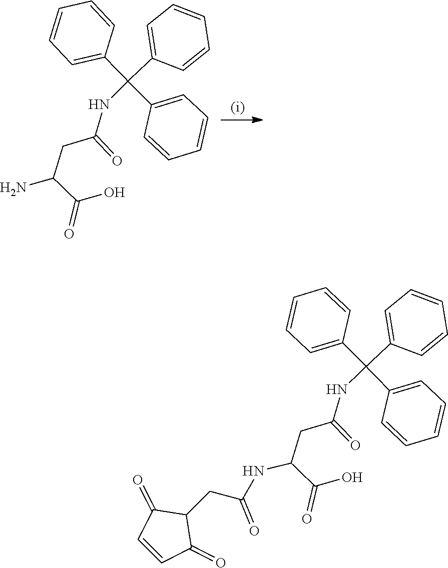 [SMILES: NC(CC(=O)NC(c1ccccc1)(c1ccccc1)c1ccccc1)C(=O)O ; O=C(CC1C(=O)C=CC1=O)NC(CC(=O)NC(c1ccccc1)(c1ccccc1)c1ccccc1)C(=O)O]

### Part B

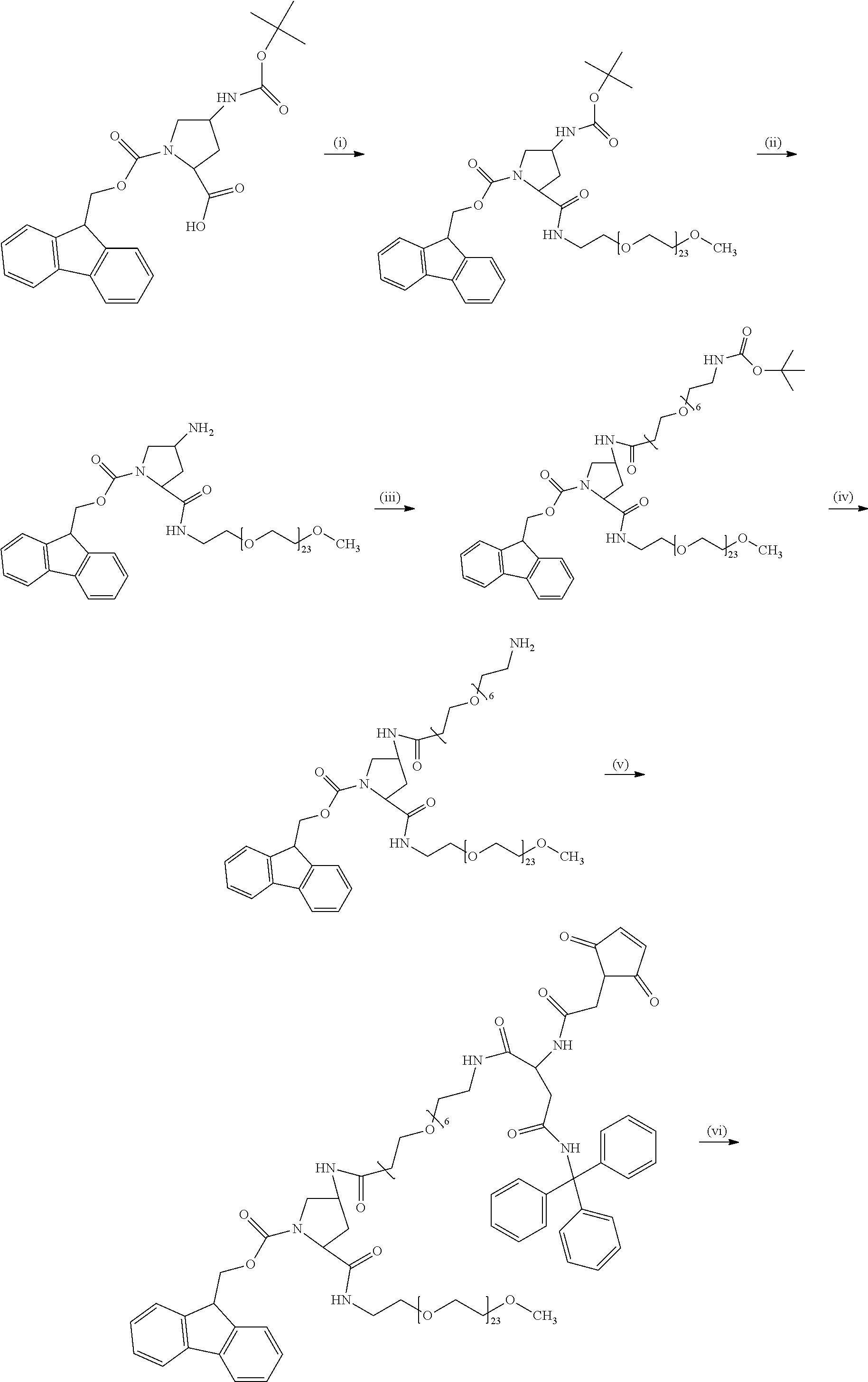

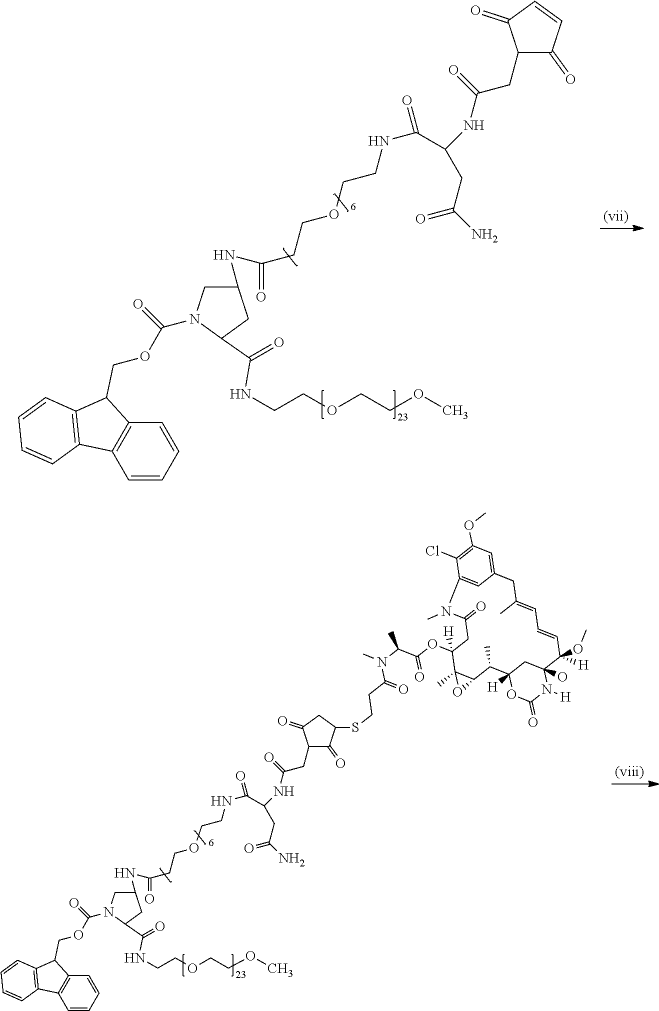

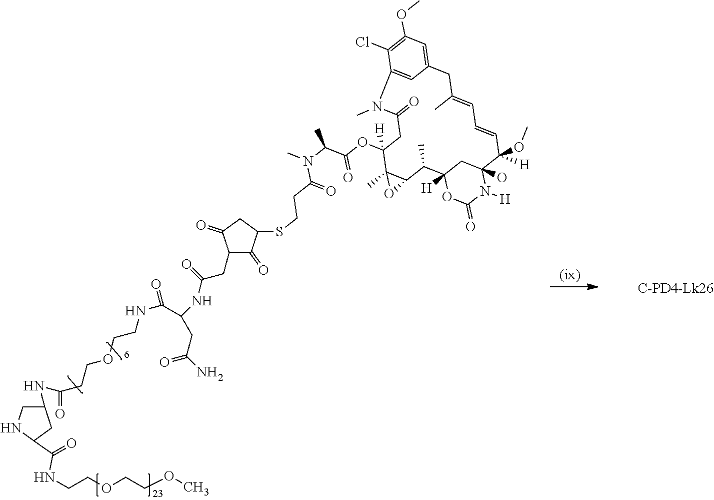

Compound 26 of this invention, i.e., MC-4AP(PEG6-ASN-AMAS-DM1)-PEG24-m, was prepared following the protocols as indicated below.

### Part A.

(i) The amide bond formation between ASN(TRT)-OH and N-Succinimidyl Maleimidoacetate (AMAS) followed the NHS protocol.

### Part B.

(i) The amide bond formation between Fmoc-4AP(Boc)-OH and amino-PEG24-m following the HATU protocol.
(ii) Removal of the Boc protection group of Fmoc-4AP(Boc)-PEG24-m followed the Boc protocol.
(iii) The amide bond formation between Fmoc-4AP(NH2)-PEG24-m and t-Boc-N-amido-PEG6-acid followed the HATU protocol.
(iv) Removal of the Boc protection group of Fmoc-4AP(t-Boc-N-amido-PEG6)-PEG24-m followed the Boc protocol.
(v) The amide bond formation between Fmoc-4AP(N-amido-PEG6)-PEG24-m and AMAS-ASN(TRT)-OH followed the HATU protocol.
(vi) Removal of the TRT protection group of Fmoc-4AP[PEG6-ASN(TRT)-AMAS]-PEG24-m followed the TRT protocol.
(vii) The maleimide-thiol bond formation between Fmoc-4AP[PEG6-ASN-AMAS]-PEG24-m and DM1 followed the maleimide-thiol protocol.
(viii) Removal of the Fmoc protection group of Fmoc-4AP[PEG6-ASN-AMAS-DM1]-PEG24-m followed the Fmoc protocol.
(ix) Amide bond formation between NH2-4AP[PEG6-ASN-AMAS-DM1]-PEG24-m and 6-Maleimidocaproic acid followed the HATU protocol.

### Example 27: Compound 27, i.e., I-PD1-LK1: (PD=Dap-TLR7/8 Agonist 1; HP=PEG12-m)

### Part A

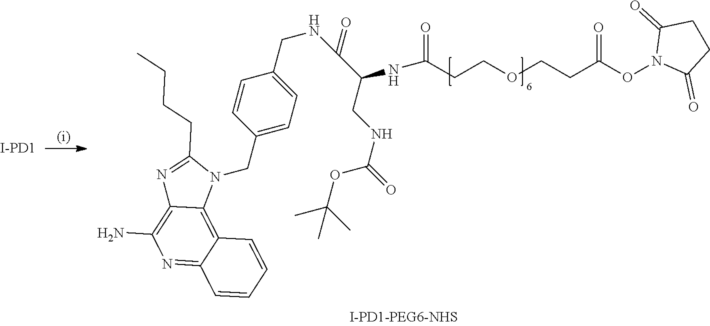

### Part B

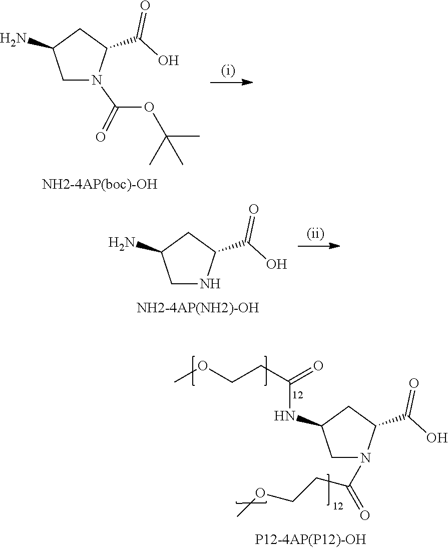

### Part C

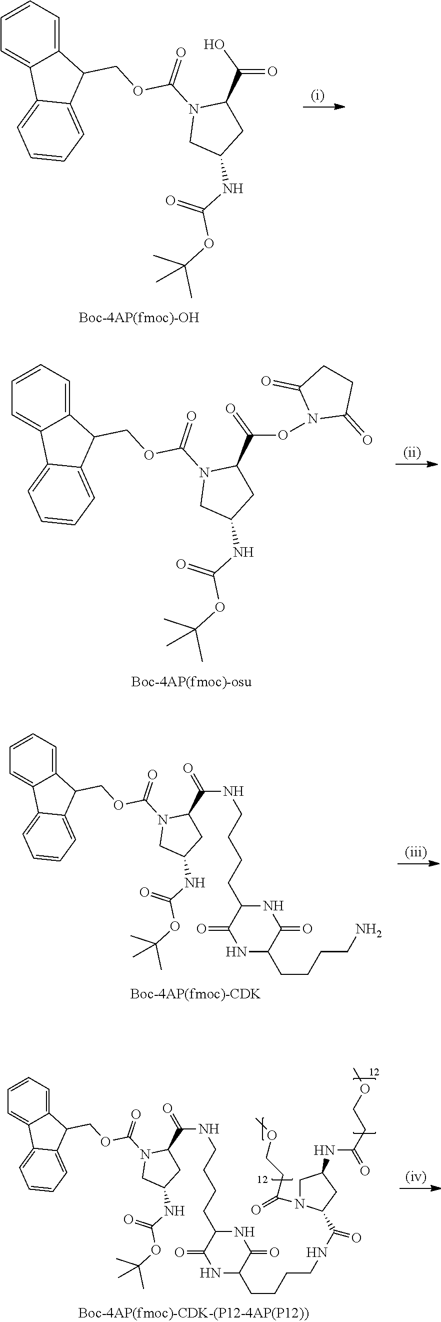

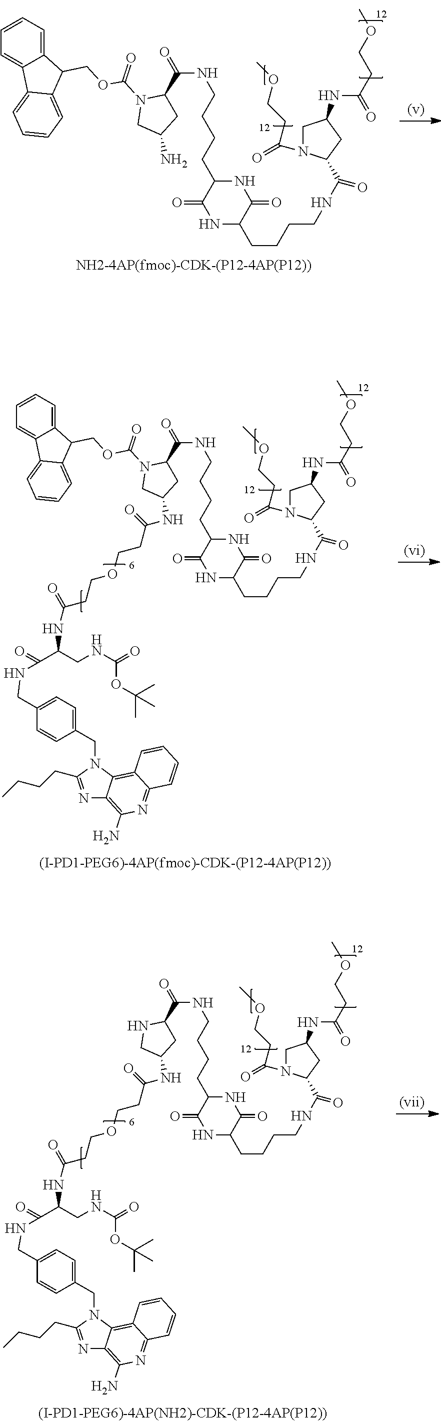

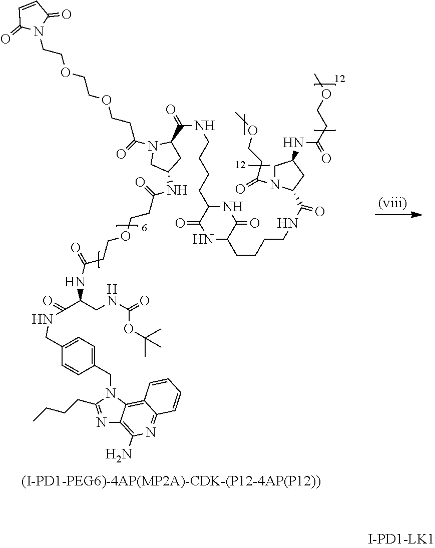

Compound 27 of this invention was prepared following the protocols as indicated below.
Part A-(i): The amide bond formation between I-PD1 and Bis-PEG6-NHS ester followed the NHS protocol.
Part B-(i): Removal of the Boc protection group of (2R,4S)-1-Boc-4-Aminopyrrolidine-2-carboxylic acid (NH2-4AP(Boc)-OH) followed the Boc protocol.
Part B-(ii): The amide bond formation between NH2-4AP(NH2)-OH and m-PEG12-NHS ester followed the NHS protocol.
Part C-(i): Synthesis of NHS ester of (2R,4S)-1-Fmoc-4-Boc-amino pyrrolidine-2-carboxylic acid (Boc-4AP(Fmoc)-OH) followed the NHS ester protocol.
Part C-(ii): The amide bond formation between Boc-4AP(Fmoc)-osu and 3,6-bis(4-aminobutyl)-2,5-piperazinedione (CDK) followed the NHS protocol.
Part C-(iii): The amide bond formation between P12-4AP(P12)—OH and Boc-4AP(Fmoc)-CDK followed the HATU protocol.
Part C-(iv): Removal of the Boc protection group of Boc-4AP(Fmoc)-CDK-(P12-4AP(P12)) followed the Boc protocol.
Part C-(v): The amide bond formation between I-PD1-PEG6-NHS and NH2-4AP(Fmoc)-CDK-(P12-4AP(P12)) followed the NHS protocol.
Part C-(vi): Removal of the Fmoc protection group of (I-PD1-PEG6)-4AP(Fmoc)-CDK-(P12-4AP(P12)) followed the Fmoc protocol.
Part C-(vii): The amide bond formation between (I-PD1-PEG6)-4AP(NH2)-CDK-(P12-4AP(P12)) and Mal-PEG2-acid followed the HATU protocol.
Part C-(viii): Removal of the Boc protection group of (I-PD1-PEG6)-4AP(MP2A)-CDK-(P12-4AP(P12)) followed the Boc protocol. Mass calculation by ESI-MS m/z for C126H211N16O44 [M+H]+: is 2652.5, in agreement with its theoretic value of 2652.5.

### Example 28: Compound 28, i.e., I-PD2-LK2 (PD=4AP-TLR7/8 Agonist 1; HP=PEG24-m)

### Part A

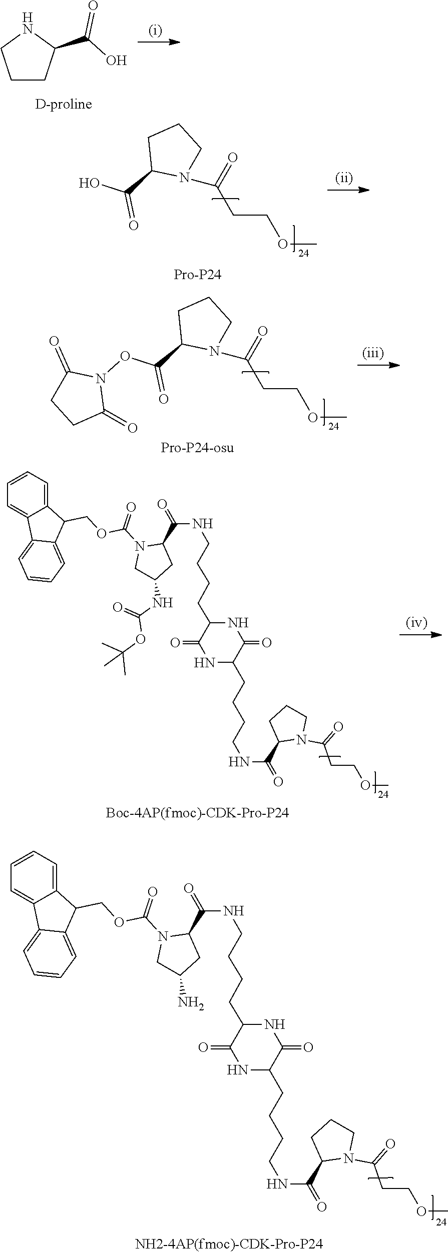

### Part B

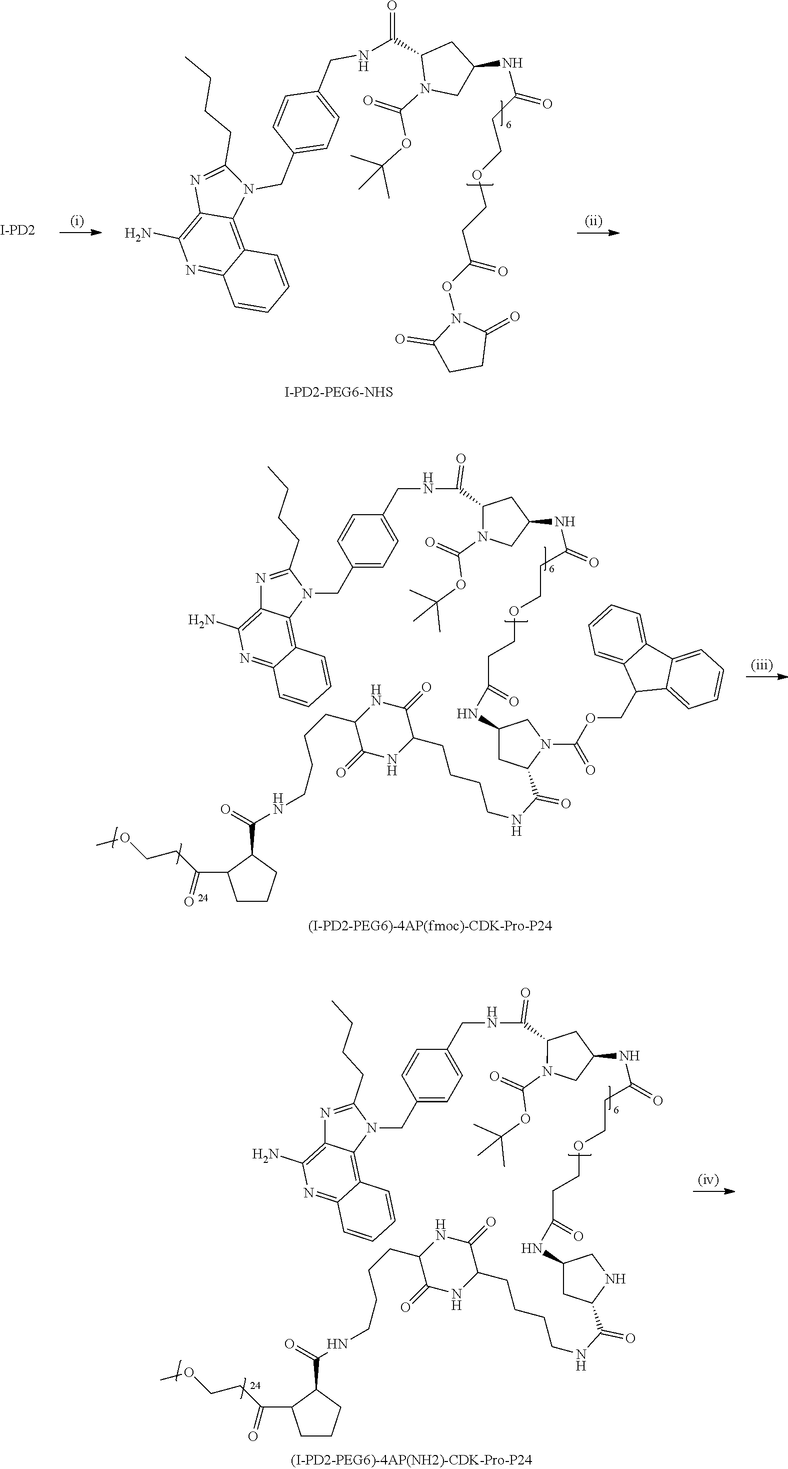

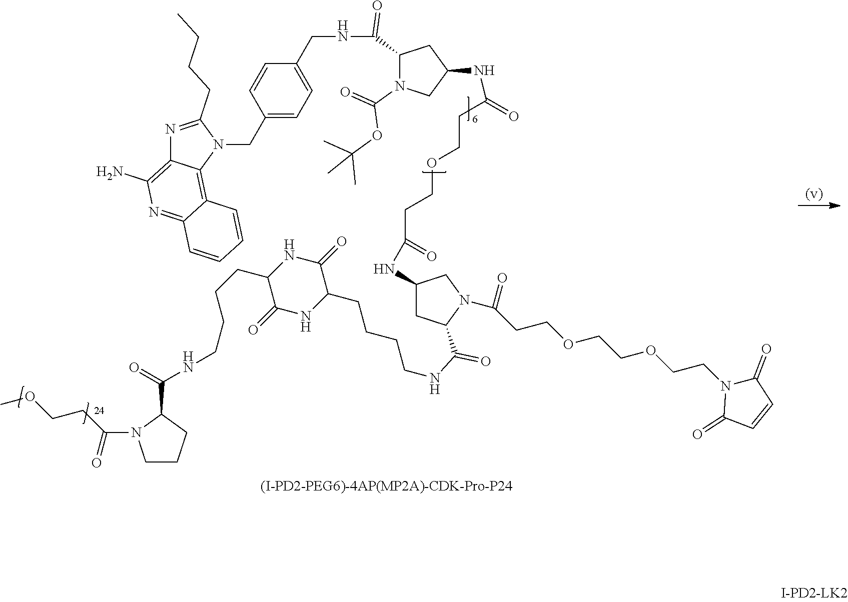

Compound 28 of this invention was prepared following the protocols as indicated below.
Part A-(i): The amide bond formation between D-proline and m-PEG24-NHS ester followed the NHS protocol.
Part A-(ii): Synthesis of NHS ester of Pro-P24 followed the NHS ester protocol.
Part A-(iii): The amide bond formation between Boc-4AP(Fmoc)-CDK and Pro-P24-osu followed the NHS protocol
Part A-(iv): Removal of the Boc protection group of Boc-4AP(Fmoc)-CDK-Pro-P24 followed the Boc protocol.
Part B-(i): The amide bond formation between I-PD2 and Bis-PEG6-NHS ester followed the NHS protocol.
Part B-(ii): The amide bond formation between NH2-4AP(Fmoc)-CDK-Pro-P24 and I-PD2-PEG6-NHS followed the NHS protocol.
Part B-(iii): Removal of the Fmoc protection group of I-PD2-PEG6-4AP(Fmoc)-CDK-Pro-P24 followed the Fmoc protocol.
Part B-(iv): The amide bond formation between I-PD2-PEG6-4AP(NH2)-CDK-Pro-P24 and Mal-PEG2-acid followed the HATU protocol.
Part B-(v): Removal of the Boc protection group of I-PD2-PEG6-4AP(MP2A)-CDK-Pro-P24 (8.5 mg) followed the Boc protocol.

ESI-MS m/z calculated for C126H210N15O43 [M+H]+: 2621.5; found 2621.5.

### Example 29: Compound 29, i.e., I-PD2-LK3 (PD=4AP-TLR7/8 Agonist 1; HP=2X PEG12-m)

### Part A

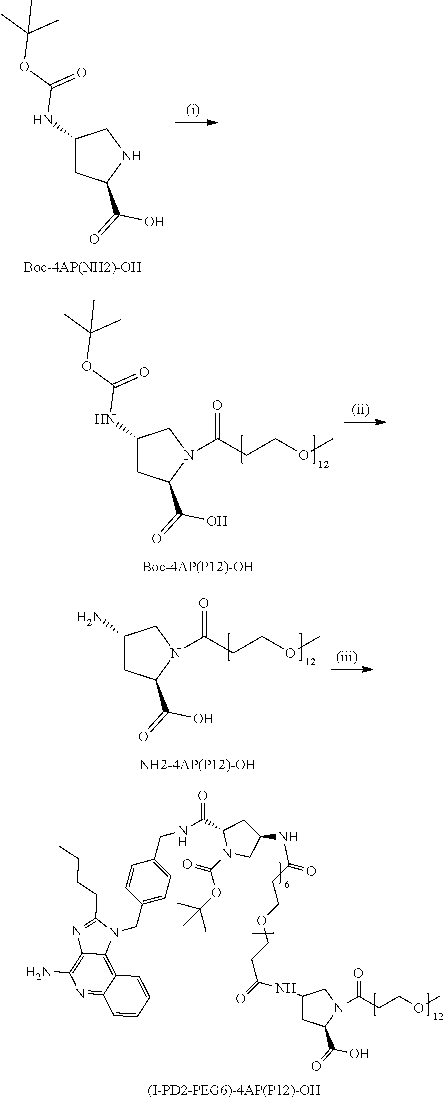

### Part B

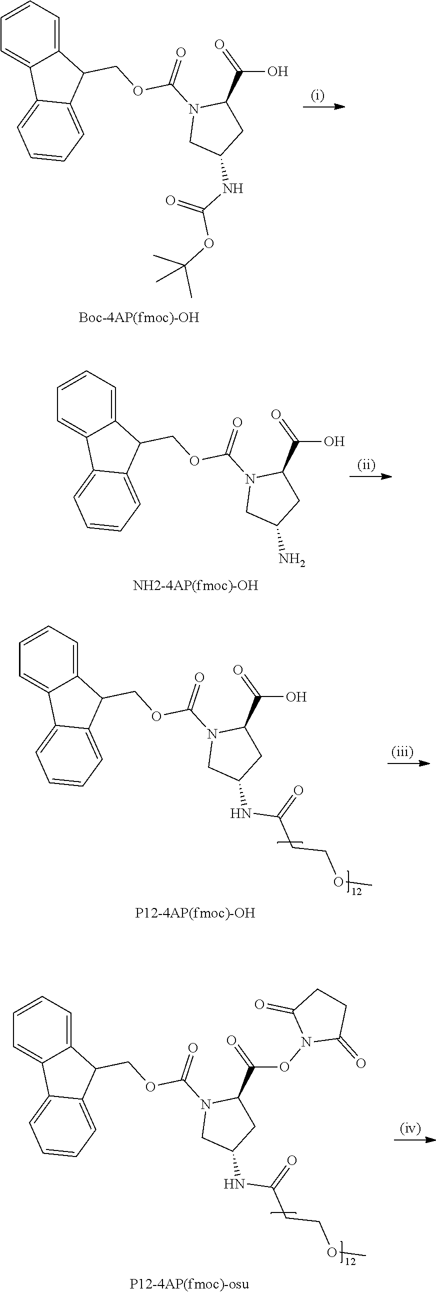

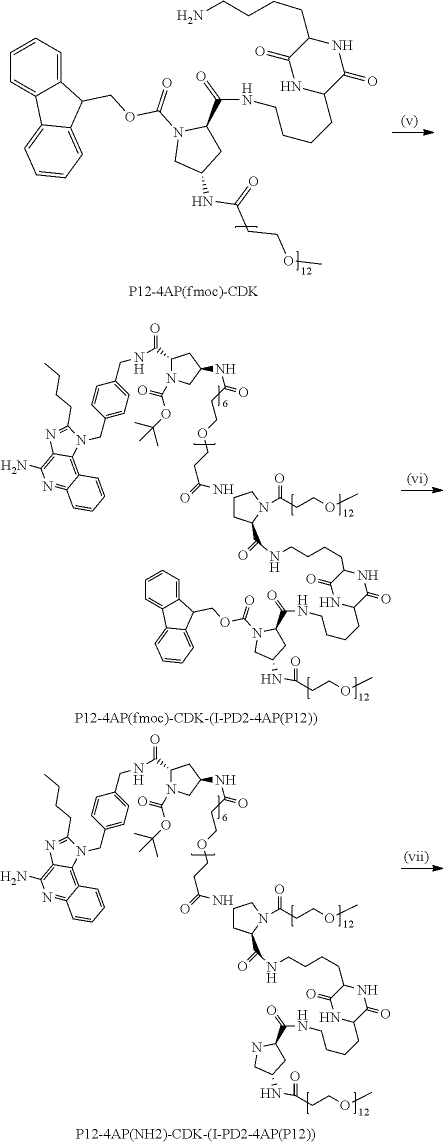

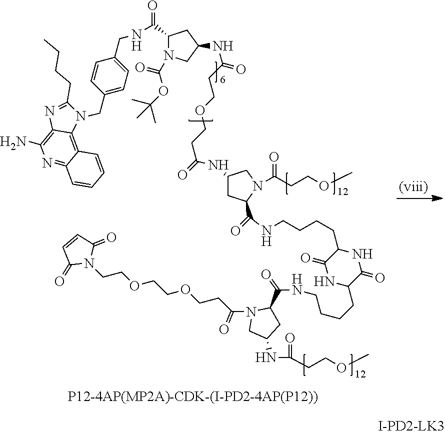

Compound 29 of this invention was prepared following the protocols as indicated below.
Part A-(i): The amide bond formation between (2R,4S)-4-((tert-Butoxycarbonyl)amino)pyrrolidine-2-carboxylic acid (Boc-4AP(NH2)-OH) and m-PEG12-NHS ester followed the NHS protocol.
Part A-(ii): Removal of the Boc protection group of Boc-4AP(P12)—OH followed the Boc protocol.
Part A-(iii): The amide bond formation between NH2-4AP(P12)—OH and I-PD2-PEG6-NHS followed the NHS protocol.
Part B-(i): Removal of the Boc protection group of (2R,4S)-1-Fmoc-4-Boc-amino pyrrolidine-2-carboxylic acid (Boc-4AP(Fmoc)-OH) followed the Boc protocol.
Part B-(ii): The amide bond formation between NH2-4AP(Fmoc)-OH and m-PEG12-NHS ester followed the NHS protocol.
Part B-(iii): Synthesis of NHS ester of P12-4AP(Fmoc)-OH followed the NHS ester protocol.
Part B-(iv): The amide bond formation between P12-4AP(Fmoc)-osu and 3,6-bis(4-aminobutyl)-2,5-piperazinedione (CDK) followed the NHS protocol.
Part B-(v): The amide bond formation between P12-4AP(Fmoc)-CDK, I-PD2-4AP(P12)—OH followed the HATU protocol.
Part B-(vi): Removal of the Fmoc protection group of P12-4AP(Fmoc)-CDK-(I-PD2-4AP(P12)) followed the Fmoc protocol.
Part B-(vii): The amide bond formation between P12-4AP(NH2)-CDK-(I-PD2-4AP(P12)) and Mal-PEG2-acid followed the HATU protocol.
Part B-(viii): Removal of the Boc protection group of P12-4AP(MP2A)-CDK-(I-PD2-4AP(P12)) followed the Boc protocol.

ESI-MS m/z calculated for C128H213N16044 [M+H]+: 2678.5; found 2678.5.

### Example 30: Compound 30, i.e., I-PD2-LK4 (PD=4AP-TLR7/8 Agonist 1; HP=2X PEG24-m)

### Part A

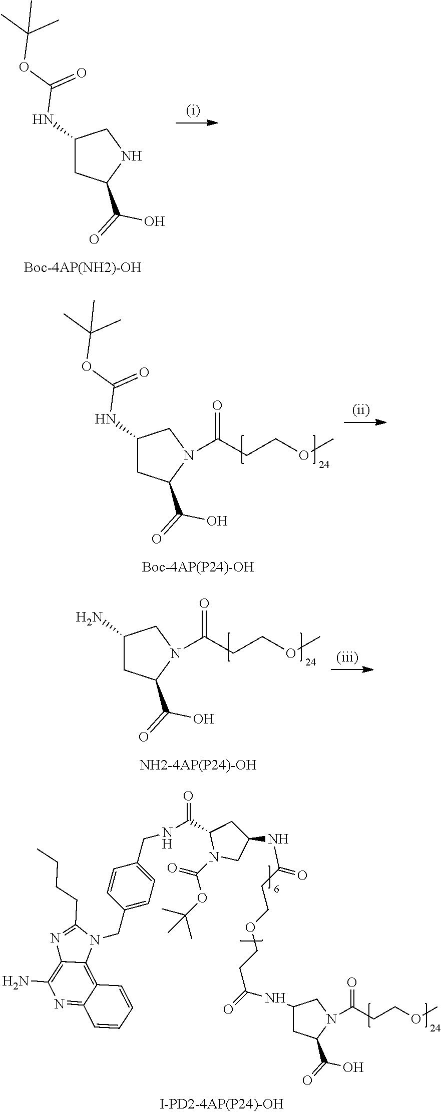

### Part B

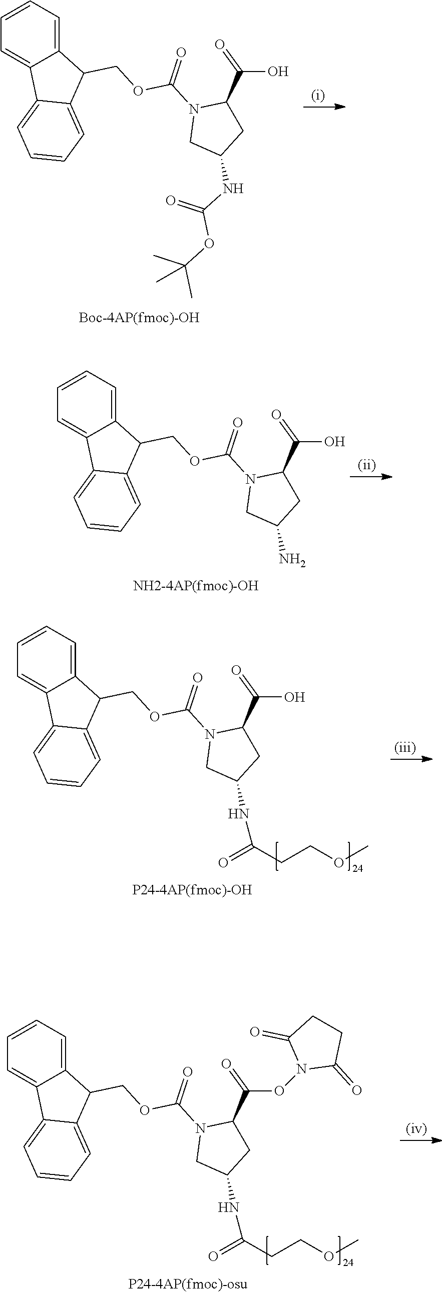

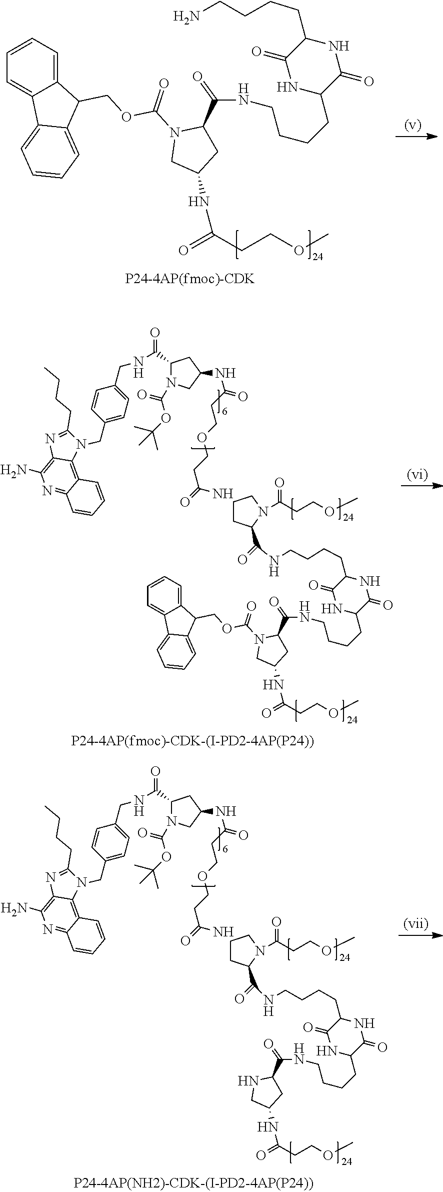

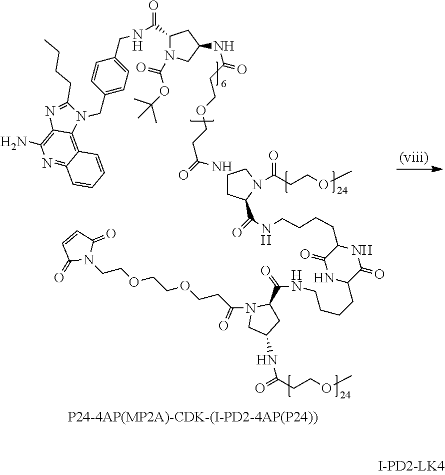

Compound 30 of this invention was prepared following the protocols as indicated below.
Part A-(i): The amide bond formation between (2R,4S)-4-((tert-Butoxycarbonyl)amino)-pyrrolidine-2-carboxylic acid (Boc-4AP(NH2)-OH) and m-PEG24-NHS ester followed the NHS protocol.
Part A-(ii): Removal of the Boc protection group of Boc-4AP(P24)—OH followed the Boc protocol.
Part A-(iii): The amide bond formation between NH2-4AP(P24)—OH and I-PD2-PEG6-NHS followed the NHS protocol.
Part B-(i): Removal of the Boc protection group of (2R,4S)-1-Fmoc-4-Boc-amino pyrrolidine-2-carboxylic acid (Boc-4AP(Fmoc)-OH) followed the Boc protocol.
Part B-(ii): The amide bond formation between NH2-4AP(Fmoc)-OH and m-PEG24-NHS ester followed the NHS protocol.
Part B-(iii): Synthesis of NHS ester of P24-4AP(Fmoc)-OH followed the NHS ester protocol.
Part B-(iv): The amide bond formation between P24-4AP(Fmoc)-osu and 3,6-bis(4-aminobutyl)-2,5-piperazinedione (CDK) followed the NHS protocol.
Part B-(v): The amide bond formation between P24-4AP(Fmoc)-CDK and I-PD2-4AP(P24)—OH followed the HATU protocol.
Part B-(vi): Removal of the Fmoc protection group of P24-4AP(Fmoc)-CDK-(I-PD2-4AP(P24)) followed the Fmoc protocol.
Part B-(vii): The amide bond formation between P24-4AP(NH2)-CDK-(I-PD2-4AP(P24)) and Mal-PEG2-acid followed the HATU protocol.
Part B-(viii): Removal of the Boc protection group of P24-4AP(MP2A)-CDK-(I-PD2-4AP(P24)) followed the Boc protocol.

ESI-MS m/z calculated for C176H309N16068 [M+H]+: 3735.1; found 3735.1.

### Example 31: Compound 31, I-PD-LK5(PD=PEG5-TLR7/8 Agonist 1; HP=2X PEG12-m)

### Part A

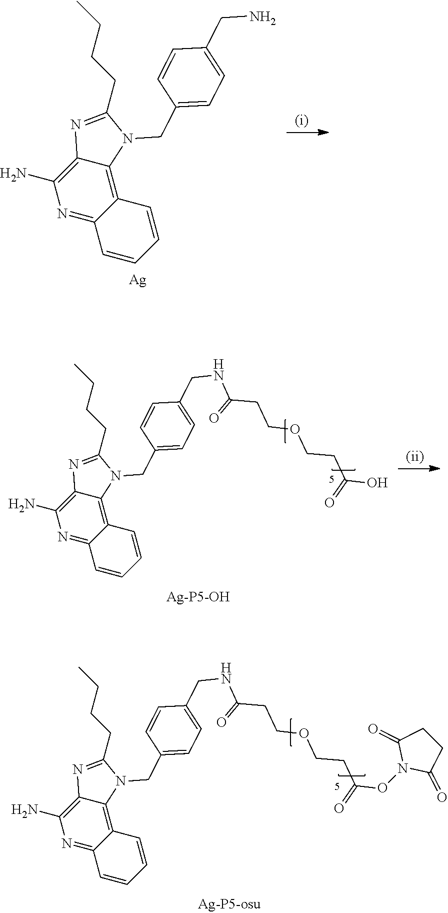

### Part B

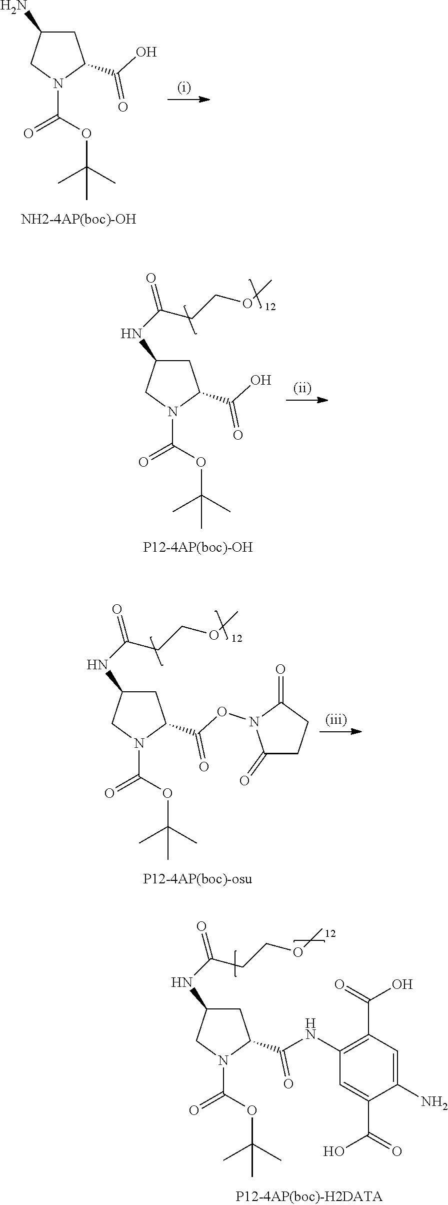

### Part C

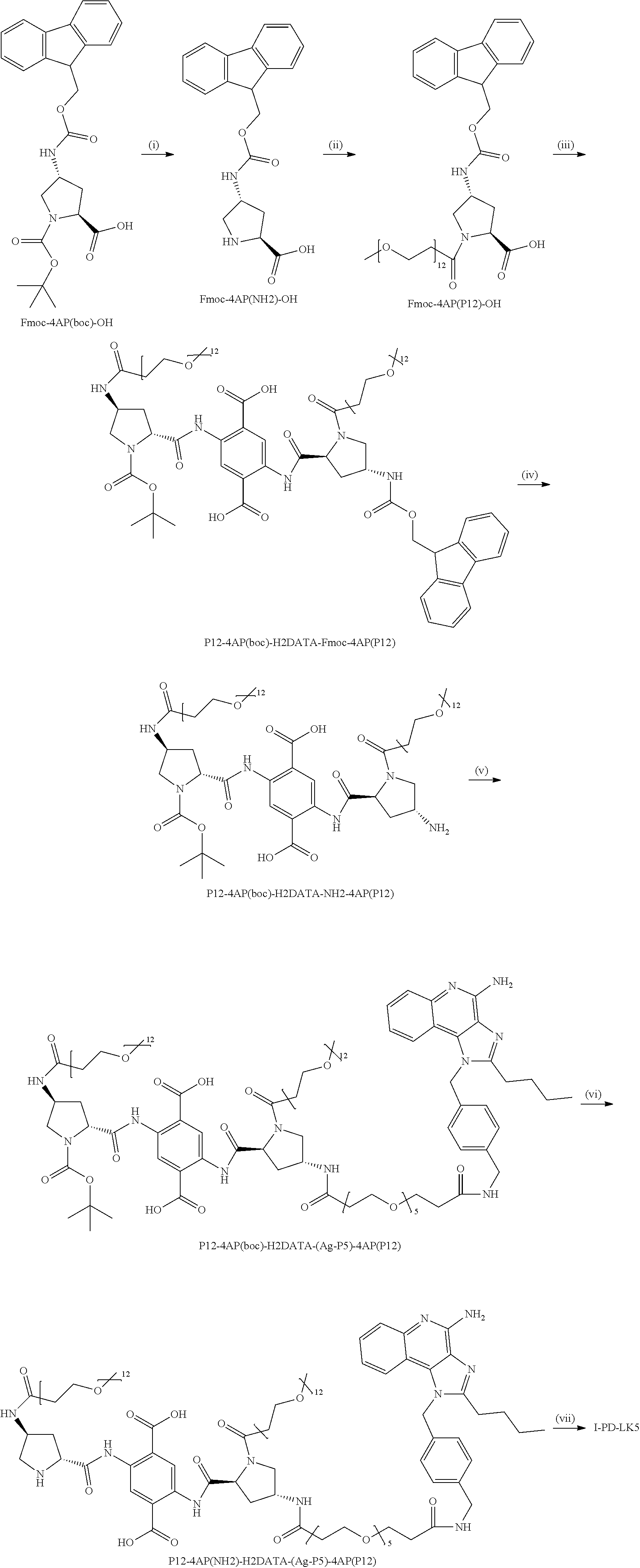

Compound 31 of this invention was prepared following the protocols as indicated below.
Part A-(i): The amide bond formation between Ag and acid-PEG5-NHS followed the NHS protocol.
Part A-(ii): Synthesis of NHS ester of Ag-P5-OH followed the NHS ester protocol.
Part B-(i): The amide bond formation between (2R,4S)-1-Boc-4-Aminopyrrolidine-2-carboxylic acid (NH2-4AP(Boc)-OH) and mPEG12-NHS followed the NHS protocol.
Part B-(ii): Synthesis of NHS ester of P12-4AP(Boc)-OH followed the NHS ester protocol.
Part B-(iii): The amide bond formation between 2,5-Diaminoterephthalic acid (H2DATA) and P12-4AP(Boc)-osu followed the NHS protocol.
Part C-(i): Removal of the Boc protection group of (2S,4R)-Fmoc-4-amino-1-Boc-pyrrolidine-2-carboxylic acid (Fmoc-4AP(Boc)-OH) followed the Boc protocol.
Part C-(ii): The amide bond formation between Fmoc-4AP(NH2)-OH and m-PEG12-NHS ester followed the NHS protocol.
Part C-(iii): The amide bond formation between Fmoc-4AP-(P12)—OH and P12-4AP(Boc)-H2DATA followed the HATU protocol.
Part C-(iv): Removal of the Fmoc protection group of P12-4AP(Boc)-H2DATA-Fmoc-4AP(P12) followed the Fmoc protocol.
Part C-(v): The amide bond formation between P12-4AP(Boc)-H2DATA-NH2-4AP(P12) and Ag-P5-osu followed the NHS protocol.
Part C-(vi): Removal of the Boc protection group of P12-4AP(Boc)-H2DATA-(Ag—P5)-4AP(P12) followed the Boc protocol.
Part C-(vii): The amide bond formation between Mal-PEG2-acid (0.8 mg) and P12-4AP(NH2)-H2DATA-(Ag—P5)-4AP(P12) followed the HATU protocol.

ESI-MS m/z calculated for C117H185N12044 [M+H]+: 2462.2; found 2462.2.

### Example 32: Compound 32, i.e., I-PD-LK6 (PD=PEG5-TLR7/8 Agonist 1; HP=2X PEG12-m)

### Part A

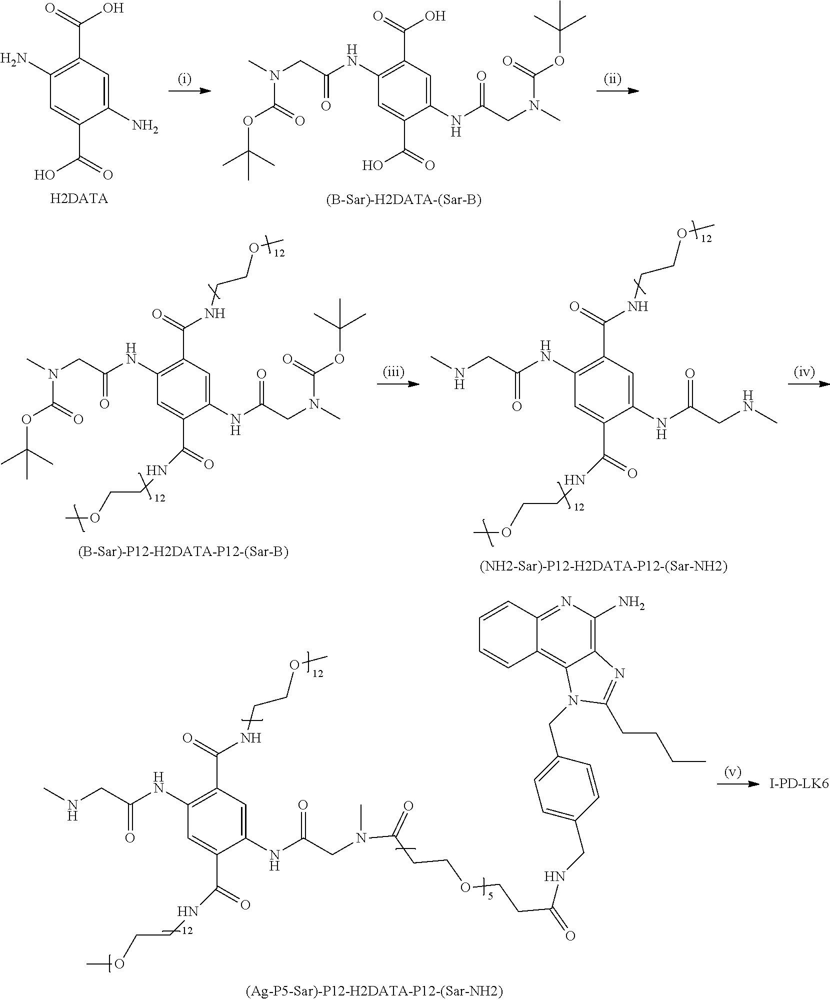

Compound 32 of this invention was prepared following the protocols as indicated below.
Part A-(i): The amide bond formation between 2,5-Diaminoterephthalic acid (H2DATA) and Boc-Sar-osu followed the NHS protocol.
Part A-(ii): The amide bond formation between (B-Sar)-H2DATA-(Sar-B) and mPEG12-amine followed the NHS ester protocol.
Part A-(iii): Removal of the Boc protection group of (B-Sar)-P12-H2DATA-P12-(Sar-B) followed the Boc protocol.
Part A-(iv): The amide bond formation between (NH2-Sar)-P12-H2DATA-P12-(Sar-NH2) and Ag-P5-OH followed the HATU protocol.
Part A-(v): The amide bond formation between (Ag-P5-Sar)-P12-H2DATA-P12-(Sar-NH2) and Mal-PEG2-acid followed the HATU protocol.

ESI-MS m/z calculated for C111H181N12040 [M+H]+: 2322.2; found 2322.2.

### Example 33: Compound 33, i.e., I-PD1-LK7 (PD=Dap-TLR7/8 Agonist 1; HP=PEG24-m)

### Part A

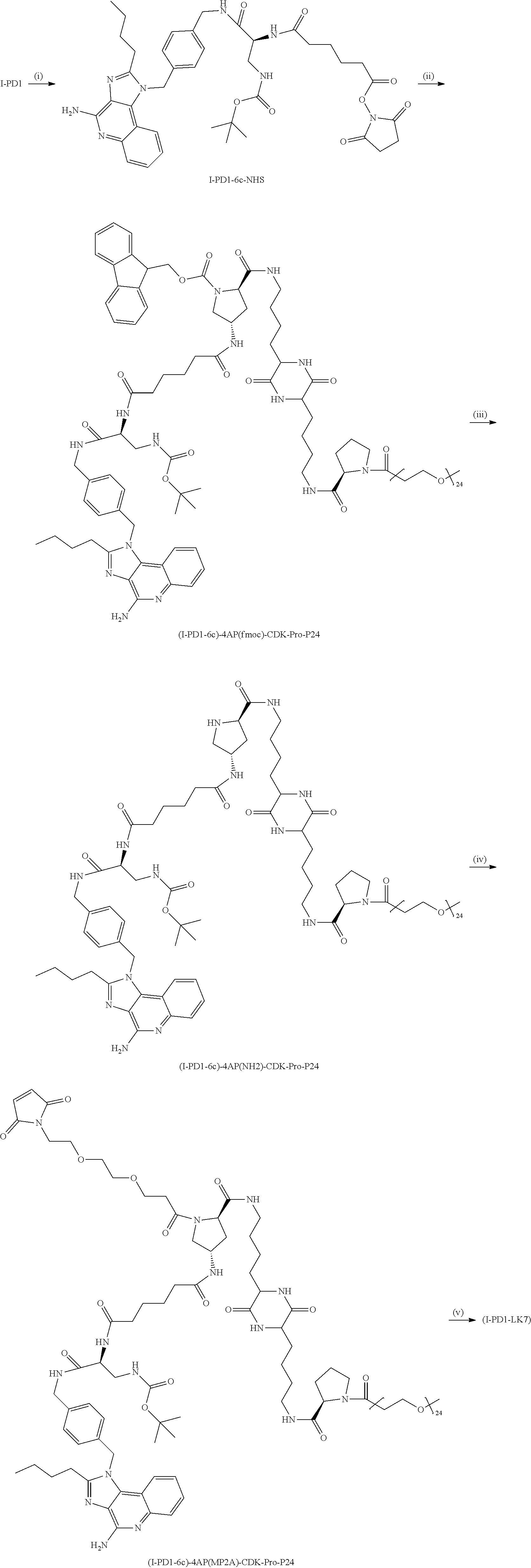

Compound 33 of this invention was prepared following the protocols as indicated below.
Part A-(i): The amide bond formation between I-PD1 and Di(N-succinimidyl) adipate (DSA) followed the NHS protocol.
Part A-(ii): The amide bond formation between NH2-4AP(Fmoc)-CDK-Pro-P24 and I-PD1-6c-NHS followed the NHS protocol.
Part-A-(iii): Removal of the Fmoc protection group of (I-PD1-6c)-4AP(Fmoc)-CDK-Pro-P24 followed the Fmoc protocol.
Part-A-(iv): The amide bond formation between (I-PD1-6c)-4AP(NH2)-CDK-Pro-P24 and Mal-PEG2-acid followed the HATU protocol.
Part A-(v): Removal of the Boc protection group of (I-PD1-6c)-4AP(MP2A)-CDK-Pro-P24 followed the Boc protocol.

ESI-MS m/z calculated for C114H188N15037 [M+H]+: 2359.3; found 2359.3.

### Example 34: Compound 34, I-PD2-LK8 (PD=4AP-TLR7/8 Agonist 1; HP=PEG24-m)

### Part A

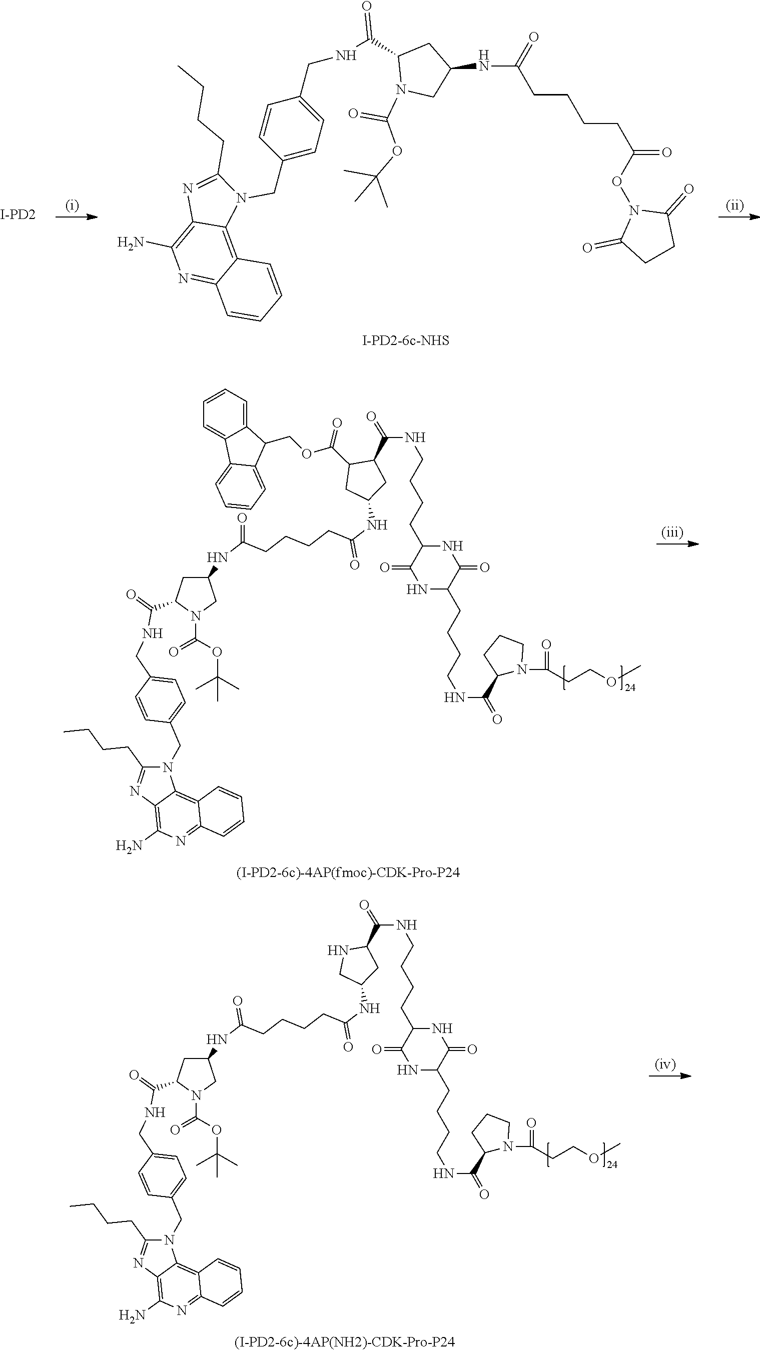

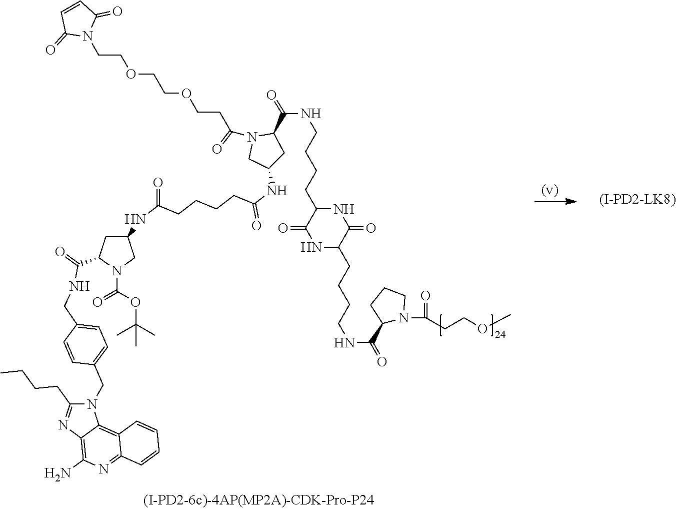

Compound 34 of this invention was prepared following the protocols as indicated below.
Part A-(i): The amide bond formation between I-PD2 (4.8 mg) and Di(N-succinimidyl)adipate (DSA) followed the NHS protocol.
Part A-(ii): The amide bond formation between NH2-4AP(Fmoc)-CDK-Pro-P24 and I-PD2-6I-NHS followed the NHS protocol.
Part-A-(iii): Removal of the Fmoc protection group of (I-PD2-6c)-4AP(Fmoc)-CDK-Pro-P24 followed the Fmoc protocol.
Part-A-(iv): The amide bond formation between (I-PD2-6c)-4AP(NH2)-CDK-Pro-P24 and Mal-PEG2-acid followed the HATU protocol.
Part A-(v): Removal of the Boc protection group of (I-PD2-6c)-4AP(MP2A)-CDK-Pro-P24 followed the Boc protocol.

ESI-MS m/z calculated for C121H198N15039 [M+H]+: 2485.4; found 2485.4.

### Example 35: Compound 35, I-PD2-LK9 (PD=4AP-TLR7/8 Agonist 1; HP=PEG24-m)

### Part A

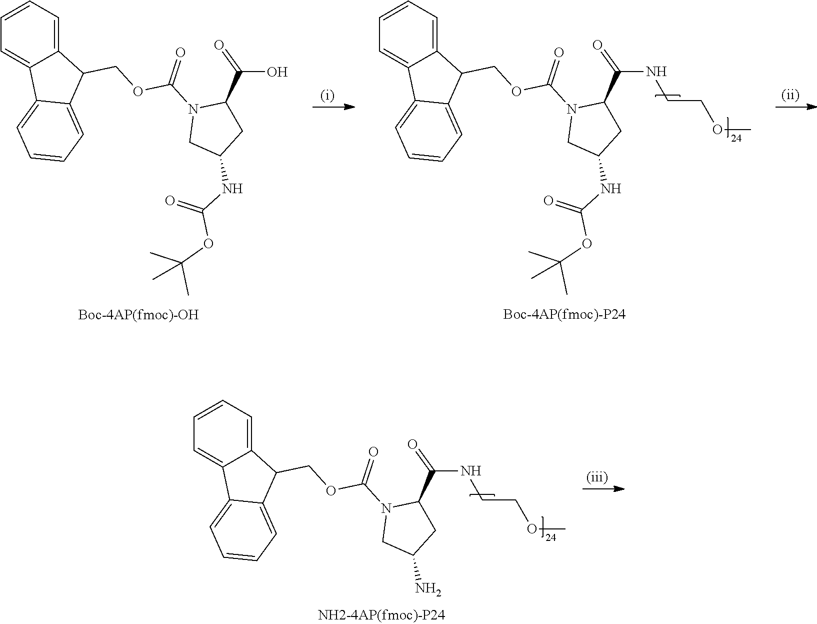

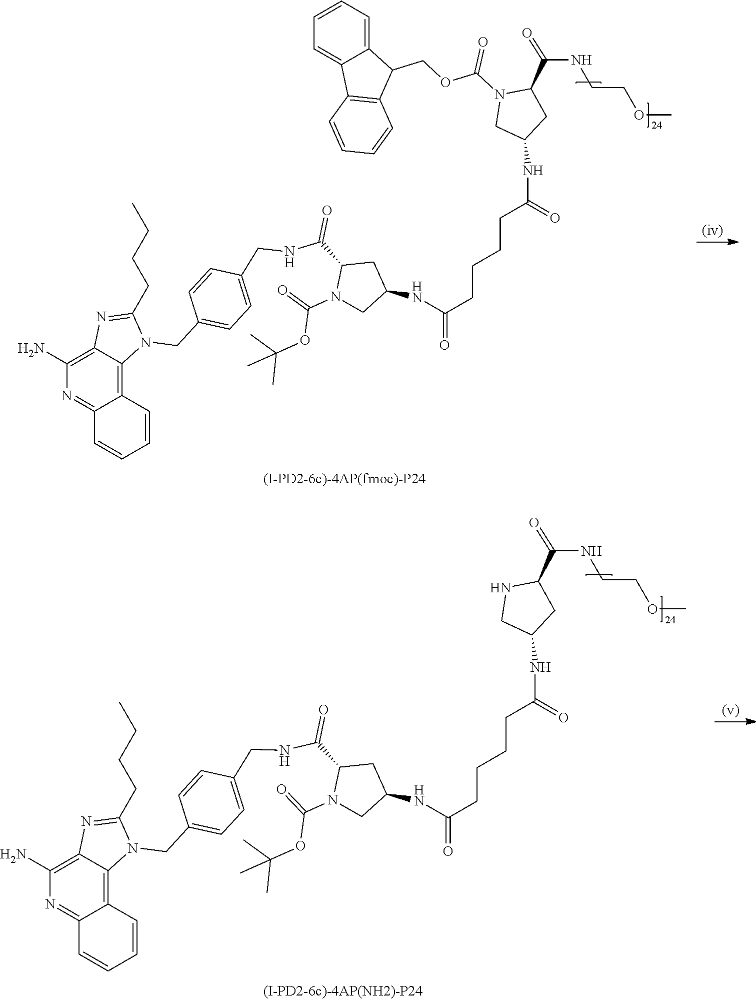

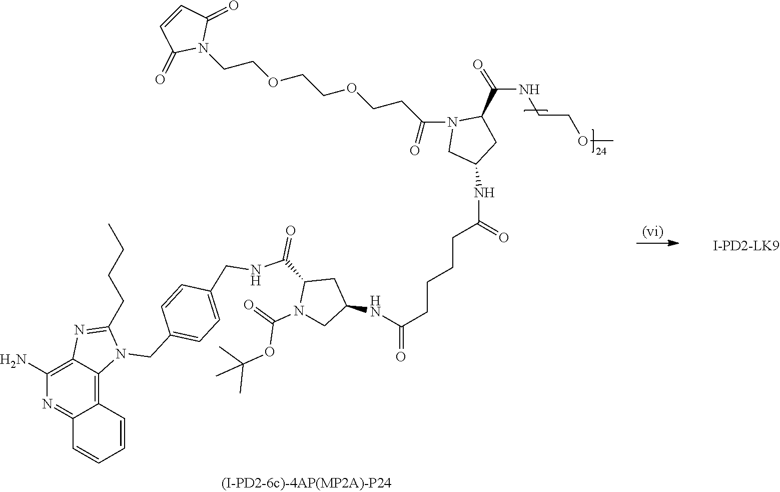

Compound 35 of this invention was prepared following the protocols as indicated below.
Part-A-(i): The amide bond formation between Boc-4AP(Fmoc)-OH and mPEG24-amine followed the HATU protocol.
Part-A-(ii): Removal of the Boc protection group of Boc-4AP(Fmoc)-P24 followed the Boc protocol.
Part-A-(iii): The amide bond formation between NH2-4AP(Fmoc)-P24 and I-PD2-6c-NHS followed the NHS protocol.
Part-A-(iv): Removal of the Fmoc protection group of (I-PD2-6c)-4AP(Fmoc)-P24 followed the Fmoc protocol.
Part-A-(v): The amide bond formation between (I-PD2-6c)-4AP(NH2)—P24 and Mal-PEG2-acid followed the HATU protocol.
Part A-(vi): Removal of the Boc protection group of (I-PD2-6c)-4AP(MP2A)-P24 followed the Boc protocol.

ESI-MS m/z calculated for C98H162N11033 [M+H]+: 2021.1; found 2021.1.

### Example 36: Compound 36, i.e., I-PD2-LK10 (PD=4AP-TLR7/8 Agonist 1; HP=polysarcosine8)

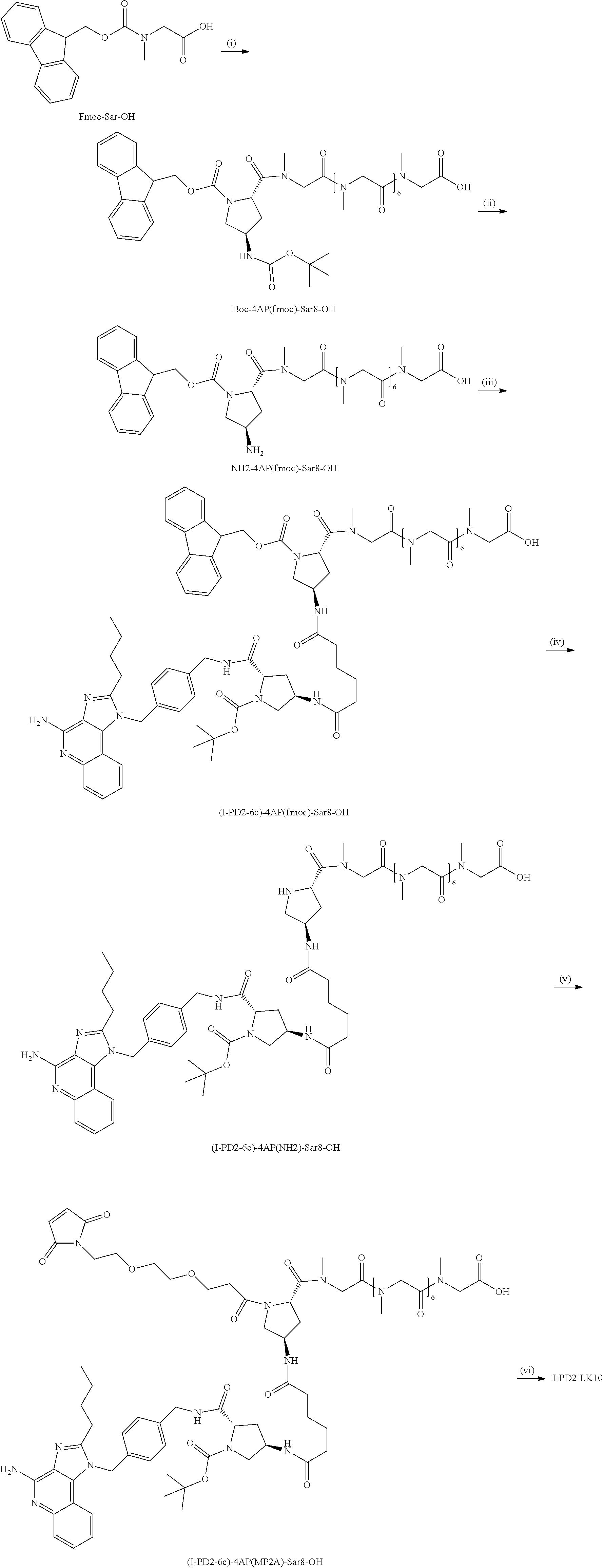

Compound 36 of this invention was prepared following the protocols as indicated below.
(i): The synthesis of boc-4AP (Fmoc)-Sar8-OH followed the SPPS protocol.
(ii): Removal of the Boc protection group of boc-4AP (Fmoc)-Sar8-OH followed the Boc protocol.
(iii): The amide bond formation between NH2-4AP (Fmoc)-Sar8-OH and I-PD2-6c-NHS followed the NHS protocol.
Part-A-(iv): Removal of the Fmoc protection group of (I-PD2-6c)-4AP (Fmoc)-Sar8-OH followed the Fmoc protocol.
Part-A-(v): The amide bond formation between (I-PD2-6c)-4AP (NH2)-Sar8-OH and Mal-PEG2-acid followed the HATU protocol.
Part A-(vi): Removal of the Boc protection group of (I-PD2-6c)-4AP(MP2A)-Sar8-OH followed the Boc protocol.

### Examples 37-64

Exemplary ligand-polar drug conjugates of this invention, i.e., LPDC 1 to LPDC 28, were prepared from a ligand and a polar drug linker (PDL) using one of the protocols described above. See Table 1 below for Examples 37 to 64.

LPDCs 1-18, 20-26 and 28 were prepared using Herceptin as the ligand following the APDC protocol. LPDC 27 was prepared using Perjeta as the ligand following the APDC protocol. LPDC 19 was prepared using albumin as the ligand following the AlbPDC protocol.

TABLE 1

| Example | | | |
|---|---|---|---|
| No. | LPDC | PDL | Product |
| 37 | 1 | Compound 1 | C-APDC1: Herceptin- |
| | | (C-PD2-LK1) | C-PD2-LK1 |
| 38 | 2 | Compound 2 | C-APDC2: Herceptin- |
| | | (C-PD5-LK2) | C-PD5-LK2 |
| 39 | 3 | Compound 5 | C-APDC3: Herceptin- |
| | | (C-PD2-LK5) | C-PD2-LK5 |
| 40 | 4 | Compound 6 | C-APDC4: Herceptin- |
| | | (C-PD5-LK6) | C-PD5-LK6 |
| 41 | 5 | Compound 12 | C-APDC5: Herceptin- |
| | | (C-PD1-LK12) | C-PD1-LK12 |
| 42 | 6 | Compound 14 | C-APDC6: Herceptin- |
| | | (C-PD1-LK14) | C-PD1-LK14 |
| 43 | 7 | Compound 25 | C-APDC7: Herceptin- |
| | | (C-PD6-LK25) | C-PD6-LK25 |
| 44 | 8 | Compound 20 | C-APDC8: Herceptin- |
| | | (C-PD1-LK20) | C-PD1-LK20 |
| 45 | 9 | Compound 7 | C-APDC9: Herceptin- |
| | | (C-PD1-LK7) | C-PD1-LK7 |
| 46 | 10 | Compound 9 | C-APDC10: Herceptin- |
| | | (C-PD1-LK9) | C-PD1-LK9 |
| 47 | 11 | Compound 17 | C-APDC11: Herceptin- |
| | | (C-PD1-LK17) | C-PD1-LK17 |
| 48 | 12 | Compound 18 | C-APDC12: Herceptin- |
| | | (C-PD1-LK18) | C-PD1-LK18 |
| 49 | 13 | Compound 19 | C-APDC13: Herceptin- |
| | | (C-PD1-LK19) | C-PD1-LK19 |
| 50 | 14 | Compound 21 | C-APDC14: Herceptin- |
| | | (C-PD1-LK21) | C-PD1-LK21 |
| 51 | 15 | Compound 22 | C-APDC15: Herceptin- |
| | | (C-PD1-LK22) | C-PD1-LK22 |
| 52 | 16 | Compound 24 | C-APDC16: Herceptin- |
| | | (C-PD1-LK24) | C-PD1-LK24 |
| 53 | 17 | Compound 15 | C-APDC17: Herceptin- |
| | | (C-PD2-LK15) | C-PD2-LK15 |
| 54 | 18 | Compound 26 | C-APDC18: Herceptin- |
| | | (C-PD4-LK26) | C-PD4-LK26 |
| 55 | 19 | Compound 15 | C-AlbPDC1: Human Serum |
| | | (C-PD2-LK15) | Albumin-C-PD2-LK15 |
| 56 | 20 | Compound 28 | I-APDC2: Herceptin- |
| | | (I-PD2-LK2) | I-PD2-LK2 |
| 57 | 21 | Compound 29 | I-APDC3: Herceptin- |
| | | (I-PD2-LK3) | I-PD2-LK3 |
| 58 | 22 | Compound 30 | I-APDC4: Herceptin- |
| | | (I-PD2-LK4) | I-PD2-LK4 |
| 59 | 23 | Compound 31 | I-APDC5: Herceptin- |
| | | (I-PD-LK5) | I-PD-LK5 |
| 60 | 24 | Compound 32 | I-APDC6: Herceptin- |
| | | (I-PD-LK6) | I-PD-LK6 |
| 61 | 25 | Compound 33 | I-APDC7: Herceptin- |
| | | (I-PD1-LK7) | I-PD1-LK7 |
| 62 | 26 | Compound 34 | I-APDC8: Herceptin- |
| | | (I-PD2-LK8) | I-PD2-LK8 |
| 63 | 27 | Compound 34 | I-APDC9: Perjeta- |
| | | (I-PD2-LK8) | I-PD2-LK8 |
| 64 | 28 | Compound 35 | I-APDC10: Herceptin- |
| | | (I-PD2-LK9) | I-PD2-LK9 |

Conjugates 1-28 of this invention were evaluated using the five assays (SEC analysis, in vitro cytotoxicity assays, toxicity study, pharmacokinetics study and xenograft tumor model experiments) as described below.

### SEC Analysis of C-APDCs

C-APDCs were diluted to 2.5 mg/mL in a buffer containing 6% sucrose, 10 mM sodium succinate dibasic hexahydrate and 0.02% Tween 20 at pH 5. Samples (24 μL) were analyzed using an Agilent HPLC system having an analytical SEC column (Agilent AdvanceBio® SEC 300 Å 2.7 μm), eluted isocratically with a phosphate-buffered saline solution (PBS) at a flow rate of 0.5 mL/min.

### SEC Analysis of I-APDCs

I-APDCs were diluted to 0.2 mg/mL in PBS. Samples (500 μL) were injected to an analytical SEC column (Biorad® SEC 650) on an AKTA PURE FPLC system, eluted isocratically with PBS at a flow rate of 1 mL/min.

### In Vitro Cytotoxicity Assays of APDCs

In vitro cytotoxicity assays were performed as previously described (Boyd and Paull, 1995; Phillips et al., 2008). GI50 was calculated as [(Ti−Tz)/(C−Tz)]×100=50, in which “Tz” represents the cell population at DO; “Ti” represents the cell populations of different ADC concentration groups at D3; and “C” represents the cell population of vehicle group at D3. IC50, the half maximal inhibitory concentration, was calculated from dose-response curves which was generated by four-parameter curve fitting.

### Toxicity Study of T-DM1 and LPDC

The toxicity of T-DM1 and an LPDC of this invention was evaluated by a single-dose injection of T-DM1 or the LPDC into mice intravenously on day 0 with vehicle alone, or T-DM1/LPDC (at 60, 70, 80, and 90 mg/kg, n=3 animals/group). Body weights were recorded on day 0, 1, 2, 3, 6 and 7.

### Pharmacokinetics Study of T-DM1 and APDC

Pharmacokinetics study was performed, using antibodies against trastuzumab and mertansine, respectively, as previously described (Dere et al., 2013).

### Xenograft Tumor Model Experiments for Cytotoxic APDCs

Experiments of xenograft tumor model with cytotoxic APDCs were performed as previously described (Jumbe et al., 2010).

### Xenograft Tumor Model Experiments for Immuno-Stimulating APDCs

Experiments of xenograft tumor model with immuno-stimulating APDCs were performed as previously described (Ackerman et al., 2021).

The results of the above five assays showed that, unexpectedly:
1. LPDCs of this invention with a DAR value around 7-9 all exhibited little aggregation. Size exclusion chromatography (SEC) was performed for nine conjugates of this invention, namely, C-APDC17, I-APDC2, I-APDC3, I-APDC4, I-APDC6, I-APDC7, I-APDC8, I-APDC9, and I-APDC10. Each conjugate, having a DAR value around 7-9, was eluted as a single peak with a molecular weight about 150 KD, indicating little aggregation of the conjugate.
2. Cell-based assays demonstrated that cytotoxic of APDC17 was more efficacious than Kadcyla and Enhertu in inhibiting cancer cell lines with low to high levels of HER2 expression and also more resistant to MDR1-mediated efflux. See the four tables below.
3.

TABLE 2

| | | IC₅₀ (nM) | | |
|---|---|---|---|---|
| | | Kadcyla | Enhertu | C-APDC17 |
| HER2+++ | SK-BR-3 | 0.060 | 0.23 | 0.017 |
| | NCI-N87 | 0.15 | 0.97 | 0.082 |
| HER2++ | JIMT-1 | 14 | >1000 | 0.93 |
| | Capan-1 | 86 | >1000 | 55 |
| HER2+ | MKN45 | 52 | 371 | 32 |
| | MDA-MB-231 | 62 | >1000 | 35 |

TABLE 3

| | | GI₅₀ (nM) | | |
|---|---|---|---|---|
| | | Kadcyla | Enhertu | C-APDC17 |
| HER2+++ | SK-BR-3 | 0.052 | 0.3 | 0.014 |
| | NCI-N87 | 0.24 | 12 | 0.16 |
| HER2++ | JIMT-1 | 15 | >1000 | 1.4 |
| | Capan-1 | 64 | 56 | 42 |
| HER2+ | MKN45 | 107 | 1041 | 76 |
| | MDA-MB-231 | 156 | >1000 | 72 |

TABLE 4

| | IC₅₀ (nM) | | |
|---|---|---|---|
| | Kadcyla | Enhertu | C-APDC17 |
| NCI-N87 | 0.38 | 3.1 | 0.47 |
| NCI-N87-hMDR1 | 2.5 | 36 | 1.9 |
| NCI-N87/NCI-N87-hMDR1 (folds) | 6.7 | 12 | 4.1 |

TABLE 5

| | GI₅₀ (nM) | | |
|---|---|---|---|
| | Kadcyla | Enhertu | C-APDC17 |
| NCI-N87 | 0.53 | 49 | 0.51 |
| NCI-N87-hMDR1 | 6.7 | >1000 | 4.6 |
| NCI-N87/NCI-N87-hMDR1 (folds) | 13 | >20 | 9 |

In Table 2 above, conjugate C-APDC17 of this invention has an IC₅₀ much lower than Kadcyla and Enhertu in all six cell lines, i.e., SK-BR-3, NCI-N87, JIMT-1, Capan-1, MKN45, MDA-MB-231, indicating that it is more effective in inhibiting these cells. Further Table 3 confirms that conjugate C-APDC17 is more effective in inhibiting the growth of these cancer cells as it has a lower GI50 than Kadcyla and Enhertu in the six cell lines.

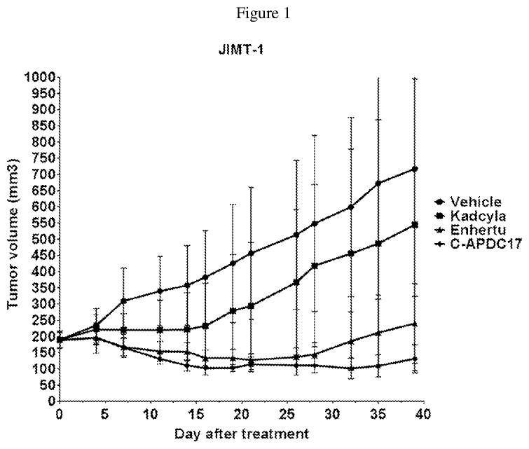

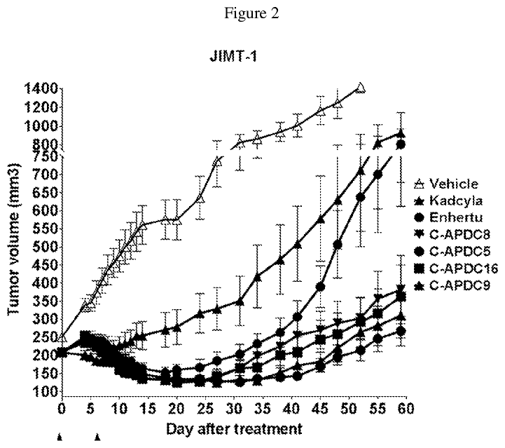

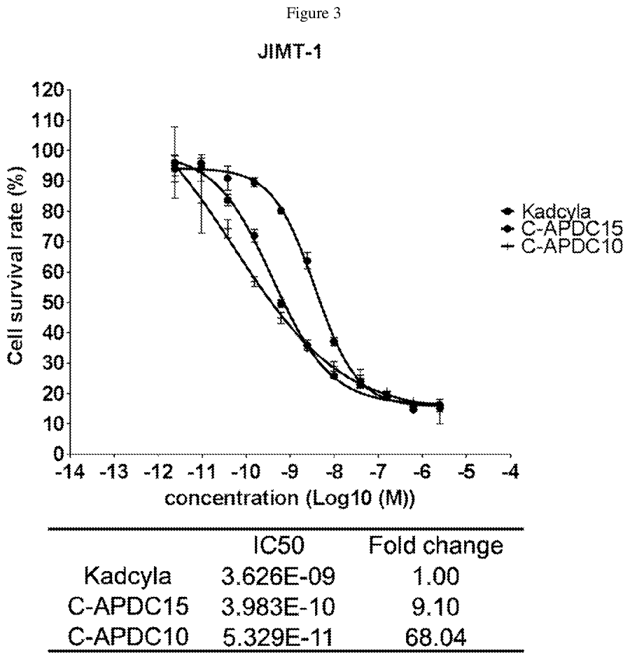

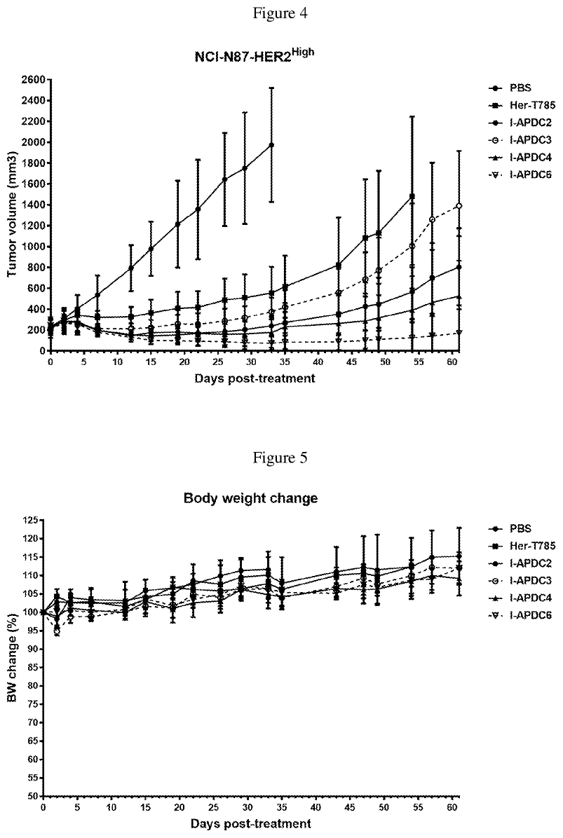

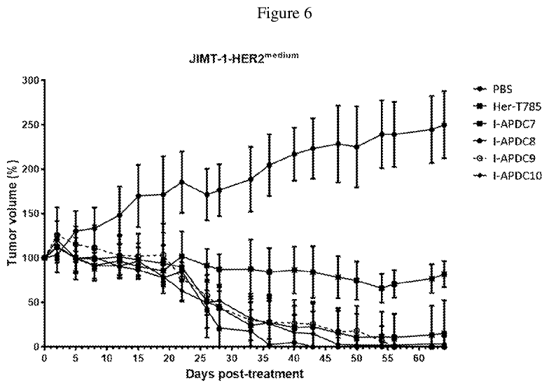

Both Tables 4 and 5 show that conjugate C-APDC17 is not only more effective than Kadcyla and Enhertu in inhibiting normal NCI-N87 cell line but also more resistant to MDR1 protein as evidenced by the study with
an MDR1-overexpressing cell line, NCI-N87-hMDR1.
4. Conjugate APDC17 was more efficacious than commercially available ADCs, such as Kadcyla and Enhertu, in a JIMT-1 xenograft model for inhibiting tumor growth (See FIG. 1). A single intravenous injection (10 mg/kg at day zero) of APDC17, Kadcyla, or Enhertu was made in the JIMT-1 xenograft model. Tumor volume, calculated as length×width²/2, was plotted as a function of time in FIG. 3. Surprisingly, the tumor volume decreased in the group treated with APDC17 while both the Kadcyla and Enhertu-treated groups showed significant tumor growth.
5. Pharmacokinetics studies also demonstrated that C-APDC17 performed better than Kadcyla in SD rats, following IV administration of C-APDC17 or Kadcyla at all three tested dosages, i.e., 1 mg/kg, 3 mg/kg, and 10 mg/kg.
6. Maximum Tolerated Dose (MTD) of C-APDC17 in BALB/c mice following IV injection was approximately 90 mg/kg, indicating that the conjugate of this invention was well tolerated. Tolerability studies on C-APDC5, C-APDC8, C-APDC9, and C-APDC16 of this invention indicated that their tolerability is similar to that of Kadcyla.
7. A single dose of cytotoxic conjugates of this invention (C-APDC5, C-APDC8, C-APDC9, and C-APDC16) outperformed two doses of marketed ADCs, Kadcyla and Enhertu in a JIMT-1 xenograft model (See FIG. 2).
8. A dual (HER2 and nucleolin)-targeting conjugate of this invention, namely C-APDC10, was much more efficacious than a monotargeting ADC, Kadcyla, or a monotargeting APDC, C-APDC15, in killing cancer cells (See FIG. 3).
9. Immune-stimulating LPDCs of this invention (i.e., I-APDC2, I-APDC3, I-APDC4, I-APDC6, I-APDC7, I-APDC-8, I-APDC9, and I-APDC10) performed significantly better than Her-T785, a Bolt's immune-stimulating ADC (See FIGS. 4-6).

### OTHER EMBODIMENTS

All of the features disclosed in this specification may be combined in any combination. Each feature disclosed herein may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present invention, and without departing from the spirit and scope thereof, can make various changes and modifications of the invention to adapt it to various usages and conditions. For example, compounds structurally analogous to the conjugates of this invention also can be made, screened for their efficacy in treating cancer.

### REFERENCES

Ackerman et al., 2021. Immune-stimulating antibody conjugates elicit robust myeloid activation and durable antitumor immunity. Nature Cancer 2, 18-33.
Akbarzadehlaleh et al., 2016. PEGylated Human Serum Albumin: Review of PEGylation, Purification and Characterization Methods. Adv Pharm Bull. 6(3): 309-317.
Bargh et al., 2019. Cleavable linkers in antibody-drug conjugates. Chem Soc Rev DOI: 10.1039/c8cs00676h, accessible via the following link: https://pubs.rsc.org/en/content/articlelanding/2019/cs/c8cs00676h.
Boyd and Paull, 1995. Some Practical Considerations and Applications of the National Cancer Institute In Vitro Anticancer Drug Discovery Screen. Drug Development Research 34: 91-109.
Calvaresi and Hergenrother, 2013. Glucose conjugation for the specific targeting and treatment of cancer. Chem Sci 4(6): 2319-2333.
Catalano et al., 2022. Multidrug Resistance (MDR): A Widespread Phenomenon in Pharmacological Therapies. Molecules 27: 616.
Chan et al., 2017. Polypeptoid polymers: Synthesis, characterization and properties. Biopolymers e23070.
Chapman 2002. PEGylated antibodies and antibody fragments for improved therapy: a review. Advanced Drug Delivery Reviews 54 (2002) 531-545.
Czajkowsky et al., 2012. Fc-fusion proteins: new developments and future perspectives. EMBO Mol Med 4, 1015-1028.
Dere et al., 2013. PK assays for antibody-drug conjugates: case study with ado-trastuzumab emtansine. Bioanalysis 5(9), 1025-1040.
Dharmatti et al., 2019. Enhancement of Binding Affinity of Folate to Its Receptor by Peptide Conjugation. Int J Mol Sci 20(9):2152.
Diamantis and Banerji, 2016. Antibody-drug conjugates—An emerging class of cancer treatment. Br. J. Cancer 114, 362-367.
Fishburn 2008. The pharmacology of PEGylation: balancing PD with PK to generate novel therapeutics. J Pharm Sci 97(10):4167-83.
Gala et al., 2020. Harnessing the therapeutic potential of anticancer drugs through amorphous solid dispersions. Biochim Biophys Acta Rev Cancer: 1873(1):188319.
Gavriel et al., 2022. Recent advances in self-immolative linkers and their applications in polymeric reporting systems. Polym. Chem., 13, 3188-3269.
Goldberg et al., 2016. Engineering a targeted delivery platform using Centyrins. Protein Eng Des Sel. 29: 563-72.
Greenwald et al., 2003. Effective drug delivery by PEGylated drug conjugates. Adv Drug Deliv Rev 55: 217-250.
Guha and Padh 2008. Cathepsins: Fundamental Effectors of Endolysosomal Proteolysis. Indian J Biochemistry & Biophysics 45, 75-90.
Gupta et al., 2019. Protein PEGylation for cancer therapy: bench to bedside. J. Cell Commun. Signal. 13: 319-330.
Hou et al., 2019. Therapeutic Protein PEPylation: The Helix of Nonfouling Synthetic Polypeptides Minimizes Antidrug Antibody Generation. ACS Cent. Sci. 5, 2, 229-236.
Hu et al., 2018. Polysarcosine as an Alternative to PEG for Therapeutic Protein Conjugation. Bioconjugate Chem. 2018, 29, 7, 2232-2238.
Jafari et al., 2017. Fc-fusion Proteins in Therapy: An Updated View. Curr Med Chem 24:1228-1237
Jiang et al., 2015. Progress and Challenges in Developing Aptamer-Functionalized Targeted Drug Delivery Systems. Int. J. Mol. Sci. 16: 23784-23822.
Jumbe et al., 2010. Modeling the efficacy of trastuzumab-DM1, an antibody drug conjugate, in mice. J Pharmacokinet Pharmacodyn 37:221-242.
Kim et al., 2020. A novel nucleolin-binding peptide for Cancer Theranostics. Theranostics. 2020; 10(20): 9153-9171.
Kuai et al., 2017. Circular Bivalent Aptamers Enable in Vivo Stability and Recognition. J. Am. Chem. Soc. 139, 27, 9128-9131.
Lam et al., 2016. Synthesis and evaluation of an 18F-labeled derivative of F3 for targeting surface-expressed nucleolin in cancer and tumor endothelial cells. J Label Compd Radiopharm 59: 492-499.
Leal et al., 2015. Preclinical Development of an anti-5T4 Antibody-Drug Conjugate: Pharmacokinetics in Mice, Rats, and NHP and Tumor/Tissue Distribution in Mice. Bioconjug Chem 2015, 26: 2223-32.
Libutti et al., 2018. Targeting the invincible barrier for drug delivery in solid cancers: interstitial fluid pressure. Oncotarget. 2018 Nov. 6; 9(87): 35723-35725.
Lin et al., 2015. Biocompatible long-circulating star carboxybetaine polymers. J Mater Chem B 3, 440-448.
Lu et al., 2014. Population pharmacokinetics of trastuzumab emtansine (T-DM1), a HER2-targeted antibody-drug conjugate, in patients with HER2-positive metastatic breast cancer: clinical implications of the effect of covariates. Cancer Chemother Pharmacol 74:399-410.
Lyon et al., 2015. Reducing hydrophobicity of homogeneous antibody drug conjugates improves pharma-cokinetics and therapeutic index. Nature Biotechnology 33: 733-736.
McDonald and Baluk. 2002. Significance of Blood Vessel Leakiness in Cancer. Cancer Res 62 (18): 5381-5385.
Mckertish and Kayser, 2021. Advances and limitations of antibody drug conjugates for cancer. Biomedicines 9, 872.
Nagy et al., 2009. Why are tumour blood vessels abnormal and why is it important to know? British Journal of Cancer 100, 865-869.
Oroudjev et al., 2010. Maytansinoid-Antibody Conjugates Induce Mitotic Arrest by Suppressing Microtubule Dynamic Instability. Mol Cancer Ther; 9: 2700-2713.
Pan et al., 2021. Recent advance of peptide-based molecules and nonpeptidic small-molecules modulating PD-1/PD-L1 protein-protein interaction or targeting PD-L1 protein degradation. European Journal of Medicinal Chemistry 213, 113170.
Phillips et al., 2008. Targeting HER2-positive breast cancer with trastuzumab-DM1, an antibody-cytotoxic drug conjugate. Cancer Res. 68: 9280-90.
Podust et al., 2016. Extension of in vivo half-life of biologically active molecules by XTEN protein polymers. J Control Release 240, 52-66.
Rathi et al., 2021. Population pharmacokinetics of belantamab mafodotin, a BCMA-targeting agent in patients with relapsed/refractory multiple myeloma. CPT Pharmacometrics Syst Pharmacol. 10:851-863.
Rogers et al., 2015. Recombinant human serum albumin fusion proteins and novel applications in drug delivery and therapy. Curr Pharm Des 21(14):1899-907.
Saw et al., 2021. Biomedical Applications of a Novel Class of High-Affinity Peptides. Acc Chem Res 54:3576-3592.
Schlapschy et al., 2013. PASylation: a biological alternative to PEGylation for extending the plasma half-life of pharmaceutically active proteins. Protein Eng Des Sel. 26: 489-501.
Schneider et al., 2020. Recent progress in transglutaminase-mediated assembly of antibody-drug conjugates. Analytical Biochemistry 595, 113615.
Sheyi et al., 2022. Linkers: An Assurance for Controlled Delivery of Antibody-Drug Conjugate. Pharmaceutics, 14(2), 396.
Su et al., 2021. Antibody-drug conjugates: Recent advances in linker chemistry. Acta Pharmaceutica Sinica B 11: 3889-3907.
Sugahara et al., 2009. Tissue-Penetrating Delivery of Compounds and Nanoparticles into Tumors. Cancer Cell 16, 510-520.
Tian et al., 2021. Hydroxyproline-derived biomimetic and biodegradable polymers. Current Opinion in Solid State & Materials Science 25: 100902.
Viricel et al., 2019. Monodisperse polysarcosine-based highly-loaded antibody-drug conjugates. Chem. Sci. 10: 4048.
Waghray and Zhang 2018. Inhibit or Evade Multidrug Resistance P-glycoprotein in Cancer Treatment. J Med Chem. 61: 5108-5121.
Wang et al., 2020b. Bioengineered Human Serum Albumin Fusion Protein as Target/Enzyme/pH Three-Stage Propulsive Drug Vehicle for Tumor Therapy. ACS Nano, 14, 12, 17405-17418.
Wang et al., 2022. Therapeutic peptides: current applications and future directions. Signal Transduction and Targeted Therapy 7:48.
Wu et al., 2010. Blastocystis Legumain Is Localized on the Cell Surface, and Specific Inhibition of Its Activity Implicates a Pro-survival Role for the Enzyme. J Biol Chem. 285(3): 1790-1798.
Zhloba and Dunaevskiǐ, 1996. Cysteine cathepsins secreted into the blood as markers of tumor growth. Vopr Onkol 42(1): 70-6.
Zhou et al., 2008. Substrates and inhibitors of human multidrug resistance associated proteins and the implications in drug development. Curr Med Chem 15: 1981-2039.
Zhu et al., 2018. Nucleolin mediates the internalization of rabbit hemorrhagic disease virus through clathrin-dependent endocytosis. PLoS Pathog 14(10): e1007383.

We claim:

1. A compound of formula I: 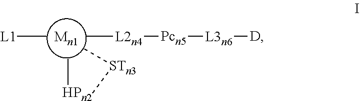
in which
the compound comprises a ligand linker L1, an Mₐ₁ linker complex, a drug linker L2, a first polar drug moiety PD1 represented by Pcₙ₅-L3ₙ₆-D, optionally one to five hydrophilic polymer moieties HP, and optionally one to five secondary targeting moieties ST;
n1 is an integer from 1 to 15;
n2 is an integer from 0 to 5;
n3 is an integer from 0 to 5;
n4 is 1 or 2;
n5 is 0 or 1;
n6 is 0 or 1;
one of ---- is a covalent bond and the other ---- is deleted;
each of L1, L2, and HP is bonded to the Mₙ₁ linker complex via a linker bond;
ST, when present, is bonded to HP or the Mₐ₁ linker complex via a linker bond,
L2 is bonded to PD via an amide bond or a glycoside bond;
L3, when present, is bonded to D via a stable bond;
Pc, when present, is bonded to L3 or D via a stable bond;
each of the monomer M within the Mₐ₁ linker complex is bonded to its adjacent monomer M via a stable bond;
D is a drug moiety;
M in each occurrence is independently a multifunctional moiety;
L1 is a bifunctional crosslinker containing a coupling moiety capable of reacting with a ligand via sulfhydryl, amino, glutamine, or formyl contained in the ligand;
L2 is a bifunctional crosslinker;
L3 is a bifunctional crosslinker;
HP in each occurrence, independently, is a hydrophilic polymer moiety;
Pc contains a polar group;
ST in each occurrence, independently, is a secondary targeting moiety;
a linker bond is an amide bond, a glycoside bond, an ester bond, a disulfide bond, a C—S bond, a C—O bond, a C—N bond, a carbonate moiety (—O—C(O)—O—), a carbamate moiety (—O—C(O)—NH—), a urea moiety (—NH—C(O)—NH—), or a triazole moiety 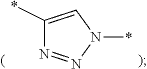 [SMILES: *c1cn(*)nn1]
and
a stable bond is a stable amide bond, a stable glycoside bond, a stable ester bond, a stable disulfide bond, a C—S bond, a C—O bond, a C—N bond, a urea moiety (—NH—C(O)—NH—), or a triazole moiety

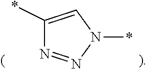 [SMILES: *c1cn(*)nn1]

2. The compound of claim 1, wherein
L1 contains amino (NH2), iodo (I), bromo (Br), a maleimide moiety, an N-hydroxysuccinimide (NHS) moiety, or an aminooxy moiety;
M in each occurrence is a monomeric unit derived from a natural amino acid, a non-natural amino acid, a monosaccharide, a bifunctional crosslinker, a polyamine having at least one secondary or tertiary amine group, 3,6-bis-(4-amino-butyl)-piperazine-2,5-dione, a cyclic peptide, or a diamino dicarboxylic acid;
Pc is a moiety derived from a monosaccharide, a polyamine, a hydroxycarboxylic acid, a linear or cyclic dipeptide, a diamino dicarboxylic acid, a natural amino acid, or a non-natural amino acid; and
ST in each occurrence, independently, is a moiety derived from glucose, folic acid, iRGD peptide, a cancer-specific peptide ligand (AGM-330), a human fibronectin extra-domain B (EDB)-specific aptide (“APTEDB”), a centyrin, an F3 peptide, a DVN peptide, pasireotide, afamelanotide, etelcalcetide, somatostatin, a PD-1/PD-L1 interaction inhibitor (“BMS-1166”), a cyclic peptide, a single-chain antibody, or an aptamer.

3. The compound of claim 1, wherein the compound comprises a second polar drug moiety PD2 represented by Pc′_{n5′}-(L₃′)ₙ₆-D′, which is bonded to the Mₙ₁ linker complex via an amide bond or a glycoside bond; n5′ is 0 or 1; n6′ is 0 or 1; D′ is a drug moiety; L3′ when present is a bifunctional crosslinker; and Pc′ when present contains a polar group.

4. The compound of claim 1, wherein M in each occurrence, independently, is a moiety derived from a diamino dicarboxylic acid, a monosaccharide, 3,6-bis-(4-amino-butyl)-piperazine-2,5-dione, a cyclic peptide, an amino acid, a dicarboxylic acid, a mercapto carboxylic acid, or an aminothiol.

5. The compound of claim 1, wherein the Mₙ₁ linker complex contains a monomeric unit derived from a diamino dicarboxylic acid, 3,6-Bis-(4-amino-butyl)-piperazine-2,5-dione, or a cyclic peptide, and the compound optionally contains, between L2 and PD, a releasable moiety having a self-immolative spacer, an enzymatically cleavable amino acid, an enzymatically cleavable peptide of 2-8 amino acids, a combination of the self-immolative spacer and the enzymatically cleavable amino acid, or a combination of the self-immolative spacer and the enzymatically cleavable peptide.

6. The compound of claim 1, wherein the Mₙ₁ linker complex contains a moiety selected from the group consisting of:

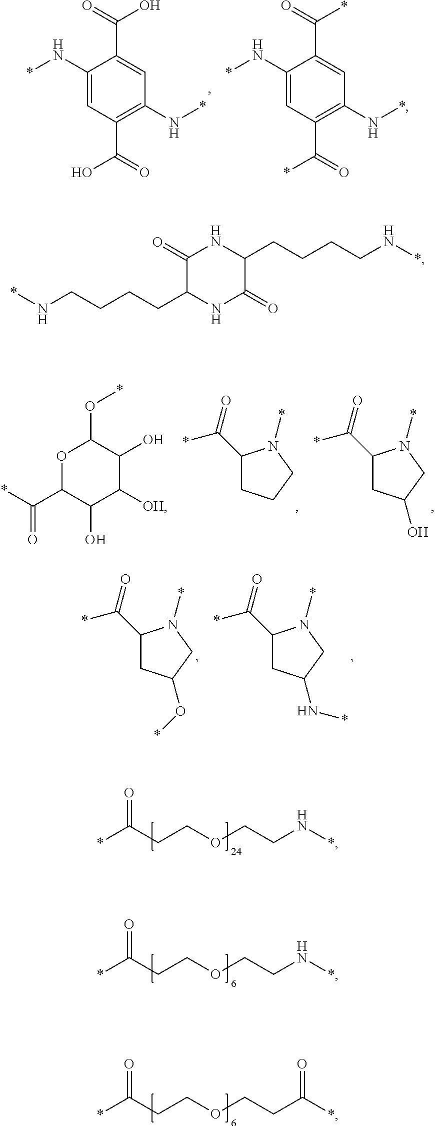

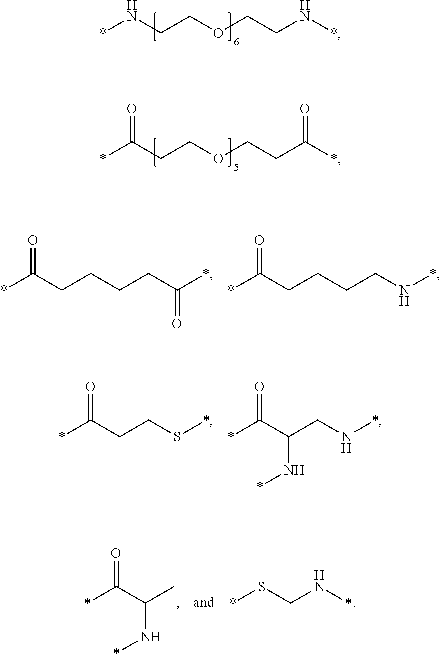

7. The compound of claim 6, wherein the M linker complex contains a moiety selected from the group consisting of:

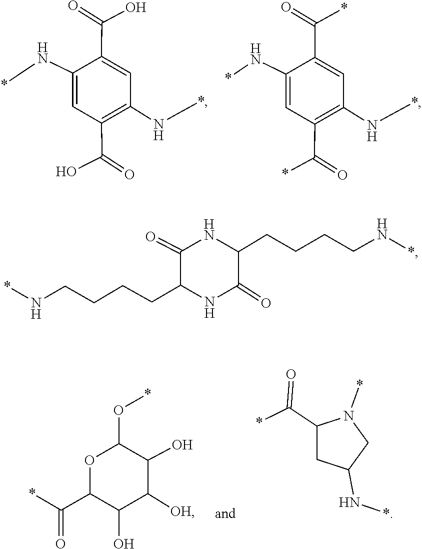

8. The compound of claim 7, wherein the Mₙ₁ linker complex contains:

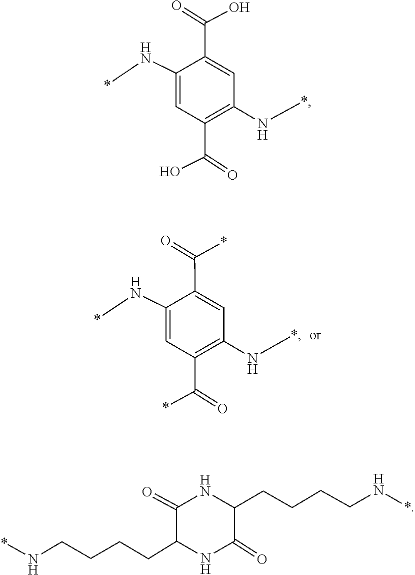

9. The compound of claim 1, wherein L1 is 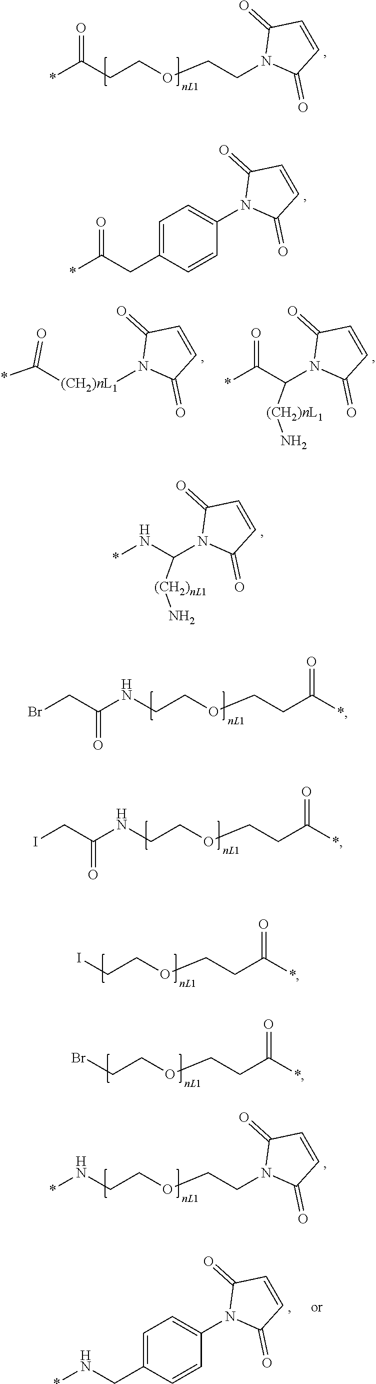 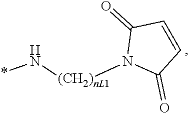
in which nL1 is an integer from 0 to 10.

10. The compound of claim 1, wherein L2 is 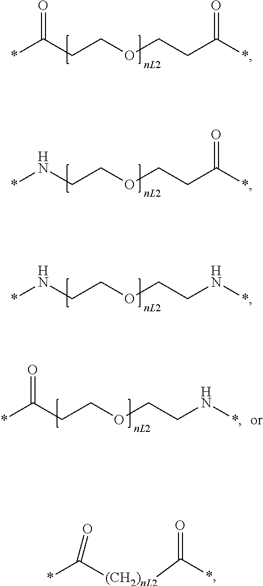
in which nL2 is an integer from 0 to 10.

11. The compound of claim 1, wherein Pc is a moiety derived from an amino acid, a linear or cyclic dipeptide, a monosaccharide, a bifunctional polyethylene glycol, or an aminophenol.

12. The compound of claim 11, wherein Pc is 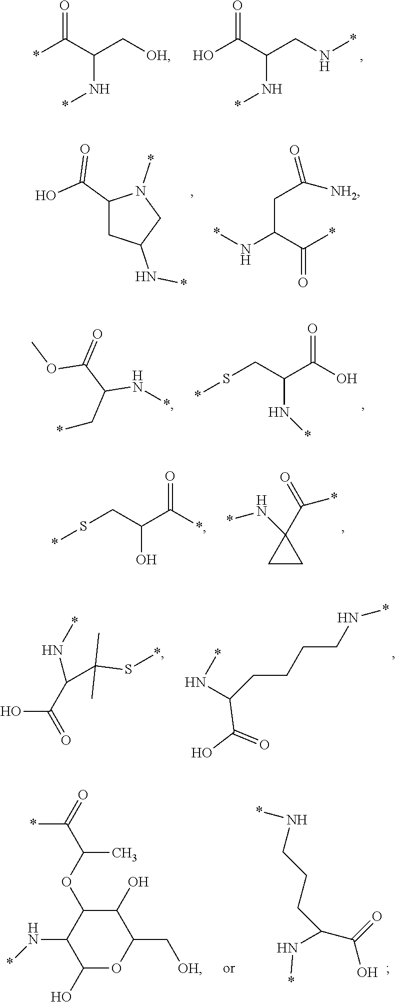
L3 is 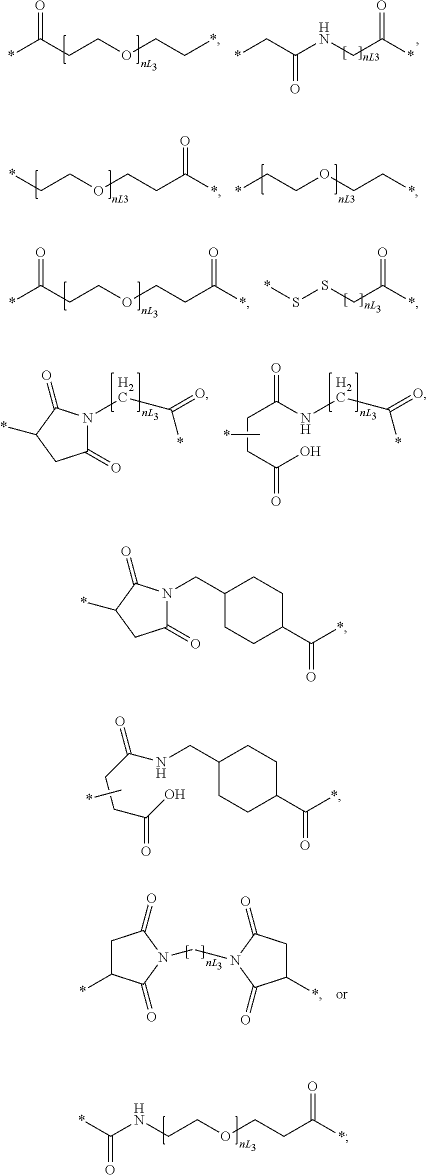
and
nL3 is 0 to 10.

13. The compound of claim 1, wherein HP in each occurrence, independently, is 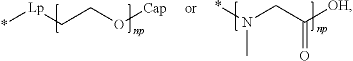
in which np is an integer from 5 to 50; Cap is (i) a reactive group capable of reacting with a ST molecule to bond the ST moiety to the HP moiety or (ii) a terminal group selected from the group consisting of C₁-C₁₀ alkyl, C₂-C₁₀ alkyl-CO₂H, C₂-C₁₀ alkyl-OH, C₂-C₁₀ alkyl-NH2, C₂-C₁₀ alkyl-NH(C₁-C₃ alkyl), and C₂-C₁₀ alkyl-N(C₁-C₃ alkyl)₂; and Lp is a linker moiety.

14. The compound of claim 13, wherein HP in each occurrence, independently, is

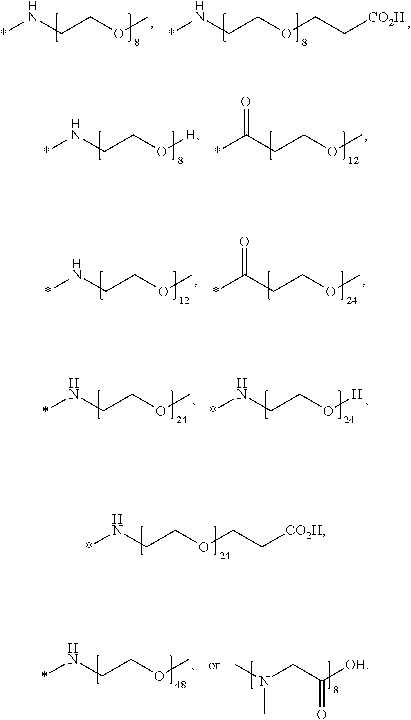

15. The compound of claim 1, wherein D is a drug moiety derived from mertansine (DM1), ravtansine (DM4), N-methyl-L-Ala-maytansinol, monomethyl auristatin E, 7-ethyl-10-hydroxycamptothecin (SN38), or TLR7/8 agonist Ag.

16. The compound of claim 1, wherein the secondary targeting moiety is derived from folic acid, glucose, or acetyl-RHGAMVYLK.

17. The compound of claim 1, wherein the compound is one of Compounds 1-36.

18. A ligand-PD conjugate comprising a ligand moiety and a moiety derived from a compound of claim 1, wherein the ligand is bonded to the compound via a covalent bond formed between a functional group from the ligand and L1 in formula I, and the functional group is sulfhydryl, amino, glutamine, or formyl.

19. The ligand-PD conjugate of claim 17, wherein the ligand is trastuzumab, albumin, or pertuzumab, and the covalent bond is formed between sulfhydryl of the ligand and the maleimide, iodo, or bromo moiety of L1.

20. The ligand-PD conjugate of claim 18, wherein the molar ratio of the ligand to the compound is between 1:1 and 1:20, preferably between 1:2 and 1:8.

21. The ligand-PD conjugate of claim 18, wherein the ligand-PD conjugate is one of Conjugates 1-28.

22. A method of treating cancer comprising administrating to a patient in need thereof an effective amount of a conjugate of claim 18.

23. A pharmaceutical composition comprising a conjugate of claim 18, and a pharmaceutically acceptable carrier, diluent, or excipient.

24. A conjugate of claim 18 for the manufacture of a medicament for treating cancer.

25. A method of preparing a conjugate of claim 18, comprising reacting a ligand with a compound of claim 1, wherein the ligand contains one or more of functional groups that are, independently, sulfhydryl, amino, glutamine, or formyl.
